(19) United States
(12) Patent Application Publication
(10) Pub. No.: US 2025/0340857 A1
(43) Pub. Date: Nov. 6, 2025

(54) IgE PROTEASES AND USES THEREOF

(71) Applicant: Seismic Therapeutic, Inc., Watertown, MA (US)

(72) Inventors: Nathan Rollins, Boston, MA (US); Jordan Anderson, Cambridge, MA (US); Ryan Peckner, Berkeley, CA (US); Jung-Eun Shin, Watertown, MA (US); Nathan Higginson-Scott, Hingham, MA (US); Kevin Lewis Otipoby, Ashland, MA (US); Ivan Mascanfroni, Worcester, MA (US); Yi Xing, Andover, MA (US); Purvi Mande, Boston, MA (US); Emily Hoyt, Boston, MA (US)

(21) Appl. No.: 19/196,457

(22) Filed: May 1, 2025

Related U.S. Application Data
(60) Provisional application No. 63/641,205, filed on May 1, 2024.
(60) Provisional application No. 63/744,760, filed on Jan. 13, 2025.
(60) Provisional application No. 63/797,927, filed on Apr. 30, 2025.

(51) Int. Cl. C12N 9/50; A61K 38/00

(52) U.S. Cl. CPC C12N 9/50; A61K 38/00

(57) ABSTRACT

Embodiments provided herein, provide for polypeptides and molecules comprising a polypeptide having protease activity, pharmaceutical compositions comprising the same, and methods that can be used to treat disorders, such as IgE mediated disorders.

### CROSS-REFERENCE TO RELATED APPLICATIONS

This application claims priority to U.S. Provisional Application No. 63/641,205, filed May 1, 2024, U.S. Provisional Application No. 63/744,760, filed Jan. 13, 2025, and U.S. Provisional Application No. 63/797,927, filed Apr. 30, 2025, each of which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Apr. 27, 2025, is named “SES-011WO_SL.xml” and is 1,316,473 bytes in size.

### FIELD

The embodiments provided herein relate to polypeptides comprising a polypeptide having protease activity, and compositions comprising the same.

### BACKGROUND

Immunoglobulin E is one of the five classes of immunoglobulins (IgM, IgG, IgD, IgA, IgE). IgE is the least abundant serum immunoglobulin and has an array of physiological functions, such as Type I hypersensitivity reactions, parasitic infections, and autoimmune processes. Pathologically, IgE induces type I hypersensitivity reactions through activation of mast cells and basophils and the induction of TH2 responses resulting in atopic diseases. The existence of autoreactive IgE antibodies in SLE has long been known. IgE has a high affinity for FcεRI, often referred to as the high-affinity Fc receptor for IgE. FcεRI is expressed on mast cells, basophils, Langerhans cells and eosinophils. Due to the high affinity of FcεRI for its IgE ligand, mast cells and basophils are covered with relatively long-lived FcεRI-IgE complexes. Upon ligation with bivalent or multivalent antigens specifically recognized by the bound IgE molecules, the FcεRI molecules transduce signals that activate the mast cells or basophils to secrete potent mediators of inflammation, such as histamine. These mediators are responsible for the symptoms associated with asthma, allergic rhinitis, and anaphylaxis. IgE is secreted by, and expressed on the surface of, B-cells (Godwin L, Sinawe H, Crane JS. Biochemistry, Immunoglobulin E. [Updated 2022 Sep. 24]. In: StatPearls [Internet]. Treasure Island (FL): StatPearls Publishing; 2024 January). IgE synthesized by B-cells is anchored to the B-cell membrane by a transmembrane domain linked to the mature IgE sequence by a short membrane binding region. IgE may also bind to B-cells (and monocytes, eosinophils and platelets) through its Fc domain to a low affinity IgE receptor (FcεRII). Upon exposure of a mammal to an allergen, B-cells are clonally amplified which synthesize IgE that binds the allergen. This IgE in turn is released into the circulation by the B-cells where it is bound by B-cells (through FcεRII) and by mast cells and basophils through the so-called high affinity receptor (FcεRI) found on the surface of the mast cells ad basophils. Such mast cells and basophils are thereby sensitized for allergen. Once initial exposure to an antigen has occurred, and an immune response has been activated, antigen-specific IgE will remain bound via the Fc-epsilon RI receptor to mast cells and basophils. It has been shown that binding alone of IgE to that receptor cause IgE-dependent upregulation of mast cell Fc-epsilon RI receptor expression, which allows these cells to bind more IgE antibodies. (Galli S J, Tsai M. IgE and mast cells in allergic disease. Nat Med. 2012 May 4; 18 (5): 693-704) IgE has a short half-life in plasma, usually less than a day. However, IgE bound to a high-affinity receptor (Fc-epsilon RI) can remain attached to mast cells in tissues for weeks to months. (Oettgen HC. Fifty years later: Emerging functions of IgE antibodies in host defense, immune regulation, and allergic diseases. J Allergy Clin Immunol. 2016 June; 137 (6): 1631-1645) IgE plays a central role in allergy sensitization and atopic disorders such as allergic rhinitis, asthma, and atopic dermatitis. To be able to effectively reduce, or eliminate such antibodies is therefore an important clinical challenge. The embodiments provided for herein fulfill this need as well as others.

### SUMMARY

In some embodiments, a polypeptide has the amino acid sequence of:

(SEQ ID NO: 917)
X₁TALGRRKAEDRIKETLWADGVKIDEKDFEHIX₉₃SSHEDYFAVEYGK
GSGYYDVNKKMDSEDX₁₂₂ALCVGX₁₂₈VVTNMFHWWVX₁₃₉QNRENIEKF
KX₁₅₀QDX₁₅₃ENNGVMKFX₁₆₂GVKEATVDELNX₁₇₄FYX₁₇₇YADQSRFFD
LIKX₁₉₆HFX₁₉₉NKALNPEELFELYFNGFYYX₂₁₉SSX₂₂₂RYIEDNX₂₂₉P
AQNNLFSKVLX₂₄₁NNKLLNPERSYGIDGFSNNVX₂₆₂NALKSHKVLGLV
X₂₇₅TTPLX₂₈₀YRHIISMWGADIDQDGKVKAIYVTDSDDREITFNDNSK
RRIGMX₃₂₄RYDVLVDDX₃₃₃GNIRFTGKGX₃₄₃THKEEGAIYAGLYSLS
X₃₆₀GDEVWEDYFKKYPEVQENLX₃₈₀,

wherein at least one X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is mutated as compared to SEQ ID NO: 1, wherein X₃₈₀ comprises the amino acid sequence having the formula as set forth in SEQ ID NO: 744 or is absent, and wherein X₁ comprises the amino acid sequence of SEQ ID NO: 248, or is absent. In some embodiments, X₁₂₈ is alanine (A), X₁₅₃ is proline (P), X₁₆₂ is glutamic acid (E), X₂₂₉ is glutamic acid (E), X₃₂₄ is isoleucine (I), and X₃₃₃ is asparagine (N). In some embodiments, X₁₂₈ is alanine (A), X₁₅₃ is proline (P), X₁₆₂ is glutamic acid (E), X₁₇₄ is glutamic acid (E), X₁₇₇ is an amino acid sequence RDNRDN (SEQ ID NO: 743), X₂₁₉ is glycine (G), X₂₂₉ is glutamic acid (E), X₂₇₅ is tyrosine (Y), X₃₂₄ is isoleucine (I), and X₃₃₃ is asparagine (N). In some embodiments, X₉₃ is aspartic acid (D), X₁₂₂ is serine(S), X₁₂₈ is alanine (A), X₁₅₀ is glutamic acid (E), X₁₅₃ is proline (P), X₁₆₂ is glutamic acid (E), X₁₇₄ is glutamic acid (E), X₁₇₇ is an amino acid sequence RDNRDN (SEQ ID NO: 743), X₁₉₉ is proline (P), X₂₁₉ is glycine (G), X₂₂₉ is glutamic acid (E), X₂₄₁ is aspartic acid (D), X₂₇₅ is tyrosine (Y), X₃₂₄ is isoleucine (I), and X₃₃₃ is asparagine (N). In some embodiments, X₉₃ is aspartic acid (D), X₁₂₂ is serine(S), X₁₂₈ is alanine (A), X₁₃₉ is glutamic acid (E), X₁₅₀ is glutamic acid (E), X₁₅₃ is proline (P), X₁₆₂ is glutamic acid (E), X₁₇₄ is glutamic acid (E), X₁₇₇ is an amino acid sequence RDNRDN (SEQ ID NO: 743), X₁₉₆ is serine(S), X₁₉₉ is proline (P), X₂₁₉ is glycine (G), X₂₂₂ is glutamine (Q), X₂₂₉ is glutamic acid (E), X₂₄₁ is aspartic acid (D), X₂₆₂ is lysine (K), X₂₇₅ is tyrosine (Y), X₂₈₀ is threonine (T), X₃₂₄ is isoleucine (I), X₃₃₃ is asparagine (N), X₃₄₃ is lysine (K), and X₃₆₀ is leucine (L). In some embodiments, the polypeptide comprises the amino acid sequence of any one of SEQ ID NO: 380, 251, 5-243, 247, 280-379, 381-528, 737, 745-823, 828-864, or 885-916.

In some embodiments, a polypeptide has IgE protease activity, wherein the polypeptide: a) has at least 50% identity to SEQ ID NO: 1; b) comprises: i) a cysteine (C) at a position in the polypeptide that corresponds to position 125 of SEQ ID NO: 1; ii) a histidine (H) at a position in the polypeptide that corresponds to position 283 of SEQ ID NO: 1; iii) an aspartic acid (D) at a position in the polypeptide that corresponds to position 305 of SEQ ID NO: 1; and iv) at least one mutation in the polypeptide as compared to SEQ ID NO: 1 that corresponds to position 68, 71, 75, 79, 84, 86, 88, 91, 93, 94, 102, 112, 115, 116, 117, 120, 122, 125, 126, 128, 139, 142, 143, 148, 149, 150, 152, 153, 160, 162, 164, 165, 166, 167, 174, 175, 177_183, 180, 182, 183, 185, 189, 196, 197, 199, 200_202, 201, 205, 206, 212, 219, 220, 221, 222, 224, 228, 229, 232, 233, 241, 242, 243, 244, 250, 251, 252, 254, 260, 262, 263, 268, 270, 271_287, 274, 275, 276, 279, 280, 281, 282, 293, 294, 295, 297, 301, 303, 303_326, 306, 307_322, 308_324, 309, 310, 310_320, 311_319, 311_321, 313, 314, 315_317, 316, 318, 319, 320, 324, 327, 329, 332, 333, 336, 343, 346, 347, 348, 351, 360, 363, 364, 366, 374, and 380 of SEQ ID NO: 1. In some embodiments, the polypeptide comprises: an aspartic acid (D) at a position in the polypeptide that corresponds to position 307 of SEQ ID NO: 1; and/or a lysine (K) at a position in the polypeptide that corresponds to position 115 of SEQ ID NO: 1. In some embodiments, the polypeptide does not comprise the contiguous sequence of amino acid residues 1-52 (1_52del) or 1-61 (1_61del) of SEQ ID NO: 1. In some embodiments, the polypeptide does not comprise contiguous sequence of amino acid residues 380-529 (380_529del) of SEQ ID NO: 1. In some embodiments, the polypeptide does not comprise the contiguous sequence of amino acid residues 1-52 and 380-529 of SEQ ID NO: 1. In some embodiments, the polypeptide does not comprise the contiguous sequence of amino acid residues 1-61 and 380-529 of SEQ ID NO: 1. In some embodiments, the polypeptide: a) has at least 50% identity to SEQ ID NO: 3, wherein X is a methionine, signal peptide, or absent; b) comprises: i) a cysteine (C) at a position in the polypeptide that corresponds to position 125 of SEQ ID NO: 1; ii) a histidine (H) at a position in the polypeptide that corresponds to position 283 of SEQ ID NO: 1; and iii) an aspartic acid (D) at a position in the polypeptide that corresponds to position 305 of SEQ ID NO: 1; and iv) at least one mutation in the polypeptide as compared to SEQ ID NO: 1 that corresponds to position 68, 71, 75, 79, 84, 86, 88, 91, 93, 94, 102, 112, 115, 116, 117, 120, 122, 125, 126, 128, 139, 142, 143, 148, 149, 150, 152, 153, 160, 162, 164, 165, 166, 167, 174, 175, 177_183, 180, 182, 183, 185, 189, 196, 197, 199, 200_202, 201, 205, 206, 212, 219, 220, 221, 222, 224, 228, 229, 232, 233, 241, 242, 243, 244, 250, 251, 252, 254, 260, 262, 263, 268, 270, 271_287, 274, 275, 276, 279, 280, 281, 282, 293, 294, 295, 297, 301, 303, 303_326, 306, 307_322, 308_324, 309, 310, 310_320, 311_319, 311_321, 313, 314, 315_317, 316, 318, 319, 320, 324, 327, 329, 332, 333, 336, 343, 346, 347, 348, 351, 360, 363, 364, 366, 374, and 380 of SEQ ID NO: 1, wherein the polypeptide does not comprise the contiguous sequence of amino acid residues 1-61 and/or 380-529 of SEQ ID NO: 1. In some embodiments, the polypeptide comprises an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, the mutation is a substitution, insertion, or deletion. In some embodiments, the polypeptide comprises one or more mutations of D71E, D71Q, D88H, D112Q, D152N, D139E, D307N, D293G, D295K, D295N, E75Y, E86D, E120D, E120G, E143D, E166Q, E241D, E310D, E310N, E310Q, E310S, E347N, E347R, E348R, E363K, E366K, F148Y, F175I, F333D, F333E, F333N, G199P, H174D, H174E, H197D, H197K, H197R, H222E, H222Q, H222S, H222T, H268G, H268R, H268S, H275L, H275Y, I84G, I84P, 184V, I262K, I301L, 1336A, K68W, K93D, K93S, K115L, K116D, K149L, K150E, K160E, K165E, K167R, K183D, K195L, K195R, K196D, K196E, K196L, K196Q, K196S, K196T, K201E, K201S, K205L, K229D, K229E, K229N, K244L, K297N, K297R, K318A, K318D, K318E, K318F, K318G, K318I, K318L, K318N, K318P, K318Q, K318S, K318V, K320W, K324I, K346P, K360R, L153P, L279G, L279T, L279W, L279Y, L329K, L329R, M117E, M117F, M323L, N162D, N162E, N173K, N182D, N182P, N219G, N219K, N219Q, N219T, N228K, N233G, N242G, N243K, N260A, P205F, P205FG, P205L, P205LG, Q180H, Q232E, Q294E, R142K, R189K, R250GGYGG (SEQ ID NO: 918), R250GYG, R256D, R282G, R282N, R280A, R280D, R280E, R280N, R280T, R280Y, R309G, R319E, R319T, R320L, R320Q, R320W, R337L, R360L, S94D, S94E, S94N, S128A, S181D, S220Y, S221E, S221N, S221Y, S251I, S286T, S306P, S317N, T122E, T122N, T122S, T167G, T176G, T276G, T276R, V102A, V102L, V164K, V270P, V274G, V303I, V303Y, V364E, Y212F, Y217W, Y224T, Y252R, Y281H, Y281S, Y281T, Y360L, 177_183delinsRDNRDN (SEQ ID NO: 919), 177_183KEYQSNKdelinsESDHG (SEQ ID NOs: 920 and 921), 177_183KEYQSNKdelinsRDNRDN (SEQ ID NOs: 920 and 919), 177_185KEYQSNKYAdelinsESPNT (SEQ ID NOs: 922 and 923), 177_318KEYQSNKNSKdelinsRDNRDN (SEQ ID NOs: 924 and 919), 177_319KEYQSNKDRdelinsRDNRDN (SEQ ID NOs: 925 and 919), 177_319KEYQSNKFNDNSKRdelinsRDNRDNIGDE (SEQ ID NOs: 926 and 927), 177_317KEYQSNKNDNSdelinsRDNRDN (SEQ ID NOs: 928 and 919), 180_185QSNKYAdelinsSG (SEQ ID NO: 929), 200_202NKAdelinsPYLSTKHL (SEQ ID NO: 930), 271_287delinsVGISGSAGGIKSVDSDGDSYI (SEQ ID NO: 932), 308_324delinsTKSVDSDGDSYKINY (SEQ ID NO: 932), 303_326delinsTGAKGNNYFGHWHFAVRVRL (SEQ ID NO: 933), 306_324delinsGHYGVYNGVTYWGHTPLHLAAMLGHLVYGLY (SEQ ID NO: 934), 306_324delinsQRTPLHLAAWADHLYR (SEQ ID NO: 935), 307_322delinsLAKGNNYFGHWHFAVM (SEQ ID NO: 936), 307_324delinsGGFAFDISIDNGNEDVY (SEQ ID NO: 937), 311_319delinsLLKPKILGYNITSGYSDEIY (SEQ ID NO: 938), 311_321delinsLSNVEALGYNITSGYSDKRY (SEQ ID NO: 939), 310_320delinsFLVFGVTYDGSTPVP (SEQ ID NO: 940), 311_321delinsLSNVEALGYNITSGYSDKRY (SEQ ID NO: 939), 86_105delinsDDMEAKGNNYFGHWHFAVATN (SEQ ID NO: 941), 88_102delinsGEIVAFDISIDNGNEDLAEILQLSTYDGGG (SEQ ID NO: 942), 91_100delinsGGGGGKSVDSDGDSYGGGGG (SEQ ID NO: 943), 93_98delinsGGGGGKSVDSDGDSYGGGG (SEQ ID NO: 944), 174_187delinsVMYKGKKLLEAARAGQPDSSE (SEQ ID NO: 945), 271_287delinsVGISGSAGGIKSVDSDGDSYI (SEQ ID NO: 932), 303_326delinsTGAKGNNYFGHWHFAVRVRL (SEQ ID NO: 933), 308_324delinsTKSVDSDGDSYKINY (SEQ ID NO: 932), and 380insENNVADSNKNSEDVEVSSVEFDFLNFKYPKNEDQVSTEDMRLTLKDTNVFNQPQ VKISFEEQNGNDWKEKDSGTFEEGKKYRLKIDVNKLALKIYGHLSKNKLNVIVNGKAV NIQNVVKKDSIETSFTIYSDSIDLEKINYWTDSFNNWS (SEQ ID NO: 744), wherein the positions correspond to SEQ ID NO: 1. In some embodiments, the polypeptide comprises any one of mutation sets as set forth in Table 3A or Table 3B. In some embodiments, the polypeptide comprises the amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 380, 251, 5-243, 247, 280-379, 381-528, 737, 745-823, 828-864, or 885-916. In some embodiments, the polypeptide comprises the amino acid sequence of any one of SEQ ID NO: 380, 251, 5-243, 247, 280-379, 381-528, 737, 745-823, 828-864, or 885-916. In some embodiments, the polypeptide is wherein the polypeptide is more effective or more selective at cleaving IgE than the polypeptide of SEQ ID NO: 1, 3, or 91 and is more selective for cleaving IgE than IgG.

In some embodiments, a polypeptide has IgE protease activity, wherein the polypeptide: a) has at least 50% identity to SEQ ID NO: 1; b) comprises: i) a cysteine (C) at a position in the polypeptide that corresponds to position 125 of SEQ ID NO: 1; ii) a histidine (H) at a position in the polypeptide that corresponds to position 283 of SEQ ID NO: 1; iii) an aspartic acid (D) at a position in the polypeptide that corresponds to position 305 of SEQ ID NO: 1; and iv) any one of mutation sets as set forth in Table 3A or Table 3B as compared to SEQ ID NO: 1. In some embodiments, the polypeptide comprises: an aspartic acid (D) at a position in the polypeptide that corresponds to position 307 of SEQ ID NO: 1; and/or a lysine (K) at a position in the polypeptide that corresponds to position 115 of SEQ ID NO: 1. In some embodiments, the polypeptide does not comprise the contiguous sequence of amino acid residues 1-52 (1_52del) or 1-61 (1_61del) of SEQ ID NO: 1. In some embodiments, the polypeptide does not comprise contiguous sequence of amino acid residues 380-529 (380_529del) of SEQ ID NO: 1. In some embodiments, the polypeptide does not comprise the contiguous sequence of amino acid residues 1-52 and 380-529 of SEQ ID NO: 1. In some embodiments, the polypeptide does not comprise the contiguous sequence of amino acid residues 1-61 and 380-529 of SEQ ID NO: 1. In some embodiments, the polypeptide: a) has at least 50% identity to SEQ ID NO: 3, wherein X is a methionine, signal peptide, or absent; b) comprises: i) a cysteine (C) at a position in the polypeptide that corresponds to position 125 of SEQ ID NO: 1; ii) a histidine (H) at a position in the polypeptide that corresponds to position 283 of SEQ ID NO: 1; and iii) an aspartic acid (D) at a position in the polypeptide that corresponds to position 305 of SEQ ID NO: 1; and iv) any one of mutation sets as set forth in Table 3A or Table 3B as compared to SEQ ID NO: 1. In some embodiments, the polypeptide comprises an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, the mutation is a substitution, insertion, or deletion. In some embodiments, the polypeptide comprises the amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 380, 251, 5-243, 247, 280-379, 381-528, 737, 745-823, 828-864, or 885-916. In some embodiments, the polypeptide comprises the amino acid sequence of any one of SEQ ID NO: 380, 251, 5-243, 247, 280-379, 381-528, 737, 745-823, 828-864, or 885-916. In some embodiments, the polypeptide is more effective or more selective at cleaving IgE than the polypeptide of SEQ ID NO: 1, 3, or 91 and is more selective for cleaving IgE than IgG.

In some embodiments, a composition comprises: a) a polypeptide having IgE protease activity, wherein the polypeptide: i) has at least 50% identity to SEQ ID NO: 1; ii) comprises: 1) a cysteine (C) at a position in the polypeptide that corresponds to position 125 of SEQ ID NO: 1; 2) a histidine (H) at a position in the polypeptide that corresponds to position 283 of SEQ ID NO: 1; 3) an aspartic acid (D) at a position in the polypeptide that corresponds to position 305 of SEQ ID NO: 1; and 4) at least one mutation in the polypeptide as compared to SEQ ID NO: 1 that corresponds to position 68, 71, 75, 79, 84, 86, 88, 91, 93, 94, 102, 112, 115, 116, 117, 120, 122, 125, 126, 128, 139, 142, 143, 148, 149, 150, 152, 153, 160, 162, 164, 165, 166, 167, 174, 175, 177_183, 180, 182, 183, 185, 189, 196, 197, 199, 200_202, 201, 205, 206, 212, 219, 220, 221, 222, 224, 228, 229, 232, 233, 241, 242, 243, 244, 250, 251, 252, 254, 260, 262, 263, 268, 270, 271_287, 274, 275, 276, 279, 280, 281, 282, 293, 294, 295, 297, 301, 303, 303_326, 306, 307_322, 308_324, 309, 310, 310_320, 311_319, 311_321, 313, 314, 315_317, 316, 318, 319, 320, 324, 327, 329, 332, 333, 336, 343, 346, 347, 348, 351, 360, 363, 364, 366, 374, and 380 of SEQ ID NO: 1; and b) an Fc domain.

In some embodiments, a composition comprises: a) a polypeptide having IgE protease activity, wherein the polypeptide: i) has at least 50% identity to SEQ ID NO: 1; ii) comprises: 1) a cysteine (C) at a position in the polypeptide that corresponds to position 125 of SEQ ID NO: 1; 2) a histidine (H) at a position in the polypeptide that corresponds to position 283 of SEQ ID NO: 1; 3) an aspartic acid (D) at a position in the polypeptide that corresponds to position 305 of SEQ ID NO: 1; and 4) any one of mutation sets as set forth in Table 3A or Table 3B as compared to SEQ ID NO: 1; and b) an Fc domain. In some embodiments, the polypeptide comprises: an aspartic acid (D) at a position in the polypeptide that corresponds to position 307 of SEQ ID NO: 1; and/or a lysine (K) at a position in the polypeptide that corresponds to position 115 of SEQ ID NO: 1. In some embodiments, the polypeptide does not comprise the contiguous sequence of amino acid residues 1-52 (1_52del) or 1-61 (1_61del) of SEQ ID NO: 1. In some embodiments, the polypeptide does not comprise contiguous sequence of amino acid residues 380-529 (380_529del) of SEQ ID NO: 1. In some embodiments, the polypeptide does not comprise the contiguous sequence of amino acid residues 1-52 and 380-529 of SEQ ID NO: 1. In some embodiments, the polypeptide does not comprise the contiguous sequence of amino acid residues 1-61 and 380-529 of SEQ ID NO: 1. In some embodiments, the polypeptide: a) has at least 50% identity to SEQ ID NO: 3, wherein X is a methionine, signal peptide, or absent; b) comprises: i) a cysteine (C) at a position in the polypeptide that corresponds to position 125 of SEQ ID NO: 1; ii) a histidine (H) at a position in the polypeptide that corresponds to position 283 of SEQ ID NO: 1; and iii) an aspartic acid (D) at a position in the polypeptide that corresponds to position 305 of SEQ ID NO: 1; and iv) at least one mutation in the polypeptide as compared to SEQ ID NO: 1 that corresponds to position 68, 71, 75, 79, 84, 86, 88, 91, 93, 94, 102, 112, 115, 116, 117, 120, 122, 125, 126, 128, 139, 142, 143, 148, 149, 150, 152, 153, 160, 162, 164, 165, 166, 167, 174, 175, 177_183, 180, 182, 183, 185, 189, 196, 197, 199, 200_202, 201, 205, 206, 212, 219, 220, 221, 222, 224, 228, 229, 232, 233, 241, 242, 243, 244, 250, 251, 252, 254, 260, 262, 263, 268, 270, 271_287, 274, 275, 276, 279, 280, 281, 282, 293, 294, 295, 297, 301, 303, 303_326, 306, 307_322, 308_324, 309, 310, 310_320, 311_319, 311_321, 313, 314, 315_317, 316, 318, 319, 320, 324, 327, 329, 332, 333, 336, 343, 346, 347, 348, 351, 360, 363, 364, 366, 374, and 380 of SEQ ID NO: 1, wherein the polypeptide does not comprise the contiguous sequence of amino acid residues 1-61 and/or 380-529 of SEQ ID NO: 1. In some embodiments, the polypeptide comprises an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, the mutation is a substitution, insertion, or deletion. In some embodiments, the polypeptide comprises one or more mutations of D71E, D71Q, D88H, D112Q, D152N, D139E, D307N, D293G, D295K, D295N, E75Y, E86D, E120D, E120G, E143D, E166Q, E241D, E310D, E310N, E310Q, E310S, E347N, E347R, E348R, E363K, E366K, F148Y, F175I, F333D, F333E, F333N, G199P, H174D, H174E, H197D, H197K, H197R, H222E, H222Q, H222S, H222T, H268G, H268R, H268S, H275L, H275Y, 184G, 184P, 184V, I262K, I301L, 1336A, K68W, K93D, K93S, K115L, K116D, K149L, K150E, K160E, K165E, K167R, K183D, K195L, K195R, K196D, K196E, K196L, K196Q, K196S, K196T, K201E, K201S, K205L, K229D, K229E, K229N, K244L, K297N, K297R, K318A, K318D, K318E, K318F, K318G, K318I, K318L, K318N, K318P, K318Q, K318S, K318V, K320W, K324I, K346P, K360R, L153P, L279G, L279T, L279W, L279Y, L329K, L329R, M117E, M117F, M323L, N162D, N162E, N173K, N182D, N182P, N219G, N219K, N219Q, N219T, N228K, N233G, N242G, N243K, N260A, P205F, P205FG, P205L, P205LG, Q180H, Q232E, Q294E, R142K, R189K, R250GGYGG (SEQ ID NO: 918), R250GYG, R256D, R282G, R282N, R280A, R280D, R280E, R280N, R280T, R280Y, R309G, R319E, R319T, R320L, R320Q, R320W, R337L, R360L, S94D, S94E, S94N, S128A, S181D, S220Y, S221E, S221N, S221Y, S251I, S286T, S306P, S317N, T122E, T122N, T122S, T167G, T176G, T276G, T276R, V102A, V102L, V164K, V270P, V274G, V303I, V303Y, V364E, Y212F, Y217W, Y224T, Y252R, Y281H, Y281S, Y281T, Y360L, 177_183delinsRDNRDN (SEQ ID NO: 919), 177_183KEYQSNKdelinsESDHG (SEQ ID NOs: 920 and 921), 177_183KEYQSNKdelinsRDNRDN (SEQ ID NOs: 920 and 919), 177_185KEYQSNKYAdelinsESPNT (SEQ ID NOs: 922 and 923), 177_318KEYQSNKNSKdelinsRDNRDN (SEQ ID NOs: 924 and 919), 177_319KEYQSNKDRdelinsRDNRDN (SEQ ID NOs: 925 and 919), 177_319KEYQSNKFNDNSKRdelinsRDNRDNIGDE (SEQ ID NOs: 926 and 927), 177_317KEYQSNKNDNSdelinsRDNRDN (SEQ ID NOs: 928 and 919), 180_185QSNKYAdelinsSG (SEQ ID NO: 929), 200_202NKAdelinsPYLSTKHL (SEQ ID NO: 930), 271_287delinsVGISGSAGGIKSVDSDGDSYI (SEQ ID NO: 932), 308_324delinsTKSVDSDGDSYKINY (SEQ ID NO: 932), 303_326delinsTGAKGNNYFGHWHFAVRVRL (SEQ ID NO: 933), 306_324delinsGHYGVYNGVTYWGHTPLHLAAMLGHLVYGLY (SEQ ID NO: 934), 306_324delinsQRTPLHLAAWADHLYR (SEQ ID NO: 935), 307_322delinsLAKGNNYFGHWHFAVM (SEQ ID NO: 936), 307_324delinsGGFAFDISIDNGNEDVY (SEQ ID NO: 937), 311_319delinsLLKPKILGYNITSGYSDEIY (SEQ ID NO: 938), 311_321delinsLSNVEALGYNITSGYSDKRY (SEQ ID NO: 939), 310_320delinsFLVFGVTYDGSTPVP (SEQ ID NO: 940), 311_321delinsLSNVEALGYNITSGYSDKRY (SEQ ID NO: 939), 86_105delinsDDMEAKGNNYFGHWHFAVATN (SEQ ID NO: 941), 88_102delinsGEIVAFDISIDNGNEDLAEILQLSTYDGGG (SEQ ID NO: 942), 91_100delinsGGGGGKSVDSDGDSYGGGGG (SEQ ID NO: 943), 93_98delinsGGGGGKSVDSDGDSYGGGG (SEQ ID NO: 944), 174_187delinsVMYKGKKLLEAARAGQPDSSE (SEQ ID NO: 945), 271_287delinsVGISGSAGGIKSVDSDGDSYI (SEQ ID NO: 932), 303_326delinsTGAKGNNYFGHWHFAVRVRL (SEQ ID NO: 933), 308_324delinsTKSVDSDGDSYKINY (SEQ ID NO: 932), and 380insENNVADSNKNSEDVEVSSVEFDFLNFKYPKNEDQVSTEDMRLTLKDTNVENQPQ VKISFEEQNGNDWKEKDSGTFEEGKKYRLKIDVNKLALKIYGHLSKNKLNVIVNGKAV NIQNVVKKDSIETSFTIYSDSIDLEKINYWTDSFNNWS (SEQ ID NO: 744), wherein the positions correspond to SEQ ID NO: 1. In some embodiments, the polypeptide comprises any one of mutation sets as set forth in Table 3A or Table 3B. In some embodiments, the polypeptide comprises the amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 380, 251, 5-243, 247, 280-379, 381-528, 737, 745-823, 828-864, or 885-916. In some embodiments, the polypeptide comprises the amino acid sequence of any one of SEQ ID NO: 380, 251, 5-243, 247, 280-379, 381-528, 737, 745-823, 828-864, or 885-916. In some embodiments, the polypeptide is more effective or more selective at cleaving IgE than the polypeptide of SEQ ID NO: 1, 3, or 91 and is more selective for cleaving IgE than IgG. In some embodiments, the Fc domain comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 721, 724, or 725. In some embodiments, the Fc domain comprises an amino acid sequence of SEQ ID NO: 721, 724, or 725. In some embodiments, the Fc domain comprises a first Fc domain comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 253, 254, or 722; and a second Fc domain comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 255, 256, or 723. In some embodiments, the Fc domain comprises: a first Fc domain comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 253; and a second Fc domain comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 255; a first Fc domain comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 254; and a second Fc domain comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 256; or a first Fc domain comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 722; and a second Fc domain comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 723. In some embodiments, the Fc domain comprises: a first Fc domain comprising an amino acid sequence of SEQ ID NO: 253; and a second Fc domain comprising an amino acid sequence of SEQ ID NO: 255; a first Fc domain comprising an amino acid sequence of SEQ ID NO: 254; and a second Fc domain comprising an amino acid sequence of SEQ ID NO: 256; or a first Fc domain comprising an amino acid sequence of SEQ ID NO: 722; and a second Fc domain comprising an amino acid sequence of SEQ ID NO: 723. In some embodiments, the composition comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 257, 258, 259, 529-720. In some embodiments, the composition comprises an amino acid sequence selected from any one of SEQ ID NO: 257, 258, 259, 529-720.

In some embodiments, a composition comprises: a) a polypeptide having IgE protease activity, wherein the polypeptide comprises an amino acid sequence of any one of SEQ ID NO: 380, 251, 5-243, 247, 280-379, 381-528, 737, 745-823, 828-864, or 885-916; and b) an Fc domain, wherein the Fc domain comprises an amino acid sequence of SEQ ID NO: 253, 254, 255, 256, 721, 722, 723, 724, or 725. In some embodiments, the polypeptide comprises: an aspartic acid (D) at a position in the polypeptide that corresponds to position 307 of SEQ ID NO: 1; and/or a lysine (K) at a position in the polypeptide that corresponds to position 115 of SEQ ID NO: 1. In some embodiments, the polypeptide does not comprise the contiguous sequence of amino acid residues 1-52 (1_52del) or 1-61 (1_61del) of SEQ ID NO: 1. In some embodiments, the polypeptide does not comprise contiguous sequence of amino acid residues 380-529 (380_529del) of SEQ ID NO: 1. In some embodiments, the polypeptide does not comprise the contiguous sequence of amino acid residues 1-52 and 380-529 of SEQ ID NO: 1. In some embodiments, the polypeptide does not comprise the contiguous sequence of amino acid residues 1-61 and 380-529 of SEQ ID NO: 1. In some embodiments, the polypeptide: a) has at least 50% identity to SEQ ID NO: 3, wherein X is a methionine, signal peptide, or absent; b) comprises: i) a cysteine (C) at a position in the polypeptide that corresponds to position 125 of SEQ ID NO: 1; ii) a histidine (H) at a position in the polypeptide that corresponds to position 283 of SEQ ID NO: 1; and iii) an aspartic acid (D) at a position in the polypeptide that corresponds to position 305 of SEQ ID NO: 1; and iv) any one of mutation sets as set forth in Table 3A or Table 3B as compared to SEQ ID NO: 1, wherein the polypeptide does not comprise the contiguous sequence of amino acid residues 1-61 and/or 380-529 of SEQ ID NO: 1. In some embodiments, the polypeptide comprises an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, the polypeptide comprises the amino acid sequence having at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 380, 251, 5-243, 247, 280-379, 381-528, 737, 745-823, 828-864, or 885-916. In some embodiments, the polypeptide comprises the amino acid sequence of any one of SEQ ID NO: 380, 251, 5-243, 247, 280-379, 381-528, 737, 745-823, 828-864, or 885-916. In some embodiments, the Fc domain comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NO: 721, 724, or 725. In some embodiments, the Fc domain comprises an amino acid sequence of SEQ ID NO: 721, 724, or 725.

In some embodiments, a polypeptide has IgE protease activity and binds to an epitope on Fc portion of IgE, wherein the epitope includes residue S332, N333, P334, R335, G336, V337, S338, A339, D363, L364, A365, P366, K368, A378, K392, R394, N395, G396, T397, R420, H423, P424, H425, L426, P427, R428, A429, M431, R432, S433, or any combination thereof. In some embodiments, the polypeptide having IgE protease activity binds to an epitope on the non-cleaved Fc portion of IgE that includes residues D363, A365, P366, K368, K392, R394, N395, G396, T397, and H425 of the non-cleaved Fc portion of IgE. In some embodiments, the polypeptide having IgE protease activity binds to an epitope on the cleaved Fc portion of IgE that includes residues S332, N333, P334, R335, G336, V337, S338, A339, D363, L364, A365, K368, A378, R420, H423, P424, H425, L426, P427, R428, A429, M431, R432, and S433 of the cleaved Fc portion of IgE.

In some embodiments, provided for here is a pharmaceutical composition comprising the polypeptide, or composition provided for herein. In some embodiments, a method of reducing IgE immunoglobulins in a subject is provided for herein, the method comprising administering the polypeptide, composition, or pharmaceutical composition provided for herein, to the subject to reduce IgE immunoglobulins in the subject. In some embodiments, a method of treating a disease or disorder in a subject, such as an IgE mediated disease, is provided for herein, the method comprising administering the polypeptide, composition, or pharmaceutical composition provided for herein to the subject to treat the disease or disorder. In some embodiments, a method of treating a subject having, or at risk, or elevated risk, for having, an IgE mediated disease or disorder, is provided for herein, the method comprising administering a therapeutically effective amount of the polypeptide, composition, or pharmaceutical composition provided for herein, thereby treating the subject. In some embodiments, the disease or disorder is selected from chronic spontaneous urticaria (CSU), atopic dermatitis, eczema, angioedema, autoimmune bullous skin diseases, bullous pemphigoid (BP), graft vs. host disease (GVHD), sarcoidosis, hypersensitivity pneumonitis, chronic obstructive pulmonary disease (COPD), asthma, allergic asthma, allergic bronchopulmonary mycosis (ABPM), allergic bronchopulmonary aspergillosis (ABPA), chronic rhinosinusitis with nasal polyps (CRSwNP), chronic rhinosinusitis without nasal polyps (CRSsNP), allergic rhinitis, allergic conjunctivitis, vernal keratoconjunctivitis, food allergies, alpha galactosidase syndrome, pollen-food allergy syndrome/oral allergy syndrome (OAS), acute allergen exposure, human seminal fluid hypersensitivity, anaphylaxis, hypersensitivity, allergy, latex allergy, occupational exposure to skin mucosa and lung sensitizing agents and allergens, mastocytosis, mast cell activation disorders, prevention of drug-induced immediate hypersensitivity, drug desensitization (e.g., chemotherapy), allergen immunotherapy desensitization, eosinophilic granulomatosis with polyangiitis (EGPA)/Churg-Strauss, hyper-IgE syndrome (HIES), or any combination thereof. In some embodiments, the subject is a subject in need thereof.

### BRIEF DESCRIPTION OF DRAWINGS

FIG. 1 shows cleaving performance of polypeptides having IgE protease activity against IgE or IgG1.

FIGS. 2A-2C show exemplary sequence alignments.

FIG. 3 illustrates exemplary molecules.

FIGS. 4A-4C show exemplary IgE protease sequence alignments.

### DETAILED DESCRIPTION

As used herein and in the appended claims, the singular forms “a”, “an” and “the” include plural reference unless the context clearly dictates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±5% and remain within the scope of the disclosed embodiments. Thus, about 100 means 95 to 105.

As used herein, the term “animal” includes, but is not limited to, humans and non-human vertebrates such as wild, domestic, and farm animals. As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “contacting” means bringing together of two elements in an in vitro system or an in vivo system. For example, “contacting” a therapeutic compound with an individual or patient or cell includes the administration of the compound to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “fused” or “linked” when used in reference to a protein or molecule having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another.

As used herein, the term “individual,” “subject,” or “patient,” used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. An result, symptom, or activity can also be inhibited if it is completely elimination or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined. In some embodiments, the pharmaceutical compositions can be ophthalmically acceptable or suitable for ophthalmic administration.

In some embodiments, the term “therapeutic molecule” can be used interchangeably with “therapeutic compound,” “molecule,” or “therapeutic,” and refers to any polypeptide, or protein described herein.

As used herein, the term “position,” is meant to refer to a location in the sequence of a polypeptide. Positions may be numbered sequentially, or according to an established format, for example the EU numbering system based on Kabat's amino acid positions. For example, position 298 is a position in the human antibody IgG1.

“Specific binding” or “specifically binds to” or is “specific for” a particular antigen, target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody having a K_{D} for an antigen or epitope of at least about 10^{-4M}, at least about 10^{-5M}, at least about 10^{-6M}, at least about 10^{-7M}, at least about 10^{-8M}, at least about 10^{-9M}, alternatively at least about 10^{-10M}, at least about 10^{-11M}, at least about 10^{-12M}, or greater, where K_{D} refers to a dissociation rate of a particular antibody-target interaction. Typically, an antibody that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody having a K_{A} or Kₐ for a target, antigen, or epitope of at least 2-, 4-, 5-, 20-, 50-, 100-, 500-, 1000-, 5,000-, 10,000- or more times greater for the target, antigen, or epitope relative to a control, where K_{A} or Kₐ refers to an association rate of a particular antibody-antigen interaction.

As provided herein, the compounds and compositions provided for herein can be used in methods of treatment as provided herein. As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For purposes of these embodiments, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival, as applicable for a specific disease, as compared to expected survival if not receiving treatment. Thus, “treatment of an autoimmune condition” or “treating autoimmunity” means an activity that alleviates or ameliorates any of the primary phenomena or secondary symptoms associated with the autoimmune condition other condition described herein when the terms “treat,” “treated,” or “treating” are used in conjunction with such condition.

As used herein, terms “variant,” “molecule,” “therapeutic,” “therapeutic compound,” “compound,” “polypeptide,” or “protein” can be used interchangeably and relate to the variants, molecules, therapeutics, therapeutic compounds, compounds, polypeptides, and proteins disclosed herein.

### Protease Variants

Prior to the present disclosure, immunoglobulin proteases that could cleave IgE or selectively IgE were not known. The present disclosure provides for the surprising and unexpected embodiments of immunoglobulin proteases that can cleave IgE and selectively cleave IgE. The present disclosure provides for polypeptides, molecules, compounds, therapeutics, and compositions comprising a polypeptide having protease activity. In some embodiments, the polypeptide having protease activity is capable of cleaving an immunoglobulin. In some embodiments, the polypeptide having protease activity is capable of cleaving an immunoglobulin E (IgE).

In some embodiments, the polypeptide having protease activity is a variant of a naturally occurring protease.

In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to:

(SEQ ID NO: 1)
MKKQSFTHSRKPKFGMRKLSIGLASCMLGMMFLTTGHVNADDVDHVGE
TITVVPWQRNNLDTALGRRKAEDRIKETLWADGVKIDEKDFEHIKSSH
EDYFAVEYGKGSGYYDVNKKMDSEDTALCVGSVVTNMFHWWVDQNREN
IEKFKKQDLENNGVMKFNGVKEATVDELNHFYKEYQSNKYADQSRFFD
LIKKHFGNKALNPEELFELYFNGFYYNSSHRYIEDNKPAQNNLFSKVL
ENNKLLNPERSYGIDGFSNNVINALKSHKVLGLVHTTPLRYRHIISMW
GADIDQDGKVKAIYVTDSDDREITFNDNSKRRIGMKRYDVLVDDFGNI
RFTGKGATHKEEGAIYAGLYSLSRGDEVWEDYFKKYPEVQENLENNVA
DSNKNSEDVEVSSVEFDFLNFKYPKNEDQVSTEDMRLTLKDTNVFNQP
QVKISFEEQNGNDWKEKDSGTFEEGKKYRLKIDVNKLALKIYGHLSKN
KLNVIVNGKAVNIQNVVKKDSIETSFTIYSDSIDLEKINYWTDSFNNW
S.

In some embodiments, the polypeptide having protease activity is a size variant of SEQ ID NO: 1. Thus, in some embodiments, the polypeptide having protease activity comprises amino acids X1-X2 of SEQ ID NO: 1, wherein X1 and X2 denote the amino acid position, or a range of positions of SEQ ID NO: 1. Accordingly, in some embodiments, the polypeptide having protease activity comprises amino acids 62-379 of SEQ ID NO: 1. in some embodiments, the polypeptide having protease activity comprises amino acids 53-379 of SEQ ID NO: 1.

In some embodiments, the polypeptide having protease activity does not comprise the contiguous amino acid residues 1-52 or 1-61 of SEQ ID NO: 1 or any contiguous 5-10 peptide fragment contained therein. In some embodiments, the polypeptide having protease activity does not comprise the contiguous amino acid residues 1-60, 1-55, or 1-50 of SEQ ID NO: 1. In some embodiments, the polypeptide having protease activity does not comprise the contiguous amino acid residues 1-60, 1-59, 1-58, 1-57, 1-56, 1-55, 1-54, 1-53, 1-52, 1-51, or 1-50 of SEQ ID NO: 1. These deletions can be illustrated by the notation of the “first residue_second residuedel”. For example, the notation “1_61del” in reference to SEQ ID NO: 1 means that the first 61 amino acid residues of SEQ ID NO: 1 are not present in the polypeptide. Thus, in some embodiments, the polypeptide having protease activity does not comprise the contiguous amino acid sequence MKKQSFTHSRKPKFGMRKLSIGLASCMLGMMFLTTGHVNADDVDHVGETITVVPWQR NNLD (SEQ ID NO: 248) in its entirety. In some embodiments, the polypeptide having protease activity does not comprise the contiguous amino acid sequence MKKQSFTHSRKPKFGMRKLSIGLASCMLGMMFLTTGHVNADDVDHVGETITV (SEQ ID NO: 249) in its entirety.

In some embodiments, the polypeptide having protease activity does not comprise the contiguous amino acid residues 380-529 (380_529del) of SEQ ID NO: 1. In some embodiments, the polypeptide having protease activity does not comprise the contiguous amino acid residues 380-520, 380-510, 380-500, 380-490, or 380-480 of SEQ ID NO: 1

In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to amino acids 62-379 of SEQ ID NO: 1. In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to amino acids 62-379 of SEQ ID NO: 1, provided that the amino acid sequence does not comprises amino acids 1-61 and/or 380-529 of SEQ ID NO: 1 or other deletions as provided for herein.

In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to amino acids 53-379 of SEQ ID NO: 1. In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to amino acids 62-379 of SEQ ID NO: 1, provided that the amino acid sequence does not comprises amino acids 1-52 and/or 380-529 of SEQ ID NO: 1, or other deletions as provided for herein.

In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 3. In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 3, provided that the amino acid sequence does not comprises amino acids 1-61 and/or 380-529 of SEQ ID NO: 1.

In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 4. In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 4, provided that the amino acid sequence does not comprises amino acids 1-52 and/or 380-529 of SEQ ID NO: 1.

In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to:

(SEQ ID NO: 2)
MRKLSIGLASCMLGMMFLTTSHVSGEVVEVWPYGQNPHSGTIGVDPGQ
QNLDTALGRRKAEERIKETLWVDGVKIDEKDFEHMSSSHEDYFAAEYG
KGSGYYDVNKKMDSGDSALCVASVVTNMFHWWVDQNRDNIEKFKKQDI
ENNGVMKFNGVRQTTVDELNHFYKEYNSNKYADQSKFFDLIKSHFGNR
ALNPEELFELYFNGFYYNSSQRYIEDNKPAQNNLFSKVLENNKLLNPE
RSYGIDGFSNNVINALKSHKVLGLVHTTPLRYRHIISMWGADIDQDGK
VKAIYVTDSDDREITFNDNSKRRIGMKRYDVLVDDFGNIRFTGKGATH
KEEGAIYAGLYSLSRGDEVWEDYFKKYPEVQENLENNVADSNKNSEDV
EVSSVEFDFLNFKYPKNEDQVSTEDMRLTLKDTNVFNQPQVKISFEEQ
NGNDWKEKDSGTFEEGKKYRLKIDVNKLALKIYGHLSKNKLNVIVNGK
AVNIQNVVKKDSIETSFTIYSDSIDLEKINYWTDSFNNWS.

In some embodiments, the polypeptide having protease activity is a size variant of SEQ ID NO: 1. Thus, in some embodiments, the polypeptide having protease activity comprises amino acids X1-X2 of SEQ ID NO: 2, wherein X1 and X2 denote the amino acid position, or a range of positions of SEQ ID NO: 2. Accordingly, in some embodiments, the polypeptide having protease activity comprises amino acids 51-370 of SEQ ID NO: 2.

In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to amino acids 51-370 of SEQ ID NO: 2. In some embodiments, the polypeptide having protease activity has an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to amino acids 51-370 of SEQ ID NO: 2, provided that the amino acid sequence does not comprises amino acids 1-50 and/or 371-520 of SEQ ID NO: 2.

In some embodiments, a polypeptide having protease activity does not comprise mutations to amino acids responsible for binding to an immunoglobulin, or amino acids responsible for the protease activity of the polypeptide. In some embodiments, a polypeptide having protease activity does not comprise mutations to amino acids responsible for binding to an IgE. In some embodiments, a polypeptide having protease activity does not comprise mutations to amino acids responsible for the IgE protease activity of the polypeptide.

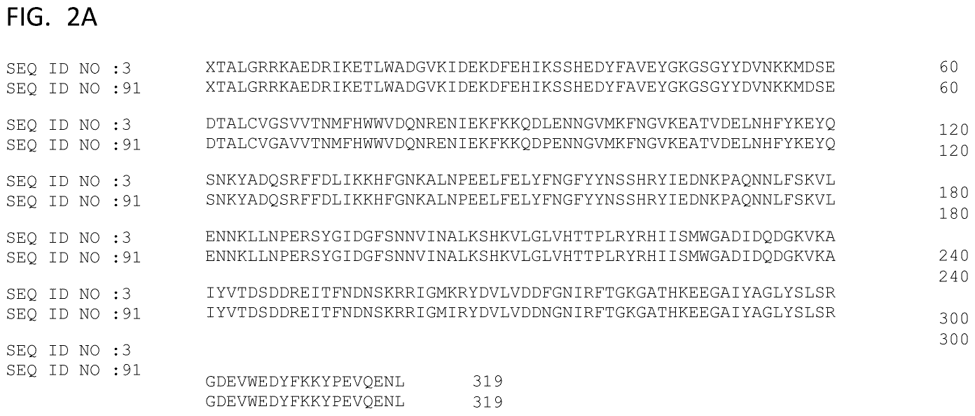

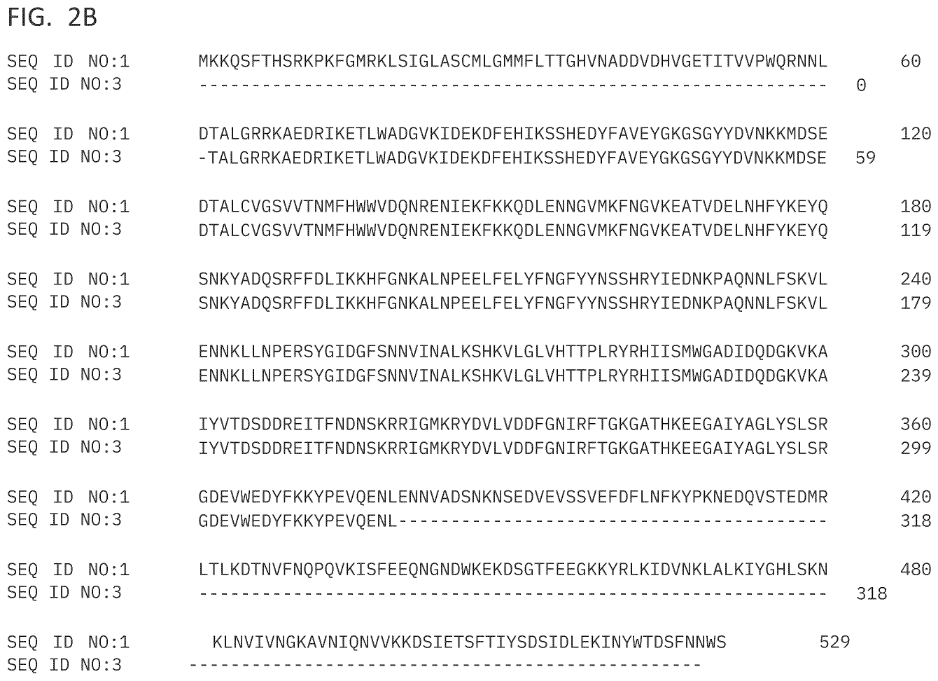

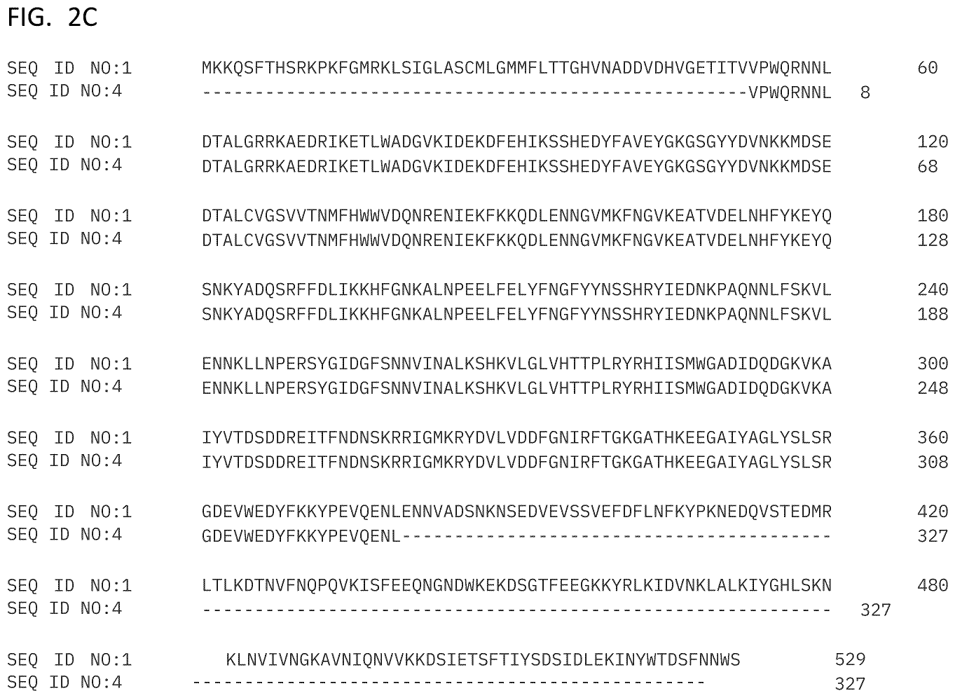

In some embodiments, a polypeptide having protease activity comprises an amino acid sequence such as those provided herein. In some embodiments, the amino acid sequence comprises residues at position that correspond to positions in SEQ ID NO: 1, when the two, or more, amino acid sequences are aligned. For example, a position in SEQ ID NO: 3 may corresponds to a position in SEQ ID NO: 1, as shown in FIG. 2B alignment. In another embodiment, a position in SEQ ID NO: 4 may corresponds to a position in SEQ ID NO: 1, as shown in FIG. 2C alignment. Thus, in some embodiments, position 1 in SEQ ID NO: 3 corresponds to position 62 in SEQ ID NO: 1; position 2 in SEQ ID NO: 3 corresponds to position 63 in SEQ ID NO: 1; and so forth, as shown in FIG. 2B alignment. In some embodiments, position 1 in SEQ ID NO: 4 corresponds to position 53 in SEQ ID NO: 1; position 2 in SEQ ID NO: 4 corresponds to position 54 in SEQ ID NO: 1; and so forth, as shown in FIG. 2C alignment.

In some embodiments, position 71 in SEQ ID NO: 1 corresponds to position 10 in SEQ ID NO: 3. In some embodiments, position 79 in SEQ ID NO: 1 corresponds to position 18 in SEQ ID NO: 3. In some embodiments, position 93 in SEQ ID NO: 1 corresponds to position 32 in SEQ ID NO: 3. In some embodiments, position 102 in SEQ ID NO: 1 corresponds to position 41 in SEQ ID NO: 3. In some embodiments, position 120 in SEQ ID NO: 1 corresponds to position 59 in SEQ ID NO: 3. In some embodiments, position 122 in SEQ ID NO: 1 corresponds to position 61 in SEQ ID NO: 3. In some embodiments, position 125 in SEQ ID NO: 1 corresponds to position 64 in SEQ ID NO: 3. In some embodiments, position 126 in SEQ ID NO: 1 corresponds to position 65 in SEQ ID NO: 3. In some embodiments, position 128 in SEQ ID NO: 1 corresponds to position 67 in SEQ ID NO: 3. In some embodiments, position 153 in SEQ ID NO: 1 corresponds to position 92 in SEQ ID NO: 3. In some embodiments, position 160 in SEQ ID NO: 1 corresponds to position 99 in SEQ ID NO: 3. In some embodiments, position 162 in SEQ ID NO: 1 corresponds to position 101 in SEQ ID NO: 3. In some embodiments, position 165 in SEQ ID NO: 1 corresponds to position 104 in SEQ ID NO: 3. In some embodiments, position 166 in SEQ ID NO: 1 corresponds to position 105 in SEQ ID NO: 3. In some embodiments, position 167 in SEQ ID NO: 1 corresponds to position 106 in SEQ ID NO: 3. In some embodiments, position 189 in SEQ ID NO: 1 corresponds to position 128 in SEQ ID NO: 3. In some embodiments, position 195 in SEQ ID NO: 1 corresponds to position 134 in SEQ ID NO: 3. In some embodiments, position 196 in SEQ ID NO: 1 corresponds to position 135 in SEQ ID NO: 3. In some embodiments, position 197 in SEQ ID NO: 1 corresponds to position 136 in SEQ ID NO: 3. In some embodiments, position 201 in SEQ ID NO: 1 corresponds to position 140 in SEQ ID NO: 3. In some embodiments, position 212 in SEQ ID NO: 1 corresponds to position 151 in SEQ ID NO: 3. In some embodiments, position 222 in SEQ ID NO: 1 corresponds to position 161 in SEQ ID NO: 3. In some embodiments, position 229 in SEQ ID NO: 1 corresponds to position 168 in SEQ ID NO: 3. In some embodiments, position 280 in SEQ ID NO: 1 corresponds to position 219 in SEQ ID NO: 3. In some embodiments, position 283 in SEQ ID NO: 1 corresponds to position 222 in SEQ ID NO: 3. In some embodiments, position 305 in SEQ ID NO: 1 corresponds to position 244 in SEQ ID NO: 3. In some embodiments, position 309 in SEQ ID NO: 1 corresponds to position 248 in SEQ ID NO: 3. In some embodiments, position 310 in SEQ ID NO: 1 corresponds to position 249 in SEQ ID NO: 3. In some embodiments, position 318 in SEQ ID NO: 1 corresponds to position 257 in SEQ ID NO: 3. In some embodiments, position 319 in SEQ ID NO: 1 corresponds to position 258 in SEQ ID NO: 3. In some embodiments, position 320 in SEQ ID NO: 1 corresponds to position 259 in SEQ ID NO: 3. In some embodiments, position 324 in SEQ ID NO: 1 corresponds to position 263 in SEQ ID NO: 3. In some embodiments, position 333 in SEQ ID NO: 1 corresponds to position 272 in SEQ ID NO: 3.

In some embodiments, position 125 in SEQ ID NO: 1 corresponds to position 73 in SEQ ID NO: 4. In some embodiments, position 283 in SEQ ID NO: 1 corresponds to position 231 in SEQ ID NO: 4. In some embodiments, position 283 in SEQ ID NO: 1 corresponds to position 253 in SEQ ID NO: 4. In some embodiments, position 122 in SEQ ID NO: 1 corresponds to position 70 in SEQ ID NO: 4. In some embodiments, position 128 in SEQ ID NO: 1 corresponds to position 76 in SEQ ID NO: 4. In some embodiments, position 153 in SEQ ID NO: 1 corresponds to position 101 in SEQ ID NO: 4.

In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, and/or an aspartic acid (D) at a position in the polypeptide that corresponds to position 305 of SEQ ID NO: 1. In some embodiments, a polypeptide having protease activity comprises a cysteine (C) at a position in the polypeptide that corresponds to position 125 of SEQ ID NO: 1. In some embodiments, a polypeptide having protease activity comprises a histidine (H) at a position in the polypeptide that corresponds to position 283 of SEQ ID NO: 1. In some embodiments, a polypeptide having protease activity comprises an aspartic acid (D) at a position in the polypeptide that corresponds to position 305 of SEQ ID NO: 1. In some embodiments, a polypeptide having protease activity comprises a cysteine (C) at a position in the polypeptide that corresponds to position 125 of SEQ ID NO: 1, and a histidine (H) at a position in the polypeptide that corresponds to position 283 of SEQ ID NO: 1. In some embodiments, a polypeptide having protease activity comprises a cysteine (C) at a position in the polypeptide that corresponds to position 125 of SEQ ID NO: 1, and an aspartic acid (D) at a position in the polypeptide that corresponds to position 305 of SEQ ID NO: 1. In some embodiments, a polypeptide having protease activity comprises a histidine (H) at a position in the polypeptide that corresponds to position 283 of SEQ ID NO: 1, and an aspartic acid (D) at a position in the polypeptide that corresponds to position 305 of SEQ ID NO: 1. In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, and an aspartic acid (D) at a position in the polypeptide that corresponds to position 305 of SEQ ID NO: 1.

In some embodiments, a cysteine (C) at a position 125 in SEQ ID NO: 1 corresponds to a cysteine (C) at position 64 in SEQ ID NO: 3. In some embodiments, a histidine (H) at a position 283 in SEQ ID NO: 1 corresponds to a histidine (H) at position 222 in SEQ ID NO: 3. In some embodiments, an aspartic acid (D) at a position 305 in SEQ ID NO: 1 corresponds to an aspartic acid (D) at position 244 in SEQ ID NO: 3.

In some embodiments, a cysteine (C) at a position 125 in SEQ ID NO: 1 corresponds to a cysteine (C) at position 73 in SEQ ID NO: 4. In some embodiments, a histidine (H) at a position 283 in SEQ ID NO: 1 corresponds to a histidine (H) at position 231 in SEQ ID NO: 4. In some embodiments, an aspartic acid (D) at a position 305 in SEQ ID NO: 1 corresponds to an aspartic acid (D) at position 253 in SEQ ID NO: 4.

In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, a polypeptide having protease activity comprises a cysteine (C) at a position in the polypeptide that corresponds to position 125 of SEQ ID NO: 1, and a lysine (K) at a position in the polypeptide that corresponds to position 115 of SEQ ID NO: 1. In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, and a lysine (K) at a position in the polypeptide that corresponds to position 115 of SEQ ID NO: 1. In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, and a lysine (K) at a position in the polypeptide that corresponds to position 115 of SEQ ID NO: 1. In some embodiments, a polypeptide having protease activity comprises a histidine (H) at a position in the polypeptide that corresponds to position 283 of SEQ ID NO: 1, an aspartic acid (D) at a position in the polypeptide that corresponds to position 305 of SEQ ID NO: 1, and a lysine (K) at a position in the polypeptide that corresponds to position 115 of SEQ ID NO: 1. In some embodiments, a polypeptide having protease activity comprises a cysteine (C) at a position in the polypeptide that corresponds to position 125 of SEQ ID NO: 1, and an aspartic acid (D) at a position in the polypeptide that corresponds to position 307 of SEQ ID NO: 1. In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, and an aspartic acid (D) at a position in the polypeptide that corresponds to position 307 of SEQ ID NO: 1. In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, and an aspartic acid (D) at a position in the polypeptide that corresponds to position 307 of SEQ ID NO: 1. In some embodiments, a polypeptide having protease activity comprises a histidine (H) at a position in the polypeptide that corresponds to position 283 of SEQ ID NO: 1, an aspartic acid (D) at a position in the polypeptide that corresponds to position 305 of SEQ ID NO: 1, and an aspartic acid (D) at a position in the polypeptide that corresponds to position 307 of SEQ ID NO: 1. In some embodiments, a polypeptide having protease activity comprises a cysteine (C) at a position in the polypeptide that corresponds to position 125 of SEQ ID NO: 1, an aspartic acid (D) at a position in the polypeptide that corresponds to position 307 of SEQ ID NO: 1, and a lysine (K) at a position in the polypeptide that corresponds to position 115 of SEQ ID NO: 1. In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 307 of SEQ ID NO: 1, and a lysine (K) at a position in the polypeptide that corresponds to position 115 of SEQ ID NO: 1. In some embodiments, a polypeptide having protease activity comprises a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and a lysine (K) at a position in the polypeptide that corresponds to position 115 of SEQ ID NO: 1. In some embodiments, a polypeptide having protease activity comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and a lysine (K) at a position in the polypeptide that corresponds to position 115 of SEQ ID NO: 1.

In some embodiments, the polypeptide having protease activity comprises at least one mutation at any position, wherein the positions correspond to SEQ ID NO: 1. In some embodiments, the polypeptide having protease activity comprises one or more mutations at any position selected from any one of position corresponding to position 68, 71, 75, 79, 84, 86, 88, 91, 93, 94, 102, 112, 115, 116, 117, 120, 122, 125, 126, 128, 139, 142, 143, 148, 149, 150, 152, 153, 160, 162, 164, 166, 167, 174, 175, 180, 181, 182, 183, 185, 189, 196, 197, 199, 200, 201, 202, 205, 206, 212, 219, 220, 221, 222, 224, 228, 229, 232, 233, 241, 242, 243, 244, 250, 251, 252, 254, 260, 262, 263, 268, 270, 271, 273, 274, 275, 276, 279, 280, 281, 282, 293, 294, 295, 297, 301, 303, 306, 307, 308, 309, 310, 311, 313, 314, 315, 316, 317, 318, 319, 320, 322, 323, 324, 326, 327, 329, 332, 333, 336, 343, 346, 347, 348, 351, 360, 363, 364, 366, 374, or 380 of SEQ ID NO: 1. 68 of SEQ ID NO: 1. In some embodiments, the polypeptide having protease activity comprises a mutation at a position corresponding to position 71 of SEQ ID NO: 1. In some embodiments, the polypeptide having protease activity comprises a mutation at a position corresponding to position 75 of SEQ ID NO: 1. In some embodiments, the polypeptide having protease activity comprises a mutation at a position corresponding to position 79 of SEQ ID NO: 1. In some embodiments, the polypeptide having protease activity comprises a mutation at a position corresponding to position 84 of SEQ ID NO: 1. In some embodiments, the polypeptide having protease activity comprises a mutation at a position corresponding to position 86 of SEQ ID NO: 1. In some embodiments, the polypeptide having protease activity comprises a mutation at a position corresponding to position 88 of SEQ ID NO: 1. In some embodiments, the polypeptide having protease activity comprises a mutation at a position corresponding to position 91 of SEQ ID NO: 1. In some embodiments, the polypeptide having protease activity comprises a mutation at a position corresponding to position 93 of SEQ ID NO: 1. In some embodiments, the polypeptide having protease activity comprises a mutation at a position corresponding to position 94 of SEQ ID NO: 1. In some embodiments, the polypeptide having protease activity comprises a mutation at a position corresponding to position 102 of SEQ ID NO: 1. In some embodiments, the polypeptide having protease activity comprises a mutation at a position corresponding to position 112 of SEQ ID NO: 1. In some embodiments, the polypeptide having protease activity comprises a mutation at a position corresponding to position 115 of SEQ ID NO: 1. In some embodiments, the polypeptide having protease activity comprises a mutation at a position corresponding to position 116 of SEQ ID NO: 1. In some embodiments, the polypeptide having protease activity comprises a mutation at a position corresponding to position 117 of SEQ ID NO: 1. In some embodiments, the polypeptide having protease activity comprises a mutation at a position corresponding to position 120 of SEQ ID NO: 1. In some embodiments, the polypeptide having protease activity comprises a mutation at a position corresponding to position 122 of SEQ ID NO: 1. In some embodiments, the polypeptide having protease activity comprises a mutation at a position corresponding to position 125 of SEQ ID NO: 1. In some embodiments, the polypeptide having protease activity comprises a mutation at a position corresponding to position 126 of SEQ ID NO: 1. In some embodiments, the polypeptide having protease activity comprises a mutation at a position corresponding to position 128 of SEQ ID NO: 1. In some embodiments, the polypeptide having protease activity comprises a mutation at a position corresponding to position 139 of SEQ ID NO: 1. In some embodiments, the polypeptide having protease activity comprises a mutation at a position corresponding to position 142 of SEQ ID NO: 1. In some embodiments, the polypeptide having protease activity comprises a mutation at a position corresponding to position 143 of SEQ ID NO: 1. In some embodiments, the polypeptide having protease activity comprises a mutation at a position corresponding to position 148 of SEQ ID NO: 1. In some embodiments, the polypeptide having protease activity comprises a mutation at a position corresponding to position 149 of SEQ ID NO: 1. In some embodiments, the polypeptide having protease activity comprises a mutation at a position corresponding to position 150 of SEQ ID NO: 1. In some embodiments, the polypeptide having protease activity comprises a mutation at a position corresponding to position 152 of SEQ ID NO: 1. In some embodiments, the polypeptide having protease activity comprises a mutation at a position corresponding to position 153 of SEQ ID NO: 1. In some embodiments, the polypeptide having protease activity comprises a mutation at a position corresponding to position 160 of SEQ ID NO: 1. In some embodiments, the polypeptide having protease activity comprises a mutation at a position corresponding to position 162 of SEQ ID NO: 1. In some embodiments, the polypeptide having protease activity comprises a mutation at a position corresponding to position 164 of SEQ ID NO: 1. In some embodiments, the polypeptide having protease activity comprises a mutation at a position corresponding to position 166 of SEQ ID NO: 1. In some embodiments, the polypeptide having protease activity comprises a mutation at a position corresponding to position 167 of SEQ ID NO: 1. In some embodiments, the polypeptide having protease activity comprises a mutation at a position corresponding to position 174 of SEQ ID NO: 1. In some embodiments, the polypeptide having protease activity comprises a mutation at a position corresponding to position 175 of SEQ ID NO: 1. In some embodiments, the polypeptide having protease activity comprises a mutation at a position corresponding to position 180 of SEQ ID NO: 1. In some embodiments, the polypeptide having protease activity comprises a mutation at a position corresponding to position 181 of SEQ ID NO: 1. In some embodiments, the polypeptide having protease activity comprises a mutation at a position corresponding to position 182 of SEQ ID NO: 1. In some embodiments, the polypeptide having protease activity comprises a mutation at a position corresponding to position 183 of SEQ ID NO: 1. In some embodiments, the polypeptide having protease activity comprises a mutation at a position corresponding to position 185 of SEQ ID NO: 1. In some embodiments, the polypeptide having protease activity comprises a mutation at a position corresponding to position 189 of SEQ ID NO: 1. In some embodiments, the polypeptide having protease activity comprises a mutation at a position corresponding to position 196 of SEQ ID NO: 1. In some embodiments, the polypeptide having protease activity comprises a mutation at a position corresponding to position 197 of SEQ ID NO: 1. In some embodiments, the polypeptide having protease activity comprises a mutation at a position corresponding to position 199 of SEQ ID NO: 1. In some embodiments, the polypeptide having protease activity comprises a mutation at a position corresponding to position 200 of SEQ ID NO: 1. In some embodiments, the polypeptide having protease activity comprises a mutation at a position corresponding to position 201 of SEQ ID NO: 1. In some embodiments, the polypeptide having protease activity comprises a mutation at a position corresponding to position 202 of SEQ ID NO: 1. In some embodiments, the polypeptide having protease activity comprises a mutation at a position corresponding to position 205 of SEQ ID NO: 1. In some embodiments, the polypeptide having protease activity comprises a mutation at a position corresponding to position 206 of SEQ ID NO: 1. In some embodiments, the polypeptide having protease activity comprises a mutation at a position corresponding to position 212 of SEQ ID NO: 1. In some embodiments, the polypeptide having protease activity comprises a mutation at a position corresponding to position 219 of SEQ ID NO: 1. In some embodiments, the polypeptide having protease activity comprises a mutation at a position corresponding to position 220 of SEQ ID NO: 1. In some embodiments, the polypeptide having protease activity comprises a mutation at a position corresponding to position 221 of SEQ ID NO: 1. In some embodiments, the polypeptide having protease activity comprises a mutation at a position corresponding to position 222 of SEQ ID NO: 1. In some embodiments, the polypeptide having protease activity comprises a mutation at a position corresponding to position 224 of SEQ ID NO: 1. In some embodiments, the polypeptide having protease activity comprises a mutation at a position corresponding to position 228 of SEQ ID NO: 1. In some embodiments, the polypeptide having protease activity comprises a mutation at a position corresponding to position 229 of SEQ ID NO: 1. In some embodiments, the polypeptide having protease activity comprises a mutation at a position corresponding to position 232 of SEQ ID NO: 1. In some embodiments, the polypeptide having protease activity comprises a mutation at a position corresponding to position 233 of SEQ ID NO: 1. In some embodiments, the polypeptide having protease activity comprises a mutation at a position corresponding to position 241 of SEQ ID NO: 1. In some embodiments, the polypeptide having protease activity comprises a mutation at a position corresponding to position 242 of SEQ ID NO: 1. In some embodiments, the polypeptide having protease activity comprises a mutation at a position corresponding to position 243 of SEQ ID NO: 1. In some embodiments, the polypeptide having protease activity comprises a mutation at a position corresponding to position 244 of SEQ ID NO: 1. In some embodiments, the polypeptide having protease activity comprises a mutation at a position corresponding to position 250 of SEQ ID NO: 1. In some embodiments, the polypeptide having protease activity comprises a mutation at a position corresponding to position 251 of SEQ ID NO: 1. In some embodiments, the polypeptide having protease activity comprises a mutation at a position corresponding to position 252 of SEQ ID NO: 1. In some embodiments, the polypeptide having protease activity comprises a mutation at a position corresponding to position 254 of SEQ ID NO: 1. In some embodiments, the polypeptide having protease activity comprises a mutation at a position corresponding to position 260 of SEQ ID NO: 1. In some embodiments, the polypeptide having protease activity comprises a mutation at a position corresponding to position 262 of SEQ ID NO: 1. In some embodiments, the polypeptide having protease activity comprises a mutation at a position corresponding to position 263 of SEQ ID NO: 1. In some embodiments, the polypeptide having protease activity comprises a mutation at a position corresponding to position 268 of SEQ ID NO: 1. In some embodiments, the polypeptide having protease activity comprises a mutation at a position corresponding to position 270 of SEQ ID NO: 1. In some embodiments, the polypeptide having protease activity comprises a mutation at a position corresponding to position 271 of SEQ ID NO: 1. In some embodiments, the polypeptide having protease activity comprises a mutation at a position corresponding to position 273 of SEQ ID NO: 1. In some embodiments, the polypeptide having protease activity comprises a mutation at a position corresponding to position 274 of SEQ ID NO: 1. In some embodiments, the polypeptide having protease activity comprises a mutation at a position corresponding to position 275 of SEQ ID NO: 1. In some embodiments, the polypeptide having protease activity comprises a mutation at a position corresponding to position 276 of SEQ ID NO: 1. In some embodiments, the polypeptide having protease activity comprises a mutation at a position corresponding to position 279 of SEQ ID NO: 1. In some embodiments, the polypeptide having protease activity comprises a mutation at a position corresponding to position 280 of SEQ ID NO: 1. In some embodiments, the polypeptide having protease activity comprises a mutation at a position corresponding to position 281 of SEQ ID NO: 1. In some embodiments, the polypeptide having protease activity comprises a mutation at a position corresponding to position 282 of SEQ ID NO: 1. In some embodiments, the polypeptide having protease activity comprises a mutation at a position corresponding to position 293 of SEQ ID NO: 1. In some embodiments, the polypeptide having protease activity comprises a mutation at a position corresponding to position 294 of SEQ ID NO: 1. In some embodiments, the polypeptide having protease activity comprises a mutation at a position corresponding to position 295 of SEQ ID NO: 1. In some embodiments, the polypeptide having protease activity comprises a mutation at a position corresponding to position 297 of SEQ ID NO: 1. In some embodiments, the polypeptide having protease activity comprises a mutation at a position corresponding to position 301 of SEQ ID NO: 1. In some embodiments, the polypeptide having protease activity comprises a mutation at a position corresponding to position 303 of SEQ ID NO: 1. In some embodiments, the polypeptide having protease activity comprises a mutation at a position corresponding to position 306 of SEQ ID NO: 1. In some embodiments, the polypeptide having protease activity comprises a mutation at a position corresponding to position 307 of SEQ ID NO: 1. In some embodiments, the polypeptide having protease activity comprises a mutation at a position corresponding to position 308 of SEQ ID NO: 1. In some embodiments, the polypeptide having protease activity comprises a mutation at a position corresponding to position 309 of SEQ ID NO: 1. In some embodiments, the polypeptide having protease activity comprises a mutation at a position corresponding to position 310 of SEQ ID NO: 1. In some embodiments, the polypeptide having protease activity comprises a mutation at a position corresponding to position 311 of SEQ ID NO: 1. In some embodiments, the polypeptide having protease activity comprises a mutation at a position corresponding to position 313 of SEQ ID NO: 1. In some embodiments, the polypeptide having protease activity comprises a mutation at a position corresponding to position 314 of SEQ ID NO: 1. In some embodiments, the polypeptide having protease activity comprises a mutation at a position corresponding to position 315 of SEQ ID NO: 1. In some embodiments, the polypeptide having protease activity comprises a mutation at a position corresponding to position 316 of SEQ ID NO: 1. In some embodiments, the polypeptide having protease activity comprises a mutation at a position corresponding to position 317 of SEQ ID NO: 1. In some embodiments, the polypeptide having protease activity comprises a mutation at a position corresponding to position 318 of SEQ ID NO: 1. In some embodiments, the polypeptide having protease activity comprises a mutation at a position corresponding to position 319 of SEQ ID NO: 1. In some embodiments, the polypeptide having protease activity comprises a mutation at a position corresponding to position 320 of SEQ ID NO: 1. In some embodiments, the polypeptide having protease activity comprises a mutation at a position corresponding to position 322 of SEQ ID NO: 1. In some embodiments, the polypeptide having protease activity comprises a mutation at a position corresponding to position 323 of SEQ ID NO: 1. In some embodiments, the polypeptide having protease activity comprises a mutation at a position corresponding to position 324 of SEQ ID NO: 1. In some embodiments, the polypeptide having protease activity comprises a mutation at a position corresponding to position 326 of SEQ ID NO: 1. In some embodiments, the polypeptide having protease activity comprises a mutation at a position corresponding to position 327 of SEQ ID NO: 1. In some embodiments, the polypeptide having protease activity comprises a mutation at a position corresponding to position 329 of SEQ ID NO: 1. In some embodiments, the polypeptide having protease activity comprises a mutation at a position corresponding to position 332 of SEQ ID NO: 1. In some embodiments, the polypeptide having protease activity comprises a mutation at a position corresponding to position 333 of SEQ ID NO: 1. In some embodiments, the polypeptide having protease activity comprises a mutation at a position corresponding to position 336 of SEQ ID NO: 1. In some embodiments, the polypeptide having protease activity comprises a mutation at a position corresponding to position 343 of SEQ ID NO: 1. In some embodiments, the polypeptide having protease activity comprises a mutation at a position corresponding to position 346 of SEQ ID NO: 1. In some embodiments, the polypeptide having protease activity comprises a mutation at a position corresponding to position 347 of SEQ ID NO: 1. In some embodiments, the polypeptide having protease activity comprises a mutation at a position corresponding to position 348 of SEQ ID NO: 1. In some embodiments, the polypeptide having protease activity comprises a mutation at a position corresponding to position 351 of SEQ ID NO: 1. In some embodiments, the polypeptide having protease activity comprises a mutation at a position corresponding to position 360 of SEQ ID NO: 1. In some embodiments, the polypeptide having protease activity comprises a mutation at a position corresponding to position 363 of SEQ ID NO: 1. In some embodiments, the polypeptide having protease activity comprises a mutation at a position corresponding to position 364 of SEQ ID NO: 1. In some embodiments, the polypeptide having protease activity comprises a mutation at a position corresponding to position 366 of SEQ ID NO: 1. In some embodiments, the polypeptide having protease activity comprises a mutation at a position corresponding to position 374 of SEQ ID NO: 1. In some embodiments, the polypeptide having protease activity comprises a mutation at a position corresponding to position 380 of SEQ ID NO: 1. In some embodiments, the polypeptide having protease activity comprises a mutation at a position corresponding to position of SEQ ID NO: 1.

In some embodiments, the polypeptide having protease activity comprises one or more mutations at positions selected from those in Table 1 below. In some embodiments, the mutation is a substitution, insertion, or deletion. Accordingly, in some embodiments, positions shown as “####delinsX” indicate deletion of a residue, or more residues, between positions indicated by “##” and insertion of sequence of “X”. In some embodiments, positions shown as “####X1delinsX2” indicate deletion of a sequence “X1” between positions indicated by “##” and insertion of sequence of “X2”.

TABLE 1
Sets of position as compared to SEQ ID NO: 1, that may comprise a mutation (such as,
but not limited to, a substitution, a deletion, or an insertion). Positions shown as “
XX_XX”, where XX indicates the position number, indicate a deletion and/or insertion.

| | | |
|---|---|---|
| 153, 189, 128 | 128, 153, 324, 333, 149, 262, | 128, 153, 162, 174, 199, 219, 229, 262, |
| | 276, 364, 205, 206, 250, 251 | 275, 324, 333, 177_183 |
| 153, 128, 333 | 128, 153, 324, 333, 149, 199, | 128, 153, 162, 174, 199, 219, 229, 275, |
| | 262, 276, 364 | 282, 324, 333, 177_183 |
| 122, 229 | 128, 153, 324, 333, 116, 149, | 93, 128, 150, 153, 162, 174, 199, 219, |
| | 182, 196, 262, 276, 364 | 229, 275, 282, 324, 333, 177_183 |
| 122, 153 | 128, 153, 324, 333, 94, 116, | 128, 139, 153, 162, 174, 199, 219, 229, |
| | 149, 182, 196, 262, 276, 336, | 241, 275, 282, 324, 333, 177_183 |
| | 364 | |
| 122, 153, 189 | 128, 153, 324, 333, 94, 116, | 128, 153, 162, 174, 199, 219, 229, 275, |
| | 149, 182, 196, 224, 262, 276, | 282, 324, 333, 343, 360, 177_183 |
| | 336, 364 | |
| 122, 153, 128 | 128, 153, 324, 333, 94, 116, | 128, 139, 153, 162, 174, 199, 219, 229, |
| | 149, 182, 196, 224, 262, 276, | 241, 275, 282, 324, 333, 343, 360, |
| | 281, 336, 364 | 177_183 |
| 122, 153, 126 | 128, 153, 324, 333, 94, 116, | 93, 128, 139, 150, 153, 162, 174, 199, |
| | 149, 182, 196, 224, 262, 276, | 219, 229, 241, 275, 282, 324, 333, |
| | 281, 336, 364, 174 | 177_183 |
| 122, 128, 333 | 128, 153, 324, 333, 94, 116, | 93, 128, 150, 153, 162, 174, 199, 219, |
| | 149, 182, 196, 224, 270, 276, | 229, 275, 282, 324, 333, 343, 360, |
| | 281, 336, 364 | 177_183 |
| 166 | 128, 153, 324, 333, 94, 116, | 128, 153, 162, 174, 199, 219, 229, 262, |
| | 149, 182, 196, 224, 270, 274, | 275, 282, 324, 333, 177_183 |
| | 281, 336, 364 | |
| 196 | 128, 153, 324, 333, 94, 116, | 93, 128, 150, 153, 162, 174, 199, 219, |
| | 149, 182, 199, 224, 270, 274, | 229, 262, 275, 282, 324, 333, 177_183 |
| | 281, 336, 364 | |
| 196 | 128, 153, 324, 333, 149, 196, | 128, 139, 153, 162, 174, 199, 219, 229, |
| | 262, 276, 364 | 241, 262, 275, 282, 324, 333, 177_183 |
| 222 | 128, 153, 324, 333, 262, 274, | 128, 153, 162, 174, 199, 219, 229, 262, |
| | 281 | 275, 282, 324, 333, 343, 360, 177_183 |
| 120 | 128, 153, 324, 333, 199, 262, | 128, 139, 153, 162, 174, 199, 219, 229, |
| | 276, 281 | 241, 262, 275, 282, 324, 333, 343, 360, |
| | | 177_183 |
| 93 | 128, 153, 324, 333, 196, 262, | 93, 128, 139, 150, 153, 162, 174, 199, |
| | 276, 281 | 219, 229, 241, 262, 275, 282, 324, 333, |
| | | 177_183 |
| 122 | 128, 153, 324, 333, 199, 224, | 93, 128, 150, 153, 162, 174, 199, 219, |
| | 262, 276, 281 | 229, 262, 275, 282, 324, 333, 343, 360, |
| | | 177_183 |
| 229 | 128, 153, 324, 333, 199, 224, | 93, 128, 139, 150, 153, 162, 174, 199, |
| | 262, 276, 281, 336 | 219, 229, 241, 262, 275, 282, 306, 324, |
| | | 333, 177_183 |
| 162 | 128, 153, 324, 333, 149, 199, | 93, 128, 150, 153, 162, 174, 199, 219, |
| | 224, 262, 276, 281, 336, 364 | 229, 262, 275, 282, 306, 324, 333, 343, |
| | | 360, 177_183 |
| 280 | 128, 153, 324, 333, 149, 196, | 93, 128, 139, 150, 153, 162, 174, 199, |
| | 224, 262, 276, 281, 336, 364 | 219, 229, 241, 262, 275, 282, 306, 324, |
| | | 333, 343, 360, 177_183 |
| 318 | 128, 153, 324, 333, 149, 199, | 93, 128, 150, 153, 162, 174, 199, 219, |
| | 224, 262, 276, 281, 336, 364, | 229, 262, 275, 282, 324, 333, 177_183 |
| | 174 | |
| 318 | 128, 153, 324, 333, 94, 116, | 93, 128, 139, 150, 153, 162, 174, 199, |
| | 149, 182, 196, 224, 262, 276, | 219, 229, 241, 262, 275, 282, 324, 333, |
| | 281, 303, 336, 364 | 177_183 |
| 122, 128, 153 | , 122, 128, 153, 333, 324, 162, | 93, 128, 139, 150, 153, 162, 174, 199, |
| | 229, 275, 219, 174, 177_183 | 219, 229, 241, 262, 275, 282, 324, 333, |
| | | 343, 360, 177_183 |
| 122, 128, 153, | 128, 153, 324, 333, 311_321 | 93, 128, 139, 150, 153, 162, 174, 199, |
| 333 | | 219, 229, 241, 262, 275, 282, 306, 324, |
| | | 333, 177_183 |
| 71, 79, 122, 128, | 128, 153, 324, 333, 310_320 | 93, 128, 150, 153, 162, 174, 199, 219, |
| 153, 333 | | 229, 262, 275, 282, 306, 324, 333, 343, |
| | | 360, 177_183 |
| 79, 122, 128, 153, | 128, 153, 324, 333, 307_322 | 93, 128, 139, 150, 153, 162, 174, 199, |
| 189, 333 | | 219, 229, 241, 262, 275, 282, 306, 324, |
| | | 333, 343, 360, 177_183 |
| 79, 122, 128, 153, | 128, 153, 324, 333, 86_105 | 128, 153, 162, 174, 199, 219, 229, 275, |
| 222, 333 | | 281, 324, 333, 177_183 |
| 122, 128, 153, | 128, 153, 324, 333, 271_287 | 93, 128, 150, 153, 162, 174, 199, 219, |
| 189, 222, 333 | | 229, 275, 281, 324, 333, 177_183 |
| 79, 122, 128, 153, | 128, 153, 324, 333, 91_100 | 128, 139, 153, 162, 174, 199, 219, 229, |
| 167, 333 | | 241, 275, 281, 324, 333, 177_183 |
| 79, 122, 128, | 128, 153, 324, 333, 308_324 | 128, 153, 162, 174, 199, 219, 229, 275, |
| 153, 189, 222 | | 281, 324, 333, 343, 360, 177_183 |
| 79, 122, 128, | 128, 153, 324, 333, 311_319 | 128, 139, 153, 162, 174, 199, 219, 229, |
| 153, 167, 222 | | 241, 275, 281, 324, 333, 343, 360, |
| | | 177_183 |
| 79, 102, 122, | 128, 153, 324, 333, 315_317 | 93, 128, 139, 150, 153, 162, 174, 199, |
| 128, 153, 167 | | 219, 229, 241, 275, 281, 324, 333, |
| | | 177_183 |
| 71, 79, 122, 128, | 128, 153, 324, 333, 303_326 | 93, 128, 150, 153, 162, 174, 199, 219, |
| 153, 167 | | 229, 275, 281, 324, 333, 343, 360, |
| | | 177_183 |
| 122, 128, 153, | 128, 153, 324, 333, 93_98 | 128, 153, 162, 174, 199, 219, 229, 262, |
| 162, 333 | | 275, 281, 324, 333, 177_183 |
| 122, 128, 153, | 128, 153, 324, 333, 174_187 | 93, 128, 150, 153, 162, 174, 199, 219, |
| 318, 333 | | 229, 262, 275, 281, 324, 333, 177_183 |
| 122, 128, 153, | 128, 153, 324, 333, 306_324 | 128, 139, 153, 162, 174, 199, 219, 229, |
| 222, 229, 280, | | 241, 262, 275, 281, 324, 333, 177_183 |
| 333 | | |
| 128, 153, 222, | 128, 153, 324, 333, 306_324 | 128, 153, 162, 174, 199, 219, 229, 262, |
| 229, 333 | | 275, 281, 324, 333, 343, 360, 177_183 |
| 128, 153, 222, | 128, 153, 324, 333, 307_324 | 128, 139, 153, 162, 174, 199, 219, 229, |
| 280, 333 | | 241, 262, 275, 281, 324, 333, 343, 360, |
| | | 177_183 |
| 128, 153, 229, | 128, 153, 324, 333, 88_102 | 93, 128, 139, 150, 153, 162, 174, 199, |
| 280, 333 | | 219, 229, 241, 262, 275, 281, 324, 333, |
| | | 177_183 |
| 122, 128, 153, | 128, 153, 324, 333, 311_321, | 93, 128, 150, 153, 162, 174, 199, 219, |
| 162, 222, 229, | 86_105,271_287 | 229, 262, 275, 281, 324, 333, 343, 360, |
| 280, 333 | | 177_183 |
| 128, 153, 162, | 128, 153, 324, 333, 311_321, | 93, 128, 139, 150, 153, 162, 174, 199, |
| 196, 333 | 86_105 | 219, 229, 241, 262, 275, 281, 306, 324, |
| | | 333, 177_183 |
| 128, 153, 162, | 128, 153, 324, 333, 311_321, | 93, 128, 150, 153, 162, 174, 199, 219, |
| 222, 333 | 91_100 | 229, 262, 275, 281, 306, 324, 333, 343, |
| | | 360, 177_183 |
| 128, 153, 162, | 128, 153, 324, 333, 311_321, | 93, 128, 139, 150, 153, 162, 174, 199, |
| 229, 333 | 271_287 | 219, 229, 241, 262, 275, 281, 306, 324, |
| | | 333, 343, 360, 177_183 |
| 128, 153, 162, | 128, 153, 324, 333, 310_320, | 93, 128, 150, 153, 162, 174, 199, 219, |
| 280, 333 | 86_105, 271_287 | 229, 262, 275, 281, 324, 333, 177_183 |
| 128, 153, 162, | 128, 153, 324, 333, 310_320, | 93, 128, 139, 150, 153, 162, 174, 199, |
| 318, 333 | 86_105 | 219, 229, 241, 262, 275, 281, 324, 333, |
| | | 177_183 |
| 128, 153, 162, | 128, 153, 324, 333, 310_320, | 93, 128, 139, 150, 153, 162, 174, 199, |
| 196, 318, 333 | 91_100 | 219, 229, 241, 262, 275, 281, 324, 333, |
| | | 343, 360, 177_183 |
| 128, 153, 196, | 128, 153, 324, 333, 310_320, | 93, 128, 139, 150, 153, 162, 174, 199, |
| 318, 333 | 271_287 | 219, 229, 241, 262, 275, 281, 306, 324, |
| | | 333, 177_183 |
| 128, 153, 162, | 128, 153, 324, 333, 307_322, | 93, 128, 150, 153, 162, 174, 199, 219, |
| 333 | 91_100 | 229, 262, 275, 281, 306, 324, 333, 343, |
| | | 360, 177_183 |
| 128, 153, 196, | 128, 153, 324, 333, 307_322, | 93, 128, 139, 150, 153, 162, 174, 199, |
| 333 | 271_287 | 219, 229, 241, 262, 275, 281, 306, 324, |
| | | 333, 343, 360, 177_183 |
| 128, 153, 318, | 128, 153, 324, 333, 308_324, | 93, 128, 150, 153, 162, 174, 199, 219, |
| 333 | 86_105 | 229, 262, 275, 324, 333, 177_183 |
| 128, 153, 196, | 117, 128, 153, 333 | 128, 139, 153, 162, 174, 199, 219, 229, |
| 310, 318, 333 | | 241, 262, 275, 324, 333, 177_183 |
| 128, 153, 162, | 117, 128, 153, 333 | 128, 153, 162, 174, 199, 219, 229, 262, |
| 196, 310, 318, | | 275, 324, 333, 343, 360, 177_183 |
| 333 | | |
| 122, 128, 153, | 128, 153, 333, 348 | 128, 139, 153, 162, 174, 199, 219, 229, |
| 162 | | 241, 262, 275, 324, 333, 343, 360, |
| | | 177_183 |
| 122, 128, 153, | 128, 153, 333, 347 | 93, 128, 139, 150, 153, 162, 174, 199, |
| 318 | | 219, 229, 241, 262, 275, 324, 333, |
| | | 177_183 |
| 122, 128, 153, | 128, 153, 333, 351 | 93, 128, 150, 153, 162, 174, 199, 219, |
| 222, 229, 280 | | 229, 262, 275, 324, 333, 343, 360, |
| | | 177_183 |
| 122, 128, 153, | 128, 153, 220, 333 | 93, 128, 139, 150, 153, 162, 174, 199, |
| 222, 229 | | 219, 229, 241, 262, 275, 306, 324, 333, |
| | | 177_183 |
| 122, 128, 153, | 128, 153, 221, 333 | 93, 128, 150, 153, 162, 174, 199, 219, |
| 222, 280 | | 229, 262, 275, 306, 324, 333, 343, 360, |
| | | 177_183 |
| 122, 128, 153, | 128, 153, 275, 333 | 93, 128, 139, 150, 153, 162, 174, 199, |
| 229, 280 | | 219, 229, 241, 262, 275, 306, 324, 333, |
| | | 343, 360, 177_183 |
| 122, 128, 153, | 128, 153, 333, 200_202 | 93, 128, 150, 153, 162, 174, 199, 219, |
| 162, 222, 229, | | 229, 262, 275, 324, 333, 177_183 |
| 280 | | |
| 122, 128, 153, | 117, 128, 153, 222, 333, 348 | 93, 128, 139, 150, 153, 162, 174, 199, |
| 162, 196 | | 219, 229, 241, 262, 275, 324, 333, |
| | | 177_183 |
| 122, 128, 153, | 125, 128, 153, 324, 333 | 93, 128, 139, 150, 153, 162, 174, 199, |
| 162, 222 | | 219, 229, 241, 262, 275, 324, 333, 343, |
| | | 360, 177_183 |
| 122, 128, 153, | 125, 128, 153, 280, 333 | 93, 128, 139, 150, 153, 162, 174, 199, |
| 162, 229 | | 219, 229, 241, 262, 275, 306, 324, 333, |
| | | 177_183 |
| 122, 128, 153, | 125, 128, 153, 162, 229, 333 | 93, 128, 150, 153, 162, 174, 199, 219, |
| 162, 280 | | 229, 262, 275, 306, 324, 333, 343, 360, |
| | | 177_183 |
| 122, 128, 153, | 125, 128, 153, 162, 229, 324, | 93, 128, 139, 150, 153, 162, 174, 199, |
| 162, 318 | 333 | 219, 229, 241, 262, 275, 306, 324, 333, |
| | | 343, 360, 177_183 |
| 122, 128, 153, | 125, 128, 153, 162, 229, 280, | 128, 153, 162, 174, 197, 219, 229, 262, |
| 162, 196, 318 | 324, 333 | 275, 324, 333, 177_183 |
| 122, 128, 153, | 128, 153, 162, 229, 324, 333, | 93, 128, 150, 153, 162, 174, 197, 219, |
| 196, 318 | 177_183 | 229, 262, 275, 324, 333, 177_183 |
| 122, 128, 153, | 128, 153, 162, 180, 183, 229, | 128, 139, 153, 162, 174, 197, 219, 229, |
| 196 | 324, 333, 176_178 | 241, 262, 275, 324, 333, 177_183 |
| 122, 128, 153, | 128, 153, 162, 229, 313, 317, | 128, 153, 162, 174, 197, 219, 229, 262, |
| 196, 212, 318 | 324, 333, 177_319 | 275, 324, 333, 343, 360, 177_183 |
| 122, 128, 153, | 128, 153, 162, 229, 324, 333, | 128, 139, 153, 162, 174, 197, 219, 229, |
| 196, 310, 318 | 177_319 | 241, 262, 275, 324, 333, 343, 360, |
| | | 177_183 |
| 122, 128, 153, | 128, 153, 162, 229, 313, 314, | 93, 128, 139, 150, 153, 162, 174, 197, |
| 162, 196, 310, | 324, 333, 177_318 | 219, 229, 241, 262, 275, 324, 333, |
| 318 | | 177_183 |
| 128, 153, 166, | 128, 153, 162, 229, 293, 294, | 93, 128, 150, 153, 162, 174, 197, 219, |
| 333 | 297, 324, 333, 177_183 | 229, 262, 275, 324, 333, 343, 360, |
| | | 177_183 |
| 128, 153, 196, | 128, 153, 162, 229, 294, 295, | 93, 128, 139, 150, 153, 162, 174, 197, |
| 333 | 324, 333, 177_183 | 219, 229, 241, 262, 275, 306, 324, 333, |
| | | 177_183 |
| 128, 153, 196, | 128, 153, 162, 229, 294, 295, | 93, 128, 150, 153, 162, 174, 197, 219, |
| 333 | 297, 324, 333, 177_183 | 229, 262, 275, 306, 324, 333, 343, 360, |
| | | 177_183 |
| 128, 153, 196, | 128, 153, 162, 229, 324, 333, | 93, 128, 139, 150, 153, 162, 174, 197, |
| 333 | 177_185 | 219, 229, 241, 262, 275, 306, 324, 333, |
| | | 343, 360, 177_183 |
| 128, 153, 222, | 128, 153, 162, 229, 324, 333, | 93, 128, 150, 153, 162, 174, 197, 219, |
| 333 | 180_185 | 229, 262, 275, 324, 333, 177_183 |
| 128, 153, 222, | 128, 153, 162, 229, 318, 324, | 93, 128, 139, 150, 153, 162, 174, 197, |
| 333 | 333, 177_317 | 219, 229, 241, 262, 275, 324, 333, |
| | | 177_183 |
| 128, 153, 222, | 128, 153, 162, 229, 324, 333, | 93, 128, 139, 150, 153, 162, 174, 197, |
| 333 | 177_317 | 219, 229, 241, 262, 275, 324, 333, 343, |
| | | 360, 177_183 |
| 120, 128, 153, | 128, 153, 199, 324, 333 | 93, 128, 139, 150, 153, 162, 174, 197, |
| 333 | | 219, 229, 241, 262, 275, 306, 324, 333, |
| | | 177_183 |
| 122, 128, 153, | 128, 153, 174, 324, 333 | 93, 128, 150, 153, 162, 174, 197, 219, |
| 333 | | 229, 262, 275, 306, 324, 333, 343, 360, |
| | | 177_183 |
| 122, 128, 153, | 128, 153, 262, 324, 333 | 93, 128, 139, 150, 153, 162, 174, 197, |
| 333 | | 219, 229, 241, 262, 275, 306, 324, 333, |
| | | 343, 360, 177_183 |
| 128, 153, 229, | 128, 153, 324, 333, 336 | 93, 128, 150, 153, 162, 174, 199, 219, |
| 333 | | 229, 275, 324, 333, 177_183 |
| 128, 153, 229, | 116, 128, 153, 324, 333 | 128, 139, 153, 162, 174, 199, 219, 229, |
| 333 | | 241, 275, 324, 333, 177_183 |
| 128, 153, 162, | 128, 149, 153, 324, 333 | 128, 153, 162, 174, 199, 219, 229, 275, |
| 333 | | 324, 333, 343, 360, 177_183 |
| 128, 153, 280, | 128, 153, 182, 324, 333 | 128, 139, 153, 162, 174, 199, 219, 229, |
| 333 | | 241, 275, 324, 333, 343, 360, 177_183 |
| 128, 153, 280, | 94, 128, 153, 324, 333 | 93, 128, 139, 150, 153, 162, 174, 199, |
| 333 | | 219, 229, 241, 275, 324, 333, 177_183 |
| 128, 153, 280, | 128, 153, 276, 324, 333 | 93, 128, 150, 153, 162, 174, 199, 219, |
| 333 | | 229, 275, 324, 333, 343, 360, 177_183 |
| 128, 153, 280, | 128, 153, 270, 324, 333 | 128, 139, 153, 162, 219, 229, 275, 324, |
| 333 | | 333, 177_183 |
| 128, 153, 280, | 128, 153, 274, 324, 333 | 128, 153, 162, 219, 229, 241, 275, 324, |
| 333 | | 333, 177_183 |
| 128, 153, 324, | 128, 153, 303, 324, 333 | 128, 153, 162, 219, 229, 275, 324, 333, |
| 333 | | 343, 177_183 |
| 128, 153, 310, | 128, 153, 324, 333, 364 | 128, 153, 162, 219, 229, 275, 324, 333, |
| 333 | | 177_183 |
| 128, 153, 310, | 128, 153, 224, 324, 333 | 128, 153, 162, 219, 229, 275, 282, 324, |
| 333 | | 333, 177_183 |
| 128, 153, 310, | 128, 153, 281, 324, 333 | 128, 153, 162, 197, 219, 229, 275, 324, |
| 333 | | 333, 177_183 |
| 128, 153, 318, | 84, 128, 153, 324, 333 | 128, 153, 162, 219, 229, 275, 324, 333, |
| 333 | | 360, 177_183 |
| 128, 153, 318, | 84, 128, 153, 324, 333 | 128, 153, 162, 219, 229, 262, 275, 324, |
| 333 | | 333, 177_183 |
| 128, 153, 318, | 102, 128, 153, 324, 333 | 128, 153, 162, 199, 219, 229, 275, 324, |
| 333 | | 333, 177_183 |
| 128, 153, 318, | 128, 153, 185, 324, 333 | 128, 153, 162, 219, 229, 275, 281, 324, |
| 333 | | 333, 177_183 |
| 128, 153, 318, | 128, 153, 182, 324, 333 | 93, 128, 153, 162, 219, 229, 275, 324, |
| 333 | | 333, 177_183 |
| 128, 153, 318, | 128, 153, 242, 324, 333 | 128, 153, 162, 219, 229, 275, 306, 324, |
| 333 | | 333, 177_183 |
| 128, 153, 318, | 128, 153, 301, 324, 333 | 128, 150, 153, 162, 219, 229, 275, 324, |
| 333 | | 333, 177_183 |
| 128, 153, 318, | 128, 153, 303, 324, 333 | 128, 153, 162, 174, 196, 219, 229, 275, |
| 333 | | 318, 324, 333, 177_183 |
| 128, 153, 318, | 128, 153, 310, 324, 333 | 128, 153, 162, 174, 219, 222, 229, 275, |
| 333 | | 280, 324, 333, 177_183 |
| 128, 153, 318, | 128, 153, 174, 324, 333 | 128, 153, 162, 174, 196, 219, 222, 229, |
| 333 | | 275, 280, 318, 324, 333, 177_183 |
| 128, 153, 201, | 128, 153, 173, 324, 333 | 128, 153, 162, 174, 219, 229, 275, 324, |
| 333 | | 332, 333, 177_183 |
| 128, 153, 201, | 128, 153, 196, 324, 333 | 128, 153, 162, 174, 219, 229, 233, 275, |
| 333 | | 324, 333, 177_183 |
| 128, 153, 197, | 128, 153, 196, 324, 333 | 128, 153, 162, 174, 219, 229, 254, 275, |
| 333 | | 324, 333, 177_183 |
| 128, 153, 160, | 128, 153, 195, 324, 333 | 128, 153, 162, 174, 219, 229, 275, 324, |
| 333 | | 333, 363, 177_183 |
| 128, 153, 165, | 128, 153, 195, 324, 333 | 128, 153, 162, 174, 219, 229, 275, 324, |
| 333 | | 333, 366, 177_183 |
| 128, 153, 309, | 128, 153, 197, 324, 333 | 128, 153, 162, 174, 219, 229, 275, 324, |
| 333 | | 333, 347, 177_183 |
| 128, 153, 319, | 128, 153, 197, 324, 333 | 122, 128, 153, 162, 174, 196, 219, 229, |
| 333 | | 275, 318, 324, 333, 177_183 |
| 128, 153, 319, | 128, 153, 197, 324, 333 | 122, 128, 153, 162, 174, 219, 222, 229, |
| 333 | | 275, 280, 324, 333, 177_183 |
| 128, 153, 320, | 128, 153, 263, 324, 333 | 122, 128, 153, 162, 174, 196, 219, 222, |
| 333 | | 229, 275, 280, 318, 324, 333, 177_183 |
| 128, 153, 320, | 128, 153, 268, 324, 333 | 128, 153, 162, 174, 219, 229, 275, 324, |
| 333 | | 333, 363, 366, 177_183 |
| 79, 128, 153, | 128, 153, 268, 324, 333 | 128, 153, 162, 174, 219, 229, 275, 324, |
| 189, 222, 333 | | 332, 333, 347, 177_183 |
| 71, 79, 128, 153, | 128, 153, 268, 324, 333 | 128, 153, 162, 174, 219, 229, 254, 275, |
| 222, 333 | | 324, 332, 333, 347, 363, 366, 177_183 |
| 71, 79, 128, 153, | 128, 153, 276, 324, 333 | 125, 128, 153, 162, 174, 219, 229, 275, |
| 189, 333 | | 324, 333, 177_183 |
| 71, 128, 153, | 128, 153, 275, 324, 333 | 128, 152, 153, 162, 174, 219, 229, 275, |
| 189, 222, 333 | | 324, 333, 177_183 |
| 128, 153, 324, | 128, 153, 279, 324, 333 | 128, 153, 162, 174, 219, 229, 275, 324, |
| 333, 162, 229 | | 333, 177_183 |
| 128, 153, 324, | 128, 153, 279, 324, 333 | 128, 153, 162, 174, 197, 219, 229, 275, |
| 333, 162, 229, | | 324, 333, 177_183 |
| 196 | | |
| 128, 153, 324, | 128, 153, 281, 324, 333 | 128, 153, 162, 174, 219, 229, 233, 275, |
| 333, 162, 229, | | 324, 332, 333, 177_183 |
| 280 | | |
| 128, 153, 324, | 128, 153, 281, 324, 333 | 128, 153, 162, 219, 229, 280, 324, 333 |
| 333, 162, 229, | | |
| 280, 196 | | |
| 128, 153, 324, | 128, 153, 282, 324, 333 | 128, 153, 162, 219, 229, 280, 307, 324, |
| 333, 196 | | 333 |
| 128, 153, 324, | 128, 153, 282, 324, 333 | 122, 128, 153, 162, 219, 229, 280, 324, |
| 333, 280 | | 333, 177_183 |
| 128, 153, 324, | 128, 153, 324, 333 | 122, 128, 153, 162, 219, 229, 275, 324, |
| 333, 280, 196 | | 333, 177_183 |
| 128, 153, 324, | 128, 153, 324, 329, 333 | 128, 153, 162, 219, 229, 275, 318, 324, |
| 333, 162, 229, | | 333, 177_183 |
| 196 | | |
| 128, 153, 324, | 128, 153, 324, 329, 333 | 128, 153, 162, 219, 229, 318, 324, 333, |
| 333, 162, 229, | | 177_183 |
| 280, 196 | | |
| 128, 153, 324, | 128, 153, 279, 324, 333 | 128, 153, 219, 275, 324, 333, 177_183 |
| 333, 162, 229, | | |
| 280, 222 | | |
| 128, 153, 324, | 128, 153, 279, 324, 333 | 122, 128, 153, 219, 324, 333, 177_183 |
| 333, 162, 229, | | |
| 280, 222, 196 | | |
| 128, 153, 324, | 71, 128, 153, 189, 324, 343 | 71, 128, 153, 219, 324, 333, 177_183 |
| 333, 162, 229, | | |
| 280, 222, 196 | | |
| 128, 153, 324, | 71, 94, 128, 143, 153, 189, 294, | 128, 153, 162, 229, 324, 333, 177_183 |
| 333, 318 | 324, 333, 343 | |
| 128, 153, 324, | 71, 84, 94, 128, 143, 153, 189, | 128, 153, 162, 229, 275, 324, 333, |
| 333, 162, 229, | 242, 294, 324, 333, 343 | 177_183 |
| 318 | | |
| 128, 153, 324, | 71, 84, 86, 91, 94, 128, 143, | 122, 128, 153, 162, 229, 324, 333, |
| 333, 162, 229, | 153, 181, 189, 228, 242, 243, | 177_183 |
| 280, 318 | 244, 256, 294, 324, 333, 343 | |
| 128, 153, 324, | 128, 153, 260, 324, 333, 346, | 128, 153, 162, 174, 199, 219, 229, 275, |
| 333, 320 | 347, 374 | 324, 333, 177_183 |
| 128, 153, 324, | 128, 153, 164, 260, 324, 333, | 128, 153, 324, 333, 318, 316 |
| 333, 162, 229, | 346, 347, 374 | |
| 320 | | |
| 128, 153, 324, | 128, 153, 175, 260, 323, 324, | 128, 153, 324, 333, 221 |
| 333, 162, 229, | 333, 346, 347, 374 | |
| 280, 320 | | |
| 128, 153, 324, | 128, 142, 153, 260, 324, 333, | 128, 153, 324, 333, 219 |
| 333, 122 | 346, 347 | |
| 128, 153, 324, | 128, 142, 153, 232, 260, 324, | 128, 153, 324, 333, 252 |
| 333, 162, 229, | 333, 337, 346, 347 | |
| 122 | | |
| 128, 153, 324, | 128, 142, 153, 217, 260, 324, | 128, 153, 324, 333, 250, 251 |
| 333, 162, 229, | 333, 337, 346, 347 | |
| 280, 122 | | |
| 128, 153, 324, | 115, 128, 142, 153, 260, 324, | 128, 153, 324, 333, 250, 251 |
| 333, 71, 68, 75, | 333, 337, 346, 347 | |
| 88 | | |
| 128, 153, 324, | 128, 142, 148, 153, 182, 260, | 128, 153, 324, 333, 205, 206 |
| 333, 162, 229, 71, | 286, 323, 324, 333, 346, 347 | |
| 68, 75, 88 | | |
| 128, 153, 324, | 128, 153, 162, 174, 219, 229, | 128, 153, 324, 333, 205, 206 |
| 333, 162, 229, | 233, 275, 324, 332, 333, | |
| 280, 71, 68, 75, | 177_183 | |
| 88 | | |
| 128, 153, 324, | 125, 128, 153, 162, 174, 219, | 128, 153, 324, 333, 205, 206 |
| 333, 162, 229, | 229, 233, 275, 324, 332, 333, | |
| 280, 196, 71, 68, | 177_183 | |
| 75,88 | | |
| 128, 153, 324, | 122, 128, 153, 162, 174, 219, | 128, 153, 324, 333, 205, 206 |
| 333, 318, 320, | 229, 233, 275, 324, 332, 333, | |
| 122, 71, 68, 75, | 177_183 | |
| 88 | | |
| 128, 153, 324, | 122, 125, 128, 153, 162, 174, | 128, 153, 324, 333, 205, 206, 250, 251 |
| 333, 162, 229, | 219, 229, 233, 275, 324, 332, | |
| 318, 320, 122, 71, | 333, 177_183 | |
| 68, 75,88 | | |
| 128, 153, 324, | 128, 153, 162, 174, 219, 229, | 128, 153, 162, 219, 229, 275, 318, 324, |
| 333, 162, 229, | 275, 324, 333, 177_183 | 333, 177_183 |
| 280, 318, 320, | | |
| 122, 71, 68, 75, | | |
| 88 | | |
| 128, 153, 324, | 125, 128, 153, 162, 174, 219, | 128, 153, 162, 219, 229, 318, 324, 333, |
| 333, 162, 229, | 229, 275, 324, 333, 177_183 | 177_183 |
| 280, 196, 318, | | |
| 320, 122, 71, 68, | | |
| 75,88 | | |
| 128, 153, 324, | 122, 128, 153, 162, 174, 219, | 128, 153, 219, 275, 324, 333, 177_183 |
| 333, 275 | 229, 275, 324, 333, 177_183 | |
| 128, 153, 324, | 122, 125, 128, 153, 162, 174, | 122, 128, 153, 219, 324, 333, 177_183 |
| 333, 162, 229, | 219, 229, 275, 324, 333, | |
| 275 | 177_183 | |
| 128, 153, 324, | 128, 153, 324, 333, 380 | 71, 128, 153, 219, 324, 333, 177_183 |
| 333, 162, 229, | | |
| 280, 275 | | |
| 128, 153, 324, | 128, 153, 162, 219, 229, 324, | 128, 153, 162, 219, 229, 324, 333, |
| 333, 162, 229, | 333, 177_183 | 177_183 |
| 280, 222, 196, | | |
| 318 | | |
| 128, 153, 324, | 128, 153, 162, 219, 229, 275, | 71, 128, 153, 162, 219, 229, 324, 333, |
| 333, 162, 229, | 324, 333, 177_183 | 177_183 |
| 280, 222, 196, | | |
| 320 | | |
| 128, 153, 324, | 122, 128, 153, 162, 219, 229, | 128, 153, 162, 219, 229, 275, 324, 333, |
| 333, 162, 229, | 324, 333, 177_183 | 177_183 |
| 280, 222, 196, | | |
| 122 | | |
| 128, 153, 324, | 128, 153, 162, 219, 229, 275, | 122, 128, 153, 162, 219, 229, 324, 333, |
| 333, 162, 229, | 280, 324, 333, 177_183 | 177_183 |
| 280, 222, 196, | | |
| 275 | | |
| 128, 153, 324, | 122, 128, 153, 162, 219, 229, | 128, 153, 162, 219, 229, 275, 280, 324, |
| 333, 162, 229, | 280, 324, 333, 177_183 | 333, 177_183 |
| 280, 222, 196, 71, | | |
| 68, 75, 88 | | |
| 128, 153, 324, | 71, 128, 153, 162, 219, 229, | 128, 153, 324, 333, 112 |
| 333, 162, 229, | 324, 333, 177_183 | |
| 280, 222, 196, | | |
| 318, 320, 122, 71, | | |
| 68, 75, 88 | | |
| 128, 153, 324, | 122, 128, 153, 162, 219, 229, | 128, 153, 324, 333, 327 |
| 333, 71 | 275, 324, 333, 177_183 | |
| 128, 153, 324, | 128, 153, 324, 333, 219 | 128, 153, 324, 333, 68 |
| 333, 219 | | |
| 128, 153, 324, | 128, 153, 324, 333, 221 | 128, 153, 324, 333, 75 |
| 333, 219 | | |
| 128, 153, 324, | 128, 153, 324, 333, 88 | |
| 333, 318, 316 | | |

In some embodiments, the polypeptide having protease activity comprises one or more mutations at positions corresponding to position 187 of SEQ ID NO: 2. In some embodiments, the polypeptide having protease activity comprises one or more mutations at positions corresponding to position 157, and 187 of SEQ ID NO: 2.

In some embodiments, the polypeptide having protease activity comprises one or more mutations selected from Table 3A or Table 3B. In some embodiments, the polypeptide having protease activity comprises one or more mutations selected from D71E, D71Q, D88H, D112Q, D152N, D139E, D307N, D293G, D295K, D295N, E75Y, E86D, E120D, E120G, E143D, E166Q, E241D, E310D, E310N, E310Q, E310S, E347N, E347R, E348R, E363K, E366K, F148Y, F175I, F333D, F333E, F333N, G199P, H174D, H174E, H197D, H197K, H197R, H222E, H222Q, H222S, H222T, H268G, H268R, H268S, H275L, H275Y, 184G, 184P, 184V, I262K, I301L, 1336A, K68W, K93D, K93S, K115L, K116D, K149L, K150E, K160E, K165E, K167R, K183D, K195L, K195R, K196D, K196E, K196L, K196Q, K196S, K196T, K201E, K201S, K205L, K229D, K229E, K229N, K244L, K297N, K297R, K318A, K318D, K318E, K318F, K318G, K318I, K318L, K318N, K318P, K318Q, K318S, K318V, K320W, K324I, K346P, K360R, L153P, L279G, L279T, L279W, L279Y, L329K, L329R, M117E, M117F, M323L, N162D, N162E, N173K, N182D, N182P, N219G, N219K, N219Q, N219T, N228K, N233G, N242G, N243K, N260A, P205F, P205FG, P205L, P205LG, Q180H, Q232E, Q294E, R142K, R189K, R250GGYGG (SEQ ID NO: 918), R250GYG, R256D, R282G, R282N, R280A, R280D, R280E, R280N, R280T, R280Y, R309G, R319E, R319T, R320L, R320Q, R320W, R337L, R360L, S94D, S94E, S94N, S128A, S181D, S220Y, S221E, S221N, S221Y, S251I, S286T, S306P, S317N, T122E, T122N, T122S, T167G, T176G, T276G, T276R, V102A, V102L, V164K, V270P, V274G, V303I, V303Y, V364E, Y212F, Y217W, Y224T, Y252R, Y281H, Y281S, Y281T, Y360L, 177_183delinsRDNRDN (SEQ ID NO: 919), 177_183KEYQSNKdelinsESDHG (SEQ ID NOs: 920 and 921), 177_183KEYQSNKdelinsRDNRDN (SEQ ID NOs: 920 and 919), 177_185KEYQSNKYAdelinsESPNT (SEQ ID NOs: 922 and 923), 177_318KEYQSNKNSKdelinsRDNRDN (SEQ ID NOs: 924 and 919), 177_319KEYQSNKDRdelinsRDNRDN (SEQ ID NOs: 925 and 919), 177_319KEYQSNKFNDNSKRdelinsRDNRDNIGDE (SEQ ID NOs: 926 and 927), 177_317KEYQSNKNDNSdelinsRDNRDN (SEQ ID NOs: 928 and 919), 180_185QSNKYAdelinsSG (SEQ ID NO: 929), 200_202NKAdelinsPYLSTKHL (SEQ ID NO: 930), 271_287delinsVGISGSAGGIKSVDSDGDSYI (SEQ ID NO: 932), 308_324delinsTKSVDSDGDSYKINY (SEQ ID NO: 932), 303_326delinsTGAKGNNYFGHWHFAVRVRL (SEQ ID NO: 933), 306_324delinsGHYGVYNGVTYWGHTPLHLAAMLGHLVYGLY (SEQ ID NO: 934), 306_324delinsQRTPLHLAAWADHLYR (SEQ ID NO: 935), 307_322delinsLAKGNNYFGHWHFAVM (SEQ ID NO: 936), 307_324delinsGGFAFDISIDNGNEDVY (SEQ ID NO: 937), 311_319delinsLLKPKILGYNITSGYSDEIY (SEQ ID NO: 938), 311_321delinsLSNVEALGYNITSGYSDKRY (SEQ ID NO: 939), 310_320delinsFLVFGVTYDGSTPVP (SEQ ID NO: 940), 311_321delinsLSNVEALGYNITSGYSDKRY (SEQ ID NO: 939), 86_105delinsDDMEAKGNNYFGHWHFAVATN (SEQ ID NO: 941), 88_102delinsGEIVAFDISIDNGNEDLAEILQLSTYDGGG (SEQ ID NO: 942), 91_100delinsGGGGGKSVDSDGDSYGGGGG (SEQ ID NO: 943), 93_98delinsGGGGGKSVDSDGDSYGGGG (SEQ ID NO: 944), 174_187delinsVMYKGKKLLEAARAGQPDSSE (SEQ ID NO: 945), 271_287delinsVGISGSAGGIKSVDSDGDSYI (SEQ ID NO: 932), 303_326delinsTGAKGNNYFGHWHFAVRVRL (SEQ ID NO: 933), 308_324delinsTKSVDSDGDSYKINY (SEQ ID NO: 932), and 380insENNVADSNKNSEDVEVSSVEFDFLNFKYPKNEDQVSTEDMRLTLKDTNVFNQPQ VKISFEEQNGNDWKEKDSGTFEEGKKYRLKIDVNKLALKIYGHLSKNKLNVIVNGKAV NIQNVVKKDSIETSFTIYSDSIDLEKINYWTDSFNNWS (SEQ ID NO: 744), wherein the positions correspond to SEQ ID NO: 1.

In some embodiments, the polypeptide having protease activity comprises an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises one or more mutations selected from Table 3A or Table 3B.

In some embodiments, the polypeptide having protease activity comprises an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises one or more mutations selected from D71E, D71Q, D88H, D112Q, D152N, D139E, D307N, D293G, D295K, D295N, E75Y, E86D, E120D, E120G, E143D, E166Q, E241D, E310D, E310N, E310Q, E310S, E347N, E347R, E348R, E363K, E366K, F148Y, F175I, F333D, F333E, F333N, G199P, H174D, H174E, H197D, H197K, H197R, H222E, H222Q, H222S, H222T, H268G, H268R, H268S, H275L, H275Y, 184G, 184P, 184V, I262K, I301L, I336A, K68W, K93D, K93S, K115L, K116D, K149L, K150E, K160E, K165E, K167R, K183D, K195L, K195R, K196D, K196E, K196L, K196Q, K196S, K196T, K201E, K201S, K205L, K229D, K229E, K229N, K244L, K297N, K297R, K318A, K318D, K318E, K318F, K318G, K318I, K318L, K318N, K318P, K318Q, K318S, K318V, K320W, K324I, K346P, K360R, L153P, L279G, L279T, L279W, L279Y, L329K, L329R, M117E, M117F, M323L, N162D, N162E, N173K, N182D, N182P, N219G, N219K, N219Q, N219T, N228K, N233G, N242G, N243K, N260A, P205F, P205FG, P205L, P205LG, Q180H, Q232E, Q294E, R142K, R189K, R250GGYGG (SEQ ID NO: 918), R250GYG, R256D, R282G, R282N, R280A, R280D, R280E, R280N, R280T, R280Y, R309G, R319E, R319T, R320L, R320Q, R320W, R337L, R360L, S94D, S94E, S94N, S128A, S181D, S220Y, S221E, S221N, S221Y, S251I, S286T, S306P, S317N, T122E, T122N, T122S, T167G, T176G, T276G, T276R, V102A, V102L, V164K, V270P, V274G, V303I, V303Y, V364E, Y212F, Y217W, Y224T, Y252R, Y281H, Y281S, Y281T, Y360L, 177_183delinsRDNRDN (SEQ ID NO: 919), 177_183KEYQSNKdelinsESDHG (SEQ ID NOs: 920 and 921), 177_183KEYQSNKdelinsRDNRDN (SEQ ID NOs: 920 and 919), 177_185KEYQSNKYAdelinsESPNT (SEQ ID NOs: 922 and 923), 177_318KEYQSNKNSKdelinsRDNRDN (SEQ ID NOs: 924 and 919), 177_319KEYQSNKDRdelinsRDNRDN (SEQ ID NOs: 925 and 919), 177_319KEYQSNKFNDNSKRdelinsRDNRDNIGDE (SEQ ID NOs: 926 and 927), 177_317KEYQSNKNDNSdelinsRDNRDN (SEQ ID NOs: 928 and 919), 180_185QSNKYAdelinsSG (SEQ ID NO: 929), 200_202NKAdelinsPYLSTKHL (SEQ ID NO: 930), 271_287delinsVGISGSAGGIKSVDSDGDSYI (SEQ ID NO: 932), 308_324delinsTKSVDSDGDSYKINY (SEQ ID NO: 932), 303_326delinsTGAKGNNYFGHWHFAVRVRL (SEQ ID NO: 933), 306_324delinsGHYGVYNGVTYWGHTPLHLAAMLGHLVYGLY (SEQ ID NO: 934), 306_324delinsQRTPLHLAAWADHLYR (SEQ ID NO: 935), 307_322delinsLAKGNNYFGHWHFAVM (SEQ ID NO: 936), 307_324delinsGGFAFDISIDNGNEDVY (SEQ ID NO: 937), 311_319delinsLLKPKILGYNITSGYSDEIY (SEQ ID NO: 938), 311_321delinsLSNVEALGYNITSGYSDKRY (SEQ ID NO: 939), 310_320delinsFLVFGVTYDGSTPVP (SEQ ID NO: 940), 311_321delinsLSNVEALGYNITSGYSDKRY (SEQ ID NO: 939), 86_105delinsDDMEAKGNNYFGHWHFAVATN (SEQ ID NO: 941), 88_102delinsGEIVAFDISIDNGNEDLAEILQLSTYDGGG (SEQ ID NO: 942), 91_100delinsGGGGGKSVDSDGDSYGGGGG (SEQ ID NO: 943), 93_98delinsGGGGGKSVDSDGDSYGGGG (SEQ ID NO: 944), 174_187delinsVMYKGKKLLEAARAGQPDSSE (SEQ ID NO: 945), 271_287delinsVGISGSAGGIKSVDSDGDSYI (SEQ ID NO: 932), 303_326delinsTGAKGNNYFGHWHFAVRVRL (SEQ ID NO: 933), 308_324delinsTKSVDSDGDSYKINY (SEQ ID NO: 932), and 380insENNVADSNKNSEDVEVSSVEFDFLNFKYPKNEDQVSTEDMRLTLKDTNVFNQPQ VKISFEEQNGNDWKEKDSGTFEEGKKYRLKIDVNKLALKIYGHLSKNKLNVIVNGKAV NIQNVVKKDSIETSFTIYSDSIDLEKINYWTDSFNNWS (SEQ ID NO: 744), wherein the positions correspond to SEQ ID NO: 1.

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of L153P, R189K, S128A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of L153P, S128A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, K229E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, L153P, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, L153P, R189K, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, L153P, S128A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, L153P, V126S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of E166Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K196S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K196E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of H222Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of E120G, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K229E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of N162E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of R280E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K318G, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K318A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, A79V, T122S, S128A, L153P, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, R189K, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, H222Q, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, R189K, H222Q, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, A167T, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, R189K, H222Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, A167T, H222Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, V102A, T122S, S128A, L153P, A167T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, A79V, T122S, S128A, L153P, A167T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K318A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, H222Q, K229E, R280E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222Q, K229E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222Q, R280E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K229E, R280E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H222Q, K229E, R280E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K196S, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H222Q, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, R280E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K318A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K196S, K318A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196S, K318A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196S, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196S, E310Q, K318A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K196S, E310Q, K318A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K318A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, H222Q, K229E, R280E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, H222Q, K229E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, H222Q, R280E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K229E, R280E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H222Q, K229E, R280E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K196S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H222Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K229E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, R280E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K318A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K196S, K318A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K196S, K318A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K196S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K196S, Y212F, K318A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K196S, E310Q, K318A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K196S, E310Q, K318A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E166D, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196T, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196D, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196N, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222S, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222T, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of E120D, S128A, L153P, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122E, S128A, L153P, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122N, S128A, L153P, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K229D, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K229N, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162D, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280D, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280A, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280Y, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280N, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280T, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E310D, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E310N, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E310S, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318N, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318D, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318Q, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318P, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318F, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318V, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318L, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318S, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K201E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K201S, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H197S, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K160E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K165E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R309G, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R319E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R319T, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R320Q, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R320L, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, S128A, L153P, R189K, H222Q, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, A79V, S128A, L153P, H222Q, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, A79V, S128A, L153P, R189K, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, S128A, L153P, R189K, H222Q, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, K196D, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K196D, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K196D, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R280T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R280T, K196D, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, K196S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K196S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196D, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K318S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, K318S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K318S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R320W, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R320W, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, R320W, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, T122S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, T122S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, T122S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K196D, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K318S, R320W, T122S, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, K318S, R320W, T122S, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K318S, R320W, T122S, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K196D, K318S, R320W, T122S, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, H275Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, H275Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H275Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, K318S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, R320W, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, T122S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, H275Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, K318S, R320W, T122S, D71Q, K68W, E75Y, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D71Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D112Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D327Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K68W, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, E75Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D88H, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N219Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S221N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N219G, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N219T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K318S, N316S, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K318S, N316Q, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S221E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N219K, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, Y252R, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R250GYG, S251I, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R250GGYGG (SEQ ID NO: 918), S251I, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205L, E206T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205F, E206T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205LG, E206T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205FG, E206T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205FG, E206T, R250GYG, S251I, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, I262K, T276G, V364E, P205FG, E206T, R250GYG, S251I, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, G199P, I262K, T276G, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K116D, K149L, N182D, K196D, I262K, T276G, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, I262K, T276G, 1336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, I262K, T276G, I336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, I262K, T276G, Y281T, 1336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, I262K, T276G, Y281T, I336A, V364E, H174E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, V270P, T276G, Y281T, 1336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, V270P, V274G, Y281T, 1336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, G199P, Y224T, V270P, V274G, Y281T, 1336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, K196D, I262K, T276G, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, I262K, V274G, Y281T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, G199P, I262K, T276G, Y281T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K196D, I262K, T276G, Y281T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, G199P, Y224T, I262K, T276G, Y281T, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, G199P, Y224T, I262K, T276G, Y281T, 1336A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, G199P, Y224T, I262K, T276G, Y281T, 1336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, K196D, Y224T, I262K, T276G, Y281T, 1336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, G199P, Y224T, I262K, T276G, Y281T, 1336A, V364E, H174E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, I262K, T276G, Y281T, V303I, 1336A, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of, T122S, S128A, L153P, F333N, K324I, N162E, K229E, H275Y, N219G, H174E, 177_183delinsRDNRDN (SEQ ID NO: 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 307_322delinsLAKGNNYFGHWHFAVM (SEQ ID NO: 936), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 86_105delinsDDMEAKGNNYFGHWHFAVATN (SEQ ID NO: 941), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 271_287delinsVGISGSAGGIKSVDSDGDSYI (SEQ ID NO: 932), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 91_100delinsGGGGGKSVDSDGDSYGGGGG (SEQ ID NO: 943), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 308_324delinsTKSVDSDGDSYKINY (SEQ ID NO: 932), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_319delinsLLKPKILGYNITSGYSDEIY (SEQ ID NO: 938), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 315_317delins VTYDGST (SEQ ID NO: 946), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 303_326delinsTGAKGNNYFGHWHFAVRVRL (SEQ ID NO: 933), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 93_98delinsGGGGGKSVDSDGDSYGGGGG (SEQ ID NO: 943), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 174_187delinsVMYKGKKLLEAARAGQPDSSE (SEQ ID NO: 945), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 306_324delinsGHYGVYNGVTYWGHTPLHLAAMLGHLVYGLY (SEQ ID NO: 934), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 306_324delinsQRTPLHLAAWADHLYR (SEQ ID NO: 935), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 307_324delinsGGFAFDISIDNGNEDVY (SEQ ID NO: 937), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 88_102delinsGEIVAFDISIDNGNEDLAEILQLSTYDGGG (SEQ ID NO: 942), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), 86_105delinsDDMEAKGNNYFGHWHFAVATN (SEQ ID NO: 941), 271_287delinsVGISGSAGGIKSVDSDGDSYI (SEQ ID NO: 932), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), 86_105delinsDDMEAKGNNYFGHWHFAVATN (SEQ ID NO: 941), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), 91_100delinsGGGGGKSVDSDGDSYGGGGG (SEQ ID NO: 943), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), 271_287delinsVGISGSAGGIKSVDSDGDSYI (SEQ ID NO: 932), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), 86_105delinsDDMEAKGNNYFGHWHFAVATN (SEQ ID NO: 941), 271_287delinsVGISGSAGGIKSVDSDGDSYI (SEQ ID NO: 932), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), 86_105delinsDDMEAKGNNYFGHWHFAVATN (SEQ ID NO: 941), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), 91_100delinsGGGGGKSVDSDGDSYGGGGG (SEQ ID NO: 943), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), 271_287delinsVGISGSAGGIKSVDSDGDSYI (SEQ ID NO: 932), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 307_322delinsLAKGNNYFGHWHFAVM (SEQ ID NO: 936), 91_100delinsGGGGGKSVDSDGDSYGGGGG (SEQ ID NO: 943), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 307_322delinsLAKGNNYFGHWHFAVM (SEQ ID NO: 936), 271_287delinsVGISGSAGGIKSVDSDGDSYI (SEQ ID NO: 932), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 308_324delinsTKSVDSDGDSYKINY (SEQ ID NO: 932), 86_105delinsDDMEAKGNNYFGHWHFAVATN (SEQ ID NO: 941), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of M117E, S128A, L153P, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of M117F, S128A, L153P, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F333N, E348R, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F333N, E347R, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F333N, 1351R, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, S220Y, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, S221Y, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H275Y, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F333N, 200_202NKAdelinsPYLSTKHL (SEQ ID NO: 930), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of M117E, S128A, L153P, H222E, F333N, E348R, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, R280T, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, K229E, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, K229E, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, K229E, R280T, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_183KEYQSNKdelinsESDHG (SEQ ID NOs: 920 and 921), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, Q180H, K183D, K229E, K324I, F333N, 176_178YKEdelinsN, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, F313T, S317N, K324I, F333N, 177_319KEYQSNKDRdelinsRDNRDN (SEQ ID NOs: 925 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_319KEYQSNKFNDNSKRdelinsRDNRDNIGDE (SEQ ID NOs: 926 and 927), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, F313I, N314G, K324I, F333N, 177_318KEYQSNKNSKdelinsRDNRDN (SEQ ID NOs: 924 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, D293G, Q294E, K297R, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, Q294E, D295K, K324I, F333N, 177_183KEYQSNKdelinsRDNRDN (SEQ ID NOS: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, Q294E, D295N, K297N, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_185KEYQSNKYAdelinsESPNT (SEQ ID NOs: 922 and 923), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 180_185QSNKYAdelinsSG (SEQ ID NO: 929), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K318G, K324I, F333N, 177_317KEYQSNKNDNSdelinsRDNRDN (SEQ ID NOs: 928 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_317KEYQSNKNDNSdelinsRDNRDN (SEQ ID NOs: 928 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, G199P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H174E, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, I262K, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 1336A, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K116D, S128A, L153P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, K149L, L153P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N182D, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S94E, S128A, L153P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, T276G, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V270P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V274G, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V303I, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, V364E, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, Y224T, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, Y281T, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of I84P, S128A, L153P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of I84G, S128A, L153P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of V102L, S128A, L153P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, A185T, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N182P, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N242G, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, I301L, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V303Y, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E310D, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H174D, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N173K, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196Q, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196L, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K195L, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K195R, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H197R, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H197K, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H197D, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N263D, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H268G, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H268R, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H268S, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, T276R, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H275L, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, L279G, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, L279T, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, Y281H, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, Y281S, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R282G, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R282N, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333D, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, L329K, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, L329R, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, L279W, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, L279Y, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, S128A, L153P, R189K, K324I, A343Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, S94D, S128A, E143D, L153P, R189K, Q294E, K324I, F333N, A343Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, I84V, S94D, S128A, E143D, L153P, R189K, N242G, Q294E, K324I, F333N, A343Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, I84V, E86D, H91K, S94D, S128A, E143D, L153P, S181D, R189K, N228K, N242G, N243K, K244L, G256D, Q294E, K324I, F333N, A343Y, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N260A, K324I, F333N, K346P, E347N, E374P, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V164K, N260A, K324I, F333N, K346P, E347N, E374P, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F175I, N260A, M323L, K324I, F333N, K346P, E347N, E374P, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, R142K, L153P, N260A, K324I, F333N, K346P, E347N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, R142K, L153P, Q232E, N260A, K324I, F333N, R337L, K346P, E347N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, R142K, L153P, Y217W, N260A, K324I, F333N, R337L, K346P, E347N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K115L, S128A, R142K, L153P, N260A, K324I, F333N, R337L, K346P, E347N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, R142K, F148Y, L153P, N182D, N260A, S286T, M323L, K324I, F333N, K346P, E347N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, C125S, S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, C125S, S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 380insENNVADSNKNSEDVEVSSVEFDFLNFKYPKNEDQVSTEDMRLTLKDTNVFNQPQ VKISFEEQNGNDWKEKDSGTFEEGKKYRLKIDVNKLALKIYGHLSKNKLNVIVNGKAV NIQNVVKKDSIETSFTIYSDSIDLEKINYWTDSFNNWS (SEQ ID NO: 744), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219G, K229E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, R280T, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219G, K229E, R280T, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71Q, S128A, L153P, N162E, N219G, K229E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K318S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, K318S, K324I, F333N, 177_183KEYQSNKdelinsRDNRDN (SEQ ID NOS: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N219G, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N219G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71Q, S128A, L153P, N219G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71Q, S128A, L153P, N162E, N219Q, K229E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219Q, K229E, K324I, F333N, 177_183KEYQSNKdelinsRDNRDN (SEQ ID NOS: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, H275Y, R280T, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219Q, K229E, R280T, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219Q, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, H275Y, K318S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, K318S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N219Q, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N219Q, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71Q, S128A, L153P, N219Q, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K229E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, R282G, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, R282G, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, R282G, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, R282G, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, R282G, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, S306P, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, S306P, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, S306P, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, S306P, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, S306P, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, S306P, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, Y281S, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, Y281S, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, Y281S, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, Y281S, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, Y281S, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, K324I, F333N, A343K, R360L, 177_183KEYQSNKdelinsRDNRDN (SEQ ID NOS: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, S306P, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, S306P, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, S306P, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, 1262K, H275Y, Y281S, S306P, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, S306P, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, S306P, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, S306P, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, S306P, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, S306P, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, S306P, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, S306P, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, S306P, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, H197D, N219G, K229E, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, H197D, N219G, K229E, E241D, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, H197D, N219G, K229E, I262K, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, H197D, N219G, K229E, E241D, I262K, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, S306P, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, S306P, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, S306P, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, S306P, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, S306P, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, S306P, K324I, F333E, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, K324I, F333N, A343K, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, E241D, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K324I, F333N, A343K, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, R282G, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H197D, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K324I, F333N, R360L, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, I262K, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, G199P, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, Y281S, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, S306P, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, K150E, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, K196S, N219G, K229E, H275Y, K318A, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, H222Q, K229E, H275Y, R280E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, K196S, N219G, H222Q, K229E, H275Y, R280E, K318A, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, D332K, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, N233G, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, I254R, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, E363K, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, E366K, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, E347N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, K196S, N219G, K229E, H275Y, K318A, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, N219G, H222Q, K229E, H275Y, R280E, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, K196S, N219G, H222Q, K229E, H275Y, R280E, K318A, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, E363K, E366K, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, D332K, F333N, E347N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, I254R, H275Y, K324I, D332K, F333N, E347N, E363K, E366K, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D152N, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333E, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, H197D, N219G, K229E, H275Y, K324I, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, R280T, K324I, F333N, wherein the positions correspond to SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, R280T, D307N, K324I, F333N, wherein the positions correspond to SEQ ID NO: 1.

In some embodiments, the polypeptide having protease activity comprises an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1.

In some embodiments, the polypeptide having protease activity comprises an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises one or more mutations selected from D71E, D71Q, D88H, D112Q, D152N, D139E, D307N, D293G, D295K, D295N, E75Y, E86D, E120D, E120G, E143D, E166Q, E241D, E310D, E310N, E310Q, E310S, E347N, E347R, E348R, E363K, E366K, F148Y, F175I, F333D, F333E, F333N, G199P, H174D, H174E, H197D, H197K, H197R, H222E, H222Q, H222S, H222T, H268G, H268R, H268S, H275L, H275Y, 184G, 184P, 184V, 1262K, I301L, I336A, K68W, K93D, K93S, K115L, K116D, K149L, K150E, K160E, K165E, K167R, K183D, K195L, K195R, K196D, K196E, K196L, K196Q, K196S, K196T, K201E, K201S, K205L, K229D, K229E, K229N, K244L, K297N, K297R, K318A, K318D, K318E, K318F, K318G, K318I, K318L, K318N, K318P, K318Q, K318S, K318V, K320W, K324I, K346P, K360R, L153P, L279G, L279T, L279W, L279Y, L329K, L329R, M117E, M117F, M323L, N162D, N162E, N173K, N182D, N182P, N219G, N219K, N219Q, N219T, N228K, N233G, N242G, N243K, N260A, P205F, P205FG, P205L, P205LG, Q180H, Q232E, Q294E, R142K, R189K, R250GGYGG (SEQ ID NO: 918), R250GYG, R256D, R282G, R282N, R280A, R280D, R280E, R280N, R280T, R280Y, R309G, R319E, R319T, R320L, R320Q, R320W, R337L, R360L, S94D, S94E, S94N, S128A, S181D, S220Y, S221E, S221N, S221Y, S251I, S286T, S306P, S317N, T122E, T122N, T122S, T167G, T176G, T276G, T276R, V102A, V102L, V164K, V270P, V274G, V303I, V303Y, V364E, Y212F, Y217W, Y224T, Y252R, Y281H, Y281S, Y281T, Y360L, 177_183delinsRDNRDN (SEQ ID NO: 919), 177_183KEYQSNKdelinsESDHG (SEQ ID NOs: 920 and 921), 177_183KEYQSNKdelinsRDNRDN (SEQ ID NOs: 920 and 919), 177_185KEYQSNKYAdelinsESPNT (SEQ ID NOs: 922 and 923), 177_318KEYQSNKNSKdelinsRDNRDN (SEQ ID NOs: 924 and 919), 177_319KEYQSNKDRdelinsRDNRDN (SEQ ID NOs: 925 and 919), 177_319KEYQSNKFNDNSKRdelinsRDNRDNIGDE (SEQ ID NOs: 926 and 927), 177_317KEYQSNKNDNSdelinsRDNRDN (SEQ ID NOs: 928 and 919), 180_185QSNKYAdelinsSG (SEQ ID NO: 929), 200_202NKAdelinsPYLSTKHL (SEQ ID NO: 930), 271_287delinsVGISGSAGGIKSVDSDGDSYI (SEQ ID NO: 932), 308_324delinsTKSVDSDGDSYKINY (SEQ ID NO: 932), 303_326delinsTGAKGNNYFGHWHFAVRVRL (SEQ ID NO: 933), 306_324delinsGHYGVYNGVTYWGHTPLHLAAMLGHLVYGLY (SEQ ID NO: 934), 306_324delinsQRTPLHLAAWADHLYR (SEQ ID NO: 935), 307_322delinsLAKGNNYFGHWHFAVM (SEQ ID NO: 936), 307_324delinsGGFAFDISIDNGNEDVY (SEQ ID NO: 937), 311_319delinsLLKPKILGYNITSGYSDEIY (SEQ ID NO: 938), 311_321delinsLSNVEALGYNITSGYSDKRY (SEQ ID NO: 939), 310_320delinsFLVFGVTYDGSTPVP (SEQ ID NO: 940), 311_321delinsLSNVEALGYNITSGYSDKRY (SEQ ID NO: 939), 86_105delinsDDMEAKGNNYFGHWHFAVATN (SEQ ID NO: 941), 88_102delinsGEIVAFDISIDNGNEDLAEILQLSTYDGGG (SEQ ID NO: 942), 91_100delinsGGGGGKSVDSDGDSYGGGGG (SEQ ID NO: 943), 93_98delinsGGGGGKSVDSDGDSYGGGG (SEQ ID NO: 944), 174_187delinsVMYKGKKLLEAARAGQPDSSE (SEQ ID NO: 945), 271_287delinsVGISGSAGGIKSVDSDGDSYI (SEQ ID NO: 932), 303_326delinsTGAKGNNYFGHWHFAVRVRL (SEQ ID NO: 933), 308_324delinsTKSVDSDGDSYKINY (SEQ ID NO: 932), and 380insENNVADSNKNSEDVEVSSVEFDFLNFKYPKNEDQVSTEDMRLTLKDTNVFNQPQ VKISFEEQNGNDWKEKDSGTFEEGKKYRLKIDVNKLALKIYGHLSKNKLNVIVNGKAV NIQNVVKKDSIETSFTIYSDSIDLEKINYWTDSFNNWS (SEQ ID NO: 744), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1.

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of L153P, R189K, S128A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of L153P, S128A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, K229E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, L153P, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, L153P, R189K, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, L153P, S128A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, L153P, V126S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of E166Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K196S, wherein the positions correspond to SEQ ID NO: 1, and provided that the amino acid sequence comprises a cysteine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K196E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of H222Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of E120G, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K229E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of N162E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of R280E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K318G, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K318A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, A79V, T122S, S128A, L153P, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, R189K, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, H222Q, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, R189K, H222Q, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, A167T, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, R189K, H222Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, T122S, S128A, L153P, A167T, H222Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, V102A, T122S, S128A, L153P, A167T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, A79V, T122S, S128A, L153P, A167T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K318A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, H222Q, K229E, R280E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222Q, K229E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222Q, R280E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K229E, R280E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H222Q, K229E, R280E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K196S, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H222Q, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, R280E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K318A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K196S, K318A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196S, K318A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196S, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196S, E310Q, K318A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K196S, E310Q, K318A, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K318A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, H222Q, K229E, R280E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, H222Q, K229E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, H222Q, R280E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K229E, R280E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H222Q, K229E, R280E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K196S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H222Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K229E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, R280E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K318A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K196S, K318A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K196S, K318A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K196S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K196S, Y212F, K318A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, K196S, E310Q, K318A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K196S, E310Q, K318A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E166D, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196T, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196D, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196N, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222S, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H222T, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of E120D, S128A, L153P, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122E, S128A, L153P, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122N, S128A, L153P, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K229D, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K229N, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162D, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280D, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280A, F333N, wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280Y, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280N, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R280T, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E310D, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E310N, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E310S, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318N, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318D, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318Q, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318P, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318F, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318V, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318L, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318S, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K318I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K201E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K201S, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H197S, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K160E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K165E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R309G, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R319E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R319T, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R320Q, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R320L, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of A79V, S128A, L153P, R189K, H222Q, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, A79V, S128A, L153P, H222Q, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, A79V, S128A, L153P, R189K, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, S128A, L153P, R189K, H222Q, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, K196D, wherein the positions correspond to SEQ ID NO: 1, and provided that the amino acid sequence comprises a cysteine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K196D, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K196D, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R280T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R280T, K196D, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, K196S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K196S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196D, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K318S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, K318S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K318S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R320W, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R320W, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, R320W, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, T122S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, T122S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, T122S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K196D, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K318S, R320W, T122S, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, K318S, R320W, T122S, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K318S, R320W, T122S, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, K196D, K318S, R320W, T122S, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, H275Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, H275Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H275Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, K318S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, R320W, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, T122S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, H275Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N162E, K229E, R280T, H222Q, K196S, K318S, R320W, T122S, D71Q, K68W, E75Y, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D71Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D112Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D327Y, wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K68W, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, E75Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, D88H, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N219Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S221N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N219G, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N219T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K318S, N316S, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K318S, N316Q, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S221E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, N219K, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, Y252R, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R250GYG, S251I, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, R250GGYGG (SEQ ID NO: 918), S251I, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205L, E206T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205F, E206T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205LG, E206T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205FG, E206T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, P205FG, E206T, R250GYG, S251I, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, I262K, T276G, V364E, P205FG, E206T, R250GYG, S251I, wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, G199P, I262K, T276G, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K116D, K149L, N182D, K196D, I262K, T276G, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, I262K, T276G, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, I262K, T276G, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, I262K, T276G, Y281T, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, I262K, T276G, Y281T, 1336A, V364E, H174E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, V270P, T276G, Y281T, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, V270P, V274G, Y281T, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, G199P, Y224T, V270P, V274G, Y281T, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, K196D, I262K, T276G, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, I262K, V274G, Y281T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, G199P, I262K, T276G, Y281T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K196D, I262K, T276G, Y281T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, G199P, Y224T, 1262K, T276G, Y281T, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, G199P, Y224T, I262K, T276G, Y281T, 1336A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, G199P, Y224T, I262K, T276G, Y281T, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, K196D, Y224T, I262K, T276G, Y281T, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, K149L, G199P, Y224T, I262K, T276G, Y281T, 1336A, V364E, H174E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, S94E, K116D, K149L, N182D, K196D, Y224T, I262K, T276G, Y281T, V303I, 1336A, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of, T122S, S128A, L153P, F333N, K324I, N162E, K229E, H275Y, N219G, H174E, 177_183delinsRDNRDN (SEQ ID NO: 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 307_322delinsLAKGNNYFGHWHFAVM (SEQ ID NO: 936), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 86_105delinsDDMEAKGNNYFGHWHFAVATN (SEQ ID NO: 941), wherein the positions correspond to SEQ ID NO: 1, and provided that the amino acid sequence comprises a cysteine aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 271_287delinsVGISGSAGGIKSVDSDGDSYI (SEQ ID NO: 932), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 91_100delinsGGGGGKSVDSDGDSYGGGGG (SEQ ID NO: 943), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 308_324delinsTKSVDSDGDSYKINY (SEQ ID NO: 932), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_319delinsLLKPKILGYNITSGYSDEIY (SEQ ID NO: 938), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 315_317delinsVTYDGST (SEQ ID NO: 946), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 303_326delinsTGAKGNNYFGHWHFAVRVRL (SEQ ID NO: 933), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 93_98delinsGGGGGKSVDSDGDSYGGGGG (SEQ ID NO: 943), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 174_187delinsVMYKGKKLLEAARAGQPDSSE (SEQ ID NO: 945), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 306_324delinsGHYGVYNGVTYWGHTPLHLAAMLGHLVYGLY (SEQ ID NO: 934), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 306_324delinsQRTPLHLAAWADHLYR (SEQ ID NO: 935), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 307_324delinsGGFAFDISIDNGNEDVY (SEQ ID NO: 937), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 88_102delinsGEIVAFDISIDNGNEDLAEILQLSTYDGGG (SEQ ID NO: 942), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), 86_105delinsDDMEAKGNNYFGHWHFAVATN (SEQ ID NO: 941), 271_287delinsVGISGSAGGIKSVDSDGDSYI (SEQ ID NO: 932), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), 86_105delinsDDMEAKGNNYFGHWHFAVATN (SEQ ID NO: 941), wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), 91_100delinsGGGGGKSVDSDGDSYGGGGG (SEQ ID NO: 943), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 311_321delinsLSNVEALGYNITSGYSDKRY (SEQ ID NO: 939), 271_287delinsVGISGSAGGIKSVDSDGDSYI (SEQ ID NO: 932), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), 86_105delinsDDMEAKGNNYFGHWHFAVATN (SEQ ID NO: 941), 271_287delinsVGISGSAGGIKSVDSDGDSYI (SEQ ID NO: 932), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), 86_105delinsDDMEAKGNNYFGHWHFAVATN (SEQ ID NO: 941), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), 91_100delinsGGGGGKSVDSDGDSYGGGGG (SEQ ID NO: 943), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 310_320delinsFLVFGVTYDGSTPVP (SEQ ID NO: 940), 271_287delinsVGISGSAGGIKSVDSDGDSYI (SEQ ID NO: 932), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of $128A, L153P, K324I, F333N, 307_322delinsLAKGNNYFGHWHFAVM (SEQ ID NO: 936), 91_100delinsGGGGGKSVDSDGDSYGGGGG (SEQ ID NO: 943), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 307_322delinsLAKGNNYFGHWHFAVM (SEQ ID NO: 936), 271_287delinsVGISGSAGGIKSVDSDGDSYI (SEQ ID NO: 932), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 308_324delinsTKSVDSDGDSYKINY (SEQ ID NO: 932), 86_105delinsDDMEAKGNNYFGHWHFAVATN (SEQ ID NO: 941), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of M117E, S128A, L153P, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of M117F, S128A, L153P, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F333N, E348R, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F333N, E347R, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F333N, 1351R, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, S220Y, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, S221Y, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H275Y, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F333N, 200_202NKAdelinsPYLSTKHL (SEQ ID NO: 930), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of M117E, S128A, L153P, H222E, F333N, E348R, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, R280T, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, K229E, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, K229E, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, K229E, R280T, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_183KEYQSNKdelinsESDHG (SEQ ID NOs: 920 and 921), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, Q180H, K183D, K229E, K324I, F333N, 176_178YKEdelinsN, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, F313T, S317N, K324I, F333N, 177_319KEYQSNKDRdelinsRDNRDN (SEQ ID NOs: 925 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_319KEYQSNKFNDNSKRdelinsRDNRDNIGDE (SEQ ID NOs: 926 and 927), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, F313I, N314G, K324I, F333N, 177_318KEYQSNKNSKdelinsRDNRDN (SEQ ID NOs: 924 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, D293G, Q294E, K297R, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, Q294E, D295K, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, Q294E, D295N, K297N, K324I, F333N, 177_183KEYQSNKdelinsRDNRDN (SEQ ID NOS: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_185KEYQSNKYAdelinsESPNT (SEQ ID NOs: 922 and 923), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 180_185QSNKYAdelinsSG (SEQ ID NO: 929), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K318G, K324I, F333N, 177_317KEYQSNKNDNSdelinsRDNRDN (SEQ ID NOs: 928 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_317KEYQSNKNDNSdelinsRDNRDN (SEQ ID NOs: 928 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, G199P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H174E, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, I262K, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 1336A, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K116D, S128A, L153P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, K149L, L153P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N182D, K324I, F333N, wherein the positions correspond to SEQ ID NO: 1, and provided that the amino acid sequence comprises a cysteine aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S94E, S128A, L153P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, T276G, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V270P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V274G, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V303I, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, V364E, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, Y224T, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, Y281T, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of 184P, S128A, L153P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of 184G, S128A, L153P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of V102L, S128A, L153P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, A185T, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N182P, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N242G, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, 1301L, K324I, F333N, wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V303Y, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, E310D, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H174D, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N173K, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196Q, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K196L, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K195L, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K195R, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H197R, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H197K, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H197D, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N263D, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H268G, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H268R, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H268S, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, T276R, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, H275L, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, L279G, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, L279T, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, Y281H, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, Y281S, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R282G, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, R282N, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333D, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, L329K, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, L329R, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, L279W, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, L279Y, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, S128A, L153P, R189K, K324I, A343Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, S94D, S128A, E143D, L153P, R189K, Q294E, K324I, F333N, A343Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, 184V, S94D, S128A, E143D, L153P, R189K, N242G, Q294E, K324I, F333N, A343Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71E, 184V, E86D, H91K, S94D, S128A, E143D, L153P, S181D, R189K, N228K, N242G, N243K, K244L, G256D, Q294E, K324I, F333N, A343Y, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N260A, K324I, F333N, K346P, E347N, E374P, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, V164K, N260A, K324I, F333N, K346P, E347N, E374P, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, F175I, N260A, M323L, K324I, F333N, K346P, E347N, E374P, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, R142K, L153P, N260A, K324I, F333N, K346P, E347N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, R142K, L153P, Q232E, N260A, K324I, F333N, R337L, K346P, E347N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, R142K, L153P, Y217W, N260A, K324I, F333N, R337L, K346P, E347N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K115L, S128A, R142K, L153P, N260A, K324I, F333N, R337L, K346P, E347N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, R142K, F148Y, L153P, N182D, N260A, S286T, M323L, K324I, F333N, K346P, E347N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, C125S, S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, 177_183KEYQSNKdelinsRDNRDN (SEQ ID NOS: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, C125S, S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, K324I, F333N, 380insENNVADSNKNSEDVEVSSVEFDFLNFKYPKNEDQVSTEDMRLTLKDTNVENQPQ VKISFEEQNGNDWKEKDSGTFEEGKKYRLKIDVNKLALKIYGHLSKNKLNVIVNGKAV NIQNVVKKDSIETSFTIYSDSIDLEKINYWTDSENNWS (SEQ ID NO: 744), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219G, K229E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, R280T, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219G, K229E, R280T, K324I, F333N, 177_183KEYQSNKdelinsRDNRDN (SEQ ID NOS: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71Q, S128A, L153P, N162E, N219G, K229E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K318S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, K318S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N219G, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N219G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71Q, S128A, L153P, N219G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71Q, S128A, L153P, N162E, N219Q, K229E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219Q, K229E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, H275Y, R280T, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219Q, K229E, R280T, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, N219Q, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, H275Y, K318S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, K318S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N219Q, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N219Q, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of D71Q, S128A, L153P, N219Q, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, K229E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, R282G, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, R282G, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, R282G, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, R282G, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, R282G, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, 1262K, H275Y, R282G, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, S306P, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, S306P, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, S306P, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, S306P, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, R282G, S306P, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, R282G, S306P, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, Y281S, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, Y281S, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, Y281S, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, Y281S, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, Y281S, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, S306P, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, S306P, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, S306P, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, Y281S, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, 1262K, H275Y, Y281S, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, Y281S, S306P, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, 1262K, H275Y, Y281S, S306P, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, 1262K, H275Y, Y281S, S306P, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, I262K, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, I262K, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, S306P, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, S306P, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, 1262K, H275Y, S306P, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, S306P, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, I262K, H275Y, S306P, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, I262K, H275Y, S306P, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, H197D, N219G, K229E, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, H197D, N219G, K229E, E241D, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, H197D, N219G, K229E, I262K, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, H197D, N219G, K229E, E241D, I262K, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, S306P, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, S306P, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, S306P, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, S306P, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, H197D, N219G, K229E, I262K, H275Y, S306P, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, H197D, N219G, K229E, E241D, I262K, H275Y, S306P, K324I, F333E, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, G199P, N219G, K229E, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, H174E, G199P, N219G, K229E, E241D, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, D139E, K150E, L153P, N162E, H174E, G199P, N219G, K229E, E241D, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, K150E, L153P, N162E, H174E, G199P, N219G, K229E, H275Y, K324I, F333N, A343K, R360L, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D139E, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, E241D, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K324I, F333N, A343K, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, R282G, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H197D, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, K324I, F333N, R360L, 177_183KEYQSNKdelinsRDNRDN (SEQ ID NOS: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, I262K, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, G199P, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, Y281S, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of K93D, S128A, L153P, N162E, N219G, K229E, H275Y, K324I, F333N, 177_183KEYQSNKdelinsRDNRDN (SEQ ID NOS: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219G, K229E, H275Y, S306P, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, K150E, L153P, N162E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, K196S, N219G, K229E, H275Y, K318A, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, H222Q, K229E, H275Y, R280E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, K196S, N219G, H222Q, K229E, H275Y, R280E, K318A, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, D332K, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, N233G, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, I254R, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, E363K, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, E366K, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, E347N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, K196S, N219G, K229E, H275Y, K318A, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, N219G, H222Q, K229E, H275Y, R280E, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of T122S, S128A, L153P, N162E, H174E, K196S, N219G, H222Q, K229E, H275Y, R280E, K318A, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333N, E363K, E366K, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, D332K, F333N, E347N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, I254R, H275Y, K324I, D332K, F333N, E347N, E363K, E366K, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of C125S, S128A, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, D152N, L153P, N162E, H174E, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, H275Y, K324I, F333E, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, H197D, N219G, K229E, H275Y, K324I, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, H174E, N219G, K229E, N233G, H275Y, K324I, D332K, F333N, 177_183KEYQSNKdelinsRDNRDN (SEQ ID NOs: 920 and 919), wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, R280T, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, provided that the amino acid sequence comprises mutations of S128A, L153P, N162E, N219Q, K229E, R280T, D307N, K324I, F333N, wherein the positions correspond to SEQ ID NO: 1, and provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1.

In some embodiments, the polypeptide having protease activity comprises an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one sequence provided in Table 2.

TABLE 2

| | Polypeptide having IgE |
|---|---|
| ID | protease activity Seq |
| PRT-1 | SEQ ID NO: 3 |
| PRT-2 | SEQ ID NO: 4 |
| PRT-3 | SEQ ID NO: 5 |
| PRT-4 | SEQ ID NO: 6 |
| PRT-5 | SEQ ID NO: 7 |
| PRT-6 | SEQ ID NO: 8 |
| PRT-7 | SEQ ID NO: 9 |
| PRT-8 | SEQ ID NO: 10 |
| PRT-9 | SEQ ID NO: 11 |
| PRT-10 | SEQ ID NO: 12 |
| PRT-11 | SEQ ID NO: 13 |
| PRT-12 | SEQ ID NO: 14 |
| PRT-13 | SEQ ID NO: 15 |
| PRT-14 | SEQ ID NO: 16 |
| PRT-15 | SEQ ID NO: 17 |
| PRT-16 | SEQ ID NO: 18 |
| PRT-17 | SEQ ID NO: 19 |
| PRT-18 | SEQ ID NO: 20 |
| PRT-19 | SEQ ID NO: 21 |
| PRT-20 | SEQ ID NO: 22 |
| PRT-21 | SEQ ID NO: 23 |
| PRT-22 | SEQ ID NO: 24 |
| PRT-23 | SEQ ID NO: 25 |
| PRT-24 | SEQ ID NO: 26 |
| PRT-25 | SEQ ID NO: 27 |
| PRT-26 | SEQ ID NO: 28 |
| PRT-27 | SEQ ID NO: 29 |
| PRT-28 | SEQ ID NO: 30 |
| PRT-29 | SEQ ID NO: 31 |
| PRT-30 | SEQ ID NO: 32 |
| PRT-31 | SEQ ID NO: 33 |
| PRT-32 | SEQ ID NO: 34 |
| PRT-33 | SEQ ID NO: 35 |
| PRT-34 | SEQ ID NO: 36 |
| PRT-35 | SEQ ID NO: 37 |
| PRT-36 | SEQ ID NO: 38 |
| PRT-37 | SEQ ID NO: 39 |
| PRT-38 | SEQ ID NO: 40 |
| PRT-39 | SEQ ID NO: 41 |
| PRT-40 | SEQ ID NO: 42 |
| PRT-41 | SEQ ID NO: 43 |
| PRT-42 | SEQ ID NO: 44 |
| PRT-43 | SEQ ID NO: 45 |
| PRT-44 | SEQ ID NO: 46 |
| PRT-45 | SEQ ID NO: 47 |
| PRT-46 | SEQ ID NO: 48 |
| PRT-47 | SEQ ID NO: 49 |
| PRT-48 | SEQ ID NO: 50 |
| PRT-49 | SEQ ID NO: 51 |
| PRT-50 | SEQ ID NO: 52 |
| PRT-51 | SEQ ID NO: 53 |
| PRT-52 | SEQ ID NO: 54 |
| PRT-53 | SEQ ID NO: 55 |
| PRT-54 | SEQ ID NO: 56 |
| PRT-55 | SEQ ID NO: 57 |
| PRT-56 | SEQ ID NO: 58 |
| PRT-57 | SEQ ID NO: 59 |
| PRT-58 | SEQ ID NO: 60 |
| PRT-59 | SEQ ID NO: 61 |
| PRT-60 | SEQ ID NO: 62 |
| PRT-61 | SEQ ID NO: 63 |
| PRT-62 | SEQ ID NO: 64 |
| PRT-63 | SEQ ID NO: 65 |
| PRT-64 | SEQ ID NO: 66 |
| PRT-65 | SEQ ID NO: 67 |
| PRT-66 | SEQ ID NO: 68 |
| PRT-67 | SEQ ID NO: 69 |
| PRT-68 | SEQ ID NO: 70 |
| PRT-69 | SEQ ID NO: 71 |
| PRT-70 | SEQ ID NO: 72 |
| PRT-71 | SEQ ID NO: 73 |
| PRT-72 | SEQ ID NO: 74 |
| PRT-73 | SEQ ID NO: 75 |
| PRT-74 | SEQ ID NO: 76 |
| PRT-75 | SEQ ID NO: 77 |
| PRT-76 | SEQ ID NO: 78 |
| PRT-77 | SEQ ID NO: 79 |
| PRT-78 | SEQ ID NO: 80 |
| PRT-79 | SEQ ID NO: 81 |
| PRT-80 | SEQ ID NO: 82 |
| PRT-81 | SEQ ID NO: 83 |
| PRT-82 | SEQ ID NO: 84 |
| PRT-83 | SEQ ID NO: 85 |
| PRT-84 | SEQ ID NO: 86 |
| PRT-85 | SEQ ID NO: 87 |
| PRT-86 | SEQ ID NO: 88 |
| PRT-87 | SEQ ID NO: 89 |
| PRT-88 | SEQ ID NO: 90 |
| PRT-89 | SEQ ID NO: 91 |
| PRT-90 | SEQ ID NO: 92 |
| PRT-91 | SEQ ID NO: 93 |
| PRT-94 | SEQ ID NO: 96 |
| PRT-95 | SEQ ID NO: 97 |
| PRT-96 | SEQ ID NO: 98 |
| PRT-97 | SEQ ID NO: 99 |
| PRT-98 | SEQ ID NO: 100 |
| PRT-99 | SEQ ID NO: 101 |
| PRT-100 | SEQ ID NO: 102 |
| PRT-101 | SEQ ID NO: 103 |
| PRT-102 | SEQ ID NO: 104 |
| PRT-103 | SEQ ID NO: 105 |
| PRT-104 | SEQ ID NO: 106 |
| PRT-105 | SEQ ID NO: 107 |
| PRT-106 | SEQ ID NO: 108 |
| PRT-107 | SEQ ID NO: 109 |
| PRT-108 | SEQ ID NO: 110 |
| PRT-109 | SEQ ID NO: 111 |
| PRT-110 | SEQ ID NO: 112 |
| PRT-111 | SEQ ID NO: 113 |
| PRT-112 | SEQ ID NO: 114 |
| PRT-113 | SEQ ID NO: 115 |
| PRT-114 | SEQ ID NO: 116 |
| PRT-115 | SEQ ID NO: 117 |
| PRT-116 | SEQ ID NO: 118 |
| PRT-117 | SEQ ID NO: 119 |
| PRT-118 | SEQ ID NO: 120 |
| PRT-119 | SEQ ID NO: 121 |
| PRT-120 | SEQ ID NO: 122 |
| PRT-121 | SEQ ID NO: 123 |
| PRT-122 | SEQ ID NO: 124 |
| PRT-123 | SEQ ID NO: 125 |
| PRT-124 | SEQ ID NO: 126 |
| PRT-125 | SEQ ID NO: 127 |
| PRT-126 | SEQ ID NO: 128 |
| PRT-127 | SEQ ID NO: 129 |
| PRT-128 | SEQ ID NO: 130 |
| PRT-129 | SEQ ID NO: 131 |
| PRT-130 | SEQ ID NO: 132 |
| PRT-131 | SEQ ID NO: 133 |
| PRT-132 | SEQ ID NO: 134 |
| PRT-133 | SEQ ID NO: 135 |
| PRT-134 | SEQ ID NO: 136 |
| PRT-135 | SEQ ID NO: 137 |
| PRT-136 | SEQ ID NO: 138 |
| PRT-137 | SEQ ID NO: 139 |
| PRT-138 | SEQ ID NO: 140 |
| PRT-139 | SEQ ID NO: 141 |
| PRT-140 | SEQ ID NO: 142 |
| PRT-141 | SEQ ID NO: 143 |
| PRT-142 | SEQ ID NO: 144 |
| PRT-143 | SEQ ID NO: 145 |
| PRT-144 | SEQ ID NO: 146 |
| PRT-145 | SEQ ID NO: 147 |
| PRT-146 | SEQ ID NO: 148 |
| PRT-147 | SEQ ID NO: 149 |
| PRT-148 | SEQ ID NO: 150 |
| PRT-149 | SEQ ID NO: 151 |
| PRT-150 | SEQ ID NO: 152 |
| PRT-151 | SEQ ID NO: 153 |
| PRT-152 | SEQ ID NO: 154 |
| PRT-153 | SEQ ID NO: 155 |
| PRT-154 | SEQ ID NO: 156 |
| PRT-155 | SEQ ID NO: 157 |
| PRT-156 | SEQ ID NO: 158 |
| PRT-157 | SEQ ID NO: 159 |
| PRT-158 | SEQ ID NO: 160 |
| PRT-159 | SEQ ID NO: 161 |
| PRT-160 | SEQ ID NO: 162 |
| PRT-161 | SEQ ID NO: 163 |
| PRT-162 | SEQ ID NO: 164 |
| PRT-163 | SEQ ID NO: 165 |
| PRT-164 | SEQ ID NO: 166 |
| PRT-165 | SEQ ID NO: 167 |
| PRT-166 | SEQ ID NO: 168 |
| PRT-167 | SEQ ID NO: 169 |
| PRT-168 | SEQ ID NO: 170 |
| PRT-169 | SEQ ID NO: 171 |
| PRT-170 | SEQ ID NO: 172 |
| PRT-171 | SEQ ID NO: 173 |
| PRT-172 | SEQ ID NO: 174 |
| PRT-173 | SEQ ID NO: 175 |
| PRT-174 | SEQ ID NO: 176 |
| PRT-175 | SEQ ID NO: 177 |
| PRT-176 | SEQ ID NO: 178 |
| PRT-177 | SEQ ID NO: 179 |
| PRT-178 | SEQ ID NO: 180 |
| PRT-179 | SEQ ID NO: 181 |
| PRT-180 | SEQ ID NO: 182 |
| PRT-181 | SEQ ID NO: 183 |
| PRT-182 | SEQ ID NO: 184 |
| PRT-183 | SEQ ID NO: 185 |
| PRT-184 | SEQ ID NO: 186 |
| PRT-185 | SEQ ID NO: 187 |
| PRT-186 | SEQ ID NO: 188 |
| PRT-187 | SEQ ID NO: 189 |
| PRT-188 | SEQ ID NO: 190 |
| PRT-189 | SEQ ID NO: 191 |
| PRT-190 | SEQ ID NO: 192 |
| PRT-191 | SEQ ID NO: 193 |
| PRT-192 | SEQ ID NO: 194 |
| PRT-193 | SEQ ID NO: 195 |
| PRT-194 | SEQ ID NO: 196 |
| PRT-195 | SEQ ID NO: 197 |
| PRT-196 | SEQ ID NO: 198 |
| PRT-197 | SEQ ID NO: 199 |
| PRT-198 | SEQ ID NO: 200 |
| PRT-199 | SEQ ID NO: 201 |
| PRT-200 | SEQ ID NO: 202 |
| PRT-201 | SEQ ID NO: 203 |
| PRT-202 | SEQ ID NO: 204 |
| PRT-203 | SEQ ID NO: 205 |
| PRT-204 | SEQ ID NO: 206 |
| PRT-205 | SEQ ID NO: 207 |
| PRT-206 | SEQ ID NO: 208 |
| PRT-207 | SEQ ID NO: 209 |
| PRT-208 | SEQ ID NO: 210 |
| PRT-209 | SEQ ID NO: 211 |
| PRT-210 | SEQ ID NO: 212 |
| PRT-211 | SEQ ID NO: 213 |
| PRT-212 | SEQ ID NO: 214 |
| PRT-213 | SEQ ID NO: 215 |
| PRT-214 | SEQ ID NO: 216 |
| PRT-215 | SEQ ID NO: 217 |
| PRT-216 | SEQ ID NO: 218 |
| PRT-217 | SEQ ID NO: 219 |
| PRT-218 | SEQ ID NO: 220 |
| PRT-219 | SEQ ID NO: 221 |
| PRT-220 | SEQ ID NO: 222 |
| PRT-221 | SEQ ID NO: 223 |
| PRT-222 | SEQ ID NO: 224 |
| PRT-223 | SEQ ID NO: 225 |
| PRT-224 | SEQ ID NO: 226 |
| PRT-225 | SEQ ID NO: 227 |
| PRT-226 | SEQ ID NO: 228 |
| PRT-227 | SEQ ID NO: 229 |
| PRT-228 | SEQ ID NO: 230 |
| PRT-229 | SEQ ID NO: 231 |
| PRT-230 | SEQ ID NO: 232 |
| PRT-231 | SEQ ID NO: 233 |
| PRT-232 | SEQ ID NO: 234 |
| PRT-233 | SEQ ID NO: 235 |
| PRT-234 | SEQ ID NO: 236 |
| PRT-235 | SEQ ID NO: 237 |
| PRT-236 | SEQ ID NO: 238 |
| PRT-237 | SEQ ID NO: 239 |
| PRT-238 | SEQ ID NO: 240 |
| PRT-239 | SEQ ID NO: 241 |
| PRT-240 | SEQ ID NO: 242 |
| PRT-241 | SEQ ID NO: 243 |
| PRT-242 | SEQ ID NO: 247 |
| PRT-243 | SEQ ID NO: 251 |
| PRT-244 | SEQ ID NO: 280 |
| PRT-245 | SEQ ID NO: 281 |
| PRT-246 | SEQ ID NO: 282 |
| PRT-247 | SEQ ID NO: 283 |
| PRT-248 | SEQ ID NO: 284 |
| PRT-249 | SEQ ID NO: 285 |
| PRT-250 | SEQ ID NO: 286 |
| PRT-251 | SEQ ID NO: 287 |
| PRT-252 | SEQ ID NO: 288 |
| PRT-253 | SEQ ID NO: 289 |
| PRT-254 | SEQ ID NO: 290 |
| PRT-255 | SEQ ID NO: 291 |
| PRT-256 | SEQ ID NO: 292 |
| PRT-257 | SEQ ID NO: 293 |
| PRT-258 | SEQ ID NO: 294 |
| PRT-259 | SEQ ID NO: 295 |
| PRT-260 | SEQ ID NO: 296 |
| PRT-261 | SEQ ID NO: 297 |
| PRT-262 | SEQ ID NO: 298 |
| PRT-263 | SEQ ID NO: 299 |
| PRT-264 | SEQ ID NO: 300 |
| PRT-265 | SEQ ID NO: 301 |
| PRT-266 | SEQ ID NO: 302 |
| PRT-267 | SEQ ID NO: 303 |
| PRT-268 | SEQ ID NO: 304 |
| PRT-269 | SEQ ID NO: 305 |
| PRT-270 | SEQ ID NO: 306 |
| PRT-271 | SEQ ID NO: 307 |
| PRT-272 | SEQ ID NO: 308 |
| PRT-273 | SEQ ID NO: 309 |
| PRT-274 | SEQ ID NO: 310 |
| PRT-275 | SEQ ID NO: 311 |
| PRT-276 | SEQ ID NO: 312 |
| PRT-277 | SEQ ID NO: 313 |
| PRT-278 | SEQ ID NO: 314 |
| PRT-279 | SEQ ID NO: 315 |
| PRT-280 | SEQ ID NO: 316 |
| PRT-281 | SEQ ID NO: 317 |
| PRT-282 | SEQ ID NO: 318 |
| PRT-283 | SEQ ID NO: 319 |
| PRT-284 | SEQ ID NO: 320 |
| PRT-285 | SEQ ID NO: 321 |
| PRT-286 | SEQ ID NO: 322 |
| PRT-287 | SEQ ID NO: 323 |
| PRT-288 | SEQ ID NO: 324 |
| PRT-289 | SEQ ID NO: 325 |
| PRT-290 | SEQ ID NO: 326 |
| PRT-291 | SEQ ID NO: 327 |
| PRT-292 | SEQ ID NO: 328 |
| PRT-293 | SEQ ID NO: 329 |
| PRT-294 | SEQ ID NO: 330 |
| PRT-295 | SEQ ID NO: 331 |
| PRT-296 | SEQ ID NO: 332 |
| PRT-297 | SEQ ID NO: 333 |
| PRT-298 | SEQ ID NO: 334 |
| PRT-299 | SEQ ID NO: 335 |
| PRT-300 | SEQ ID NO: 336 |
| PRT-301 | SEQ ID NO: 337 |
| PRT-302 | SEQ ID NO: 338 |
| PRT-303 | SEQ ID NO: 339 |
| PRT-304 | SEQ ID NO: 340 |
| PRT-305 | SEQ ID NO: 341 |
| PRT-306 | SEQ ID NO: 342 |
| PRT-307 | SEQ ID NO: 343 |
| PRT-308 | SEQ ID NO: 344 |
| PRT-309 | SEQ ID NO: 345 |
| PRT-310 | SEQ ID NO: 346 |
| PRT-311 | SEQ ID NO: 347 |
| PRT-312 | SEQ ID NO: 348 |
| PRT-313 | SEQ ID NO: 349 |
| PRT-314 | SEQ ID NO: 350 |
| PRT-315 | SEQ ID NO: 351 |
| PRT-316 | SEQ ID NO: 352 |
| PRT-317 | SEQ ID NO: 353 |
| PRT-318 | SEQ ID NO: 354 |
| PRT-319 | SEQ ID NO: 355 |
| PRT-320 | SEQ ID NO: 356 |
| PRT-321 | SEQ ID NO: 357 |
| PRT-322 | SEQ ID NO: 358 |
| PRT-323 | SEQ ID NO: 359 |
| PRT-324 | SEQ ID NO: 360 |
| PRT-325 | SEQ ID NO: 361 |
| PRT-326 | SEQ ID NO: 362 |
| PRT-327 | SEQ ID NO: 363 |
| PRT-328 | SEQ ID NO: 364 |
| PRT-329 | SEQ ID NO: 365 |
| PRT-330 | SEQ ID NO: 366 |
| PRT-331 | SEQ ID NO: 367 |
| PRT-332 | SEQ ID NO: 368 |
| PRT-333 | SEQ ID NO: 369 |
| PRT-334 | SEQ ID NO: 370 |
| PRT-335 | SEQ ID NO: 371 |
| PRT-336 | SEQ ID NO: 372 |
| PRT-337 | SEQ ID NO: 373 |
| PRT-338 | SEQ ID NO: 374 |
| PRT-339 | SEQ ID NO: 375 |
| PRT-340 | SEQ ID NO: 376 |
| PRT-341 | SEQ ID NO: 377 |
| PRT-342 | SEQ ID NO: 378 |
| PRT-343 | SEQ ID NO: 379 |
| PRT-344 | SEQ ID NO: 380 |
| PRT-345 | SEQ ID NO: 381 |
| PRT-346 | SEQ ID NO: 382 |
| PRT-347 | SEQ ID NO: 383 |
| PRT-348 | SEQ ID NO: 384 |
| PRT-349 | SEQ ID NO: 385 |
| PRT-350 | SEQ ID NO: 386 |
| PRT-351 | SEQ ID NO: 387 |
| PRT-352 | SEQ ID NO: 388 |
| PRT-353 | SEQ ID NO: 389 |
| PRT-354 | SEQ ID NO: 390 |
| PRT-355 | SEQ ID NO: 391 |
| PRT-356 | SEQ ID NO: 392 |
| PRT-357 | SEQ ID NO: 393 |
| PRT-358 | SEQ ID NO: 394 |
| PRT-359 | SEQ ID NO: 395 |
| PRT-360 | SEQ ID NO: 396 |
| PRT-361 | SEQ ID NO: 397 |
| PRT-362 | SEQ ID NO: 398 |
| PRT-363 | SEQ ID NO: 399 |
| PRT-364 | SEQ ID NO: 400 |
| PRT-365 | SEQ ID NO: 401 |
| PRT-366 | SEQ ID NO: 402 |
| PRT-367 | SEQ ID NO: 403 |
| PRT-368 | SEQ ID NO: 404 |
| PRT-369 | SEQ ID NO: 405 |
| PRT-370 | SEQ ID NO: 406 |
| PRT-371 | SEQ ID NO: 407 |
| PRT-372 | SEQ ID NO: 408 |
| PRT-373 | SEQ ID NO: 409 |
| PRT-374 | SEQ ID NO: 410 |
| PRT-375 | SEQ ID NO: 411 |
| PRT-376 | SEQ ID NO: 412 |
| PRT-377 | SEQ ID NO: 413 |
| PRT-378 | SEQ ID NO: 414 |
| PRT-379 | SEQ ID NO: 415 |
| PRT-380 | SEQ ID NO: 416 |
| PRT-381 | SEQ ID NO: 417 |
| PRT-382 | SEQ ID NO: 418 |
| PRT-383 | SEQ ID NO: 419 |
| PRT-384 | SEQ ID NO: 420 |
| PRT-385 | SEQ ID NO: 421 |
| PRT-386 | SEQ ID NO: 422 |
| PRT-387 | SEQ ID NO: 423 |
| PRT-388 | SEQ ID NO: 424 |
| PRT-389 | SEQ ID NO: 425 |
| PRT-390 | SEQ ID NO: 426 |
| PRT-391 | SEQ ID NO: 427 |
| PRT-392 | SEQ ID NO: 428 |
| PRT-393 | SEQ ID NO: 429 |
| PRT-394 | SEQ ID NO: 430 |
| PRT-395 | SEQ ID NO: 431 |
| PRT-396 | SEQ ID NO: 432 |
| PRT-397 | SEQ ID NO: 433 |
| PRT-398 | SEQ ID NO: 434 |
| PRT-399 | SEQ ID NO: 435 |
| PRT-400 | SEQ ID NO: 436 |
| PRT-401 | SEQ ID NO: 437 |
| PRT-402 | SEQ ID NO: 438 |
| PRT-403 | SEQ ID NO: 439 |
| PRT-404 | SEQ ID NO: 440 |
| PRT-405 | SEQ ID NO: 441 |
| PRT-406 | SEQ ID NO: 442 |
| PRT-407 | SEQ ID NO: 443 |
| PRT-408 | SEQ ID NO: 444 |
| PRT-409 | SEQ ID NO: 445 |
| PRT-410 | SEQ ID NO: 446 |
| PRT-411 | SEQ ID NO: 447 |
| PRT-412 | SEQ ID NO: 448 |
| PRT-413 | SEQ ID NO: 449 |
| PRT-414 | SEQ ID NO: 450 |
| PRT-415 | SEQ ID NO: 451 |
| PRT-416 | SEQ ID NO: 452 |
| PRT-417 | SEQ ID NO: 453 |
| PRT-418 | SEQ ID NO: 454 |
| PRT-419 | SEQ ID NO: 455 |
| PRT-420 | SEQ ID NO: 456 |
| PRT-421 | SEQ ID NO: 457 |
| PRT-422 | SEQ ID NO: 458 |
| PRT-423 | SEQ ID NO: 459 |
| PRT-424 | SEQ ID NO: 460 |
| PRT-425 | SEQ ID NO: 461 |
| PRT-426 | SEQ ID NO: 462 |
| PRT-427 | SEQ ID NO: 463 |
| PRT-428 | SEQ ID NO: 464 |
| PRT-429 | SEQ ID NO: 465 |
| PRT-430 | SEQ ID NO: 466 |
| PRT-431 | SEQ ID NO: 467 |
| PRT-432 | SEQ ID NO: 468 |
| PRT-433 | SEQ ID NO: 469 |
| PRT-434 | SEQ ID NO: 470 |
| PRT-435 | SEQ ID NO: 471 |
| PRT-436 | SEQ ID NO: 472 |
| PRT-437 | SEQ ID NO: 473 |
| PRT-438 | SEQ ID NO: 474 |
| PRT-439 | SEQ ID NO: 475 |
| PRT-440 | SEQ ID NO: 476 |
| PRT-441 | SEQ ID NO: 477 |
| PRT-442 | SEQ ID NO: 478 |
| PRT-443 | SEQ ID NO: 479 |
| PRT-444 | SEQ ID NO: 480 |
| PRT-445 | SEQ ID NO: 481 |
| PRT-446 | SEQ ID NO: 482 |
| PRT-447 | SEQ ID NO: 483 |
| PRT-448 | SEQ ID NO: 484 |
| PRT-449 | SEQ ID NO: 485 |
| PRT-450 | SEQ ID NO: 486 |
| PRT-451 | SEQ ID NO: 487 |
| PRT-452 | SEQ ID NO: 488 |
| PRT-453 | SEQ ID NO: 489 |
| PRT-454 | SEQ ID NO: 490 |
| PRT-455 | SEQ ID NO: 491 |
| PRT-456 | SEQ ID NO: 492 |
| PRT-457 | SEQ ID NO: 493 |
| PRT-458 | SEQ ID NO: 494 |
| PRT-459 | SEQ ID NO: 495 |
| PRT-460 | SEQ ID NO: 496 |
| PRT-461 | SEQ ID NO: 497 |
| PRT-462 | SEQ ID NO: 498 |
| PRT-463 | SEQ ID NO: 499 |
| PRT-464 | SEQ ID NO: 500 |
| PRT-465 | SEQ ID NO: 501 |
| PRT-466 | SEQ ID NO: 502 |
| PRT-467 | SEQ ID NO: 503 |
| PRT-468 | SEQ ID NO: 504 |
| PRT-469 | SEQ ID NO: 505 |
| PRT-470 | SEQ ID NO: 506 |
| PRT-471 | SEQ ID NO: 507 |
| PRT-472 | SEQ ID NO: 508 |
| PRT-473 | SEQ ID NO: 509 |
| PRT-474 | SEQ ID NO: 510 |
| PRT-475 | SEQ ID NO: 511 |
| PRT-476 | SEQ ID NO: 512 |
| PRT-477 | SEQ ID NO: 513 |
| PRT-478 | SEQ ID NO: 514 |
| PRT-479 | SEQ ID NO: 515 |
| PRT-480 | SEQ ID NO: 516 |
| PRT-481 | SEQ ID NO: 517 |
| PRT-482 | SEQ ID NO: 518 |
| PRT-483 | SEQ ID NO: 519 |
| PRT-484 | SEQ ID NO: 520 |
| PRT-485 | SEQ ID NO: 521 |
| PRT-486 | SEQ ID NO: 522 |
| PRT-487 | SEQ ID NO: 523 |
| PRT-488 | SEQ ID NO: 524 |
| PRT-489 | SEQ ID NO: 525 |
| PRT-490 | SEQ ID NO: 526 |
| PRT-491 | SEQ ID NO: 527 |
| PRT-492 | SEQ ID NO: 528 |
| PRT-493 | SEQ ID NO: 737 |

X in the sequences provided in the rows above may be methionine M, a signal peptide, or absent. The signal sequence may be, for example, the amino acid sequence of METDTLLLWVLLLWVPGSTG (SEQ ID NO: 244).

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 380, 251, 5-243, 247, 280-379, 381-528, 737, 745-823, 828-864, or 885-916. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 380, 251, 5-243, 247, 280-379, 381-528, 737, 745-823, 828-864, or 885-916, wherein X comprises a Methionine (M), a signal peptide, or is absent. In some embodiments, the signal peptide comprises an amino acid sequence of METDTLLLWVLLLWVPGSTG (SEQ ID NO: 244).

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 380, 251, 5-243, 247, 280-379, 381-528, 737, 745-823, 828-864, or 885-916, wherein X comprises a Methionine (M).

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 3-243, 247, 251, 280-373, 382-528, or 737, wherein X comprises a signal peptide having an amino acid sequence of SEQ ID NO: 244.

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 3-243, 247, 251, 280-373, 382-528, or 737, wherein X is absent.

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 3-243, 247, 251, 280-373, 382-528, or 737, wherein X comprises a Methionine (M), a signal peptide, or is absent, and wherein the polypeptide further comprises a His-tag at the C-terminal of the polypeptide. In some embodiments, the His-tag comprises an amino acid sequence of LEHHHHHH (SEQ ID NO: 245). In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 3-243, 247, 251, 280-373, 382-528, or 737, wherein X comprises a Methionine (M), a signal peptide, or is absent, and wherein the polypeptide further comprises a His-tag having the amino acid sequence of SEQ ID NO: 245 at the C-terminal of the polypeptide.

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 380, 251, 5-243, 247, 280-379, 381-528, 737, 745-823, 828-864, or 885-916.

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 4. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 5. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 6. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 7. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 8. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 9. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 10. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 11. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 12. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 13. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 14. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 15. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 16. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 17. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 18. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 19. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 20. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 21. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 22. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 23. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 24. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 25. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 26. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 27. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 28. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 29. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 30. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 31. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 32. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 33. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 34. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 35. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 36. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 37. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 38. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 39. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 40. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 41. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 42. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 43. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 44. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 45. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 46. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 47. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 48. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 49. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 50. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 51. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 52. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 53. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 54. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 55. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 56. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 57. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 58. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 59. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 60. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 61. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 62. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 63. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 64. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 65. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 66. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 67. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 68. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 69. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 70. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 71. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 72. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 73. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 74. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 75. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 76. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 77. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 78. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 79. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 80. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 81. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 82. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 83. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 84. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 85. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 86. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 87. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 88. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 89. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 90. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 91. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 92. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 93. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 96. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 97. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 98. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 99. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 100. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 101. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 102. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 103. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 104. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 105. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 106. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 107. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 108. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 109. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 110. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 111. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 112. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 113. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 114. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 115. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 116. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 117. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 118. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 119. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 120. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 121. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 122. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 123. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 124. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 125. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 126. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 127. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 128. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 129. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 130. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 131. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 132. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 133. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 134. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 135. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 136. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 137. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 138. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 139. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 140. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 141. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 142. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 143. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 144. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 145. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 146. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 147. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 148. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 149. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 150. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 151. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 152. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 153. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 154. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 155. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 156. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 157. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 158. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 159. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 160. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 161. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 162. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 163. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 164. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 165. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 166. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 167. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 168. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 169. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 170. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 171. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 172. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 173. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 174. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 175. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 176. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 177. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 178. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 179. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 180. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 181. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 182. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 183. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 184. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 185. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 186. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 187. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 188. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 189. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 190. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 191. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 192. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 193. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 194. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 195. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 196. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 197. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 198. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 199. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 200. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 201. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 202. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 203. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 204. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 205. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 206. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 207. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 208. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 209. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 210. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 211. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 212. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 213. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 214. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 215. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 216. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 217. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 218. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 219. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 220. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 221. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 222. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 223. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 224. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 225. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 226. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 227. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 228. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 229. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 230. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 231. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 232. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 233. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 234. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 235. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 236. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 237. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 238. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 239. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 240. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 241. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 242. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 243. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 247. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 251. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 280. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 281. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 282. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 283. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 284. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 285. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 286. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 287. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 288. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 289. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 290. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 291. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 292. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 293. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 294. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 295. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 296. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 297. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 298. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 299. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 300. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 301. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 302. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 303. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 304. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 305. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 306. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 307. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 308. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 309. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 310. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 311. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 312. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 313. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 314. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 315. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 316. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 317. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 318. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 319. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 320. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 321. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 322. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 323. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 324. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 325. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 326. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 327. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 328. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 329. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 330. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 331. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 332. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 333. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 334. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 335. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 336. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 337. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 338. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 339. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 340. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 341. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 342. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 343. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 344. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 345. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 346. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 347. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 348. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 349. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 350. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 351. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 352. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 353. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 354. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 355. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 356. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 357. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 358. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 359. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 360. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 361. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 362. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 363. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 364. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 365. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 366. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 367. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 368. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 369. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 370. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 371. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 372. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 373. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 374. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 375. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 376. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 377. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 378. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 379. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 380. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 381. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 382. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 383. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 384. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 385. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 386. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 387. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 388. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 389. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 390. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 391. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 392. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 393. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 394. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 395. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 396. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 397. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 398. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 399. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 400. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 401. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 402. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 403. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 404. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 405. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 406. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 407. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 408. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 409. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 410. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 411. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 412. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 413. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 414. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 415. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 416. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 417. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 418. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 419. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 420. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 421. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 422. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 423. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 424. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 425. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 426. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 427. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 428. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 429. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 430. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 431. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 432. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 433. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 434. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 435. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 436. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 437. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 438. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 439. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 440. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 441. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 442. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 443. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 444. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 445. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 446. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 447. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 448. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 449. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 450. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 451. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 452. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 453. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 454. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 455. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 456. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 457. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 458. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 459. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 460. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 461. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 462. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 463. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 464. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 465. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 466. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 467. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 468. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 469. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 470. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 471. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 472. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 473. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 474. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 475. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 476. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 477. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 478. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 479. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 480. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 481. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 482. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 483. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 484. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 485. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 486. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 487. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 488. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 489. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 490. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 491. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 492. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 493. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 494. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 495. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 496. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 497. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 498. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 499. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 500. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 501. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 502. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 503. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 504. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 505. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 506. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 507. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 508. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 509. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 510. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 511. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 512. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 513. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 514. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 515. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 516. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 517. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 518. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 519. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 520. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 521. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 522. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 523. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 524. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 525. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 526. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 527. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 528. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 737. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 745. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 746. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 747. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 748. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 749. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 750. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 751. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 752. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 753. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 754. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 755. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 756. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 757. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 758. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 759. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 760. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 761. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 762. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 763. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 764. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 765. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 766. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 767. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 768. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 769. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 770. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 771. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 772. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 773. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 774. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 775. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 776. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 777. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 778. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 779. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 780. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 781. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 782. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 783. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 784. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 785. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 786. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 787. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 788. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 789. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 790. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 791. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 792. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 793. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 794. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 795. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 796. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 797. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 798. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 799. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 800. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 801. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 802. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 803. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 804. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 805. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 806. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 807. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 808. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 809. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 810. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 811. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 812. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 813. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 814. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 815. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 816. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 817. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 818. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 819. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 820. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 821. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 822. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 823. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 828. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 829. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 830. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 831. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 832. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 833. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 834. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 835. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 836. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 837. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 838. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 839. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 840. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 841. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 842. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 843. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 844. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 845. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 846. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 847. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 848. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 849. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 850. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 851. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 852. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 853. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 854. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 855. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 856. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 857. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 858. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 859. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 860. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 861. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 862. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 863. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 864. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 885. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 886. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 887. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 888. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 889. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 890. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 891. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 892. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 893. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 894. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 895. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 896. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 897. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 898. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 899. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 900. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 901. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 902. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 903. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 904. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 905. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 906. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 907. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 908. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 909. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 910. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 911. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 912. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 913. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 914. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 915. In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 916.

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 380, 251, 5-243, 247, 280-379, 381-528, 737, 745-823, 828-864, or 885-916, provided that the amino acid sequence comprises any one mutation selected from Table 3A or Table 3B.

TABLE 3A

| ID | Polypeptide Seq | Mutation (s) or deletions (as compared to SEQ ID NO: 1) |
|---|---|---|
| PRT-4 | SEQ ID NO: 3 | 1_61del and 380_529del |
| PRT-2 | SEQ ID NO: 4 | 1_52del and 380_529_del |
| PRT-3 | SEQ ID NO: 5 | 1_61del and 380_529del; L153P, R189K, S128A |
| PRT-4 | SEQ ID NO: 6 | 1_61del and 380_529del; L153P, S128A, F333N |
| PRT-5 | SEQ ID NO: 7 | 1_61del and 380_529del; T122S, K229E |
| PRT-6 | SEQ ID NO: 8 | 1_61del and 380_529del; T122S, L153P |
| PRT-7 | SEQ ID NO: 9 | 1_61del and 380_529del; T122S, L153P, R189K |
| PRT-8 | SEQ ID NO: 10 | 1_61del and 380_529del; T122S, L153P, S128A |
| PRT-9 | SEQ ID NO: 11 | 1_61del and 380_529del; T122S, L153P, VI26S |
| PRT-10 | SEQ ID NO: 12 | 1_61del and 380_529del; T122S, S128A, F333N |
| PRT-11 | SEQ ID NO: 13 | 1_61del and 380_529del; E166Q |
| PRT-12 | SEQ ID NO: 14 | 1_61del and 380_529del; K196S |
| PRT-13 | SEQ ID NO: 15 | 1_61del and 380_529del; K196E |
| PRT-14 | SEQ ID NO: 16 | 1_61del and 380_529del; H222Q |
| PRT-15 | SEQ ID NO: 17 | 1_61del and 380_529del; E120G |
| PRT-16 | SEQ ID NO: 18 | 1_61del and 380_529del; K93S |
| PRT-17 | SEQ ID NO: 19 | 1_61del and 380_529del; T122S |
| PRT-18 | SEQ ID NO: 20 | 1_61del and 380_529del; K229E |
| PRT-19 | SEQ ID NO: 21 | 1_61del and 380_529del; N162E |
| PRT-20 | SEQ ID NO: 22 | 1_61del and 380_529del; R280E |
| PRT-21 | SEQ ID NO: 23 | 1_61del and 380_529del; K318G |
| PRT-22 | SEQ ID NO: 24 | 1_61del and 380_529del; K318A |
| PRT-23 | SEQ ID NO: 25 | 1_52del and 380_529 del; T122S, S128A, L153P |
| PRT-24 | SEQ ID NO: 26 | 1_61del and 380_529del; T122S, S128A, L153P, F333N |
| PRT-25 | SEQ ID NO: 27 | 1_61del and 380_529del; D71E, A79V, T122S, S128A, L153P, |
| | | F333N |
| PRT-26 | SEQ ID NO: 28 | 1_61del and 380_529del; A79V, T122S, S128A, L153P, |
| | | R189K, F333N |
| PRT-27 | SEQ ID NO: 29 | 1_61del and 380_529del; A79V, T122S, S128A, L153P, |
| | | H222Q, F333N |
| PRT-28 | SEQ ID NO: 30 | 1_61del and 380_529del; T122S, S128A, L153P, R189K, |
| | | H222Q, F333N |
| PRT-29 | SEQ ID NO: 31 | 1_61del and 380_529del; A79V, T122S, S128A, L153P, |
| | | A167T, F333N |
| PRT-30 | SEQ ID NO: 32 | 1_61del and 380_529del; A79V, T122S, S128A, L153P, |
| | | R189K, H222Q |
| PRT-31 | SEQ ID NO: 33 | 1_61del and 380_529del; A79V, T122S, S128A, L153P, |
| | | A167T, H222Q |
| PRT-32 | SEQ ID NO: 34 | 1_61del and 380_529del; A79V, V102A, T122S, S128A, |
| | | L153P, A167T |
| PRT-33 | SEQ ID NO: 35 | 1_61del and 380_529del; D71E, A79V, T122S, S128A, |
| | | L153P, A167T |
| PRT-34 | SEQ ID NO: 36 | 1_61del and 380_529del; T122S, S128A, L153P, N162E, |
| | | F333N |
| PRT-35 | SEQ ID NO: 37 | 1_61del and 380_529del; T122S, S128A, L153P, K318A, |
| | | F333N |
| PRT-36 | SEQ ID NO: 38 | 1_61del and 380_529del; T122S, S128A, L153P, H222Q, |
| | | K229E, R280E, F333N |
| PRT-37 | SEQ ID NO: 39 | 1_61del and 380_529del; S128A, L153P, H222Q, K229E, |
| | | F333N |
| PRT-38 | SEQ ID NO: 40 | 1_61del and 380_529del; S128A, L153P, H222Q, R280E, |
| | | F333N |
| PRT-39 | SEQ ID NO: 41 | 1_61del and 380_529del; S128A, L153P, K229E, R280E, |
| | | F333N |
| PRT-40 | SEQ ID NO: 42 | 1_61del and 380_529del; T122S, S128A, L153P, N162E, |
| | | H222Q, K229E, R280E, F333N |
| PRT-41 | SEQ ID NO: 43 | 1_61del and 380_529del; S128A, L153P, N162E, K196S, |
| | | F333N |
| PRT-42 | SEQ ID NO: 44 | 1_61del and 380_529del; S128A, L153P, N162E, H222Q, |
| | | F333N |
| PRT-43 | SEQ ID NO: 45 | 1_61del and 380_529del; S128A, L153P, N162E, K229E, |
| | | F333N |
| PRT-44 | SEQ ID NO: 46 | 1_61del and 380_529del; S128A, L153P, N162E, R280E, |
| | | F333N |
| PRT-45 | SEQ ID NO: 47 | 1_61del and 380_529del; S128A, L153P, N162E, K318A, |
| | | F333N |
| PRT-46 | SEQ ID NO: 48 | 1_61del and 380_529del; S128A, L153P, N162E, K196S, |
| | | K318A, F333N |
| PRT-47 | SEQ ID NO: 49 | 1_61del and 380_529del; S128A, L153P, K196S, K318A, |
| | | F333N |
| PRT-48 | SEQ ID NO: 50 | 1_61del and 380_529del; S128A, L153P, N162E, F333N |
| PRT-49 | SEQ ID NO: 51 | 1_61del and 380_529del; S128A, L153P, K196S, F333N |
| PRT-50 | SEQ ID NO: 52 | 1_61del and 380_529del; S128A, L153P, K318A, F333N |
| PRT-51 | SEQ ID NO: 53 | 1_61del and 380_529del; S128A, L153P, K196S, E310Q, |
| | | K318A, F333N |
| PRT-52 | SEQ ID NO: 54 | 1_61del and 380_529del; S128A, L153P, N162E, K196S, |
| | | E310Q, K318A, F333N |
| PRT-53 | SEQ ID NO: 55 | 1_61del and 380_529del; T122S, S128A, L153P, N162E |
| PRT-54 | SEQ ID NO: 56 | 1_61del and 380_529del; T122S, S128A, L153P, K318A |
| PRT-55 | SEQ ID NO: 57 | 1_61del and 380_529del; T122S, S128A, L153P, H222Q, |
| | | K229E, R280E |
| PRT-56 | SEQ ID NO: 58 | 1_61del and 380_529del; T122S, S128A, L153P, H222Q, |
| | | K229E |
| PRT-57 | SEQ ID NO: 59 | 1_61del and 380_529del; T122S, S128A, L153P, H222Q, |
| | | R280E |
| PRT-58 | SEQ ID NO: 60 | 1_61del and 380_529del; T122S, S128A, L153P, K229E, |
| | | R280E |
| PRT-59 | SEQ ID NO: 61 | 1_61del and 380_529del; T122S, S128A, L153P, N162E, |
| | | H222Q, K229E, R280E |
| PRT-60 | SEQ ID NO: 62 | 1_61del and 380_529del; T122S, S128A, L153P, N162E, |
| | | K196S |
| PRT-61 | SEQ ID NO: 63 | 1_61del and 380_529del; T122S, S128A, L153P, N162E, |
| | | H222Q |
| PRT-62 | SEQ ID NO: 64 | 1_61del and 380_529del; T122S, S128A, L153P, N162E, |
| | | K229E |
| PRT-63 | SEQ ID NO: 65 | 1_61del and 380_529del; T122S, S128A, L153P, N162E, |
| | | R280E |
| PRT-64 | SEQ ID NO: 66 | 1_61del and 380_529del; T122S, S128A, L153P, N162E, |
| | | K318A |
| PRT-65 | SEQ ID NO: 67 | 1_61del and 380_529del; T122S, S128A, L153P, N162E, |
| | | K196S, K318A |
| PRT-66 | SEQ ID NO: 68 | 1_61del and 380_529del; T122S, S128A, L153P, K196S, |
| | | K318A |
| PRT-67 | SEQ ID NO: 69 | 1_61del and 380_529del; T122S, S128A, L153P, K196S |
| PRT-68 | SEQ ID NO: 70 | 1_61del and 380_529del; T122S, S128A, L153P, K196S, |
| | | Y212F, K318A |
| PRT-69 | SEQ ID NO: 71 | 1_61del and 380_529del; T122S, S128A, L153P, K196S, |
| | | E310Q, K318A |
| PRT-70 | SEQ ID NO: 72 | 1_61del and 380_529del; T122S, S128A, L153P, N162E, |
| | | K196S, E310Q, K318A |
| PRT-71 | SEQ ID NO: 73 | 1_61del and 380_529del; S128A, L153P, E166D, F333N |
| PRT-72 | SEQ ID NO: 74 | 1_61del and 380_529del; S128A, L153P, K196T, F333N |
| PRT-73 | SEQ ID NO: 75 | 1_61del and 380_529del; S128A, L153P, K196D, F333N |
| PRT-74 | SEQ ID NO: 76 | 1_61del and 380_529del; S128A, L153P, K196N, F333N |
| PRT-75 | SEQ ID NO: 77 | 1_61del and 380_529del; S128A, L153P, H222S, F333N |
| PRT-76 | SEQ ID NO: 78 | 1_61del and 380_529del; S128A, L153P, H222E, F333N |
| PRT-77 | SEQ ID NO: 79 | 1_61del and 380_529del; S128A, L153P, H222T, F333N |
| PRT-78 | SEQ ID NO: 80 | 1_61del and 380_529del; E120D, S128A, L153P, F333N |
| PRT-79 | SEQ ID NO: 81 | 1_61del and 380_529del; T122E, S128A, L153P, F333N |
| PRT-80 | SEQ ID NO: 82 | 1_61del and 380_529del; T122N, S128A, L153P, F333N |
| PRT-81 | SEQ ID NO: 83 | 1_61del and 380_529del; S128A, L153P, K229D, F333N |
| PRT-82 | SEQ ID NO: 84 | 1_61del and 380_529del; S128A, L153P, K229N, F333N |
| PRT-83 | SEQ ID NO: 85 | 1_61del and 380_529del; S128A, L153P, N162D, F333N |
| PRT-84 | SEQ ID NO: 86 | 1_61DEL AND 380_529del; S128A, L153P, R280D, F333N |
| PRT-85 | SEQ ID NO: 87 | 1_61DEL AND 380_529del; S128A, L153P, R280A, F333N |
| PRT-86 | SEQ ID NO: 88 | 1_61DEL AND 380_529del; S128A, L153P, R280Y, F333N |
| PRT-87 | SEQ ID NO: 89 | 1_61DEL AND 380_529del; S128A, L153P, R280N, F333N |
| PRT-88 | SEQ ID NO: 90 | 1_61DEL AND 380_529del; S128A, L153P, R280T, F333N |
| PRT-89 | SEQ ID NO: 91 | 1_61DEL AND 380_529del; S128A, L153P, K324I, F333N |
| PRT-90 | SEQ ID NO: 92 | 1_61DEL AND 380_529del; S128A, L153P, E310D, F333N |
| PRT-91 | SEQ ID NO: 93 | 1_61DEL AND 380_529del; S128A, L153P, E310N, F333N |
| PRT-94 | SEQ ID NO: 96 | 1_61DEL AND 380_529del; S128A, L153P, E310S, F333N |
| PRT-95 | SEQ ID NO: 97 | 1_61DEL AND 380_529del; S128A, L153P, K318N, F333N |
| PRT-96 | SEQ ID NO: 98 | 1_61DEL AND 380_529del; S128A, L153P, K318E, F333N |
| PRT-97 | SEQ ID NO: 99 | 1_61DEL AND 380_529del; S128A, L153P, K318D, F333N |
| PRT-98 | SEQ ID NO: 100 | 1_61DEL AND 380_529del; S128A, L153P, K318Q, F333N |
| PRT-99 | SEQ ID NO: 101 | 1_61DEL AND 380_529del; S128A, L153P, K318P, F333N |
| PRT-100 | SEQ ID NO: 102 | 1_61DEL AND 380_529del; S128A, L153P, K318F, F333N |
| PRT-101 | SEQ ID NO: 103 | 1_61DEL AND 380_529del; S128A, L153P, K318V, F333N |
| PRT-102 | SEQ ID NO: 104 | 1_61DEL AND 380_529del; S128A, L153P, K318L, F333N |
| PRT-103 | SEQ ID NO: 105 | 1_61DEL AND 380_529del; S128A, L153P, K318S, F333N |
| PRT-104 | SEQ ID NO: 106 | 1_61DEL AND 380_529del; S128A, L153P, K318I, F333N |
| PRT-105 | SEQ ID NO: 107 | 1_61DEL AND 380_529del; S128A, L153P, K201E, F333N |
| PRT-106 | SEQ ID NO: 108 | 1_61DEL AND 380_529del; S128A, L153P, K201S, F333N |
| PRT-107 | SEQ ID NO: 109 | 1_61DEL AND 380_529del; S128A, L153P, H197S, F333N |
| PRT-108 | SEQ ID NO: 110 | 1_61DEL AND 380_529del; S128A, L153P, K160E, F333N |
| PRT-109 | SEQ ID NO: 111 | 1_61DEL AND 380_529del; S128A, L153P, K165E, F333N |
| PRT-110 | SEQ ID NO: 112 | 1_61DEL AND 380_529del; S128A, L153P, R309G, F333N |
| PRT-111 | SEQ ID NO: 113 | 1_61DEL AND 380_529del; S128A, L153P, R319E, F333N |
| PRT-112 | SEQ ID NO: 114 | 1_61DEL AND 380_529del; S128A, L153P, R319T, F333N |
| PRT-113 | SEQ ID NO: 115 | 1_61DEL AND 380_529del; S128A, L153P, R320Q, F333N |
| PRT-114 | SEQ ID NO: 116 | 1_61DEL AND 380_529del; S128A, L153P, R320L, F333N |
| PRT-115 | SEQ ID NO: 117 | 1_61del and 380_529del; A79V, S128A, L153P, R189K, |
| | | H222Q, F333N |
| PRT-116 | SEQ ID NO: 118 | 1_61del and 380_529del; D71E, A79V, S128A, L153P, |
| | | H222Q, F333N |
| PRT-117 | SEQ ID NO: 119 | 1_61del and 380_529del; D71E, A79V, S128A, L153P, |
| | | R189K, F333N |
| PRT-118 | SEQ ID NO: 120 | 1_61del and 380_529del; D71E, S128A, L153P, R189K, |
| | | H222Q, F333N |
| PRT-119 | SEQ ID NO: 121 | 1_61DEL AND 380_529del; S128A, L153P, K324I, F333N, |
| | | N162E, K229E |
| PRT-120 | SEQ ID NO: 122 | 1_61DEL AND 380_529del; S128A, L153P, K324I, F333N, |
| | | N162E, K229E, K196D |
| PRT-121 | SEQ ID NO: 123 | 1_61DEL AND 380_529del; S128A, L153P, K324I, F333N, |
| | | N162E, K229E, R280T |
| PRT-122 | SEQ ID NO: 124 | 1_61DEL AND 380_529del; S128A, L153P, K324I, F333N, |
| | | N162E, K229E, R280T, K196D |
| PRT-123 | SEQ ID NO: 125 | 1_61DEL AND 380_529del; S128A, L153P, K324I, F333N, |
| | | K196D |
| PRT-124 | SEQ ID NO: 126 | 1_61DEL AND 380_529del; S128A, L153P, K324I, F333N, |
| | | R280T |
| PRT-125 | SEQ ID NO: 127 | 1_61DEL AND 380_529del; S128A, L153P, K324I, F333N, |
| | | R280T, K196D |
| PRT-126 | SEQ ID NO: 128 | 1_61DEL AND 380_529del; S128A, L153P, K324I, F333N, |
| | | N162E, K229E, K196S |
| PRT-127 | SEQ ID NO: 129 | 1_61DEL AND 380_529del; S128A, L153P, K324I, F333N, |
| | | N162E, K229E, R280T, K196S |
| PRT-128 | SEQ ID NO: 130 | 1_61DEL AND 380_529del; S128A, L153P, K324I, F333N, |
| | | N162E, K229E, R280T, H222Q |
| PRT-129 | SEQ ID NO: 131 | 1_61DEL AND 380_529del; S128A, L153P, K324I, F333N, |
| | | N162E, K229E, R280T, H222Q, K196D |
| PRT-130 | SEQ ID NO: 132 | 1_61DEL AND 380_529del; S128A, L153P, K324I, F333N, |
| | | N162E, K229E, R280T, H222Q, K196S |
| PRT-131 | SEQ ID NO: 133 | 1_61DEL AND 380_529del; S128A, L153P, K324I, F333N, |
| | | K318S |
| PRT-132 | SEQ ID NO: 134 | 1_61DEL AND 380_529del; S128A, L153P, K324I, F333N, |
| | | N162E, K229E, K318S |
| PRT-133 | SEQ ID NO: 135 | 1_61DEL AND 380_529del; S128A, L153P, K324I, F333N, |
| | | N162E, K229E, R280T, K318S |
| PRT-134 | SEQ ID NO: 136 | 1_61DEL AND 380_529del; S128A, L153P, K324I, F333N, |
| | | R320W |
| PRT-135 | SEQ ID NO: 137 | 1_61DEL AND 380_529del; S128A, L153P, K324I, F333N, |
| | | N162E, K229E, R320W |
| PRT-136 | SEQ ID NO: 138 | 1_61DEL AND 380_529del; S128A, L153P, K324I, F333N, |
| | | N162E, K229E, R280T, R320W |
| PRT-137 | SEQ ID NO: 139 | 1_61DEL AND 380_529del; S128A, L153P, K324I, F333N, |
| | | T122S |
| PRT-138 | SEQ ID NO: 140 | 1_61DEL AND 380_529del; S128A, L153P, K324I, F333N, |
| | | N162E, K229E, T122S |
| PRT-139 | SEQ ID NO: 141 | 1_61DEL AND 380_529del; S128A, L153P, K324I, F333N, |
| | | N162E, K229E, R280T, T122S |
| PRT-140 | SEQ ID NO: 142 | 1_61DEL AND 380_529del; S128A, L153P, K324I, F333N, |
| | | D71Q, K68W, E75Y, D88H |
| PRT-141 | SEQ ID NO: 143 | 1_61DEL AND 380_529del; S128A, L153P, K324I, F333N, |
| | | N162E, K229E, D71Q, K68W, E75Y, D88H |
| PRT-142 | SEQ ID NO: 144 | 1_61DEL AND 380_529del; S128A, L153P, K324I, F333N, |
| | | N162E, K229E, R280T, D71Q, K68W, E75Y, D88H |
| PRT-143 | SEQ ID NO: 145 | 1_61DEL AND 380_529del; S128A, L153P, K324I, F333N, |
| | | N162E, K229E, R280T, K196D, D71Q, K68W, E75Y, D88H |
| PRT-144 | SEQ ID NO: 146 | 1_61DEL AND 380_529del; S128A, L153P, K324I, F333N, |
| | | K318S, R320W, T122S, D71Q, K68W, E75Y, D88H |
| PRT-145 | SEQ ID NO: 147 | 1_61DEL AND 380_529del; S128A, L153P, K324I, F333N, |
| | | N162E, K229E, K318S, R320W, T122S, D71Q, K68W, E75Y, |
| | | D88H |
| PRT-146 | SEQ ID NO: 148 | 1_61DEL AND 380_529del; S128A, L153P, K324I, F333N, |
| | | N162E, K229E, R280T, K318S, R320W, T122S, D71Q, K68W, |
| | | E75Y, D88H |
| PRT-147 | SEQ ID NO: 149 | 1_61DEL AND 380_529del; S128A, L153P, K324I, F333N, |
| | | N162E, K229E, R280T, K196D, K318S, R320W, T122S, D71Q, |
| | | K68W, E75Y, D88H |
| PRT-148 | SEQ ID NO: 150 | 1_61DEL AND 380_529del; S128A, L153P, K324I, F333N, |
| | | H275Y |
| PRT-149 | SEQ ID NO: 151 | 1_61DEL AND 380_529del; S128A, L153P, K324I, F333N, |
| | | N162E, K229E, H275Y |
| PRT-150 | SEQ ID NO: 152 | 1_61DEL AND 380_529del; S128A, L153P, K324I, F333N, |
| | | N162E, K229E, R280T, H275Y |
| PRT-151 | SEQ ID NO: 153 | 1_61DEL AND 380_529del; S128A, L153P, K324I, F333N, |
| | | N162E, K229E, R280T, H222Q, K196S, K318S |
| PRT-152 | SEQ ID NO: 154 | 1_61DEL AND 380_529del; S128A, L153P, K324I, F333N, |
| | | N162E, K229E, R280T, H222Q, K196S, R320W |
| PRT-153 | SEQ ID NO: 155 | 1_61DEL AND 380_529del; S128A, L153P, K324I, F333N, |
| | | N162E, K229E, R280T, H222Q, K196S, T122S |
| PRT-154 | SEQ ID NO: 156 | 1_61DEL AND 380_529del; S128A, L153P, K324I, F333N, |
| | | N162E, K229E, R280T, H222Q, K196S, H275Y |
| PRT-155 | SEQ ID NO: 157 | 1_61DEL AND 380_529del; S128A, L153P, K324I, F333N, |
| | | N162E, K229E, R280T, H222Q, K196S, D71Q, K68W, E75Y, |
| | | D88H |
| PRT-156 | SEQ ID NO: 158 | 1_61DEL AND 380_529del; S128A, L153P, K324I, F333N, |
| | | N162E, K229E, R280T, H222Q, K196S, K318S, R320W, T122S, |
| | | D71Q, K68W, E75Y, D88H |
| PRT-157 | SEQ ID NO: 159 | 1_61DEL AND 380_529del; S128A, L153P, K324I, F333N, |
| | | D71Q |
| PRT-158 | SEQ ID NO: 160 | 1_61DEL AND 380_529del; S128A, L153P, K324I, F333N, |
| | | D112Q |
| PRT-159 | SEQ ID NO: 161 | 1_61DEL AND 380_529del; S128A, L153P, K324I, F333N, |
| | | D327Y |
| PRT-160 | SEQ ID NO: 162 | 1_61DEL AND 380_529del; S128A, L153P, K324I, F333N, |
| | | K68W |
| PRT-161 | SEQ ID NO: 163 | 1_61DEL AND 380_529del; S128A, L153P, K324I, F333N, |
| | | E75Y |
| PRT-162 | SEQ ID NO: 164 | 1_61DEL AND 380_529del; S128A, L153P, K324I, F333N, |
| | | D88H |
| PRT-163 | SEQ ID NO: 165 | 1_61DEL AND 380_529del; S128A, L153P, K324I, F333N, |
| | | N219Q |
| PRT-164 | SEQ ID NO: 166 | 1_61DEL AND 380_529del; S128A, L153P, K324I, F333N, |
| | | S221N |
| PRT-165 | SEQ ID NO: 167 | 1_61DEL AND 380_529del; S128A, L153P, K324I, F333N, |
| | | N219G |
| PRT-166 | SEQ ID NO: 168 | 1_61DEL AND 380_529del; S128A, L153P, K324I, F333N, |
| | | N219T |
| PRT-167 | SEQ ID NO: 169 | 1_61DEL AND 380_529del; S128A, L153P, K324I, F333N, |
| | | K318S, N316S |
| PRT-168 | SEQ ID NO: 170 | 1_61DEL AND 380_529del; S128A, L153P, K324I, F333N, |
| | | K318S, N316Q |
| PRT-183 | SEQ ID NO: 185 | 1_61DEL AND 380_529del; S128A, L153P, K324I, F333N, |
| | | S221E |
| PRT-184 | SEQ ID NO: 186 | 1_61DEL AND 380_529del; S128A, L153P, K324I, F333N, |
| | | N219K |
| PRT-185 | SEQ ID NO: 187 | 1_61DEL AND 380_529del; S128A, L153P, K324I, F333N, |
| | | Y252R |
| PRT-186 | SEQ ID NO: 188 | 1_61DEL AND 380_529del; S128A, L153P, K324I, F333N, |
| | | R250GYG, S251I |
| PRT-187 | SEQ ID NO: 189 | 1_61DEL AND 380_529del; S128A, L153P, K324I, F333N, |
| | | R250GGYGG (SEQ ID NO: 918), S251I |
| PRT-188 | SEQ ID NO: 190 | 1_61DEL AND 380_529del; S128A, L153P, K324I, F333N, |
| | | P205L, E206T |
| PRT-189 | SEQ ID NO: 191 | 1_61DEL AND 380_529del; S128A, L153P, K324I, F333N, |
| | | P205F, E206T |
| PRT-190 | SEQ ID NO: 192 | 1_61DEL AND 380_529del; S128A, L153P, K324I, F333N, |
| | | P205LG, E206T |
| PRT-191 | SEQ ID NO: 193 | 1_61DEL AND 380_529del; S128A, L153P, K324I, F333N, |
| | | P205FG, E206T |
| PRT-192 | SEQ ID NO: 194 | 1_61DEL AND 380_529del; S128A, L153P, K324I, F333N, |
| | | P205FG, E206T, R250GYG, S251I |
| PRT-193 | SEQ ID NO: 195 | 1_61DEL AND 380_529del; S128A, L153P, K324I, F333N, |
| | | K149L, I262K, T276G, V364E, P205FG, E206T, R250GYG, |
| | | S251I |
| PRT-194 | SEQ ID NO: 196 | 1_61DEL AND 380_529del; S128A, L153P, K324I, F333N, |
| | | K149L, G199P, I262K, T276G, V364E |
| PRT-195 | SEQ ID NO: 197 | 1_61DEL AND 380_529del; S128A, L153P, K324I, F333N, |
| | | K116D, K149L, N182D, K196D, I262K, T276G, V364E |
| PRT-196 | SEQ ID NO: 198 | 1_61DEL AND 380_529del; S128A, L153P, K324I, F333N, |
| | | S94E, K116D, K149L, N182D, K196D, I262K, T276G, I336A, |
| | | V364E |
| PRT-197 | SEQ ID NO: 199 | 1_61DEL AND 380_529del; S128A, L153P, K324I, F333N, |
| | | S94E, K116D, K149L, N182D, K196D, Y224T, I262K, T276G, |
| | | I336A, V364E |
| PRT-198 | SEQ ID NO: 200 | 1_61DEL AND 380_529del; S128A, L153P, K324I, F333N, |
| | | S94E, K116D, K149L, N182D, K196D, Y224T, I262K, T276G, |
| | | Y281T, I336A, V364E |
| PRT-199 | SEQ ID NO: 201 | 1_61DEL AND 380_529del; S128A, L153P, K324I, F333N, |
| | | S94E, K116D, K149L, N182D, K196D, Y224T, I262K, T276G, |
| | | Y281T, I336A, V364E, H174E |
| PRT-200 | SEQ ID NO: 202 | 1_61DEL AND 380_529del; S128A, L153P, K324I, F333N, |
| | | S94E, K116D, K149L, N182D, K196D, Y224T, V270P, T276G, |
| | | Y281T, I336A, V364E |
| PRT-201 | SEQ ID NO: 203 | 1_61DEL AND 380_529del; S128A, L153P, K324I, F333N, |
| | | S94E, K116D, K149L, N182D, K196D, Y224T, V270P, V274G, |
| | | Y281T, I336A, V364E |
| PRT-202 | SEQ ID NO: 204 | 1_61DEL AND 380_529del; S128A, L153P, K324I, F333N, |
| | | S94E, K116D, K149L, N182D, G199P, Y224T, V270P, V274G, |
| | | Y281T, I336A, V364E |
| PRT-203 | SEQ ID NO: 205 | 1_61DEL AND 380_529del; S128A, L153P, K324I, F333N, |
| | | K149L, K196D, I262K, T276G, V364E |
| PRT-204 | SEQ ID NO: 206 | 1_61DEL AND 380_529del; S128A, L153P, K324I, F333N, |
| | | I262K, V274G, Y281T |
| PRT-205 | SEQ ID NO: 207 | 1_61DEL AND 380_529del; S128A, L153P, K324I, F333N, |
| | | G199P, I262K, T276G, Y281T |
| PRT-206 | SEQ ID NO: 208 | 1_61DEL AND 380_529del; S128A, L153P, K324I, F333N, |
| | | K196D, I262K, T276G, Y281T |
| PRT-207 | SEQ ID NO: 209 | 1_61DEL AND 380_529del; S128A, L153P, K324I, F333N, |
| | | G199P, Y224T, I262K, T276G, Y281T |
| PRT-208 | SEQ ID NO: 210 | 1_61DEL AND 380_529del; S128A, L153P, K324I, F333N, |
| | | G199P, Y224T, I262K, T276G, Y281T, I336A |
| PRT-209 | SEQ ID NO: 211 | 1_61DEL AND 380_529del; S128A, L153P, K324I, F333N, |
| | | K149L, G199P, Y224T, I262K, T276G, Y281T, I336A, V364E |
| PRT-210 | SEQ ID NO: 212 | 1_61DEL AND 380_529del; S128A, L153P, K324I, F333N, |
| | | K149L, K196D, Y224T, I262K, T276G, Y281T, I336A, V364E |
| PRT-211 | SEQ ID NO: 213 | 1_61DEL AND 380_529del; S128A, L153P, K324I, F333N, |
| | | K149L, G199P, Y224T, I262K, T276G, Y281T, I336A, V364E, |
| | | H174E |
| PRT-212 | SEQ ID NO: 214 | 1_61DEL AND 380_529del; S128A, L153P, K324I, F333N, |
| | | S94E, K116D, K149L, N182D, K196D, Y224T, I262K, T276G, |
| | | Y281T, V303I, I336A, V364E |
| PRT-243 | SEQ ID NO: 251 | 1_61DEL AND 380_529del; T122S, S128A, L153P, F333N, |
| | | K324I, N162E, K229E, H275Y, N219G, H174E, |
| | | 177_183delinsRDNRDN (SEQ ID NO: 919) |
| PRT-213 | SEQ ID NO: 215 | S128A, L153P, K324I, F333N, |
| | | 311_321delinsLSNVEALGYNITSGYSDKRY (SEQ ID NO: 939) |
| PRT-214 | SEQ ID NO: 216 | 1_61DEL AND 380_529del; S128A, L153P, K324I, F333N, |
| | | 310_320delinsFLVFGVTYDGSTPVP (SEQ ID NO: 940) |
| PRT-215 | SEQ ID NO: 217 | 1_61DEL AND 380_529del; S128A, L153P, K324I, F333N, |
| | | 307_322delinsLAKGNNYFGHWHFAVM (SEQ ID NO: 936) |
| PRT-216 | SEQ ID NO: 218 | 1_61DEL AND 380_529del; S128A, L153P, K324I, F333N, |
| | | 86_105delinsDDMEAKGNNYFGHWHFAVATN (SEQ ID NO: 941) |
| PRT-217 | SEQ ID NO: 219 | 1_61DEL AND 380_529del; S128A, L153P, K324I, F333N, |
| | | 271_287delinsVGISGSAGGIKSVDSDGDSYI (SEQ ID NO: 932) |
| PRT-218 | SEQ ID NO: 220 | 1_61DEL AND 380_529del; S128A, L153P, K324I, F333N, |
| | | 91_100delinsGGGGGKSVDSDGDSYGGGGG (SEQ ID NO: 943) |
| PRT-219 | SEQ ID NO: 221 | 1_61DEL AND 380_529del; S128A, L153P, K324I, F333N, |
| | | 308_324delinsTKSVDSDGDSYKINY (SEQ ID NO: 932) |
| PRT-220 | SEQ ID NO: 222 | 1_61DEL AND 380_529del; S128A, L153P, K324I, F333N, |
| | | 311_319delinsLLKPKILGYNITSGYSDEIY (SEQ ID NO: 938) |
| PRT-221 | SEQ ID NO: 223 | 1_61DEL AND 380_529del; S128A, L153P, K324I, F333N, |
| | | 315_317delinsVTYDGST (SEQ ID NO: 946) |
| PRT-222 | SEQ ID NO: 224 | 1_61DEL AND 380_529del; S128A, L153P, K324I, F333N, |
| | | 303_326delinsTGAKGNNYFGHWHFAVRVRL (SEQ ID NO: 933) |
| PRT-223 | SEQ ID NO: 225 | 1_61DEL AND 380_529del; S128A, L153P, K324I, F333N, |
| | | 93_98delinsGGGGGKSVDSDGDSYGGGGG (SEQ ID NO: 943) |
| PRT-224 | SEQ ID NO: 226 | 1_61DEL AND 380_529del; S128A, L153P, K324I, F333N, |
| | | 174_187delinsVMYKGKKLLEAARAGQPDSSE (SEQ ID NO: 945) |
| PRT-225 | SEQ ID NO: 227 | 1_61DEL AND 380_529del; S128A, L153P, K324I, F333N, |
| | | 306_324delinsGHYGVYNGVTYWGHTPLHLAAMLGHLVYGLY (SEQ ID |
| | | NO: 934) |
| PRT-226 | SEQ ID NO: 228 | 1_61DEL AND 380_529del; S128A, L153P, K324I, F333N, |
| | | 306_324delinsQRTPLHLAAWADHLYR (SEQ ID NO: 935) |
| PRT-227 | SEQ ID NO: 229 | 1_61DEL AND 380_529del; S128A, L153P, K324I, F333N, |
| | | 307_324delinsGGFAFDISIDNGNEDVY (SEQ ID NO: 937) |
| PRT-228 | SEQ ID NO: 230 | 1_61DEL AND 380_529del; S128A, L153P, K324I, F333N, |
| | | 88_102delinsGEIVAFDISIDNGNEDLAEILQLSTYDGGG (SEQ ID NO: |
| | | 942) |
| PRT-229 | SEQ ID NO: 231 | 1_61DEL AND 380_529del; S128A, L153P, K324I, F333N, |
| | | 311_321delinsLSNVEALGYNITSGYSDKRY (SEQ ID NO: 939), |
| | | 86_105delinsDDMEAKGNNYFGHWHFAVATN (SEQ ID NO: |
| | | 941), 271_287delinsVGISGSAGGIKSVDSDGDSYI (SEQ ID NO: |
| | | 932) |
| PRT-230 | SEQ ID NO: 232 | 1_61DEL AND 380_529del; S128A, L153P, K324I, F333N, |
| | | 311_321delinsLSNVEALGYNITSGYSDKRY (SEQ ID NO: 939), |
| | | 86_105delinsDDMEAKGNNYFGHWHFAVATN (SEQ ID NO: 941) |
| PRT-231 | SEQ ID NO: 233 | 1_61DEL AND 380_529del; S128A, L153P, K324I, F333N, |
| | | 311_321delinsLSNVEALGYNITSGYSDKRY (SEQ ID NO: 939), |
| | | 91_100delinsGGGGGKSVDSDGDSYGGGGG (SEQ ID NO: 943) |
| PRT-232 | SEQ ID NO: 234 | 1_61DEL AND 380_529del; S128A, L153P, K324I, F333N, |
| | | 311_321delinsLSNVEALGYNITSGYSDKRY (SEQ ID NO: 939), |
| | | 271_287delinsVGISGSAGGIKSVDSDGDSYI (SEQ ID NO: 932) |
| PRT-233 | SEQ ID NO: 235 | 1_61DEL AND 380_529del; S128A, L153P, K324I, F333N, |
| | | 310_320delinsFLVFGVTYDGSTPVP (SEQ ID NO: 940), |
| | | 86_105delinsDDMEAKGNNYFGHWHFAVATN (SEQ ID NO: 941), |
| | | 271_287delinsVGISGSAGGIKSVDSDGDSYI (SEQ ID NO: 932) |
| PRT-234 | SEQ ID NO: 236 | 1_61DEL AND 380_529del; S128A, L153P, K324I, F333N, |
| | | 310_320delinsFLVFGVTYDGSTPVP (SEQ ID NO: 940), |
| | | 86_105delinsDDMEAKGNNYFGHWHFAVATN (SEQ ID NO: 941) |
| PRT-235 | SEQ ID NO: 237 | 1_61DEL AND 380_529del; S128A, L153P, K324I, F333N, |
| | | 310_320delinsFLVFGVTYDGSTPVP (SEQ ID NO: 940), |
| | | 91_100delinsGGGGGKSVDSDGDSYGGGGG (SEQ ID NO: 943) |
| PRT-236 | SEQ ID NO: 238 | 1_61DEL AND 380_529del; S128A, L153P, K324I, F333N, |
| | | 310_320delinsFLVFGVTYDGSTPVP (SEQ ID NO: 940), |
| | | 271_287delinsVGISGSAGGIKSVDSDGDSYI (SEQ ID NO: 932) |
| PRT-237 | SEQ ID NO: 239 | 1_61DEL AND 380_529del; S128A, L153P, K324I, F333N, |
| | | 307_322delinsLAKGNNYFGHWHFAVM (SEQ ID NO: 936), |
| | | 91_100delinsGGGGGKSVDSDGDSYGGGGG (SEQ ID NO: 943) |
| PRT-238 | SEQ ID NO: 240 | 1_61DEL AND 380_529del; S128A, L153P, K324I, F333N, |
| | | 307_322delinsLAKGNNYFGHWHFAVM (SEQ ID NO: 936), |
| | | 271_287delinsVGISGSAGGIKSVDSDGDSYI (SEQ ID NO: 932) |
| PRT-239 | SEQ ID NO: 241 | 1_61DEL AND 380_529del; S128A, L153P, K324I, F333N, |
| | | 308_324delinsTKSVDSDGDSYKINY (SEQ ID NO: 932), |
| | | 86_105delinsDDMEAKGNNYFGHWHFAVATN (SEQ ID NO: 941) |
| PRT-244 | SEQ ID NO: 280 | 1_61DEL AND 380_529del; M117E, S128A, L153P, F333N |
| PRT-245 | SEQ ID NO: 281 | 1_61DEL AND 380_529del; M117F, S128A, L153P, F333N |
| PRT-246 | SEQ ID NO: 282 | 1_61DEL AND 380_529del; S128A, L153P, F333N, E348R |
| PRT-247 | SEQ ID NO: 283 | 1_61DEL AND 380_529del; S128A, L153P, F333N, E347R |
| PRT-248 | SEQ ID NO: 284 | 1_61DEL AND 380_529del; S128A, L153P, F333N, I351R |
| PRT-249 | SEQ ID NO: 285 | 1_61DEL AND 380_529del; S128A, L153P, S220Y, F333N |
| PRT-250 | SEQ ID NO: 286 | 1_61DEL AND 380_529del; S128A, L153P, S221Y, F333N |
| PRT-251 | SEQ ID NO: 287 | 1_61DEL AND 380_529del; S128A, L153P, H275Y, F333N |
| PRT-252 | SEQ ID NO: 288 | 1_61DEL AND 380_529del; S128A, L153P, F333N, |
| | | 200_202NKAdelinsPYLSTKHL (SEQ ID NO: 930) |
| PRT-253 | SEQ ID NO: 289 | 1_61DEL AND 380_529del; M117E, S128A, L153P, H222E, |
| | | F333N, E348R |
| PRT-254 | SEQ ID NO: 290 | 1_61DEL AND 380_529del; C125S, S128A, L153P, K324I, |
| | | F333N |
| PRT-255 | SEQ ID NO: 291 | 1_61DEL AND 380_529del; C125S, S128A, L153P, R280T, |
| | | F333N |
| PRT-256 | SEQ ID NO: 292 | 1_61DEL AND 380_529del; C125S, S128A, L153P, N162E, |
| | | K229E, F333N |
| PRT-257 | SEQ ID NO: 293 | 1_61DEL AND 380_529del; C125S, S128A, L153P, |
| | | K229E, K324I, F333N |
| PRT-258 | SEQ ID NO: 294 | 1_61DEL AND 380_529del; C125S, S128A, L153P, N162E, |
| | | K229E, R280T, K324I, F333N |
| PRT-259 | SEQ ID NO: 295 | 1_61DEL AND 380_529del; S128A, L153P, N162E, K229E, |
| | | K324I, F333N, 177_183KEYQSNKdelinsESDHG (SEQ ID NOs: |
| | | 920 and 921) |
| PRT-260 | SEQ ID NO: 296 | 1_61DEL AND 380_529del; S128A, L153P, N162E, Q180H, |
| | | K183D, K229E, K324I, F333N, 176_178YKEdelinsN |
| PRT-261 | SEQ ID NO: 297 | 1_61DEL AND 380_529del; S128A, L153P, N162E, K229E, |
| | | F313T, S317N, K324I, F333N, |
| | | 177_319KEYQSNKDRdelinsRDNRDN (SEQ ID NOs: 925 and 919) |
| PRT-262 | SEQ ID NO: 298 | 1_61DEL AND 380_529del; S128A, L153P, N162E, K229E, |
| | | K324I, F333N, 177_319KEYQSNKFNDNSKRdelinsRDNRDNIGDE |
| | | (SEQ ID NOs: 926 and 927) |
| PRT-263 | SEQ ID NO: 299 | 1_61DEL AND 380_529del; S128A, L153P, N162E, K229E, |
| | | F313I, N314G, K324I, F333N, |
| | | 177_318KEYQSNKNSKdelinsRDNRDN (SEQ ID NOs: 924 and 919) |
| PRT-264 | SEQ ID NO: 300 | 1_61DEL AND 380_529del; S128A, L153P, N162E, K229E, |
| | | D293G, Q294E, K297R, K324I, F333N, |
| | | 177_183KEYQSNKdelinsRDNRDN (SEQ ID NOs: 920 and 919) |
| PRT-265 | SEQ ID NO: 301 | 1_61DEL AND 380_529del; S128A, L153P, N162E, K229E, |
| | | Q294E, D295K, K324I, F333N, 177_183KEYQSNKdelinsRDNRDN |
| | | (SEQ ID NOs: 920 and 919) |
| PRT-266 | SEQ ID NO: 302 | 1_61DEL AND 380_529del; S128A, L153P, N162E, K229E, |
| | | Q294E, D295N, K297N, K324I, F333N, |
| | | 177_183KEYQSNKdelinsRDNRDN (SEQ ID NOs: 920 and 919) |
| PRT-267 | SEQ ID NO: 303 | 1_61DEL AND 380_529del; S128A, L153P, N162E, K229E, |
| | | K324I, F333N, 177_185KEYQSNKYAdelinsESPNT (SEQ ID NOs: |
| | | 922 and 923) |
| PRT-268 | SEQ ID NO: 304 | 1_61DEL AND 380_529del; S128A, L153P, N162E, K229E, |
| | | K324I, F333N, 180_185QSNKYAdelinsSG (SEQ ID NO: 929) |
| PRT-269 | SEQ ID NO: 305 | 1_61DEL AND 380_529del; S128A, L153P, N162E, K229E, |
| | | K318G, K324I, F333N, 177_317KEYQSNKNDNSdelinsRDNRDN |
| | | (SEQ ID NOs: 928 and 919) |
| PRT-270 | SEQ ID NO: 306 | 1_61DEL AND 380_529del; S128A, L153P, N162E, K229E, |
| | | K324I, F333N, 177_317KEYQSNKNDNSdelinsRDNRDN (SEQ ID |
| | | NOs: 928 and 919) |
| PRT-271 | SEQ ID NO: 307 | 1_61DEL AND 380_529del; S128A, L153P, G199P, K324I, |
| | | F333N |
| PRT-272 | SEQ ID NO: 308 | 1_61DEL AND 380_529del; S128A, L153P, H174E, K324I, |
| | | F333N |
| PRT-273 | SEQ ID NO: 309 | 1_61DEL AND 380_529del; S128A, L153P, I262K, K324I, |
| | | F333N |
| PRT-274 | SEQ ID NO: 310 | 1_61DEL AND 380_529del; S128A, L153P, K324I, F333N, |
| | | I336A |
| PRT-275 | SEQ ID NO: 311 | 1_61DEL AND 380_529del; K116D, S128A, L153P, K324I, |
| | | F333N |
| PRT-276 | SEQ ID NO: 312 | 1_61DEL AND 380_529del; S128A, K149L, L153P, K324I, |
| | | F333N |
| PRT-277 | SEQ ID NO: 313 | 1_61DEL AND 380_529del; S128A, L153P, N182D, K324I, |
| | | F333N |
| PRT-278 | SEQ ID NO: 314 | 1_61DEL AND 380_529del; S94E, S128A, L153P, K324I, |
| | | F333N |
| PRT-279 | SEQ ID NO: 315 | 1_61DEL AND 380_529del; S128A, L153P, T276G, K324I, |
| | | F333N |
| PRT-280 | SEQ ID NO: 316 | 1_61DEL AND 380_529del; S128A, L153P, V270P, K324I, |
| | | F333N |
| PRT-281 | SEQ ID NO: 317 | 1_61DEL AND 380_529del; S128A, L153P, V274G, K324I, |
| | | F333N |
| PRT-282 | SEQ ID NO: 318 | 1_61DEL AND 380_529del; S128A, L153P, V303I, K324I, |
| | | F333N |
| PRT-283 | SEQ ID NO: 319 | 1_61DEL AND 380_529del; S128A, L153P, K324I, F333N, |
| | | V364E |
| PRT-284 | SEQ ID NO: 320 | 1_61DEL AND 380_529del; S128A, L153P, Y224T, K324I, |
| | | F333N |
| PRT-285 | SEQ ID NO: 321 | 1_61DEL AND 380_529del; S128A, L153P, Y281T, K324I, |
| | | F333N |
| PRT-286 | SEQ ID NO: 322 | 1_61DEL AND 380_529del; I84P, S128A, L153P, K324I, |
| | | F333N |
| PRT-287 | SEQ ID NO: 323 | 1_61DEL AND 380_529del; I84G, S128A, L153P, K324I, |
| | | F333N |
| PRT-288 | SEQ ID NO: 324 | 1_61DEL AND 380_529del; V102L, S128A, L153P, K324I, |
| | | F333N |
| PRT-289 | SEQ ID NO: 325 | 1_61DEL AND 380_529del; S128A, L153P, A185T, K324I, |
| | | F333N |
| PRT-290 | SEQ ID NO: 326 | 1_61DEL AND 380_529del; S128A, L153P, N182P, K324I, |
| | | F333N |
| PRT-291 | SEQ ID NO: 327 | 1_61DEL AND 380_529del; S128A, L153P, N242G, K324I, |
| | | F333N |
| PRT-292 | SEQ ID NO: 328 | 1_61DEL AND 380_529del; S128A, L153P, 1301L, K324I, |
| | | F333N |
| PRT-293 | SEQ ID NO: 329 | 1_61DEL AND 380_529del; S128A, L153P, V303Y, K324I, |
| | | F333N |
| PRT-294 | SEQ ID NO: 330 | 1_61DEL AND 380_529del; S128A, L153P, E310D, K324I, |
| | | F333N |
| PRT-295 | SEQ ID NO: 331 | 1_61DEL AND 380_529del; S128A, L153P, H174D, K324I, |
| | | F333N |
| PRT-296 | SEQ ID NO: 332 | 1_61DEL AND 380_529del; S128A, L153P, N173K, K324I, |
| | | F333N |
| PRT-297 | SEQ ID NO: 333 | 1_61DEL AND 380_529del; S128A, L153P, K196Q, K324I, |
| | | F333N |
| PRT-298 | SEQ ID NO: 334 | 1_61DEL AND 380_529del; S128A, L153P, K196L, K324I, |
| | | F333N |
| PRT-299 | SEQ ID NO: 335 | 1_61DEL AND 380_529del; S128A, L153P, K195L, K324I, |
| | | F333N |
| PRT-300 | SEQ ID NO: 336 | 1_61DEL AND 380_529del; S128A, L153P, K195R, K324I, |
| | | F333N |
| PRT-301 | SEQ ID NO: 337 | 1_61DEL AND 380_529del; S128A, L153P, H197R, K324I, |
| | | F333N |
| PRT-302 | SEQ ID NO: 338 | 1_61DEL AND 380_529del; S128A, L153P, H197K, K324I, |
| | | F333N |
| PRT-303 | SEQ ID NO: 339 | 1_61DEL AND 380_529del; S128A, L153P, H197D, K324I, |
| | | F333N |
| PRT-304 | SEQ ID NO: 340 | 1_61DEL AND 380_529del; S128A, L153P, N263D, K324I, |
| | | F333N |
| PRT-305 | SEQ ID NO: 341 | 1_61DEL AND 380_529del; S128A, L153P, H268G, K324I, |
| | | F333N |
| PRT-306 | SEQ ID NO: 342 | 1_61DEL AND 380_529del; S128A, L153P, H268R, K324I, |
| | | F333N |
| PRT-307 | SEQ ID NO: 343 | 1_61DEL AND 380_529del; S128A, L153P, H268S, K324I, |
| | | F333N |
| PRT-308 | SEQ ID NO: 344 | 1_61DEL AND 380_529del; S128A, L153P, T276R, K324I, |
| | | F333N |
| PRT-309 | SEQ ID NO: 345 | 1_61DEL AND 380_529del; S128A, L153P, H275L, K324I, |
| | | F333N |
| PRT-310 | SEQ ID NO: 346 | 1_61DEL AND 380_529del; S128A, L153P, L279G, K324I, |
| | | F333N |
| PRT-311 | SEQ ID NO: 347 | 1_61DEL AND 380_529del; S128A, L153P, L279T, K324I, |
| | | F333N |
| PRT-312 | SEQ ID NO: 348 | 1_61DEL AND 380_529del; S128A, L153P, Y281H, K324I, |
| | | F333N |
| PRT-313 | SEQ ID NO: 349 | 1_61DEL AND 380_529del; S128A, L153P, Y281S, K324I, |
| | | F333N |
| PRT-314 | SEQ ID NO: 350 | 1_61DEL AND 380_529del; S128A, L153P, R282G, K324I, |
| | | F333N |
| PRT-315 | SEQ ID NO: 351 | 1_61DEL AND 380_529del; S128A, L153P, R282N, K324I, |
| | | F333N |
| PRT-316 | SEQ ID NO: 352 | 1_61DEL AND 380_529del; S128A, L153P, K324I, F333D |
| PRT-317 | SEQ ID NO: 353 | 1_61DEL AND 380_529del; S128A, L153P, K324I, L329K, |
| | | F333N |
| PRT-318 | SEQ ID NO: 354 | 1_61DEL AND 380_529del; S128A, L153P, K324I, L329R, |
| | | F333N |
| PRT-319 | SEQ ID NO: 355 | 1_61DEL AND 380_529del; S128A, L153P, L279W, K324I, |
| | | F333N |
| PRT-320 | SEQ ID NO: 356 | 1_61DEL AND 380_529del; S128A, L153P, L279Y, K324I, |
| | | F333N |
| PRT-321 | SEQ ID NO: 357 | 1_61DEL AND 380_529del; D71E, S128A, L153P, R189K, |
| | | K324I, A343Y |
| PRT-322 | SEQ ID NO: 358 | 1_61DEL AND 380_529del; D71E, S94D, S128A, E143D, |
| | | L153P, R189K, Q294E, K324I, F333N, A343Y |
| PRT-323 | SEQ ID NO: 359 | 1_61DEL AND 380_529del; D71E, 184V, S94D, S128A, E143D, |
| | | L153P, R189K, N242G, Q294E, K324I, F333N, A343Y |
| PRT-324 | SEQ ID NO: 360 | 1_61DEL AND 380_529del; D71E, 184V, E86D, H91K, S94D, |
| | | S128A, E143D, L153P, S181D, R189K, N228K, N242G, N243K, |
| | | K244L, G256D, Q294E, K324I, F333N, A343Y |
| PRT-325 | SEQ ID NO: 361 | 1_61DEL AND 380_529del; S128A, L153P, N260A, K324I, |
| | | F333N, K346P, E347N, E374P |
| PRT-326 | SEQ ID NO: 362 | 1_61DEL AND 380_529del; S128A, L153P, V164K, N260A, |
| | | K324I, F333N, K346P, E347N, E374P |
| PRT-327 | SEQ ID NO: 363 | 1_61DEL AND 380_529del; S128A, L153P, F175I, N260A, |
| | | M323L, K324I, F333N, K346P, E347N, E374P |
| PRT-328 | SEQ ID NO: 364 | 1_61DEL AND 380_529del; S128A, R142K, L153P, N260A, |
| | | K324I, F333N, K346P, E347N |
| PRT-329 | SEQ ID NO: 365 | 1_61DEL AND 380_529del; S128A, R142K, L153P, Q232E, |
| | | N260A, K324I, F333N, R337L, K346P, E347N |
| PRT-330 | SEQ ID NO: 366 | 1_61DEL AND 380_529del; S128A, R142K, L153P, Y217W, |
| | | N260A, K324I, F333N, R337L, K346P, E347N |
| PRT-331 | SEQ ID NO: 367 | 1_61DEL AND 380_529del; K115L, S128A, R142K, L153P, |
| | | N260A, K324I, F333N, R337L, K346P, E347N |
| PRT-332 | SEQ ID NO: 368 | 1_61DEL AND 380_529del; S128A, R142K, F148Y, L153P, |
| | | N182D, N260A, S286T, M323L, K324I, F333N, K346P, E347N |
| PRT-338 | SEQ ID NO: 374 | 1_61DEL AND 380_529del; S128A, L153P, N162E, H174E, |
| | | N219G, K229E, N233G, H275Y, K324I, D332K, F333N, |
| | | 177_183KEYQSNKdelinsRDNRDN (SEQ ID NOs: 920 and 919) |
| PRT-339 | SEQ ID NO: 375 | 1_61DEL AND 380_529del; C125S, S128A, L153P, N162E, |
| | | H174E, N219G, K229E, N233G, H275Y, K324I, D332K, F333N, |
| | | 177_183KEYQSNKdelinsRDNRDN (SEQ ID NOs: 920 and 919) |
| PRT-340 | SEQ ID NO: 376 | 1_61DEL AND 380_529del; T122S, S128A, L153P, N162E, |
| | | H174E, N219G, K229E, N233G, H275Y, K324I, D332K, F333N, |
| | | 177_183KEYQSNKdelinsRDNRDN (SEQ ID NOs: 920 and 919) |
| PRT-341 | SEQ ID NO: 377 | 1_61DEL AND 380_529del; T122S, C125S, S128A, L153P, |
| | | N162E, H174E, N219G, K229E, N233G, H275Y, K324I, D332K, |
| | | F333N, 177_183KEYQSNKdelinsRDNRDN (SEQ ID NOs: 920 and |
| | | 919) |
| PRT-342 | SEQ ID NO: 378 | 1_61DEL AND 380_529del; S128A, L153P, N162E, H174E, |
| | | N219G, K229E, H275Y, K324I, F333N, |
| | | 177_183KEYQSNKdelinsRDNRDN (SEQ ID NOs: 920 and 919) |
| PRT-343 | SEQ ID NO: 379 | 1_61DEL AND 380_529del; C125S, S128A, L153P, N162E, |
| | | H174E, N219G, K229E, H275Y, K324I, F333N, |
| | | 177_183KEYQSNKdelinsRDNRDN (SEQ ID NOs: 920 and 919) |
| PRT-344 | SEQ ID NO: 380 | 1_61DEL AND 380_529del; T122S, S128A, L153P, N162E, |
| | | H174E, N219G, K229E, H275Y, K324I, F333N, |
| | | 177_183KEYQSNKdelinsRDNRDN (SEQ ID NOs: 920 and 919) |
| PRT-345 | SEQ ID NO: 381 | 1_61DEL AND 380_529del; T122S, C125S, S128A, L153P, |
| | | N162E, H174E, N219G, K229E, H275Y, K324I, F333N, |
| | | 177_183KEYQSNKdelinsRDNRDN (SEQ ID NOs: 920 and 919) |
| PRT-346 | SEQ ID NO: 382 | 1_61DEL AND 380_529del; S128A, L153P, K324I, F333N, |
| | | 380insENNVADSNKNSEDVEVSSVEFDFLNFKYPKNEDQVSTEDMRLTLKDTNV |
| | | FNQPQVKISFEEQNGNDWKEKDSGTFEEGKKYRLKIDVNKLALKIYGHLSKNKLN |
| | | VIVNGKAVNIQNVVKKDSIETSFTIYSDSIDLEKINYWIDSENNWS (SEQ ID |
| | | NO: 744) |
| PRT-347 | SEQ ID NO: 383 | 1_61DEL AND 380_529del; S128A, L153P, N162E, N219G, |
| | | K229E, K324I, F333N, 177_183KEYQSNKdelinsRDNRDN (SEQ ID |
| | | NOs: 920 and 919) |
| PRT-348 | SEQ ID NO: 384 | 1_61DEL AND 380_529del; S128A, L153P, N162E, N219G, |
| | | K229E, H275Y, K324I, F333N, 177_183KEYQSNKdelinsRDNRDN |
| | | (SEQ ID NOs: 920 and 919) |
| PRT-349 | SEQ ID NO: 385 | 1_61DEL AND 380_529del; T122S, S128A, L153P, N162E, |
| | | N219G, K229E, K324I, F333N, 177_183KEYQSNKdelinsRDNRDN |
| | | (SEQ ID NOs: 920 and 919) |
| PRT-350 | SEQ ID NO: 386 | 1_61DEL AND 380_529del; S128A, L153P, N162E, N219G, |
| | | K229E, H275Y, R280T, K324I, F333N, |
| | | 177_183KEYQSNKdelinsRDNRDN (SEQ ID NOs: 920 and 919) |
| PRT-351 | SEQ ID NO: 387 | 1_61DEL AND 380_529del; T122S, S128A, L153P, N162E, |
| | | N219G, K229E, R280T, K324I, F333N, |
| | | 177_183KEYQSNKdelinsRDNRDN (SEQ ID NOs: 920 and 919) |
| PRT-352 | SEQ ID NO: 388 | 1_61DEL AND 380_529del; D71Q, S128A, L153P, N162E, |
| | | N219G, K229E, K324I, F333N, 177_183KEYQSNKdelinsRDNRDN |
| | | (SEQ ID NOs: 920 and 919) |
| PRT-353 | SEQ ID NO: 389 | 1_61DEL AND 380_529del; T122S, S128A, L153P, N162E, |
| | | N219G, K229E, H275Y, K324I, F333N, |
| | | 177_183KEYQSNKdelinsRDNRDN (SEQ ID NOs: 920 and 919) |
| PRT-354 | SEQ ID NO: 390 | 1_61DEL AND 380_529del; S128A, L153P, N162E, N219G, |
| | | K229E, H275Y, K318S, K324I, F333N, |
| | | 177_183KEYQSNKdelinsRDNRDN (SEQ ID NOs: 920 and 919) |
| PRT-355 | SEQ ID NO: 391 | 1_61DEL AND 380_529del; S128A, L153P, N162E, N219G, |
| | | K229E, K318S, K324I, F333N, 177_183KEYQSNKdelinsRDNRDN |
| | | (SEQ ID NOs: 920 and 919) |
| PRT-356 | SEQ ID NO: 392 | 1_61DEL AND 380_529del; S128A, L153P, N219G, H275Y, |
| | | K324I, F333N, 177_183KEYQSNKdelinsRDNRDN (SEQ ID NOs: |
| | | 920 and 919) |
| PRT-357 | SEQ ID NO: 393 | 1_61DEL AND 380_529del; T122S, S128A, L153P, N219G, |
| | | K324I, F333N, 177_183KEYQSNKdelinsRDNRDN (SEQ ID NOs: |
| | | 920 and 919) |
| PRT-358 | SEQ ID NO: 394 | 1_61DEL AND 380_529del; D71Q, S128A, L153P, N219G, |
| | | K324I, F333N, 177_183KEYQSNKdelinsRDNRDN (SEQ ID NOs: |
| | | 920 and 919) |
| PRT-359 | SEQ ID NO: 395 | 1_61DEL AND 380_529del; S128A, L153P, N162E, N219Q, |
| | | K229E, K324I, F333N, 177_183KEYQSNKdelinsRDNRDN (SEQ ID |
| | | NOs: 920 and 919) |
| PRT-360 | SEQ ID NO: 396 | 1_61DEL AND 380_529del; D71Q, S128A, L153P, N162E, |
| | | N219Q, K229E, K324I, F333N, 177_183KEYQSNKdelinsRDNRDN |
| | | (SEQ ID NOs: 920 and 919) |
| PRT-361 | SEQ ID NO: 397 | 1_61DEL AND 380_529del; S128A, L153P, N162E, N219Q, |
| | | K229E, H275Y, K324I, F333N, 177_183KEYQSNKdelinsRDNRDN |
| | | (SEQ ID NOs: 920 and 919) |
| PRT-362 | SEQ ID NO: 398 | 1_61DEL AND 380_529del; T122S, S128A, L153P, N162E, |
| | | N219Q, K229E, K324I, F333N, 177_183KEYQSNKdelinsRDNRDN |
| | | (SEQ ID NOs: 920 and 919) |
| PRT-363 | SEQ ID NO: 399 | 1_61DEL AND 380_529del; S128A, L153P, N162E, N219Q, |
| | | K229E, H275Y, R280T, K324I, F333N, |
| | | 177_183KEYQSNKdelinsRDNRDN (SEQ ID NOs: 920 and 919) |
| PRT-364 | SEQ ID NO: 400 | 1_61DEL AND 380_529del; T122S, S128A, L153P, N162E, |
| | | N219Q, K229E, R280T, K324I, F333N, |
| | | 177_183KEYQSNKdelinsRDNRDN (SEQ ID NOs: 920 and 919) |
| PRT-365 | SEQ ID NO: 401 | 1_61DEL AND 380_529del; T122S, S128A, L153P, N162E, |
| | | N219Q, K229E, H275Y, K324I, F333N, |
| | | 177_183KEYQSNKdelinsRDNRDN (SEQ ID NOs: 920 and 919) |
| PRT-366 | SEQ ID NO: 402 | 1_61DEL AND 380_529del; S128A, L153P, N162E, N219Q, |
| | | K229E, H275Y, K318S, K324I, F333N, |
| | | 177_183KEYQSNKdelinsRDNRDN (SEQ ID NOs: 920 and 919) |
| PRT-367 | SEQ ID NO: 403 | 1_61DEL AND 380_529del; S128A, L153P, N162E, N219Q, |
| | | K229E, K318S, K324I, F333N, 177_183KEYQSNKdelinsRDNRDN |
| | | (SEQ ID NOs: 920 and 919) |
| PRT-368 | SEQ ID NO: 404 | 1_61DEL AND 380_529del; S128A, L153P, N219Q, H275Y, |
| | | K324I, F333N, 177_183KEYQSNKdelinsRDNRDN (SEQ ID NOs: |
| | | 920 and 919) |
| PRT-369 | SEQ ID NO: 405 | 1_61DEL AND 380_529del; T122S, S128A, L153P, N219Q, |
| | | K324I, F333N, 177_183KEYQSNKdelinsRDNRDN (SEQ ID NOs: |
| | | 920 and 919) |
| PRT-370 | SEQ ID NO: 406 | 1_61DEL AND 380_529del; D71Q, S128A, L153P, N219Q, |
| | | K324I, F333N, 177_183KEYQSNKdelinsRDNRDN (SEQ ID NOs: |
| | | 920 and 919) |
| PRT-371 | SEQ ID NO: 407 | 1_61DEL AND 380_529del; S128A, L153P, N162E, K229E, |
| | | K324I, F333N, 177_183KEYQSNKdelinsRDNRDN (SEQ ID NOs: |
| | | 920 and 919) |
| PRT-372 | SEQ ID NO: 408 | 1_61DEL AND 380_529del; S128A, L153P, N162E, K229E, |
| | | H275Y, K324I, F333N, 177_183KEYQSNKdelinsRDNRDN (SEQ ID |
| | | NOs: 920 and 919) |
| PRT-373 | SEQ ID NO: 409 | 1_61DEL AND 380_529del; T122S, S128A, L153P, N162E, |
| | | K229E, K324I, F333N, 177_183KEYQSNKdelinsRDNRDN (SEQ ID |
| | | NOs: 920 and 919) |
| PRT-374 | SEQ ID NO: 410 | 1_61DEL AND 380_529del; S128A, L153P, N162E, H174E, |
| | | G199P, N219G, K229E, H275Y, K324I, F333N, |
| | | 177_183KEYQSNKdelinsRDNRDN (SEQ ID NOs: 920 and 919) |
| PRT-375 | SEQ ID NO: 411 | 1_61DEL AND 380_529del; S128A, L153P, N162E, H174E, |
| | | G199P, N219G, K229E, I262K, H275Y, K324I, F333N, |
| | | 177_183KEYQSNKdelinsRDNRDN (SEQ ID NOs: 920 and 919) |
| PRT-376 | SEQ ID NO: 412 | 1_61DEL AND 380_529del; S128A, L153P, N162E, H174E, |
| | | G199P, N219G, K229E, H275Y, R282G, K324I, F333N, |
| | | 177_183KEYQSNKdelinsRDNRDN (SEQ ID NOs: 920 and 919) |
| PRT-377 | SEQ ID NO: 413 | 1_61DEL AND 380_529del; K93D, S128A, K150E, L153P, |
| | | N162E, H174E, G199P, N219G, K229E, H275Y, R282G, K324I, |
| | | F333N, 177_183KEYQSNKdelinsRDNRDN (SEQ ID NOs: 920 and |
| | | 919) |
| PRT-378 | SEQ ID NO: 414 | 1_61DEL AND 380_529del; S128A, D139E, L153P, N162E, |
| | | H174E, G199P, N219G, K229E, E241D, H275Y, R282G, K324I, |
| | | F333N, 177_183KEYQSNKdelinsRDNRDN (SEQ ID NOs: 920 and |
| | | 919) |
| PRT-379 | SEQ ID NO: 415 | 1_61DEL AND 380_529del; S128A, L153P, N162E, H174E, |
| | | G199P, N219G, K229E, H275Y, R282G, K324I, F333N, A343K, |
| | | R360L, 177_183KEYQSNKdelinsRDNRDN (SEQ ID NOs: 920 and |
| | | 919) |
| PRT-380 | SEQ ID NO: 416 | 1_61DEL AND 380_529del; S128A, D139E, L153P, N162E, |
| | | H174E, G199P, N219G, K229E, E241D, H275Y, R282G, K324I, |
| | | F333N, A343K, R360L, 177_183KEYQSNKdelinsRDNRDN (SEQ ID |
| | | NOs: 920 and 919) |
| PRT-381 | SEQ ID NO: 417 | 1_61DEL AND 380_529del; K93D, S128A, D139E, K150E, |
| | | L153P, N162E, H174E, G199P, N219G, K229E, E241D, H275Y, |
| | | R282G, K324I, F333N, 177_183KEYQSNKdelinsRDNRDN (SEQ ID |
| | | NOs: 920 and 919) |
| PRT-382 | SEQ ID NO: 418 | 1_61DEL AND 380_529del; K93D, S128A, K150E, L153P, |
| | | N162E, H174E, G199P, N219G, K229E, H275Y, R282G, K324I, |
| | | F333N, A343K, R360L, 177_183KEYQSNKdelinsRDNRDN (SEQ ID |
| | | NOs: 920 and 919) |
| PRT-383 | SEQ ID NO: 419 | 1_61DEL AND 380_529del; S128A, L153P, N162E, H174E, |
| | | G199P, N219G, K229E, I262K, H275Y, R282G, K324I, F333N, |
| | | 177_183KEYQSNKdelinsRDNRDN (SEQ ID NOs: 920 and 919) |
| PRT-384 | SEQ ID NO: 420 | 1_61DEL AND 380_529del; K93D, S128A, K150E, L153P, |
| | | N162E, H174E, G199P, N219G, K229E, I262K, H275Y, R282G, |
| | | K324I, F333N, 177_183KEYQSNKdelinsRDNRDN (SEQ ID NOs: |
| | | 920 and 919) |
| PRT-385 | SEQ ID NO: 421 | 1_61DEL AND 380_529del; S128A, D139E, L153P, N162E, |
| | | H174E, G199P, N219G, K229E, E241D, I262K, H275Y, R282G, |
| | | K324I, F333N, 177_183KEYQSNKdelinsRDNRDN (SEQ ID NOs: |
| | | 920 and 919) |
| PRT-386 | SEQ ID NO: 422 | 1_61DEL AND 380_529del; S128A, L153P, N162E, H174E, |
| | | G199P, N219G, K229E, I262K, H275Y, R282G, K324I, F333N, |
| | | A343K, R360L, 177_183KEYQSNKdelinsRDNRDN (SEQ ID NOs: |
| | | 920 and 919) |
| PRT-387 | SEQ ID NO: 423 | 1_61DEL AND 380_529del; S128A, D139E, L153P, N162E, |
| | | H174E, G199P, N219G, K229E, E241D, I262K, H275Y, R282G, |
| | | K324I, F333N, A343K, R360L, 177_183KEYQSNKdelinsRDNRDN |
| | | (SEQ ID NOs: 920 and 919) |
| PRT-388 | SEQ ID NO: 424 | 1_61DEL AND 380_529del; K93D, S128A, D139E, K150E, |
| | | L153P, N162E, H174E, G199P, N219G, K229E, E241D, I262K, |
| | | H275Y, R282G, K324I, F333N, 177_183KEYQSNKdelinsRDNRDN |
| | | (SEQ ID NOs: 920 and 919) |
| PRT-389 | SEQ ID NO: 425 | 1_61DEL AND 380_529del; K93D, S128A, K150E, L153P, |
| | | N162E, H174E, G199P, N219G, K229E, I262K, H275Y, R282G, |
| | | K324I, F333N, A343K, R360L, 177_183KEYQSNKdelinsRDNRDN |
| | | (SEQ ID NOs: 920 and 919) |
| PRT-390 | SEQ ID NO: 426 | 1_61DEL AND 380_529del; K93D, S128A, D139E, K150E, |
| | | L153P, N162E, H174E, G199P, N219G, K229E, E241D, I262K, |
| | | H275Y, R282G, S306P, K324I, F333N, |
| | | 177_183KEYQSNKdelinsRDNRDN (SEQ ID NOs: 920 and 919) |
| PRT-391 | SEQ ID NO: 427 | 1_61DEL AND 380_529del; K93D, S128A, K150E, L153P, |
| | | N162E, H174E, G199P, N219G, K229E, I262K, H275Y, R282G, |
| | | S306P, K324I, F333N, A343K, R360L, |
| | | 177_183KEYQSNKdelinsRDNRDN (SEQ ID NOs: 920 and 919) |
| PRT-392 | SEQ ID NO: 428 | 1_61DEL AND 380_529del; K93D, S128A, D139E, K150E, |
| | | L153P, N162E, H174E, G199P, N219G, K229E, E241D, I262K, |
| | | H275Y, R282G, S306P, K324I, F333N, A343K, R360L, |
| | | 177_183KEYQSNKdelinsRDNRDN (SEQ ID NOs: 920 and 919) |
| PRT-393 | SEQ ID NO: 429 | 1_61DEL AND 380_529del; K93D, S128A, K150E, L153P, |
| | | N162E, H174E, G199P, N219G, K229E, I262K, H275Y, R282G, |
| | | K324I, F333E, 177_183KEYQSNKdelinsRDNRDN (SEQ ID NOs: |
| | | 920 and 919) |
| PRT-394 | SEQ ID NO: 430 | 1_61DEL AND 380_529del; K93D, S128A, D139E, K150E, |
| | | L153P, N162E, H174E, G199P, N219G, K229E, E241D, I262K, |
| | | H275Y, R282G, K324I, F333E, 177_183KEYQSNKdelinsRDNRDN |
| | | (SEQ ID NOs: 920 and 919) |
| PRT-395 | SEQ ID NO: 431 | 1_61DEL AND 380_529del; K93D, S128A, D139E, K150E, |
| | | L153P, N162E, H174E, G199P, N219G, K229E, E241D, I262K, |
| | | H275Y, R282G, K324I, F333E, A343K, R360L, |
| | | 177_183KEYQSNKdelinsRDNRDN (SEQ ID NOs: 920 and 919) |
| PRT-396 | SEQ ID NO: 432 | 1_61DEL AND 380_529del; K93D, S128A, D139E, K150E, |
| | | L153P, N162E, H174E, G199P, N219G, K229E, E241D, I262K, |
| | | H275Y, R282G, S306P, K324I, F333E, |
| | | 177_183KEYQSNKdelinsRDNRDN (SEQ ID NOs: 920 and 919) |
| PRT-397 | SEQ ID NO: 433 | 1_61DEL AND 380_529del; K93D, S128A, K150E, L153P, |
| | | N162E, H174E, G199P, N219G, K229E, I262K, H275Y, R282G, |
| | | S306P, K324I, F333E, A343K, R360L, |
| | | 177_183KEYQSNKdelinsRDNRDN (SEQ ID NOs: 920 and 919) |
| PRT-398 | SEQ ID NO: 434 | 1_61DEL AND 380_529del; K93D, S128A, D139E, K150E, |
| | | L153P, N162E, H174E, G199P, N219G, K229E, E241D, I262K, |
| | | H275Y, R282G, S306P, K324I, F333E, A343K, R360L, |
| | | 177_183KEYQSNKdelinsRDNRDN (SEQ ID NOs: 920 and 919) |
| PRT-399 | SEQ ID NO: 435 | 1_61DEL AND 380_529del; S128A, L153P, N162E, H174E, |
| | | G199P, N219G, K229E, H275Y, Y281S, K324I, F333N, |
| | | 177_183KEYQSNKdelinsRDNRDN (SEQ ID NOs: 920 and 919) |
| PRT-400 | SEQ ID NO: 436 | 1_61DEL AND 380_529del; K93D, S128A, K150E, L153P, |
| | | N162E, H174E, G199P, N219G, K229E, H275Y, Y281S, K324I, |
| | | F333N, 177_183KEYQSNKdelinsRDNRDN (SEQ ID NOs: 920 and |
| | | 919) |
| PRT-401 | SEQ ID NO: 437 | 1_61DEL AND 380_529del; S128A, D139E, L153P, N162E, |
| | | H174E, G199P, N219G, K229E, E241D, H275Y, Y281S, K324I, |
| | | F333N, 177_183KEYQSNKdelinsRDNRDN (SEQ ID NOs: 920 and |
| | | 919) |
| PRT-402 | SEQ ID NO: 438 | 1_61DEL AND 380_529del; S128A, L153P, N162E, H174E, |
| | | G199P, N219G, K229E, H275Y, Y281S, K324I, F333N, A343K, |
| | | R360L, 177_183KEYQSNKdelinsRDNRDN (SEQ ID NOs: 920 and |
| | | 919) |
| PRT-403 | SEQ ID NO: 439 | 1_61DEL AND 380_529del; S128A, D139E, L153P, N162E, |
| | | H174E, G199P, N219G, K229E, E241D, H275Y, Y281S, K324I, |
| | | F333N, A343K, R360L, 177_183KEYQSNKdelinsRDNRDN (SEQ ID |
| | | NOs: 920 and 919) |
| PRT-404 | SEQ ID NO: 440 | 1_61DEL AND 380_529del; K93D, S128A, D139E, K150E, |
| | | L153P, N162E, H174E, G199P, N219G, K229E, E241D, H275Y, |
| | | Y281S, K324I, F333N, 177_183KEYQSNKdelinsRDNRDN (SEQ ID |
| | | NOs: 920 and 919) |
| PRT-405 | SEQ ID NO: 441 | 1_61DEL AND 380_529del; K93D, S128A, K150E, L153P, |
| | | N162E, H174E, G199P, N219G, K229E, H275Y, Y281S, K324I, |
| | | F333N, A343K, R360L, 177_183KEYQSNKdelinsRDNRDN (SEQ ID |
| | | NOs: 920 and 919) |
| PRT-406 | SEQ ID NO: 442 | 1_61DEL AND 380_529del; S128A, L153P, N162E, H174E, |
| | | G199P, N219G, K229E, I262K, H275Y, Y281S, K324I, F333N, |
| | | 177_183KEYQSNKdelinsRDNRDN (SEQ ID NOs: 920 and 919) |
| PRT-407 | SEQ ID NO: 443 | 1_61DEL AND 380_529del; K93D, S128A, K150E, L153P, |
| | | N162E, H174E, G199P, N219G, K229E, I262K, H275Y, Y281S, |
| | | K324I, F333N, 177_183KEYQSNKdelinsRDNRDN (SEQ ID NOs: |
| | | 920 and 919) |
| PRT-408 | SEQ ID NO: 444 | 1_61DEL AND 380_529del; S128A, D139E, L153P, N162E, |
| | | H174E, G199P, N219G, K229E, E241D, I262K, H275Y, Y281S, |
| | | K324I, F333N, 177_183KEYQSNKdelinsRDNRDN (SEQ ID NOs: |
| | | 920 and 919) |
| PRT-409 | SEQ ID NO: 445 | 1_61DEL AND 380_529del; S128A, L153P, N162E, H174E, |
| | | G199P, N219G, K229E, I262K, H275Y, Y281S, K324I, F333N, |
| | | A343K, R360L, 177_183KEYQSNKdelinsRDNRDN (SEQ ID NOs: |
| | | 920 and 919) |
| PRT-410 | SEQ ID NO: 446 | 1_61DEL AND 380_529del; S128A, D139E, L153P, N162E, |
| | | H174E, G199P, N219G, K229E, E241D, I262K, H275Y, Y281S, |
| | | K324I, F333N, A343K, R360L, 177_183KEYQSNKdelinsRDNRDN |
| | | (SEQ ID NOs: 920 and 919) |
| PRT-411 | SEQ ID NO: 447 | 1_61DEL AND 380_529del; K93D, S128A, D139E, K150E, |
| | | L153P, N162E, H174E, G199P, N219G, K229E, E241D, I262K, |
| | | H275Y, Y281S, K324I, F333N, 177_183KEYQSNKdelinsRDNRDN |
| | | (SEQ ID NOs: 920 and 919) |
| PRT-412 | SEQ ID NO: 448 | 1_61DEL AND 380_529del; K93D, S128A, K150E, L153P, |
| | | N162E, H174E, G199P, N219G, K229E, I262K, H275Y, Y281S, |
| | | K324I, F333N, A343K, R360L, 177_183KEYQSNKdelinsRDNRDN |
| | | (SEQ ID NOs: 920 and 919) |
| PRT-413 | SEQ ID NO: 449 | 1_61DEL AND 380_529del; K93D, S128A, D139E, K150E, |
| | | L153P, N162E, H174E, G199P, N219G, K229E, E241D, I262K, |
| | | H275Y, Y281S, S306P, K324I, F333N, |
| | | 177_183KEYQSNKdelinsRDNRDN (SEQ ID NOs: 920 and 919) |
| PRT-414 | SEQ ID NO: 450 | 1_61DEL AND 380_529del; K93D, S128A, K150E, L153P, |
| | | N162E, H174E, G199P, N219G, K229E, I262K, H275Y, Y281S, |
| | | S306P, K324I, F333N, A343K, R360L, |
| | | 177_183KEYQSNKdelinsRDNRDN (SEQ ID NOs: 920 and 919) |
| PRT-415 | SEQ ID NO: 451 | 1_61DEL AND 380_529del; K93D, S128A, D139E, K150E, |
| | | L153P, N162E, H174E, G199P, N219G, K229E, E241D, I262K, |
| | | H275Y, Y281S, S306P, K324I, F333N, A343K, R360L, |
| | | 177_183KEYQSNKdelinsRDNRDN (SEQ ID NOs: 920 and 919) |
| PRT-416 | SEQ ID NO: 452 | 1_61DEL AND 380_529del; K93D, S128A, K150E, L153P, |
| | | N162E, H174E, G199P, N219G, K229E, I262K, H275Y, Y281S, |
| | | K324I, F333E, 177_183KEYQSNKdelinsRDNRDN (SEQ ID NOs: |
| | | 920 and 919) |
| PRT-417 | SEQ ID NO: 453 | 1_61DEL AND 380_529del; K93D, S128A, D139E, K150E, |
| | | L153P, N162E, H174E, G199P, N219G, K229E, E241D, I262K, |
| | | H275Y, Y281S, K324I, F333E, 177_183KEYQSNKdelinsRDNRDN |
| | | (SEQ ID NOs: 920 and 919) |
| PRT-418 | SEQ ID NO: 454 | 1_61DEL AND 380_529del; K93D, S128A, D139E, K150E, |
| | | L153P, N162E, H174E, G199P, N219G, K229E, E241D, I262K, |
| | | H275Y, Y281S, K324I, F333E, A343K, R360L, |
| | | 177_183KEYQSNKdelinsRDNRDN (SEQ ID NOs: 920 and 919) |
| PRT-419 | SEQ ID NO: 455 | 1_61DEL AND 380_529del; K93D, S128A, D139E, K150E, |
| | | L153P, N162E, H174E, G199P, N219G, K229E, E241D, I262K, |
| | | H275Y, Y281S, S306P, K324I, F333E, |
| | | 177_183KEYQSNKdelinsRDNRDN (SEQ ID NOs: 920 and 919) |
| PRT-420 | SEQ ID NO: 456 | 1_61DEL AND 380_529del; K93D, S128A, K150E, L153P, |
| | | N162E, H174E, G199P, N219G, K229E, I262K, H275Y, Y281S, |
| | | S306P, K324I, F333E, A343K, R360L, |
| | | 177_183KEYQSNKdelinsRDNRDN (SEQ ID NOs: 920 and 919) |
| PRT-421 | SEQ ID NO: 457 | 1_61DEL AND 380_529del; K93D, S128A, D139E, K150E, |
| | | L153P, N162E, H174E, G199P, N219G, K229E, E241D, I262K, |
| | | H275Y, Y281S, S306P, K324I, F333E, A343K, R360L, |
| | | 177_183KEYQSNKdelinsRDNRDN (SEQ ID NOs: 920 and 919) |
| PRT-422 | SEQ ID NO: 458 | 1_61DEL AND 380_529del; K93D, S128A, K150E, L153P, |
| | | N162E, H174E, G199P, N219G, K229E, I262K, H275Y, K324I, |
| | | F333N, 177_183KEYQSNKdelinsRDNRDN (SEQ ID NOs: 920 and |
| | | 919) |
| PRT-423 | SEQ ID NO: 459 | 1_61DEL AND 380_529del; S128A, D139E, L153P, N162E, |
| | | H174E, G199P, N219G, K229E, E241D, I262K, H275Y, K324I, |
| | | F333N, 177_183KEYQSNKdelinsRDNRDN (SEQ ID NOs: 920 and |
| | | 919) |
| PRT-424 | SEQ ID NO: 460 | 1_61DEL AND 380_529del; S128A, L153P, N162E, H174E, |
| | | G199P, N219G, K229E, I262K, H275Y, K324I, F333N, A343K, |
| | | R360L, 177_183KEYQSNKdelinsRDNRDN (SEQ ID NOs: 920 and |
| | | 919) |
| PRT-425 | SEQ ID NO: 461 | 1_61DEL AND 380_529del; S128A, D139E, L153P, N162E, |
| | | H174E, G199P, N219G, K229E, E241D, I262K, H275Y, K324I, |
| | | F333N, A343K, R360L, 177_183KEYQSNKdelinsRDNRDN (SEQ ID |
| | | NOs: 920 and 919) |
| PRT-426 | SEQ ID NO: 462 | 1_61DEL AND 380_529del; K93D, S128A, D139E, K150E, |
| | | L153P, N162E, H174E, G199P, N219G, K229E, E241D, I262K, |
| | | H275Y, K324I, F333N, 177_183KEYQSNKdelinsRDNRDN (SEQ ID |
| | | NOs: 920 and 919) |
| PRT-427 | SEQ ID NO: 463 | 1_61DEL AND 380_529del; K93D, S128A, K150E, L153P, |
| | | N162E, H174E, G199P, N219G, K229E, I262K, H275Y, K324I, |
| | | F333N, A343K, R360L, 177_183KEYQSNKdelinsRDNRDN (SEQ ID |
| | | NOs: 920 and 919) |
| PRT-428 | SEQ ID NO: 464 | 1_61DEL AND 380_529del; K93D, S128A, D139E, K150E, |
| | | L153P, N162E, H174E, G199P, N219G, K229E, E241D, I262K, |
| | | H275Y, S306P, K324I, F333N, 177_183KEYQSNKdelinsRDNRDN |
| | | (SEQ ID NOs: 920 and 919) |
| PRT-429 | SEQ ID NO: 465 | 1_61DEL AND 380_529del; K93D, S128A, K150E, L153P, |
| | | N162E, H174E, G199P, N219G, K229E, I262K, H275Y, S306P, |
| | | K324I, F333N, A343K, R360L, 177_183KEYQSNKdelinsRDNRDN |
| | | (SEQ ID NOs: 920 and 919) |
| PRT-430 | SEQ ID NO: 466 | 1_61DEL AND 380_529del; K93D, S128A, D139E, K150E, |
| | | L153P, N162E, H174E, G199P, N219G, K229E, E241D, I262K, |
| | | H275Y, S306P, K324I, F333N, A343K, R360L, |
| | | 177_183KEYQSNKdelinsRDNRDN (SEQ ID NOs: 920 and 919) |
| PRT-431 | SEQ ID NO: 467 | 1_61DEL AND 380_529del; K93D, S128A, K150E, L153P, |
| | | N162E, H174E, G199P, N219G, K229E, I262K, H275Y, K324I, |
| | | F333E, 177_183KEYQSNKdelinsRDNRDN (SEQ ID NOs: 920 and |
| | | 919) |
| PRT-432 | SEQ ID NO: 468 | 1_61DEL AND 380_529del; K93D, S128A, D139E, K150E, |
| | | L153P, N162E, H174E, G199P, N219G, K229E, E241D, I262K, |
| | | H275Y, K324I, F333E, 177_183KEYQSNKdelinsRDNRDN (SEQ ID |
| | | NOs: 920 and 919) |
| PRT-433 | SEQ ID NO: 469 | 1_61DEL AND 380_529del; K93D, S128A, D139E, K150E, |
| | | L153P, N162E, H174E, G199P, N219G, K229E, E241D, I262K, |
| | | H275Y, K324I, F333E, A343K, R360L, |
| | | 177_183KEYQSNKdelinsRDNRDN (SEQ ID NOs: 920 and 919) |
| PRT-434 | SEQ ID NO: 470 | 1_61DEL AND 380_529del; K93D, S128A, D139E, K150E, |
| | | L153P, N162E, H174E, G199P, N219G, K229E, E241D, I262K, |
| | | H275Y, S306P, K324I, F333E, 177_183KEYQSNKdelinsRDNRDN |
| | | (SEQ ID NOs: 920 and 919) |
| PRT-435 | SEQ ID NO: 471 | 1_61DEL AND 380_529del; K93D, S128A, K150E, L153P, |
| | | N162E, H174E, G199P, N219G, K229E, I262K, H275Y, S306P, |
| | | K324I, F333E, A343K, R360L, 177_183KEYQSNKdelinsRDNRDN |
| | | (SEQ ID NOs: 920 and 919) |
| PRT-436 | SEQ ID NO: 472 | 1_61DEL AND 380_529del; K93D, S128A, D139E, K150E, |
| | | L153P, N162E, H174E, G199P, N219G, K229E, E241D, I262K, |
| | | H275Y, S306P, K324I, F333E, A343K, R360L, |
| | | 177_183KEYQSNKdelinsRDNRDN (SEQ ID NOs: 920 and 919) |
| PRT-437 | SEQ ID NO: 473 | 1_61DEL AND 380_529del; S128A, L153P, N162E, H174E, |
| | | H197D, N219G, K229E, I262K, H275Y, K324I, F333N, |
| | | 177_183KEYQSNKdelinsRDNRDN (SEQ ID NOs: 920 and 919) |
| PRT-438 | SEQ ID NO: 474 | 1_61DEL AND 380_529del; K93D, S128A, K150E, L153P, |
| | | N162E, H174E, H197D, N219G, K229E, I262K, H275Y, K324I, |
| | | F333N, 177_183KEYQSNKdelinsRDNRDN (SEQ ID NOs: 920 and |
| | | 919) |
| PRT-439 | SEQ ID NO: 475 | 1_61DEL AND 380_529del; S128A, D139E, L153P, N162E, |
| | | H174E, H197D, N219G, K229E, E241D, I262K, H275Y, K324I, |
| | | F333N, 177_183KEYQSNKdelinsRDNRDN (SEQ ID NOs: 920 and |
| | | 919) |
| PRT-440 | SEQ ID NO: 476 | 1_61DEL AND 380_529del; S128A, L153P, N162E, H174E, |
| | | H197D, N219G, K229E, I262K, H275Y, K324I, F333N, A343K, |
| | | R360L, 177_183KEYQSNKdelinsRDNRDN (SEQ ID NOs: 920 and |
| | | 919) |
| PRT-441 | SEQ ID NO: 477 | 1_61DEL AND 380_529del; S128A, D139E, L153P, N162E, |
| | | H174E, H197D, N219G, K229E, E241D, I262K, H275Y, K324I, |
| | | F333N, A343K, R360L, 177_183KEYQSNKdelinsRDNRDN (SEQ ID |
| | | NOs: 920 and 919) |
| PRT-442 | SEQ ID NO: 478 | 1_61DEL AND 380_529del; K93D, S128A, D139E, K150E, |
| | | L153P, N162E, H174E, H197D, N219G, K229E, E241D, I262K, |
| | | H275Y, K324I, F333N, 177_183KEYQSNKdelinsRDNRDN (SEQ ID |
| | | NOs: 920 and 919) |
| PRT-443 | SEQ ID NO: 479 | 1_61DEL AND 380_529del; K93D, S128A, K150E, L153P, |
| | | N162E, H174E, H197D, N219G, K229E, I262K, H275Y, K324I, |
| | | F333N, A343K, R360L, 177_183KEYQSNKdelinsRDNRDN (SEQ ID |
| | | NOs: 920 and 919) |
| PRT-444 | SEQ ID NO: 480 | 1_61DEL AND 380_529del; K93D, S128A, D139E, K150E, |
| | | L153P, N162E, H174E, H197D, N219G, K229E, E241D, I262K, |
| | | H275Y, S306P, K324I, F333N, 177_183KEYQSNKdelinsRDNRDN |
| | | (SEQ ID NOs: 920 and 919) |
| PRT-445 | SEQ ID NO: 481 | 1_61DEL AND 380_529del; K93D, S128A, K150E, L153P, |
| | | N162E, H174E, H197D, N219G, K229E, I262K, H275Y, S306P, |
| | | K324I, F333N, A343K, R360L, 177_183KEYQSNKdelinsRDNRDN |
| | | (SEQ ID NOs: 920 and 919) |
| PRT-446 | SEQ ID NO: 482 | 1_61DEL AND 380_529del; K93D, S128A, D139E, K150E, |
| | | L153P, N162E, H174E, H197D, N219G, K229E, E241D, I262K, |
| | | H275Y, S306P, K324I, F333N, A343K, R360L, |
| | | 177_183KEYQSNKdelinsRDNRDN (SEQ ID NOs: 920 and 919) |
| PRT-447 | SEQ ID NO: 483 | 1_61DEL AND 380_529del; K93D, S128A, K150E, L153P, |
| | | N162E, H174E, H197D, N219G, K229E, I262K, H275Y, K324I, |
| | | F333E, 177_183KEYQSNKdelinsRDNRDN (SEQ ID NOs: 920 and |
| | | 919) |
| PRT-448 | SEQ ID NO: 484 | 1_61DEL AND 380_529del; K93D, S128A, D139E, K150E, |
| | | L153P, N162E, H174E, H197D, N219G, K229E, E241D, I262K, |
| | | H275Y, K324I, F333E, 177_183KEYQSNKdelinsRDNRDN (SEQ ID |
| | | NOs: 920 and 919) |
| PRT-449 | SEQ ID NO: 485 | 1_61DEL AND 380_529del; K93D, S128A, D139E, K150E, |
| | | L153P, N162E, H174E, H197D, N219G, K229E, E241D, I262K, |
| | | H275Y, K324I, F333E, A343K, R360L, |
| | | 177_183KEYQSNKdelinsRDNRDN (SEQ ID NOs: 920 and 919) |
| PRT-450 | SEQ ID NO: 486 | 1_61DEL AND 380_529del; K93D, S128A, D139E, K150E, |
| | | L153P, N162E, H174E, H197D, N219G, K229E, E241D, I262K, |
| | | H275Y, S306P, K324I, F333E, 177_183KEYQSNKdelinsRDNRDN |
| | | (SEQ ID NOs: 920 and 919) |
| PRT-451 | SEQ ID NO: 487 | 1_61DEL AND 380_529del; K93D, S128A, K150E, L153P, |
| | | N162E, H174E, H197D, N219G, K229E, I262K, H275Y, S306P, |
| | | K324I, F333E, A343K, R360L, 177_183KEYQSNKdelinsRDNRDN |
| | | (SEQ ID NOs: 920 and 919) |
| PRT-452 | SEQ ID NO: 488 | 1_61DEL AND 380_529del; K93D, S128A, D139E, K150E, |
| | | L153P, N162E, H174E, H197D, N219G, K229E, E241D, I262K, |
| | | H275Y, S306P, K324I, F333E, A343K, R360L, |
| | | 177_183KEYQSNKdelinsRDNRDN (SEQ ID NOs: 920 and 919) |
| PRT-453 | SEQ ID NO: 489 | 1_61DEL AND 380_529del; K93D, S128A, K150E, L153P, |
| | | N162E, H174E, G199P, N219G, K229E, H275Y, K324I, F333N, |
| | | 177_183KEYQSNKdelinsRDNRDN (SEQ ID NOs: 920 and 919) |
| PRT-454 | SEQ ID NO: 490 | 1_61DEL AND 380_529del; S128A, D139E, L153P, N162E, |
| | | H174E, G199P, N219G, K229E, E241D, H275Y, K324I, F333N, |
| | | 177_183KEYQSNKdelinsRDNRDN (SEQ ID NOs: 920 and 919) |
| PRT-455 | SEQ ID NO: 491 | 1_61DEL AND 380_529del; S128A, L153P, N162E, H174E, |
| | | G199P, N219G, K229E, H275Y, K324I, F333N, A343K, R360L, |
| | | 177_183KEYQSNKdelinsRDNRDN (SEQ ID NOs: 920 and 919) |
| PRT-456 | SEQ ID NO: 492 | 1_61DEL AND 380_529del; S128A, D139E, L153P, N162E, |
| | | H174E, G199P, N219G, K229E, E241D, H275Y, K324I, F333N, |
| | | A343K, R360L, 177_183KEYQSNKdelinsRDNRDN (SEQ ID NOs: |
| | | 920 and 919) |
| PRT-457 | SEQ ID NO: 493 | 1_61DEL AND 380_529del; K93D, S128A, D139E, K150E, |
| | | L153P, N162E, H174E, G199P, N219G, K229E, E241D, H275Y, |
| | | K324I, F333N, 177_183KEYQSNKdelinsRDNRDN (SEQ ID NOs: |
| | | 920 and 919) |
| PRT-458 | SEQ ID NO: 494 | 1_61DEL AND 380_529del; K93D, S128A, K150E, L153P, |
| | | N162E, H174E, G199P, N219G, K229E, H275Y, K324I, F333N, |
| | | A343K, R360L, 177_183KEYQSNKdelinsRDNRDN (SEQ ID NOs: |
| | | 920 and 919) |
| PRT-459 | SEQ ID NO: 495 | 1_61DEL AND 380_529del; S128A, D139E, L153P, N162E, |
| | | N219G, K229E, H275Y, K324I, F333N, |
| | | 177_183KEYQSNKdelinsRDNRDN (SEQ ID NOs: 920 and 919) |
| PRT-460 | SEQ ID NO: 496 | 1_61DEL AND 380_529del; S128A, L153P, N162E, N219G, |
| | | K229E, E241D, H275Y, K324I, F333N, |
| | | 177_183KEYQSNKdelinsRDNRDN (SEQ ID NOs: 920 and 919) |
| PRT-461 | SEQ ID NO: 497 | 1_61DEL AND 380_529del; S128A, L153P, N162E, N219G, |
| | | K229E, H275Y, K324I, F333N, A343K, |
| | | 177_183KEYQSNKdelinsRDNRDN (SEQ ID NOs: 920 and 919) |
| PRT-462 | SEQ ID NO: 498 | 1_61DEL AND 380_529del; S128A, L153P, N162E, N219G, |
| | | K229E, H275Y, K324I, F333E, 177_183KEYQSNKdelinsRDNRDN |
| | | (SEQ ID NOs: 920 and 919) |
| PRT-463 | SEQ ID NO: 499 | 1_61DEL AND 380_529del; S128A, L153P, N162E, N219G, |
| | | K229E, H275Y, R282G, K324I, F333N, |
| | | 177_183KEYQSNKdelinsRDNRDN (SEQ ID NOs: 920 and 919) |
| PRT-464 | SEQ ID NO: 500 | 1_61DEL AND 380_529del; S128A, L153P, N162E, H197D, |
| | | N219G, K229E, H275Y, K324I, F333N, |
| | | 177_183KEYQSNKdelinsRDNRDN (SEQ ID NOs: 920 and 919) |
| PRT-465 | SEQ ID NO: 501 | 1_61DEL AND 380_529del; S128A, L153P, N162E, N219G, |
| | | K229E, H275Y, K324I, F333N, R360L, |
| | | 177_183KEYQSNKdelinsRDNRDN (SEQ ID NOs: 920 and 919) |
| PRT-466 | SEQ ID NO: 502 | 1_61DEL AND 380_529del; S128A, L153P, N162E, N219G, |
| | | K229E, I262K, H275Y, K324I, F333N, |
| | | 177_183KEYQSNKdelinsRDNRDN (SEQ ID NOs: 920 and 919) |
| PRT-467 | SEQ ID NO: 503 | 1_61DEL AND 380_529del; S128A, L153P, N162E, G199P, |
| | | N219G, K229E, H275Y, K324I, F333N, |
| | | 177_183KEYQSNKdelinsRDNRDN (SEQ ID NOs: 920 and 919) |
| PRT-468 | SEQ ID NO: 504 | 1_61DEL AND 380_529del; S128A, L153P, N162E, N219G, |
| | | K229E, H275Y, Y281S, K324I, F333N, |
| | | 177_183KEYQSNKdelinsRDNRDN (SEQ ID NOs: 920 and 919) |
| PRT-469 | SEQ ID NO: 505 | 1_61DEL AND 380_529del; K93D, S128A, L153P, N162E, |
| | | N219G, K229E, H275Y, K324I, F333N, |
| | | 177_183KEYQSNKdelinsRDNRDN (SEQ ID NOs: 920 and 919) |
| PRT-470 | SEQ ID NO: 506 | 1_61DEL AND 380_529del; S128A, L153P, N162E, N219G, |
| | | K229E, H275Y, S306P, K324I, F333N, |
| | | 177_183KEYQSNKdelinsRDNRDN (SEQ ID NOs: 920 and 919) |
| PRT-471 | SEQ ID NO: 507 | 1_61DEL AND 380_529del; S128A, K150E, L153P, N162E, |
| | | N219G, K229E, H275Y, K324I, F333N, |
| | | 177_183KEYQSNKdelinsRDNRDN (SEQ ID NOs: 920 and 919) |
| PRT-472 | SEQ ID NO: 508 | 1_61DEL AND 380_529del; S128A, L153P, N162E, H174E, |
| | | K196S, N219G, K229E, H275Y, K318A, K324I, F333N, |
| | | 177_183KEYQSNKdelinsRDNRDN (SEQ ID NOs: 920 and 919) |
| PRT-473 | SEQ ID NO: 509 | 1_61DEL AND 380_529del; S128A, L153P, N162E, H174E, |
| | | N219G, H222Q, K229E, H275Y, R280E, K324I, F333N, |
| | | 177_183KEYQSNKdelinsRDNRDN (SEQ ID NOs: 920 and 919) |
| PRT-474 | SEQ ID NO: 510 | 1_61DEL AND 380_529del; S128A, L153P, N162E, H174E, |
| | | K196S, N219G, H222Q, K229E, H275Y, R280E, K318A, K324I, |
| | | F333N, 177_183KEYQSNKdelinsRDNRDN (SEQ ID NOs: 920 and |
| | | 919) |
| PRT-475 | SEQ ID NO: 511 | 1_61DEL AND 380_529del; S128A, L153P, N162E, H174E, |
| | | N219G, K229E, H275Y, K324I, D332K, F333N, |
| | | 177_183KEYQSNKdelinsRDNRDN (SEQ ID NOs: 920 and 919) |
| PRT-476 | SEQ ID NO: 512 | 1_61DEL AND 380_529del; S128A, L153P, N162E, H174E, |
| | | N219G, K229E, N233G, H275Y, K324I, F333N, |
| | | 177_183KEYQSNKdelinsRDNRDN (SEQ ID NOs: 920 and 919) |
| PRT-477 | SEQ ID NO: 513 | 1_61DEL AND 380_529del; S128A, L153P, N162E, H174E, |
| | | N219G, K229E, I254R, H275Y, K324I, F333N, |
| | | 177_183KEYQSNKdelinsRDNRDN (SEQ ID NOs: 920 and 919) |
| PRT-478 | SEQ ID NO: 514 | 1_61DEL AND 380_529del; S128A, L153P, N162E, H174E, |
| | | N219G, K229E, H275Y, K324I, F333N, E363K, |
| | | 177_183KEYQSNKdelinsRDNRDN (SEQ ID NOs: 920 and 919) |
| PRT-479 | SEQ ID NO: 515 | 1_61DEL AND 380_529del; S128A, L153P, N162E, H174E, |
| | | N219G, K229E, H275Y, K324I, F333N, E366K, |
| | | 177_183KEYQSNKdelinsRDNRDN (SEQ ID NOs: 920 and 919) |
| PRT-480 | SEQ ID NO: 516 | 1_61DEL AND 380_529del; S128A, L153P, N162E, H174E, |
| | | N219G, K229E, H275Y, K324I, F333N, E347N, |
| | | 177_183KEYQSNKdelinsRDNRDN (SEQ ID NOs: 920 and 919) |
| PRT-481 | SEQ ID NO: 517 | 1_61DEL AND 380_529del; T122S, S128A, L153P, N162E, |
| | | H174E, K196S, N219G, K229E, H275Y, K318A, K324I, F333N, |
| | | 177_183KEYQSNKdelinsRDNRDN (SEQ ID NOs: 920 and 919) |
| PRT-482 | SEQ ID NO: 518 | 1_61DEL AND 380_529del; T122S, S128A, L153P, N162E, |
| | | H174E, N219G, H222Q, K229E, H275Y, R280E, K324I, F333N, |
| | | 177_183KEYQSNKdelinsRDNRDN (SEQ ID NOs: 920 and 919) |
| PRT-483 | SEQ ID NO: 519 | 1_61DEL AND 380_529del; T122S, S128A, L153P, N162E, |
| | | H174E, K196S, N219G, H222Q, K229E, H275Y, R280E, K318A, |
| | | K324I, F333N, 177_183KEYQSNKdelinsRDNRDN (SEQ ID NOs: |
| | | 920 and 919) |
| PRT-484 | SEQ ID NO: 520 | 1_61DEL AND 380_529del; S128A, L153P, N162E, H174E, |
| | | N219G, K229E, H275Y, K324I, F333N, E363K, E366K, |
| | | 177_183KEYQSNKdelinsRDNRDN (SEQ ID NOs: 920 and 919) |
| PRT-485 | SEQ ID NO: 521 | 1_61DEL AND 380_529del; S128A, L153P, N162E, H174E, |
| | | N219G, K229E, H275Y, K324I, D332K, F333N, E347N, |
| | | 177_183KEYQSNKdelinsRDNRDN (SEQ ID NOs: 920 and 919) |
| PRT-486 | SEQ ID NO: 522 | 1_61DEL AND 380_529del; S128A, L153P, N162E, H174E, |
| | | N219G, K229E, I254R, H275Y, K324I, D332K, F333N, E347N, |
| | | E363K, E366K, 177_183KEYQSNKdelinsRDNRDN (SEQ ID NOs: |
| | | 920 and 919) |
| PRT-487 | SEQ ID NO: 523 | 1_61DEL AND 380_529del; C125S, S128A, L153P, N162E, |
| | | H174E, N219G, K229E, H275Y, K324I, F333N, |
| | | 177_183KEYQSNKdelinsRDNRDN (SEQ ID NOs: 920 and 919) |
| PRT-488 | SEQ ID NO: 524 | 1_61DEL AND 380_529del; S128A, D152N, L153P, N162E, |
| | | H174E, N219G, K229E, H275Y, K324I, F333N, |
| | | 177_183KEYQSNKdelinsRDNRDN (SEQ ID NOs: 920 and 919) |
| PRT-489 | SEQ ID NO: 525 | 1_61DEL AND 380_529del; S128A, L153P, N162E, H174E, |
| | | N219G, K229E, H275Y, K324I, F333E, |
| | | 177_183KEYQSNKdelinsRDNRDN (SEQ ID NOs: 920 and 919) |
| PRT-490 | SEQ ID NO: 526 | 1_61DEL AND 380_529del; S128A, L153P, N162E, H174E, |
| | | H197D, N219G, K229E, H275Y, K324I, F333N, |
| | | 177_183KEYQSNKdelinsRDNRDN (SEQ ID NOs: 920 and 919) |
| PRT-491 | SEQ ID NO: 527 | 1_61DEL AND 380_529del; S128A, L153P, N162E, H174E, |
| | | N219G, K229E, N233G, H275Y, K324I, D332K, F333N, |
| | | 177_183KEYQSNKdelinsRDNRDN (SEQ ID NOs: 920 and 919) |
| PRT-492 | SEQ ID NO: 528 | 1_61DEL AND 380_529del; S128A, L153P, N162E, N219Q, |
| | | K229E, R280T, K324I, F333N |
| PRT-493 | SEQ ID NO: 737 | 1_61DEL AND 380_529del; S128A, L153P, N162E, N219Q, |
| | | K229E, R280T, D307N, K324I, F333N |

TABLE 3B

| | | Mutation(s) or deletions |
|---|---|---|
| ID | Polypeptide Seq | (as compared to SEQ ID NO: 2) |
| PRT-242 | SEQ ID NO; 247 | 1_50del and 371_520del |
| PRT-240 | SEQ ID NO: 242 | 1_50del and 371_520del; S187K |
| PRT-241 | SEQ ID NO: 243 | 1_50del and 371_520del; Q157E, S187K |

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 380, 251, 5-243, 247, 280-379, 381-528, 737, 745-823, 828-864, or 885-916, provided that the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1, and wherein X comprises a Methionine (M), a signal peptide, or is absent.

In some embodiments, the polypeptide having protease activity comprises an amino acid sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 380, 251, 5-243, 247, 280-379, 381-528, 737, 745-823, 828-864, or 885-916, provided that: 1) the amino acid sequence comprises any one mutation selected from Table 3A or Table 3B; and 2) the amino acid sequence comprises a cysteine (C) at a position in the polypeptide that corresponds to position 125 of SEQ ID NO: 1, a histidine (H) at a position in the polypeptide that corresponds to position 283 of SEQ ID NO: 1, an aspartic acid (D) at a position in the polypeptide that corresponds to position 305 of SEQ ID NO: 1, an aspartic acid (D) at a position in the polypeptide that corresponds to position 307 of SEQ ID NO: 1, and/or a lysine (K) at a position in the polypeptide that corresponds to position 115 of SEQ ID NO: 1, and wherein X comprises a Methionine (M), a signal peptide, or is absent.

In some embodiments, the polypeptide has the amino acid sequence of:

(SEQ ID NO: 738)
TALGRRKAEDRIKETLWADGVKIDEKDFEHIKSSHEDYFAVEYGKG
SGYYDVNKKMDSEDTALCVGX₁₂₈VVTNMFHWWVDQNRENIEKFKKQ
DX₁₅₃ENNGVMKFX₁₆₂GVKEATVDELNHFYKEYQSNKYADQSRFFDL
IKKHFGNKALNPEELFELYFNGFYYNSSHRYIEDNX₂₂₉PAQNNLFS
KVLENNKLLNPERSYGIDGFSNNVINALKSHKVLGLVHTTPLRYRH
IISMWGADIDQDGKVKAIYVTDSDDREITFNDNSKRRIGMX₃₂₄RYD
VLVDDX₃₃₃GNIRFTGKGATHKEEGAIYAGLYSLSRGDEVWEDYFKK
YPEVQENL,

wherein at least one X₁₂₈, X₁₅₃, X₁₆₂, X₂₂₉, X₃₂₄, and X₃₃₃ is mutated as compared to SEQ ID NO: 1. In some embodiments, the polypeptide has the amino acid sequence of SEQ ID NO: 738, wherein at least one of X₁₂₈, X₁₅₃, X₁₆₂, X₂₂₉, X₃₂₄, and X₃₃₃ is mutated as compared to SEQ ID NO: 1.

In some embodiments, the polypeptide has the amino acid sequence of:

(SEQ ID NO: 739)
TALGRRKAEDRIKETLWADGVKIDEKDFEHIKSSHEDYFAVEYGKG
SGYYDVNKKMDSEDTALCVGX₁₂₈VVTNMFHWWVDQNRENIEKFKKQ
DX₁₅₃ENNGVMKFX₁₆₂GVKEATVDELNX₁₇₄FYX₁₇₇YADQSRFFDLIK
KHFGNKALNPEELFELYFNGFYYX₂₁₉SSHRYIEDNX₂₂₉PAQNNLFS
KVLENNKLLNPERSYGIDGFSNNVINALKSHKVLGLVX₂₇₅TTPLRY
RHIISMWGADIDQDGKVKAIYVTDSDDREITFNDNSKRRIGMX₃₂₄R
YDVLVDDX₃₃₃GNIRFTGKGATHKEEGAIYAGLYSLSRGDEVWEDYF
KKYPEVQENL,

wherein at least one X₁₂₈, X₁₅₃, X₁₆₂, X₁₇₄, X₁₇₇, X₂₁₉, X₂₂₉, X₂₇₅, X₃₂₄, and X₃₃₃ is mutated as compared to SEQ ID NO: 1. In some embodiments, the polypeptide has the amino acid sequence of SEQ ID NO: 739, wherein at least one of X₁₂₈, X₁₅₃, X₁₆₂, X₁₇₄, X₁₇₇, X₂₁₉, X₂₂₉, X₂₇₅, X₃₂₄, and X₃₃₃ is mutated as compared to SEQ ID NO: 1.

In some embodiments, the polypeptide has the amino acid sequence of:

(SEQ ID NO: 740)
TALGRRKAEDRIKETLWADGVKIDEKDFEHIX₉₃SSHEDYFAVEYGK
GSGYYDVNKKMDSEDX₁₂₂ALCVGX₁₂₈VVTNMFHWWVDQNRENIEKF
KX₁₅₀QDX₁₅₃ENNGVMKFX₁₆₂GVKEATVDELNX₁₇₄FYX₁₇₇YADQSRF
FDLIKKHFX₁₉₉NKALNPEELFELYFNGFYYX₂₁₉SSHRYIEDNX₂₂₉P
AQNNLFSKVLX₂₄₁NNKLLNPERSYGIDGFSNNVX262NALKSHKVLG
LVX₂₇₅TTPLRYRHIISMWGADIDQDGKVKAIYVTDSDDREITFNDN
SKRRIGMX₃₂₄RYDVLVDDX₃₃₃GNIRFTGKGATHKEEGAIYAGLYSL
SRGDEVWEDYFKKYPEVQENL,

wherein at least one X₉₃, X₁₂₂, X₁₂₈, X₁₅₀, X₁₅₃, X₁₆₂, X₁₇₄, X₁₇₇, X₁₉₉, X₂₁₉, X₂₂₉, X₂₄₁, X₂₇₅, X₃₂₄, and X₃₃₃ is mutated as compared to SEQ ID NO: 1. In some embodiments, the polypeptide has the amino acid sequence of SEQ ID NO: 740, wherein at least one of X₉₃, X₁₂₂, X₁₂₈, X₁₅₀, X₁₅₃, X₁₆₂, X₁₇₄, X₁₇₇, X₁₉₉, X₂₁₉, X₂₂₉, X₂₄₁, X₂₇₅, X₃₂₄, and X₃₃₃ is mutated as compared to SEQ ID NO: 1.

In some embodiments, the polypeptide has the amino acid sequence of:

(SEQ ID NO: 741)
TALGRRKAEDRIKETLWADGVKIDEKDFEHIX₉₃SSHEDYFAVEYGK
GSGYYDVNKKMDSEDX₁₂₂ALCVGX₁₂₈VVTNMFHWWVX₁₃₉QNRENIE
KFKX₁₅₀QDX₁₅₃ENNGVMKFX₁₆₂GVKEATVDELNX₁₇₄FYX₁₇₇YADQS
RFFDLIKX₁₉₆HFX₁₉₉NKALNPEELFELYFNGFYYX₂₁₉SSX₂₂₂RYIE
DNX₂₂₉PAQNNLFSKVLX₂₄₁NNKLLNPERSYGIDGFSNNVX₂₆₂NALK
SHKVLGLVX₂₇₅TTPLX₂₈₀YRHIISMWGADIDQDGKVKAIYVTDSDD
REITFNDNSKRRIGMX₃₂₄RYDVLVDDX₃₃₃GNIRFTGKGX₃₄₃THKEE
GAIYAGLYSLSX₃₆₀GDEVWEDYFKKYPEVQENL,

wherein at least one X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is mutated as compared to SEQ ID NO: 1. In some embodiments, the polypeptide has the amino acid sequence of SEQ ID NO: 741, wherein at least one of X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is mutated as compared to SEQ ID NO: 1.

In some embodiments, the polypeptide has the amino acid sequence of:

(SEQ ID NO: 917)
X₁TALGRRKAEDRIKETLWADGVKIDEKDFEHIX₉₃SSHEDYFAVEY
GKGSGYYDVNKKMDSEDX₁₂₂ALCVGX₁₂₈VVTNMFHWWVX₁₃₉QNREN
IEKFKX₁₅₀QDX₁₅₃ENNGVMKFX₁₆₂GVKEATVDELNX₁₇₄FYX₁₇₇YAD
QSRFFDLIKX₁₉₆HFX₁₉₉NKALNPEELFELYFNGFYYX₂₁₉SSX₂₂₂RY
IEDNX₂₂₉PAQNNLFSKVLX₂₄₁NNKLLNPERSYGIDGFSNNVX₂₆₂NA
LKSHKVLGLVX₂₇₅TTPLX₂₈₀YRHIISMWGADIDQDGKVKAIYVTDS
DDREITFNDNSKRRIGMX₃₂₄RYDVLVDDX₃₃₃GNIRFTGKGX₃₄₃THK
EEGAIYAGLYSLSX₃₆₀GDEVWEDYFKKYPEVQENLX₃₈₀,

at least one X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is mutated as compared to SEQ ID NO: 1, wherein X₃₈₀ is either SEQ ID NO: 744, or is absent, and wherein X₁ is SEQ ID NO: 248, or is absent.

In some embodiments, X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is any amino acid, provided that at least one of X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is mutated as compared to SEQ ID NO: 1. In some embodiments, at least one of X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is not a wild-type residue as compared to SEQ ID NO: 1. In some embodiments, X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is any amino acid, provided that at least one of X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is not a wild-type residue as compared to SEQ ID NO: 1.

In some embodiments, X₉₃ is aspartic acid (D). In some embodiments, X₁₂₂ is serine(S). In some embodiments, X₁₂₈ is alanine (A). In some embodiments, X₁₃₉ is glutamic acid (E). In some embodiments, X₁₅₀ is glutamic acid (E). In some embodiments, X₁₅₃ is proline (P). In some embodiments, X₁₆₂ is glutamic acid (E). In some embodiments, X₁₇₄ is glutamic acid (E). In some embodiments, X₁₇₇ is an amino acid sequence KEYQSNK (SEQ ID NO: 742), or an amino acid sequence RDNRDN (SEQ ID NO: 743). In some embodiments, X₁₇₇ is an amino acid sequence RDNRDN (SEQ ID NO: 743). In some embodiments, X₁₇₇ is an amino acid sequence KEYQSNK (SEQ ID NO: 742). In some embodiments, X₁₉₆ is serine(S). In some embodiments, X₁₉₉ is proline (P). In some embodiments, X₂₁₉ is glycine (G). In some embodiments, X₂₂₂ is glutamine (Q). In some embodiments, X₂₂₉ is glutamic acid (E). In some embodiments, X₂₄₁ is aspartic acid (D). In some embodiments, X₂₆₂ is lysine (K). In some embodiments, X₂₇₅ is tyrosine (Y). In some embodiments, X₂₈₀ is threonine (T). In some embodiments, X₃₂₄ is isoleucine (I). In some embodiments, X₃₃₃ is asparagine (N). In some embodiments, X₃₄₃ is lysine (K). In some embodiments, X₃₆₀ is leucine (L).

In some embodiments, X₁₅₃ is proline (P), and X₁₂₈ is alanine (A). In some embodiments, X₁₅₃ is proline (P), X₁₂₈ is alanine (A), and X₃₃₃ is asparagine (N). In some embodiments, X₁₅₃ is proline (P), X₁₂₈ is alanine (A), X₃₃₃ is asparagine (N), and X₃₂₄ is isoleucine (I). In some embodiments, X₁₅₃ is proline (P), X₁₂₈ is alanine (A), X₃₃₃ is asparagine (N), X₃₂₄ is isoleucine (I), and X₁₆₂ is glutamic acid (E). In some embodiments, X₁₅₃ is proline (P), X₁₂₈ is alanine (A), X₃₃₃ is asparagine (N), X₃₂₄ is isoleucine (I), X₁₆₂ is glutamic acid (E), and X₂₂₉ is glutamic acid (E). In some embodiments, X₁₅₃ is proline (P), X₁₂₈ is alanine (A), X₃₃₃ is asparagine (N), X₃₂₄ is isoleucine (I), and X₂₂₉ is glutamic acid (E). In some embodiments, X₁₅₃ is proline (P), X₁₂₈ is alanine (A), X₃₃₃ is asparagine (N), and X₁₆₂ is glutamic acid (E). In some embodiments, X₁₅₃ is proline (P), X₁₂₈ is alanine (A), X₃₃₃ is asparagine (N), X₁₆₂ is glutamic acid (E), and X₂₂₉ is glutamic acid (E). In some embodiments, X₁₅₃ is proline (P), X₁₂₈ is alanine (A), X₃₃₃ is asparagine (N), and X₂₂₉ is glutamic acid (E). In some embodiments, X₁₅₃ is proline (P), X₁₂₈ is alanine (A), and X₃₂₄ is isoleucine (I). In some embodiments, X₁₅₃ is proline (P), X₁₂₈ is alanine (A), X₃₂₄ is isoleucine (I), and X₁₆₂ is glutamic acid (E). In some embodiments, X₁₅₃ is proline (P), X₁₂₈ is alanine (A), X₃₂₄ is isoleucine (I), X₁₆₂ is glutamic acid (E), and X₂₂₉ is glutamic acid (E). In some embodiments, X₁₅₃ is proline (P), X₁₂₈ is alanine (A), X₃₂₄ is isoleucine (I), and X₂₂₉ is glutamic acid (E). In some embodiments, X₁₅₃ is proline (P), X₁₂₈ is alanine (A), and X₁₆₂ is glutamic acid (E). In some embodiments, X₁₅₃ is proline (P), X₁₂₈ is alanine (A), X₁₆₂ is glutamic acid (E), and X₂₂₉ is glutamic acid (E). In some embodiments, X₁₅₃ is proline (P), X₁₂₈ is alanine (A), and X₂₂₉ is glutamic acid (E). In some embodiments, X₁₅₃ is proline (P), and X₃₃₃ is asparagine (N). In some embodiments, X₁₅₃ is proline (P), X₃₃₃ is asparagine (N), and X₃₂₄ is isoleucine (I). In some embodiments, X₁₅₃ is proline (P), X₃₃₃ is asparagine (N), X₃₂₄ is isoleucine (I), and X₁₆₂ is glutamic acid (E). In some embodiments, X₁₅₃ is proline (P), X₃₃₃ is asparagine (N), X₃₂₄ is isoleucine (I), X₁₆₂ is glutamic acid (E), and X₂₂₉ is glutamic acid (E). In some embodiments, X₁₅₃ is proline (P), X₃₃₃ is asparagine (N), X₃₂₄ is isoleucine (I), and X₂₂₉ is glutamic acid (E). In some embodiments, X₁₅₃ is proline (P), X₃₃₃ is asparagine (N), and X₁₆₂ is glutamic acid (E). In some embodiments, X₁₅₃ is proline (P), X₃₃₃ is asparagine (N), X₁₆₂ is glutamic acid (E), and X₂₂₉ is glutamic acid (E). In some embodiments, X₁₅₃ is proline (P), X₃₃₃ is asparagine (N), and X₂₂₉ is glutamic acid (E). In some embodiments, X₁₅₃ is proline (P), and X₃₂₄ is isoleucine (I). In some embodiments, X₁₅₃ is proline (P), X₃₂₄ is isoleucine (I), and X₁₆₂ is glutamic acid (E). In some embodiments, X₁₅₃ is proline (P), X₃₂₄ is isoleucine (I), X₁₆₂ is glutamic acid (E), and X₂₂₉ is glutamic acid (E). In some embodiments, X₁₅₃ is proline (P), X₃₂₄ is isoleucine (I), and X₂₂₉ is glutamic acid (E). In some embodiments, X₁₅₃ is proline (P), and X₁₆₂ is glutamic acid (E). In some embodiments, X₁₅₃ is proline (P), X₁₆₂ is glutamic acid (E), and X₂₂₉ is glutamic acid (E). In some embodiments, X₁₅₃ is proline (P), and X₂₂₉ is glutamic acid (E). In some embodiments, X₁₂₈ is alanine (A). In some embodiments, X₁₂₈ is alanine (A), and X₃₃₃ is asparagine (N). In some embodiments, X₁₂₈ is alanine (A), X₃₃₃ is asparagine (N), and X₃₂₄ is isoleucine (I). In some embodiments, X₁₂₈ is alanine (A), X₃₃₃ is asparagine (N), X₃₂₄ is isoleucine (I), and X₁₆₂ is glutamic acid (E). In some embodiments, X₁₂₈ is alanine (A), X₃₃₃ is asparagine (N), X₃₂₄ is isoleucine (I), X₁₆₂ is glutamic acid (E), and X₂₂₉ is glutamic acid (E). In some embodiments, X₁₂₈ is alanine (A), X₃₃₃ is asparagine (N), X₃₂₄ is isoleucine (I), and X₂₂₉ is glutamic acid (E). In some embodiments, X₁₂₈ is alanine (A), X₃₃₃ is asparagine (N), and X₁₆₂ is glutamic acid (E). In some embodiments, X₁₂₈ is alanine (A), X₃₃₃ is asparagine (N), X₁₆₂ is glutamic acid (E), and X₂₂₉ is glutamic acid (E). In some embodiments, X₁₂₈ is alanine (A), X₃₃₃ is asparagine (N), and X₂₂₉ is glutamic acid (E). In some embodiments, X₁₂₈ is alanine (A), and X₃₂₄ is isoleucine (I). In some embodiments, X₁₂₈ is alanine (A), X₃₂₄ is isoleucine (I), and X₁₆₂ is glutamic acid (E). In some embodiments, X₁₂₈ is alanine (A), X₃₂₄ is isoleucine (I), X₁₆₂ is glutamic acid (E), and X₂₂₉ is glutamic acid (E). In some embodiments, X₁₂₈ is alanine (A), X₃₂₄ is isoleucine (I), and X₂₂₉ is glutamic acid (E). In some embodiments, X₁₂₈ is alanine (A), and X₁₆₂ is glutamic acid (E). In some embodiments, X₁₂₈ is alanine (A), X₁₆₂ is glutamic acid (E), and X₂₂₉ is glutamic acid (E). In some embodiments, X₁₂₈ is alanine (A), and X₂₂₉ is glutamic acid (E). In some embodiments, X₃₃₃ is asparagine (N), and X₃₂₄ is isoleucine (I). In some embodiments, X₃₃₃ is asparagine (N), X₃₂₄ is isoleucine (I), and X₁₆₂ is glutamic acid (E). In some embodiments, X₃₃₃ is asparagine (N), X₃₂₄ is isoleucine (I), X₁₆₂ is glutamic acid (E), and X₂₂₉ is glutamic acid (E). In some embodiments, X₃₃₃ is asparagine (N), X₃₂₄ is isoleucine (I), and X₂₂₉ is glutamic acid (E). In some embodiments, X₃₃₃ is asparagine (N), and X₁₆₂ is glutamic acid (E). In some embodiments, X₃₃₃ is asparagine (N), X₁₆₂ is glutamic acid (E), and X₂₂₉ is glutamic acid (E). In some embodiments, X₃₃₃ is asparagine (N), and X₂₂₉ is glutamic acid (E). In some embodiments, X₃₂₄ is isoleucine (I), and X₁₆₂ is glutamic acid (E). In some embodiments, X₃₂₄ is isoleucine (I), X₁₆₂ is glutamic acid (E), and X₂₂₉ is glutamic acid (E). In some embodiments, X₃₂₄ is isoleucine (I), and X₂₂₉ is glutamic acid (E). In some embodiments, X₁₆₂ is glutamic acid (E), and X₂₂₉ is glutamic acid (E).

In some embodiments, X₁₂₈ is alanine (A), X₁₅₃ is proline (P), X₁₆₂ is glutamic acid (E), X₂₂₉ is glutamic acid (E), X₃₂₄ is isoleucine (I), and X₃₃₃ is asparagine (N).

In some embodiments, X₁₂₈ is alanine (A), X₁₅₃ is proline (P), X₁₆₂ is glutamic acid (E), X₁₇₄ is glutamic acid (E), X₁₇₇ is an amino acid sequence RDNRDN (SEQ ID NO: 743), X₂₁₉ is glycine (G), X₂₂₉ is glutamic acid (E), X₂₇₅ is tyrosine (Y), X₃₂₄ is isoleucine (I), and X₃₃₃ is asparagine (N).

In some embodiments, X₉₃ is aspartic acid (D), X₁₂₂ is serine(S), X₁₂₈ is alanine (A), X₁₅₀ is glutamic acid (E), X₁₅₃ is proline (P), X₁₆₂ is glutamic acid (E), X₁₇₄ is glutamic acid (E), X₁₇₇ is an amino acid sequence RDNRDN (SEQ ID NO: 743), X₁₉₉ is proline (P), X₂₁₉ is glycine (G), X₂₂₉ is glutamic acid (E), X₂₄₁ is aspartic acid (D), X₂₇₅ is tyrosine (Y), X₃₂₄ is isoleucine (I), and X₃₃₃ is asparagine (N).

In some embodiments, X₉₃ is aspartic acid (D), X₁₂₂ is serine(S), X₁₂₈ is alanine (A), X₁₃₉ is glutamic acid (E), X₁₅₀ is glutamic acid (E), X₁₅₃ is proline (P), X₁₆₂ is glutamic acid (E), X₁₇₄ is glutamic acid (E), X₁₇₇ is an amino acid sequence RDNRDN (SEQ ID NO: 743), X₁₉₆ is serine(S), X₁₉₉ is proline (P), X₂₁₉ is glycine (G), X₂₂₂ is glutamine (Q), X₂₂₉ is glutamic acid (E), X₂₄₁ is aspartic acid (D), X₂₆₂ is lysine (K), X₂₇₅ is tyrosine (Y), X₂₈₀ is threonine (T), X₃₂₄ is isoleucine (I), X₃₃₃ is asparagine (N), X₃₄₃ is lysine (K), and X₃₆₀ is leucine (L).

In some embodiments, the polypeptide having protease activity comprises an amino acid sequence of any one of SEQ ID NO: 380, 251, 5-243, 247, 280-379, 381-528, 737, 745-823, 828-864, or 885-916. In some embodiments, the polypeptide having protease activity comprises an amino acid sequence of SEQ ID NO: 3. In some embodiments, the polypeptide having protease activity comprises an amino acid sequence of SEQ ID NO: 4. In some embodiments, the polypeptide having protease activity comprises an amino acid sequence of SEQ ID NO: 5. In some embodiments, the polypeptide having protease activity comprises an amino acid sequence of SEQ ID NO: 6. In some embodiments, the polypeptide having protease activity comprises an amino acid sequence of SEQ ID NO: 7. In some embodiments, the polypeptide having protease activity comprises an amino acid sequence of SEQ ID NO: 8. In some embodiments, the polypeptide having protease activity comprises an amino acid sequence of SEQ ID NO: 9. In some embodiments, the polypeptide having protease activity comprises an amino acid sequence of SEQ ID NO: 10. In some embodiments, the polypeptide having protease activity comprises an amino acid sequence of SEQ ID NO: 11. In some embodiments, the polypeptide having protease activity comprises an amino acid sequence of SEQ ID NO: 12. In some embodiments, the polypeptide having protease activity comprises an amino acid sequence of SEQ ID NO: 13. In some embodiments, the polypeptide having protease activity comprises an amino acid sequence of SEQ ID NO: 14. In some embodiments, the polypeptide having protease activity comprises an amino acid sequence of SEQ ID NO: 15. In some embodiments, the polypeptide having protease activity comprises an amino acid sequence of SEQ ID NO: 16. In some embodiments, the polypeptide having protease activity comprises an amino acid sequence of SEQ ID NO: 17. In some embodiments, the polypeptide having protease activity comprises an amino acid sequence of SEQ ID NO: 18. In some embodiments, the polypeptide having protease activity comprises an amino acid sequence of SEQ ID NO: 19. In some embodiments, the polypeptide having protease activity comprises an amino acid sequence of SEQ ID NO: 20. In some embodiments, the polypeptide having protease activity comprises an amino acid sequence of SEQ ID NO: 21. In some embodiments, the polypeptide having protease activity comprises an amino acid sequence of SEQ ID NO: 22. In some embodiments, the polypeptide having protease activity comprises an amino acid sequence of SEQ ID NO: 23. In some embodiments, the polypeptide having protease activity comprises an amino acid sequence of SEQ ID NO: 24. In some embodiments, the polypeptide having protease activity comprises an amino acid sequence of SEQ ID NO: 25. In some embodiments, the polypeptide having protease activity comprises an amino acid sequence of SEQ ID NO: 26. In some embodiments, the polypeptide having protease activity comprises an amino acid sequence of SEQ ID NO: 27. In some embodiments, the polypeptide having protease activity comprises an amino acid sequence of SEQ ID NO: 28. In some embodiments, the polypeptide having protease activity comprises an amino acid sequence of SEQ ID NO: 29. In some embodiments, the polypeptide having protease activity comprises an amino acid sequence of SEQ ID NO: 30. In some embodiments, the polypeptide having protease activity comprises an amino acid sequence of SEQ ID NO: 31. In some embodiments, the polypeptide having protease activity comprises an amino acid sequence of SEQ ID NO: 32. In some embodiments, the polypeptide having protease activity comprises an amino acid sequence of SEQ ID NO: 33. In some embodiments, the polypeptide having protease activity comprises an amino acid sequence of SEQ ID NO: 34. In some embodiments, the polypeptide having protease activity comprises an amino acid sequence of SEQ ID NO: 35. In some embodiments, the polypeptide having protease activity comprises an amino acid sequence of SEQ ID NO: 36. In some embodiments, the polypeptide having protease activity comprises an amino acid sequence of SEQ ID NO: 37. In some embodiments, the polypeptide having protease activity comprises an amino acid sequence of SEQ ID NO: 38. In some embodiments, the polypeptide having protease activity comprises an amino acid sequence of SEQ ID NO: 39. In some embodiments, the polypeptide having protease activity comprises an amino acid sequence of SEQ ID NO: 40. In some embodiments, the polypeptide having protease activity comprises an amino acid sequence of SEQ ID NO: 41. In some embodiments, the polypeptide having protease activity comprises an amino acid sequence of SEQ ID NO: 42. In some embodiments, the polypeptide having protease activity comprises an amino acid sequence of SEQ ID NO: 43. In some embodiments, the polypeptide having protease activity comprises an amino acid sequence of SEQ ID NO: 44. In some embodiments, the polypeptide having protease activity comprises an amino acid sequence of SEQ ID NO: 45. In some embodiments, the polypeptide having protease activity comprises an amino acid sequence of SEQ ID NO: 46. In some embodiments, the polypeptide having protease activity comprises an amino acid sequence of SEQ ID NO: 47. In some embodiments, the polypeptide having protease activity comprises an amino acid sequence of SEQ ID NO: 48. In some embodiments, the polypeptide having protease activity comprises an amino acid sequence of SEQ ID NO: 49. In some embodiments, the polypeptide having protease activity comprises an amino acid sequence of SEQ ID NO: 50. In some embodiments, the polypeptide having protease activity comprises an amino acid sequence of SEQ ID NO: 51. In some embodiments, the polypeptide having protease activity comprises an amino acid sequence of SEQ ID NO: 52. In some embodiments, the polypeptide having protease activity comprises an amino acid sequence of SEQ ID NO: 53. In some embodiments, the polypeptide having protease activity comprises an amino acid sequence of SEQ ID NO: 54. In some embodiments, the polypeptide having protease activity comprises an amino acid sequence of SEQ ID NO: 55. In some embodiments, the polypeptide having protease activity comprises an amino acid sequence of SEQ ID NO: 56. In some embodiments, the polypeptide having protease activity comprises an amino acid sequence of SEQ ID NO: 57. In some embodiments, the polypeptide having protease activity comprises an amino acid sequence of SEQ ID NO: 58. In some embodiments, the polypeptide having protease activity comprises an amino acid sequence of SEQ ID NO: 59. In some embodiments, the polypeptide having protease activity comprises an amino acid sequence of SEQ ID NO: 60. In some embodiments, the polypeptide having protease activity comprises an amino acid sequence of SEQ ID NO: 61. In some embodiments, the polypeptide having protease activity comprises an amino acid sequence of SEQ ID NO: 62. In some embodiments, the polypeptide having protease activity comprises an amino acid sequence of SEQ ID NO: 63. In some embodiments, the polypeptide having protease activity comprises an amino acid sequence of SEQ ID NO: 64. In some embodiments, the polypeptide having protease activity comprises an amino acid sequence of SEQ ID NO: 65. In some embodiments, the polypeptide having protease activity comprises an amino acid sequence of SEQ ID NO: 66. In some embodiments, the polypeptide having protease activity comprises an amino acid sequence of SEQ ID NO: 67. In some embodiments, the polypeptide having protease activity comprises an amino acid sequence of SEQ ID NO: 68. In some embodiments, the polypeptide having protease activity comprises an amino acid sequence of SEQ ID NO: 69. In some embodiments, the polypeptide having protease activity comprises an amino acid sequence of SEQ ID NO: 70. In some embodiments, the polypeptide having protease activity comprises an amino acid sequence of SEQ ID NO: 71. In some embodiments, the polypeptide having protease activity comprises an amino acid sequence of SEQ ID NO: 72. In some embodiments, the polypeptide having protease activity comprises an amino acid sequence of SEQ ID NO: 73. In some embodiments, the polypeptide having protease activity comprises an amino acid sequence of SEQ ID NO: 74. In some embodiments, the polypeptide having protease activity comprises an amino acid sequence of SEQ ID NO: 75. In some embodiments, the polypeptide having protease activity comprises an amino acid sequence of SEQ ID NO: 76. In some embodiments, the polypeptide having protease activity comprises an amino acid sequence of SEQ ID NO: 77. In some embodiments, the polypeptide having protease activity comprises an amino acid sequence of SEQ ID NO: 78. In some embodiments, the polypeptide having protease activity comprises an amino acid sequence of SEQ ID NO: 79. In some embodiments, the polypeptide having protease activity comprises an amino acid sequence of SEQ ID NO: 80. In some embodiments, the polypeptide having protease activity comprises an amino acid sequence of SEQ ID NO: 81. In some embodiments, the polypeptide having protease activity comprises an amino acid sequence of SEQ ID NO: 82. In some embodiments, the polypeptide having protease activity comprises an amino acid sequence of SEQ ID NO: 83. In some embodiments, the polypeptide having protease activity comprises an amino acid sequence of SEQ ID NO: 84. In some embodiments, the polypeptide having protease activity comprises an amino acid sequence of SEQ ID NO: 85. In some embodiments, the polypeptide having protease activity comprises an amino acid sequence of SEQ ID NO: 86. In some embodiments, the polypeptide having protease activity comprises an amino acid sequence of SEQ ID NO: 87. In some embodiments, the polypeptide having protease activity comprises an amino acid sequence of SEQ ID NO: 88. In some embodiments, the polypeptide having protease activity comprises an amino acid sequence of SEQ ID NO: 89. In some embodiments, the polypeptide having protease activity comprises an amino acid sequence of SEQ ID NO: 90. In some embodiments, the polypeptide having protease activity comprises an amino acid sequence of SEQ ID NO: 91. In some embodiments, the polypeptide having protease activity comprises an amino acid sequence of SEQ ID NO: 92. In some embodiments, the polypeptide having protease activity comprises an amino acid sequence of SEQ ID NO: 93. In some embodiments, the polypeptide having protease activity comprises an amino acid sequence of SEQ ID NO: 96. In some embodiments, the polypeptide having protease activity comprises an amino acid sequence of SEQ ID NO: 97. In some embodiments, the polypeptide having protease activity comprises an amino acid sequence of SEQ ID NO: 98. In some embodiments, the polypeptide having protease activity comprises an amino acid sequence of SEQ ID NO: 99. In some embodiments, the polypeptide having protease activity comprises an amino acid sequence of SEQ ID NO: 100. In some embodiments, the polypeptide having protease activity comprises an amino acid sequence of SEQ ID NO: 101. In some embodiments, the polypeptide having protease activity comprises an amino acid sequence of SEQ ID NO: 102. In some embodiments, the polypeptide having protease activity comprises an amino acid sequence of SEQ ID NO: 103. In some embodiments, the polypeptide having protease activity comprises an amino acid sequence of SEQ ID NO: 104. In some embodiments, the polypeptide having protease activity comprises an amino acid sequence of SEQ ID NO: 105. In some embodiments, the polypeptide having protease activity comprises an amino acid sequence of SEQ ID NO: 106. In some embodiments, the polypeptide having protease activity comprises an amino acid sequence of SEQ ID NO: 107. In some embodiments, the polypeptide having protease activity comprises an amino acid sequence of SEQ ID NO: 108. In some embodiments, the polypeptide having protease activity comprises an amino acid sequence of SEQ ID NO: 109. In some embodiments, the polypeptide having protease activity comprises an amino acid sequence of SEQ ID NO: 110. In some embodiments, the polypeptide having protease activity comprises an amino acid sequence of SEQ ID NO: 111. In some embodiments, the polypeptide having protease activity comprises an amino acid sequence of SEQ ID NO: 112. In some embodiments, the polypeptide having protease activity comprises an amino acid sequence of SEQ ID NO: 113. In some embodiments, the polypeptide having protease activity comprises an amino acid sequence of SEQ ID NO: 114. In some embodiments, the polypeptide having protease activity comprises an amino acid sequence of SEQ ID NO: 115. In some embodiments, the polypeptide having protease activity comprises an amino acid sequence of SEQ ID NO: 116. In some embodiments, the polypeptide having protease activity comprises an amino acid sequence of SEQ ID NO: 117. In some embodiments, the polypeptide having protease activity comprises an amino acid sequence of SEQ ID NO: 118. In some embodiments, the polypeptide having protease activity comprises an amino acid sequence of SEQ ID NO: 119. In some embodiments, the polypeptide having protease activity comprises an amino acid sequence of SEQ ID NO: 120. In some embodiments, the polypeptide having protease activity comprises an amino acid sequence of SEQ ID NO: 121. In some embodiments, the polypeptide having protease activity comprises an amino acid sequence of SEQ ID NO: 122. In some embodiments, the polypeptide having protease activity comprises an amino acid sequence of SEQ ID NO: 123. In some embodiments, the polypeptide having protease activity comprises an amino acid sequence of SEQ ID NO: 124. In some embodiments, the polypeptide having protease activity comprises an amino acid sequence of SEQ ID NO: 125. In some embodiments, the polypeptide having protease activity comprises an amino acid sequence of SEQ ID NO: 126. In some embodiments, the polypeptide having protease activity comprises an amino acid sequence of SEQ ID NO: 127. In some embodiments, the polypeptide having protease activity comprises an amino acid sequence of SEQ ID NO: 128. In some embodiments, the polypeptide having protease activity comprises an amino acid sequence of SEQ ID NO: 129. In some embodiments, the polypeptide having protease activity comprises an amino acid sequence of SEQ ID NO: 130. In some embodiments, the polypeptide having protease activity comprises an amino acid sequence of SEQ ID NO: 131. In some embodiments, the polypeptide having protease activity comprises an amino acid sequence of SEQ ID NO: 132. In some embodiments, the polypeptide having protease activity comprises an amino acid sequence of SEQ ID NO: 133. In some embodiments, the polypeptide having protease activity comprises an amino acid sequence of SEQ ID NO: 134. In some embodiments, the polypeptide having protease activity comprises an amino acid sequence of SEQ ID NO: 135. In some embodiments, the polypeptide having protease activity comprises an amino acid sequence of SEQ ID NO: 136. In some embodiments, the polypeptide having protease activity comprises an amino acid sequence of SEQ ID NO: 137. In some embodiments, the polypeptide having protease activity comprises an amino acid sequence of SEQ ID NO: 138. In some embodiments, the polypeptide having protease activity comprises an amino acid sequence of SEQ ID NO: 139. In some embodiments, the polypeptide having protease activity comprises an amino acid sequence of SEQ ID NO: 140. In some embodiments, the polypeptide having protease activity comprises an amino acid sequence of SEQ ID NO: 141. In some embodiments, the polypeptide having protease activity comprises an amino acid sequence of SEQ ID NO: 142. In some embodiments, the polypeptide having protease activity comprises an amino acid sequence of SEQ ID NO: 143. In some embodiments, the polypeptide having protease activity comprises an amino acid sequence of SEQ ID NO: 144. In some embodiments, the polypeptide having protease activity comprises an amino acid sequence of SEQ ID NO: 145. In some embodiments, the polypeptide having protease activity comprises an amino acid sequence of SEQ ID NO: 146. In some embodiments, the polypeptide having protease activity comprises an amino acid sequence of SEQ ID NO: 147. In some embodiments, the polypeptide having protease activity comprises an amino acid sequence of SEQ ID NO: 148. In some embodiments, the polypeptide having protease activity comprises an amino acid sequence of SEQ ID NO: 149. In some embodiments, the polypeptide having protease activity comprises an amino acid sequence of SEQ ID NO: 150. In some embodiments, the polypeptide having protease activity comprises an amino acid sequence of SEQ ID NO: 151. In some embodiments, the polypeptide having protease activity comprises an amino acid sequence of SEQ ID NO: 152. In some embodiments, the polypeptide having protease activity comprises an amino acid sequence of SEQ ID NO: 153. In some embodiments, the polypeptide having protease activity comprises an amino acid sequence of SEQ ID NO: 154. In some embodiments, the polypeptide having protease activity comprises an amino acid sequence of SEQ ID NO: 155. In some embodiments, the polypeptide having protease activity comprises an amino acid sequence of SEQ ID NO: 156. In some embodiments, the polypeptide having protease activity comprises an amino acid sequence of SEQ ID NO: 157. In some embodiments, the polypeptide having protease activity comprises an amino acid sequence of SEQ ID NO: 158. In some embodiments, the polypeptide having protease activity comprises an amino acid sequence of SEQ ID NO: 159. In some embodiments, the polypeptide having protease activity comprises an amino acid sequence of SEQ ID NO: 160. In some embodiments, the polypeptide having protease activity comprises an amino acid sequence of SEQ ID NO: 161. In some embodiments, the polypeptide having protease activity comprises an amino acid sequence of SEQ ID NO: 162. In some embodiments, the polypeptide having protease activity comprises an amino acid sequence of SEQ ID NO: 163. In some embodiments, the polypeptide having protease activity comprises an amino acid sequence of SEQ ID NO: 164. In some embodiments, the polypeptide having protease activity comprises an amino acid sequence of SEQ ID NO: 165. In some embodiments, the polypeptide having protease activity comprises an amino acid sequence of SEQ ID NO: 166. In some embodiments, the polypeptide having protease activity comprises an amino acid sequence of SEQ ID NO: 167. In some embodiments, the polypeptide having protease activity comprises an amino acid sequence of SEQ ID NO: 168. In some embodiments, the polypeptide having protease activity comprises an amino acid sequence of SEQ ID NO: 169. In some embodiments, the polypeptide having protease activity comprises an amino acid sequence of SEQ ID NO: 170. In some embodiments, the polypeptide having protease activity comprises an amino acid sequence of SEQ ID NO: 171. In some embodiments, the polypeptide having protease activity comprises an amino acid sequence of SEQ ID NO: 172. In some embodiments, the polypeptide having protease activity comprises an amino acid sequence of SEQ ID NO: 173. In some embodiments, the polypeptide having protease activity comprises an amino acid sequence of SEQ ID NO: 174. In some embodiments, the polypeptide having protease activity comprises an amino acid sequence of SEQ ID NO: 175. In some embodiments, the polypeptide having protease activity comprises an amino acid sequence of SEQ ID NO: 176. In some embodiments, the polypeptide having protease activity comprises an amino acid sequence of SEQ ID NO: 177. In some embodiments, the polypeptide having protease activity comprises an amino acid sequence of SEQ ID NO: 178. In some embodiments, the polypeptide having protease activity comprises an amino acid sequence of SEQ ID NO: 179. In some embodiments, the polypeptide having protease activity comprises an amino acid sequence of SEQ ID NO: 180. In some embodiments, the polypeptide having protease activity comprises an amino acid sequence of SEQ ID NO: 181. In some embodiments, the polypeptide having protease activity comprises an amino acid sequence of SEQ ID NO: 182. In some embodiments, the polypeptide having protease activity comprises an amino acid sequence of SEQ ID NO: 183. In some embodiments, the polypeptide having protease activity comprises an amino acid sequence of SEQ ID NO: 184. In some embodiments, the polypeptide having protease activity comprises an amino acid sequence of SEQ ID NO: 185. In some embodiments, the polypeptide having protease activity comprises an amino acid sequence of SEQ ID NO: 186. In some embodiments, the polypeptide having protease activity comprises an amino acid sequence of SEQ ID NO: 187. In some embodiments, the polypeptide having protease activity comprises an amino acid sequence of SEQ ID NO: 188. In some embodiments, the polypeptide having protease activity comprises an amino acid sequence of SEQ ID NO: 189. In some embodiments, the polypeptide having protease activity comprises an amino acid sequence of SEQ ID NO: 190. In some embodiments, the polypeptide having protease activity comprises an amino acid sequence of SEQ ID NO: 191. In some embodiments, the polypeptide having protease activity comprises an amino acid sequence of SEQ ID NO: 192. In some embodiments, the polypeptide having protease activity comprises an amino acid sequence of SEQ ID NO: 193. In some embodiments, the polypeptide having protease activity comprises an amino acid sequence of SEQ ID NO: 194. In some embodiments, the polypeptide having protease activity comprises an amino acid sequence of SEQ ID NO: 195. In some embodiments, the polypeptide having protease activity comprises an amino acid sequence of SEQ ID NO: 196. In some embodiments, the polypeptide having protease activity comprises an amino acid sequence of SEQ ID NO: 197. In some embodiments, the polypeptide having protease activity comprises an amino acid sequence of SEQ ID NO: 198. In some embodiments, the polypeptide having protease activity comprises an amino acid sequence of SEQ ID NO: 199. In some embodiments, the polypeptide having protease activity comprises an amino acid sequence of SEQ ID NO: 200. In some embodiments, the polypeptide having protease activity comprises an amino acid sequence of SEQ ID NO: 201. In some embodiments, the polypeptide having protease activity comprises an amino acid sequence of SEQ ID NO: 202. In some embodiments, the polypeptide having protease activity comprises an amino acid sequence of SEQ ID NO: 203. In some embodiments, the polypeptide having protease activity comprises an amino acid sequence of SEQ ID NO: 204. In some embodiments, the polypeptide having protease activity comprises an amino acid sequence of SEQ ID NO: 205. In some embodiments, the polypeptide having protease activity comprises an amino acid sequence of SEQ ID NO: 206. In some embodiments, the polypeptide having protease activity comprises an amino acid sequence of SEQ ID NO: 207. In some embodiments, the polypeptide having protease activity comprises an amino acid sequence of SEQ ID NO: 208. In some embodiments, the polypeptide having protease activity comprises an amino acid sequence of SEQ ID NO: 209. In some embodiments, the polypeptide having protease activity comprises an amino acid sequence of SEQ ID NO: 210. In some embodiments, the polypeptide having protease activity comprises an amino acid sequence of SEQ ID NO: 211. In some embodiments, the polypeptide having protease activity comprises an amino acid sequence of SEQ ID NO: 212. In some embodiments, the polypeptide having protease activity comprises an amino acid sequence of SEQ ID NO: 213. In some embodiments, the polypeptide having protease activity comprises an amino acid sequence of SEQ ID NO: 214. In some embodiments, the polypeptide having protease activity comprises an amino acid sequence of SEQ ID NO: 215. In some embodiments, the polypeptide having protease activity comprises an amino acid sequence of SEQ ID NO: 216. In some embodiments, the polypeptide having protease activity comprises an amino acid sequence of SEQ ID NO: 217. In some embodiments, the polypeptide having protease activity comprises an amino acid sequence of SEQ ID NO: 218. In some embodiments, the polypeptide having protease activity comprises an amino acid sequence of SEQ ID NO: 219. In some embodiments, the polypeptide having protease activity comprises an amino acid sequence of SEQ ID NO: 220. In some embodiments, the polypeptide having protease activity comprises an amino acid sequence of SEQ ID NO: 221. In some embodiments, the polypeptide having protease activity comprises an amino acid sequence of SEQ ID NO: 222. In some embodiments, the polypeptide having protease activity comprises an amino acid sequence of SEQ ID NO: 223. In some embodiments, the polypeptide having protease activity comprises an amino acid sequence of SEQ ID NO: 224. In some embodiments, the polypeptide having protease activity comprises an amino acid sequence of SEQ ID NO: 225. In some embodiments, the polypeptide having protease activity comprises an amino acid sequence of SEQ ID NO: 226. In some embodiments, the polypeptide having protease activity comprises an amino acid sequence of SEQ ID NO: 227. In some embodiments, the polypeptide having protease activity comprises an amino acid sequence of SEQ ID NO: 228. In some embodiments, the polypeptide having protease activity comprises an amino acid sequence of SEQ ID NO: 229. In some embodiments, the polypeptide having protease activity comprises an amino acid sequence of SEQ ID NO: 230. In some embodiments, the polypeptide having protease activity comprises an amino acid sequence of SEQ ID NO: 231. In some embodiments, the polypeptide having protease activity comprises an amino acid sequence of SEQ ID NO: 232. In some embodiments, the polypeptide having protease activity comprises an amino acid sequence of SEQ ID NO: 233. In some embodiments, the polypeptide having protease activity comprises an amino acid sequence of SEQ ID NO: 234. In some embodiments, the polypeptide having protease activity comprises an amino acid sequence of SEQ ID NO: 235. In some embodiments, the polypeptide having protease activity comprises an amino acid sequence of SEQ ID NO: 236. In some embodiments, the polypeptide having protease activity comprises an amino acid sequence of SEQ ID NO: 237. In some embodiments, the polypeptide having protease activity comprises an amino acid sequence of SEQ ID NO: 238. In some embodiments, the polypeptide having protease activity comprises an amino acid sequence of SEQ ID NO: 239. In some embodiments, the polypeptide having protease activity comprises an amino acid sequence of SEQ ID NO: 240. In some embodiments, the polypeptide having protease activity comprises an amino acid sequence of SEQ ID NO: 241. In some embodiments, the polypeptide having protease activity comprises an amino acid sequence of SEQ ID NO: 242. In some embodiments, the polypeptide having protease activity comprises an amino acid sequence of SEQ ID NO: 243. In some embodiments, the polypeptide having protease activity comprises an amino acid sequence of SEQ ID NO: 247. In some embodiments, the polypeptide having protease activity comprises an amino acid sequence of SEQ ID NO: 251. In some embodiments, the polypeptide having protease activity comprises an amino acid sequence of SEQ ID NO: 280. In some embodiments, the polypeptide having protease activity comprises an amino acid sequence of SEQ ID NO: 281. In some embodiments, the polypeptide having protease activity comprises an amino acid sequence of SEQ ID NO: 282. In some embodiments, the polypeptide having protease activity comprises an amino acid sequence of SEQ ID NO: 283. In some embodiments, the polypeptide having protease activity comprises an amino acid sequence of SEQ ID NO: 284. In some embodiments, the polypeptide having protease activity comprises an amino acid sequence of SEQ ID NO: 285. In some embodiments, the polypeptide having protease activity comprises an amino acid sequence of SEQ ID NO: 286. In some embodiments, the polypeptide having protease activity comprises an amino acid sequence of SEQ ID NO: 287. In some embodiments, the polypeptide having protease activity comprises an amino acid sequence of SEQ ID NO: 288. In some embodiments, the polypeptide having protease activity comprises an amino acid sequence of SEQ ID NO: 289. In some embodiments, the polypeptide having protease activity comprises an amino acid sequence of SEQ ID NO: 290. In some embodiments, the polypeptide having protease activity comprises an amino acid sequence of SEQ ID NO: 291. In some embodiments, the polypeptide having protease activity comprises an amino acid sequence of SEQ ID NO: 292. In some embodiments, the polypeptide having protease activity comprises an amino acid sequence of SEQ ID NO: 293. In some embodiments, the polypeptide having protease activity comprises an amino acid sequence of SEQ ID NO: 294. In some embodiments, the polypeptide having protease activity comprises an amino acid sequence of SEQ ID NO: 295. In some embodiments, the polypeptide having protease activity comprises an amino acid sequence of SEQ ID NO: 296. In some embodiments, the polypeptide having protease activity comprises an amino acid sequence of SEQ ID NO: 297. In some embodiments, the polypeptide having protease activity comprises an amino acid sequence of SEQ ID NO: 298. In some embodiments, the polypeptide having protease activity comprises an amino acid sequence of SEQ ID NO: 299. In some embodiments, the polypeptide having protease activity comprises an amino acid sequence of SEQ ID NO: 300. In some embodiments, the polypeptide having protease activity comprises an amino acid sequence of SEQ ID NO: 301. In some embodiments, the polypeptide having protease activity comprises an amino acid sequence of SEQ ID NO: 302. In some embodiments, the polypeptide having protease activity comprises an amino acid sequence of SEQ ID NO: 303. In some embodiments, the polypeptide having protease activity comprises an amino acid sequence of SEQ ID NO: 304. In some embodiments, the polypeptide having protease activity comprises an amino acid sequence of SEQ ID NO: 305. In some embodiments, the polypeptide having protease activity comprises an amino acid sequence of SEQ ID NO: 306. In some embodiments, the polypeptide having protease activity comprises an amino acid sequence of SEQ ID NO: 307. In some embodiments, the polypeptide having protease activity comprises an amino acid sequence of SEQ ID NO: 308. In some embodiments, the polypeptide having protease activity comprises an amino acid sequence of SEQ ID NO: 309. In some embodiments, the polypeptide having protease activity comprises an amino acid sequence of SEQ ID NO: 310. In some embodiments, the polypeptide having protease activity comprises an amino acid sequence of SEQ ID NO: 311. In some embodiments, the polypeptide having protease activity comprises an amino acid sequence of SEQ ID NO: 312. In some embodiments, the polypeptide having protease activity comprises an amino acid sequence of SEQ ID NO: 313. In some embodiments, the polypeptide having protease activity comprises an amino acid sequence of SEQ ID NO: 314. In some embodiments, the polypeptide having protease activity comprises an amino acid sequence of SEQ ID NO: 315. In some embodiments, the polypeptide having protease activity comprises an amino acid sequence of SEQ ID NO: 316. In some embodiments, the polypeptide having protease activity comprises an amino acid sequence of SEQ ID NO: 317. In some embodiments, the polypeptide having protease activity comprises an amino acid sequence of SEQ ID NO: 318. In some embodiments, the polypeptide having protease activity comprises an amino acid sequence of SEQ ID NO: 319. In some embodiments, the polypeptide having protease activity comprises an amino acid sequence of SEQ ID NO: 320. In some embodiments, the polypeptide having protease activity comprises an amino acid sequence of SEQ ID NO: 321. In some embodiments, the polypeptide having protease activity comprises an amino acid sequence of SEQ ID NO: 322. In some embodiments, the polypeptide having protease activity comprises an amino acid sequence of SEQ ID NO: 323. In some embodiments, the polypeptide having protease activity comprises an amino acid sequence of SEQ ID NO: 324. In some embodiments, the polypeptide having protease activity comprises an amino acid sequence of SEQ ID NO: 325. In some embodiments, the polypeptide having protease activity comprises an amino acid sequence of SEQ ID NO: 326. In some embodiments, the polypeptide having protease activity comprises an amino acid sequence of SEQ ID NO: 327. In some embodiments, the polypeptide having protease activity comprises an amino acid sequence of SEQ ID NO: 328. In some embodiments, the polypeptide having protease activity comprises an amino acid sequence of SEQ ID NO: 329. In some embodiments, the polypeptide having protease activity comprises an amino acid sequence of SEQ ID NO: 330. In some embodiments, the polypeptide having protease activity comprises an amino acid sequence of SEQ ID NO: 331. In some embodiments, the polypeptide having protease activity comprises an amino acid sequence of SEQ ID NO: 332. In some embodiments, the polypeptide having protease activity comprises an amino acid sequence of SEQ ID NO: 333. In some embodiments, the polypeptide having protease activity comprises an amino acid sequence of SEQ ID NO: 334. In some embodiments, the polypeptide having protease activity comprises an amino acid sequence of SEQ ID NO: 335. In some embodiments, the polypeptide having protease activity comprises an amino acid sequence of SEQ ID NO: 336. In some embodiments, the polypeptide having protease activity comprises an amino acid sequence of SEQ ID NO: 337. In some embodiments, the polypeptide having protease activity comprises an amino acid sequence of SEQ ID NO: 338. In some embodiments, the polypeptide having protease activity comprises an amino acid sequence of SEQ ID NO: 339. In some embodiments, the polypeptide having protease activity comprises an amino acid sequence of SEQ ID NO: 340. In some embodiments, the polypeptide having protease activity comprises an amino acid sequence of SEQ ID NO: 341. In some embodiments, the polypeptide having protease activity comprises an amino acid sequence of SEQ ID NO: 342. In some embodiments, the polypeptide having protease activity comprises an amino acid sequence of SEQ ID NO: 343. In some embodiments, the polypeptide having protease activity comprises an amino acid sequence of SEQ ID NO: 344. In some embodiments, the polypeptide having protease activity comprises an amino acid sequence of SEQ ID NO: 345. In some embodiments, the polypeptide having protease activity comprises an amino acid sequence of SEQ ID NO: 346. In some embodiments, the polypeptide having protease activity comprises an amino acid sequence of SEQ ID NO: 347. In some embodiments, the polypeptide having protease activity comprises an amino acid sequence of SEQ ID NO: 348. In some embodiments, the polypeptide having protease activity comprises an amino acid sequence of SEQ ID NO: 349. In some embodiments, the polypeptide having protease activity comprises an amino acid sequence of SEQ ID NO: 350. In some embodiments, the polypeptide having protease activity comprises an amino acid sequence of SEQ ID NO: 351. In some embodiments, the polypeptide having protease activity comprises an amino acid sequence of SEQ ID NO: 352. In some embodiments, the polypeptide having protease activity comprises an amino acid sequence of SEQ ID NO: 353. In some embodiments, the polypeptide having protease activity comprises an amino acid sequence of SEQ ID NO: 354. In some embodiments, the polypeptide having protease activity comprises an amino acid sequence of SEQ ID NO: 355. In some embodiments, the polypeptide having protease activity comprises an amino acid sequence of SEQ ID NO: 356. In some embodiments, the polypeptide having protease activity comprises an amino acid sequence of SEQ ID NO: 357. In some embodiments, the polypeptide having protease activity comprises an amino acid sequence of SEQ ID NO: 358. In some embodiments, the polypeptide having protease activity comprises an amino acid sequence of SEQ ID NO: 359. In some embodiments, the polypeptide having protease activity comprises an amino acid sequence of SEQ ID NO: 360. In some embodiments, the polypeptide having protease activity comprises an amino acid sequence of SEQ ID NO: 361. In some embodiments, the polypeptide having protease activity comprises an amino acid sequence of SEQ ID NO: 362. In some embodiments, the polypeptide having protease activity comprises an amino acid sequence of SEQ ID NO: 363. In some embodiments, the polypeptide having protease activity comprises an amino acid sequence of SEQ ID NO: 364. In some embodiments, the polypeptide having protease activity comprises an amino acid sequence of SEQ ID NO: 365. In some embodiments, the polypeptide having protease activity comprises an amino acid sequence of SEQ ID NO: 366. In some embodiments, the polypeptide having protease activity comprises an amino acid sequence of SEQ ID NO: 367. In some embodiments, the polypeptide having protease activity comprises an amino acid sequence of SEQ ID NO: 368. In some embodiments, the polypeptide having protease activity comprises an amino acid sequence of SEQ ID NO: 369. In some embodiments, the polypeptide having protease activity comprises an amino acid sequence of SEQ ID NO: 370. In some embodiments, the polypeptide having protease activity comprises an amino acid sequence of SEQ ID NO: 371. In some embodiments, the polypeptide having protease activity comprises an amino acid sequence of SEQ ID NO: 372. In some embodiments, the polypeptide having protease activity comprises an amino acid sequence of SEQ ID NO: 373. In some embodiments, the polypeptide having protease activity comprises an amino acid sequence of SEQ ID NO: 374. In some embodiments, the polypeptide having protease activity comprises an amino acid sequence of SEQ ID NO: 375. In some embodiments, the polypeptide having protease activity comprises an amino acid sequence of SEQ ID NO: 376. In some embodiments, the polypeptide having protease activity comprises an amino acid sequence of SEQ ID NO: 377. In some embodiments, the polypeptide having protease activity comprises an amino acid sequence of SEQ ID NO: 378. In some embodiments, the polypeptide having protease activity comprises an amino acid sequence of SEQ ID NO: 379. In some embodiments, the polypeptide having protease activity comprises an amino acid sequence of SEQ ID NO: 380. In some embodiments, the polypeptide having protease activity comprises an amino acid sequence of SEQ ID NO: 381. In some embodiments, the polypeptide having protease activity comprises an amino acid sequence of SEQ ID NO: 382. In some embodiments, the polypeptide having protease activity comprises an amino acid sequence of SEQ ID NO: 383. In some embodiments, the polypeptide having protease activity comprises an amino acid sequence of SEQ ID NO: 384. In some embodiments, the polypeptide having protease activity comprises an amino acid sequence of SEQ ID NO: 385. In some embodiments, the polypeptide having protease activity comprises an amino acid sequence of SEQ ID NO: 386. In some embodiments, the polypeptide having protease activity comprises an amino acid sequence of SEQ ID NO: 387. In some embodiments, the polypeptide having protease activity comprises an amino acid sequence of SEQ ID NO: 388. In some embodiments, the polypeptide having protease activity comprises an amino acid sequence of SEQ ID NO: 389. In some embodiments, the polypeptide having protease activity comprises an amino acid sequence of SEQ ID NO: 390. In some embodiments, the polypeptide having protease activity comprises an amino acid sequence of SEQ ID NO: 391. In some embodiments, the polypeptide having protease activity comprises an amino acid sequence of SEQ ID NO: 392. In some embodiments, the polypeptide having protease activity comprises an amino acid sequence of SEQ ID NO: 393. In some embodiments, the polypeptide having protease activity comprises an amino acid sequence of SEQ ID NO: 394. In some embodiments, the polypeptide having protease activity comprises an amino acid sequence of SEQ ID NO: 395. In some embodiments, the polypeptide having protease activity comprises an amino acid sequence of SEQ ID NO: 396. In some embodiments, the polypeptide having protease activity comprises an amino acid sequence of SEQ ID NO: 397. In some embodiments, the polypeptide having protease activity comprises an amino acid sequence of SEQ ID NO: 398. In some embodiments, the polypeptide having protease activity comprises an amino acid sequence of SEQ ID NO: 399. In some embodiments, the polypeptide having protease activity comprises an amino acid sequence of SEQ ID NO: 400. In some embodiments, the polypeptide having protease activity comprises an amino acid sequence of SEQ ID NO: 401. In some embodiments, the polypeptide having protease activity comprises an amino acid sequence of SEQ ID NO: 402. In some embodiments, the polypeptide having protease activity comprises an amino acid sequence of SEQ ID NO: 403. In some embodiments, the polypeptide having protease activity comprises an amino acid sequence of SEQ ID NO: 404. In some embodiments, the polypeptide having protease activity comprises an amino acid sequence of SEQ ID NO: 405. In some embodiments, the polypeptide having protease activity comprises an amino acid sequence of SEQ ID NO: 406. In some embodiments, the polypeptide having protease activity comprises an amino acid sequence of SEQ ID NO: 407. In some embodiments, the polypeptide having protease activity comprises an amino acid sequence of SEQ ID NO: 408. In some embodiments, the polypeptide having protease activity comprises an amino acid sequence of SEQ ID NO: 409. In some embodiments, the polypeptide having protease activity comprises an amino acid sequence of SEQ ID NO: 410. In some embodiments, the polypeptide having protease activity comprises an amino acid sequence of SEQ ID NO: 411. In some embodiments, the polypeptide having protease activity comprises an amino acid sequence of SEQ ID NO: 412. In some embodiments, the polypeptide having protease activity comprises an amino acid sequence of SEQ ID NO: 413. In some embodiments, the polypeptide having protease activity comprises an amino acid sequence of SEQ ID NO: 414. In some embodiments, the polypeptide having protease activity comprises an amino acid sequence of SEQ ID NO: 415. In some embodiments, the polypeptide having protease activity comprises an amino acid sequence of SEQ ID NO: 416. In some embodiments, the polypeptide having protease activity comprises an amino acid sequence of SEQ ID NO: 417. In some embodiments, the polypeptide having protease activity comprises an amino acid sequence of SEQ ID NO: 418. In some embodiments, the polypeptide having protease activity comprises an amino acid sequence of SEQ ID NO: 419. In some embodiments, the polypeptide having protease activity comprises an amino acid sequence of SEQ ID NO: 420. In some embodiments, the polypeptide having protease activity comprises an amino acid sequence of SEQ ID NO: 421. In some embodiments, the polypeptide having protease activity comprises an amino acid sequence of SEQ ID NO: 422. In some embodiments, the polypeptide having protease activity comprises an amino acid sequence of SEQ ID NO: 423. In some embodiments, the polypeptide having protease activity comprises an amino acid sequence of SEQ ID NO: 424. In some embodiments, the polypeptide having protease activity comprises an amino acid sequence of SEQ ID NO: 425. In some embodiments, the polypeptide having protease activity comprises an amino acid sequence of SEQ ID NO: 426. In some embodiments, the polypeptide having protease activity comprises an amino acid sequence of SEQ ID NO: 427. In some embodiments, the polypeptide having protease activity comprises an amino acid sequence of SEQ ID NO: 428. In some embodiments, the polypeptide having protease activity comprises an amino acid sequence of SEQ ID NO: 429. In some embodiments, the polypeptide having protease activity comprises an amino acid sequence of SEQ ID NO: 430. In some embodiments, the polypeptide having protease activity comprises an amino acid sequence of SEQ ID NO: 431. In some embodiments, the polypeptide having protease activity comprises an amino acid sequence of SEQ ID NO: 432. In some embodiments, the polypeptide having protease activity comprises an amino acid sequence of SEQ ID NO: 433. In some embodiments, the polypeptide having protease activity comprises an amino acid sequence of SEQ ID NO: 434. In some embodiments, the polypeptide having protease activity comprises an amino acid sequence of SEQ ID NO: 435. In some embodiments, the polypeptide having protease activity comprises an amino acid sequence of SEQ ID NO: 436. In some embodiments, the polypeptide having protease activity comprises an amino acid sequence of SEQ ID NO: 437. In some embodiments, the polypeptide having protease activity comprises an amino acid sequence of SEQ ID NO: 438. In some embodiments, the polypeptide having protease activity comprises an amino acid sequence of SEQ ID NO: 439. In some embodiments, the polypeptide having protease activity comprises an amino acid sequence of SEQ ID NO: 440. In some embodiments, the polypeptide having protease activity comprises an amino acid sequence of SEQ ID NO: 441. In some embodiments, the polypeptide having protease activity comprises an amino acid sequence of SEQ ID NO: 442. In some embodiments, the polypeptide having protease activity comprises an amino acid sequence of SEQ ID NO: 443. In some embodiments, the polypeptide having protease activity comprises an amino acid sequence of SEQ ID NO: 444. In some embodiments, the polypeptide having protease activity comprises an amino acid sequence of SEQ ID NO: 445. In some embodiments, the polypeptide having protease activity comprises an amino acid sequence of SEQ ID NO: 446. In some embodiments, the polypeptide having protease activity comprises an amino acid sequence of SEQ ID NO: 447. In some embodiments, the polypeptide having protease activity comprises an amino acid sequence of SEQ ID NO: 448. In some embodiments, the polypeptide having protease activity comprises an amino acid sequence of SEQ ID NO: 449. In some embodiments, the polypeptide having protease activity comprises an amino acid sequence of SEQ ID NO: 450. In some embodiments, the polypeptide having protease activity comprises an amino acid sequence of SEQ ID NO: 451. In some embodiments, the polypeptide having protease activity comprises an amino acid sequence of SEQ ID NO: 452. In some embodiments, the polypeptide having protease activity comprises an amino acid sequence of SEQ ID NO: 453. In some embodiments, the polypeptide having protease activity comprises an amino acid sequence of SEQ ID NO: 454. In some embodiments, the polypeptide having protease activity comprises an amino acid sequence of SEQ ID NO: 455. In some embodiments, the polypeptide having protease activity comprises an amino acid sequence of SEQ ID NO: 456. In some embodiments, the polypeptide having protease activity comprises an amino acid sequence of SEQ ID NO: 457. In some embodiments, the polypeptide having protease activity comprises an amino acid sequence of SEQ ID NO: 458. In some embodiments, the polypeptide having protease activity comprises an amino acid sequence of SEQ ID NO: 459. In some embodiments, the polypeptide having protease activity comprises an amino acid sequence of SEQ ID NO: 460. In some embodiments, the polypeptide having protease activity comprises an amino acid sequence of SEQ ID NO: 461. In some embodiments, the polypeptide having protease activity comprises an amino acid sequence of SEQ ID NO: 462. In some embodiments, the polypeptide having protease activity comprises an amino acid sequence of SEQ ID NO: 463. In some embodiments, the polypeptide having protease activity comprises an amino acid sequence of SEQ ID NO: 464. In some embodiments, the polypeptide having protease activity comprises an amino acid sequence of SEQ ID NO: 465. In some embodiments, the polypeptide having protease activity comprises an amino acid sequence of SEQ ID NO: 466. In some embodiments, the polypeptide having protease activity comprises an amino acid sequence of SEQ ID NO: 467. In some embodiments, the polypeptide having protease activity comprises an amino acid sequence of SEQ ID NO: 468. In some embodiments, the polypeptide having protease activity comprises an amino acid sequence of SEQ ID NO: 469. In some embodiments, the polypeptide having protease activity comprises an amino acid sequence of SEQ ID NO: 470. In some embodiments, the polypeptide having protease activity comprises an amino acid sequence of SEQ ID NO: 471. In some embodiments, the polypeptide having protease activity comprises an amino acid sequence of SEQ ID NO: 472. In some embodiments, the polypeptide having protease activity comprises an amino acid sequence of SEQ ID NO: 473. In some embodiments, the polypeptide having protease activity comprises an amino acid sequence of SEQ ID NO: 474. In some embodiments, the polypeptide having protease activity comprises an amino acid sequence of SEQ ID NO: 475. In some embodiments, the polypeptide having protease activity comprises an amino acid sequence of SEQ ID NO: 476. In some embodiments, the polypeptide having protease activity comprises an amino acid sequence of SEQ ID NO: 477. In some embodiments, the polypeptide having protease activity comprises an amino acid sequence of SEQ ID NO: 478. In some embodiments, the polypeptide having protease activity comprises an amino acid sequence of SEQ ID NO: 479. In some embodiments, the polypeptide having protease activity comprises an amino acid sequence of SEQ ID NO: 480. In some embodiments, the polypeptide having protease activity comprises an amino acid sequence of SEQ ID NO: 481. In some embodiments, the polypeptide having protease activity comprises an amino acid sequence of SEQ ID NO: 482. In some embodiments, the polypeptide having protease activity comprises an amino acid sequence of SEQ ID NO: 483. In some embodiments, the polypeptide having protease activity comprises an amino acid sequence of SEQ ID NO: 484. In some embodiments, the polypeptide having protease activity comprises an amino acid sequence of SEQ ID NO: 485. In some embodiments, the polypeptide having protease activity comprises an amino acid sequence of SEQ ID NO: 486. In some embodiments, the polypeptide having protease activity comprises an amino acid sequence of SEQ ID NO: 487. In some embodiments, the polypeptide having protease activity comprises an amino acid sequence of SEQ ID NO: 488. In some embodiments, the polypeptide having protease activity comprises an amino acid sequence of SEQ ID NO: 489. In some embodiments, the polypeptide having protease activity comprises an amino acid sequence of SEQ ID NO: 490. In some embodiments, the polypeptide having protease activity comprises an amino acid sequence of SEQ ID NO: 491. In some embodiments, the polypeptide having protease activity comprises an amino acid sequence of SEQ ID NO: 492. In some embodiments, the polypeptide having protease activity comprises an amino acid sequence of SEQ ID NO: 493. In some embodiments, the polypeptide having protease activity comprises an amino acid sequence of SEQ ID NO: 494. In some embodiments, the polypeptide having protease activity comprises an amino acid sequence of SEQ ID NO: 495. In some embodiments, the polypeptide having protease activity comprises an amino acid sequence of SEQ ID NO: 496. In some embodiments, the polypeptide having protease activity comprises an amino acid sequence of SEQ ID NO: 497. In some embodiments, the polypeptide having protease activity comprises an amino acid sequence of SEQ ID NO: 498. In some embodiments, the polypeptide having protease activity comprises an amino acid sequence of SEQ ID NO: 499. In some embodiments, the polypeptide having protease activity comprises an amino acid sequence of SEQ ID NO: 500. In some embodiments, the polypeptide having protease activity comprises an amino acid sequence of SEQ ID NO: 501. In some embodiments, the polypeptide having protease activity comprises an amino acid sequence of SEQ ID NO: 502. In some embodiments, the polypeptide having protease activity comprises an amino acid sequence of SEQ ID NO: 503. In some embodiments, the polypeptide having protease activity comprises an amino acid sequence of SEQ ID NO: 504. In some embodiments, the polypeptide having protease activity comprises an amino acid sequence of SEQ ID NO: 505. In some embodiments, the polypeptide having protease activity comprises an amino acid sequence of SEQ ID NO: 506. In some embodiments, the polypeptide having protease activity comprises an amino acid sequence of SEQ ID NO: 507. In some embodiments, the polypeptide having protease activity comprises an amino acid sequence of SEQ ID NO: 508. In some embodiments, the polypeptide having protease activity comprises an amino acid sequence of SEQ ID NO: 509. In some embodiments, the polypeptide having protease activity comprises an amino acid sequence of SEQ ID NO: 510. In some embodiments, the polypeptide having protease activity comprises an amino acid sequence of SEQ ID NO: 511. In some embodiments, the polypeptide having protease activity comprises an amino acid sequence of SEQ ID NO: 512. In some embodiments, the polypeptide having protease activity comprises an amino acid sequence of SEQ ID NO: 513. In some embodiments, the polypeptide having protease activity comprises an amino acid sequence of SEQ ID NO: 514. In some embodiments, the polypeptide having protease activity comprises an amino acid sequence of SEQ ID NO: 515. In some embodiments, the polypeptide having protease activity comprises an amino acid sequence of SEQ ID NO: 516. In some embodiments, the polypeptide having protease activity comprises an amino acid sequence of SEQ ID NO: 517. In some embodiments, the polypeptide having protease activity comprises an amino acid sequence of SEQ ID NO: 518. In some embodiments, the polypeptide having protease activity comprises an amino acid sequence of SEQ ID NO: 519. In some embodiments, the polypeptide having protease activity comprises an amino acid sequence of SEQ ID NO: 520. In some embodiments, the polypeptide having protease activity comprises an amino acid sequence of SEQ ID NO: 521. In some embodiments, the polypeptide having protease activity comprises an amino acid sequence of SEQ ID NO: 522. In some embodiments, the polypeptide having protease activity comprises an amino acid sequence of SEQ ID NO: 523. In some embodiments, the polypeptide having protease activity comprises an amino acid sequence of SEQ ID NO: 524. In some embodiments, the polypeptide having protease activity comprises an amino acid sequence of SEQ ID NO: 525. In some embodiments, the polypeptide having protease activity comprises an amino acid sequence of SEQ ID NO: 526. In some embodiments, the polypeptide having protease activity comprises an amino acid sequence of SEQ ID NO: 527. In some embodiments, the polypeptide having protease activity comprises an amino acid sequence of SEQ ID NO: 528. In some embodiments, the polypeptide having protease activity comprises an amino acid sequence of SEQ ID NO: 737. In some embodiments, the polypeptide having protease activity comprises an amino acid sequence of SEQ ID NO: 745. In some embodiments, the polypeptide having protease activity comprises an amino acid sequence of SEQ ID NO: 746. In some embodiments, the polypeptide having protease activity comprises an amino acid sequence of SEQ ID NO: 747. In some embodiments, the polypeptide having protease activity comprises an amino acid sequence of SEQ ID NO: 748. In some embodiments, the polypeptide having protease activity comprises an amino acid sequence of SEQ ID NO: 749. In some embodiments, the polypeptide having protease activity comprises an amino acid sequence of SEQ ID NO: 750. In some embodiments, the polypeptide having protease activity comprises an amino acid sequence of SEQ ID NO: 751. In some embodiments, the polypeptide having protease activity comprises an amino acid sequence of SEQ ID NO: 752. In some embodiments, the polypeptide having protease activity comprises an amino acid sequence of SEQ ID NO: 753. In some embodiments, the polypeptide having protease activity comprises an amino acid sequence of SEQ ID NO: 754. In some embodiments, the polypeptide having protease activity comprises an amino acid sequence of SEQ ID NO: 755. In some embodiments, the polypeptide having protease activity comprises an amino acid sequence of SEQ ID NO: 756. In some embodiments, the polypeptide having protease activity comprises an amino acid sequence of SEQ ID NO: 757. In some embodiments, the polypeptide having protease activity comprises an amino acid sequence of SEQ ID NO: 758. In some embodiments, the polypeptide having protease activity comprises an amino acid sequence of SEQ ID NO: 759. In some embodiments, the polypeptide having protease activity comprises an amino acid sequence of SEQ ID NO: 760. In some embodiments, the polypeptide having protease activity comprises an amino acid sequence of SEQ ID NO: 761. In some embodiments, the polypeptide having protease activity comprises an amino acid sequence of SEQ ID NO: 762. In some embodiments, the polypeptide having protease activity comprises an amino acid sequence of SEQ ID NO: 763. In some embodiments, the polypeptide having protease activity comprises an amino acid sequence of SEQ ID NO: 764. In some embodiments, the polypeptide having protease activity comprises an amino acid sequence of SEQ ID NO: 765. In some embodiments, the polypeptide having protease activity comprises an amino acid sequence of SEQ ID NO: 766. In some embodiments, the polypeptide having protease activity comprises an amino acid sequence of SEQ ID NO: 767. In some embodiments, the polypeptide having protease activity comprises an amino acid sequence of SEQ ID NO: 768. In some embodiments, the polypeptide having protease activity comprises an amino acid sequence of SEQ ID NO: 769. In some embodiments, the polypeptide having protease activity comprises an amino acid sequence of SEQ ID NO: 770. In some embodiments, the polypeptide having protease activity comprises an amino acid sequence of SEQ ID NO: 771. In some embodiments, the polypeptide having protease activity comprises an amino acid sequence of SEQ ID NO: 772. In some embodiments, the polypeptide having protease activity comprises an amino acid sequence of SEQ ID NO: 773. In some embodiments, the polypeptide having protease activity comprises an amino acid sequence of SEQ ID NO: 774. In some embodiments, the polypeptide having protease activity comprises an amino acid sequence of SEQ ID NO: 775. In some embodiments, the polypeptide having protease activity comprises an amino acid sequence of SEQ ID NO: 776. In some embodiments, the polypeptide having protease activity comprises an amino acid sequence of SEQ ID NO: 777. In some embodiments, the polypeptide having protease activity comprises an amino acid sequence of SEQ ID NO: 778. In some embodiments, the polypeptide having protease activity comprises an amino acid sequence of SEQ ID NO: 779. In some embodiments, the polypeptide having protease activity comprises an amino acid sequence of SEQ ID NO: 780. In some embodiments, the polypeptide having protease activity comprises an amino acid sequence of SEQ ID NO: 781. In some embodiments, the polypeptide having protease activity comprises an amino acid sequence of SEQ ID NO: 782. In some embodiments, the polypeptide having protease activity comprises an amino acid sequence of SEQ ID NO: 783. In some embodiments, the polypeptide having protease activity comprises an amino acid sequence of SEQ ID NO: 784. In some embodiments, the polypeptide having protease activity comprises an amino acid sequence of SEQ ID NO: 785. In some embodiments, the polypeptide having protease activity comprises an amino acid sequence of SEQ ID NO: 786. In some embodiments, the polypeptide having protease activity comprises an amino acid sequence of SEQ ID NO: 787. In some embodiments, the polypeptide having protease activity comprises an amino acid sequence of SEQ ID NO: 788. In some embodiments, the polypeptide having protease activity comprises an amino acid sequence of SEQ ID NO: 789. In some embodiments, the polypeptide having protease activity comprises an amino acid sequence of SEQ ID NO: 790. In some embodiments, the polypeptide having protease activity comprises an amino acid sequence of SEQ ID NO: 791. In some embodiments, the polypeptide having protease activity comprises an amino acid sequence of SEQ ID NO: 792. In some embodiments, the polypeptide having protease activity comprises an amino acid sequence of SEQ ID NO: 793. In some embodiments, the polypeptide having protease activity comprises an amino acid sequence of SEQ ID NO: 794. In some embodiments, the polypeptide having protease activity comprises an amino acid sequence of SEQ ID NO: 795. In some embodiments, the polypeptide having protease activity comprises an amino acid sequence of SEQ ID NO: 796. In some embodiments, the polypeptide having protease activity comprises an amino acid sequence of SEQ ID NO: 797. In some embodiments, the polypeptide having protease activity comprises an amino acid sequence of SEQ ID NO: 798. In some embodiments, the polypeptide having protease activity comprises an amino acid sequence of SEQ ID NO: 799. In some embodiments, the polypeptide having protease activity comprises an amino acid sequence of SEQ ID NO: 800. In some embodiments, the polypeptide having protease activity comprises an amino acid sequence of SEQ ID NO: 801. In some embodiments, the polypeptide having protease activity comprises an amino acid sequence of SEQ ID NO: 802. In some embodiments, the polypeptide having protease activity comprises an amino acid sequence of SEQ ID NO: 803. In some embodiments, the polypeptide having protease activity comprises an amino acid sequence of SEQ ID NO: 804. In some embodiments, the polypeptide having protease activity comprises an amino acid sequence of SEQ ID NO: 805. In some embodiments, the polypeptide having protease activity comprises an amino acid sequence of SEQ ID NO: 806. In some embodiments, the polypeptide having protease activity comprises an amino acid sequence of SEQ ID NO: 807. In some embodiments, the polypeptide having protease activity comprises an amino acid sequence of SEQ ID NO: 808. In some embodiments, the polypeptide having protease activity comprises an amino acid sequence of SEQ ID NO: 809. In some embodiments, the polypeptide having protease activity comprises an amino acid sequence of SEQ ID NO: 810. In some embodiments, the polypeptide having protease activity comprises an amino acid sequence of SEQ ID NO: 811. In some embodiments, the polypeptide having protease activity comprises an amino acid sequence of SEQ ID NO: 812. In some embodiments, the polypeptide having protease activity comprises an amino acid sequence of SEQ ID NO: 813. In some embodiments, the polypeptide having protease activity comprises an amino acid sequence of SEQ ID NO: 814. In some embodiments, the polypeptide having protease activity comprises an amino acid sequence of SEQ ID NO: 815. In some embodiments, the polypeptide having protease activity comprises an amino acid sequence of SEQ ID NO: 816. In some embodiments, the polypeptide having protease activity comprises an amino acid sequence of SEQ ID NO: 817. In some embodiments, the polypeptide having protease activity comprises an amino acid sequence of SEQ ID NO: 818. In some embodiments, the polypeptide having protease activity comprises an amino acid sequence of SEQ ID NO: 819. In some embodiments, the polypeptide having protease activity comprises an amino acid sequence of SEQ ID NO: 820. In some embodiments, the polypeptide having protease activity comprises an amino acid sequence of SEQ ID NO: 821. In some embodiments, the polypeptide having protease activity comprises an amino acid sequence of SEQ ID NO: 822. In some embodiments, the polypeptide having protease activity comprises an amino acid sequence of SEQ ID NO: 823. In some embodiments, the polypeptide having protease activity comprises an amino acid sequence of SEQ ID NO: 828. In some embodiments, the polypeptide having protease activity comprises an amino acid sequence of SEQ ID NO: 829. In some embodiments, the polypeptide having protease activity comprises an amino acid sequence of SEQ ID NO: 830. In some embodiments, the polypeptide having protease activity comprises an amino acid sequence of SEQ ID NO: 831. In some embodiments, the polypeptide having protease activity comprises an amino acid sequence of SEQ ID NO: 832. In some embodiments, the polypeptide having protease activity comprises an amino acid sequence of SEQ ID NO: 833. In some embodiments, the polypeptide having protease activity comprises an amino acid sequence of SEQ ID NO: 834. In some embodiments, the polypeptide having protease activity comprises an amino acid sequence of SEQ ID NO: 835. In some embodiments, the polypeptide having protease activity comprises an amino acid sequence of SEQ ID NO: 836. In some embodiments, the polypeptide having protease activity comprises an amino acid sequence of SEQ ID NO: 837. In some embodiments, the polypeptide having protease activity comprises an amino acid sequence of SEQ ID NO: 838. In some embodiments, the polypeptide having protease activity comprises an amino acid sequence of SEQ ID NO: 839. In some embodiments, the polypeptide having protease activity comprises an amino acid sequence of SEQ ID NO: 840. In some embodiments, the polypeptide having protease activity comprises an amino acid sequence of SEQ ID NO: 841. In some embodiments, the polypeptide having protease activity comprises an amino acid sequence of SEQ ID NO: 842. In some embodiments, the polypeptide having protease activity comprises an amino acid sequence of SEQ ID NO: 843. In some embodiments, the polypeptide having protease activity comprises an amino acid sequence of SEQ ID NO: 844. In some embodiments, the polypeptide having protease activity comprises an amino acid sequence of SEQ ID NO: 845. In some embodiments, the polypeptide having protease activity comprises an amino acid sequence of SEQ ID NO: 846. In some embodiments, the polypeptide having protease activity comprises an amino acid sequence of SEQ ID NO: 847. In some embodiments, the polypeptide having protease activity comprises an amino acid sequence of SEQ ID NO: 848. In some embodiments, the polypeptide having protease activity comprises an amino acid sequence of SEQ ID NO: 849. In some embodiments, the polypeptide having protease activity comprises an amino acid sequence of SEQ ID NO: 850. In some embodiments, the polypeptide having protease activity comprises an amino acid sequence of SEQ ID NO: 851. In some embodiments, the polypeptide having protease activity comprises an amino acid sequence of SEQ ID NO: 852. In some embodiments, the polypeptide having protease activity comprises an amino acid sequence of SEQ ID NO: 853. In some embodiments, the polypeptide having protease activity comprises an amino acid sequence of SEQ ID NO: 854. In some embodiments, the polypeptide having protease activity comprises an amino acid sequence of SEQ ID NO: 855. In some embodiments, the polypeptide having protease activity comprises an amino acid sequence of SEQ ID NO: 856. In some embodiments, the polypeptide having protease activity comprises an amino acid sequence of SEQ ID NO: 857. In some embodiments, the polypeptide having protease activity comprises an amino acid sequence of SEQ ID NO: 858. In some embodiments, the polypeptide having protease activity comprises an amino acid sequence of SEQ ID NO: 859. In some embodiments, the polypeptide having protease activity comprises an amino acid sequence of SEQ ID NO: 860. In some embodiments, the polypeptide having protease activity comprises an amino acid sequence of SEQ ID NO: 861. In some embodiments, the polypeptide having protease activity comprises an amino acid sequence of SEQ ID NO: 862. In some embodiments, the polypeptide having protease activity comprises an amino acid sequence of SEQ ID NO: 863. In some embodiments, the polypeptide having protease activity comprises an amino acid sequence of SEQ ID NO: 864. In some embodiments, the polypeptide having protease activity comprises an amino acid sequence of SEQ ID NO: 885. In some embodiments, the polypeptide having protease activity comprises an amino acid sequence of SEQ ID NO: 886. In some embodiments, the polypeptide having protease activity comprises an amino acid sequence of SEQ ID NO: 887. In some embodiments, the polypeptide having protease activity comprises an amino acid sequence of SEQ ID NO: 888. In some embodiments, the polypeptide having protease activity comprises an amino acid sequence of SEQ ID NO: 889. In some embodiments, the polypeptide having protease activity comprises an amino acid sequence of SEQ ID NO: 890. In some embodiments, the polypeptide having protease activity comprises an amino acid sequence of SEQ ID NO: 891. In some embodiments, the polypeptide having protease activity comprises an amino acid sequence of SEQ ID NO: 892. In some embodiments, the polypeptide having protease activity comprises an amino acid sequence of SEQ ID NO: 893. In some embodiments, the polypeptide having protease activity comprises an amino acid sequence of SEQ ID NO: 894. In some embodiments, the polypeptide having protease activity comprises an amino acid sequence of SEQ ID NO: 895. In some embodiments, the polypeptide having protease activity comprises an amino acid sequence of SEQ ID NO: 896. In some embodiments, the polypeptide having protease activity comprises an amino acid sequence of SEQ ID NO: 897. In some embodiments, the polypeptide having protease activity comprises an amino acid sequence of SEQ ID NO: 898. In some embodiments, the polypeptide having protease activity comprises an amino acid sequence of SEQ ID NO: 899. In some embodiments, the polypeptide having protease activity comprises an amino acid sequence of SEQ ID NO: 900. In some embodiments, the polypeptide having protease activity comprises an amino acid sequence of SEQ ID NO: 901. In some embodiments, the polypeptide having protease activity comprises an amino acid sequence of SEQ ID NO: 902. In some embodiments, the polypeptide having protease activity comprises an amino acid sequence of SEQ ID NO: 903. In some embodiments, the polypeptide having protease activity comprises an amino acid sequence of SEQ ID NO: 904. In some embodiments, the polypeptide having protease activity comprises an amino acid sequence of SEQ ID NO: 905. In some embodiments, the polypeptide having protease activity comprises an amino acid sequence of SEQ ID NO: 906. In some embodiments, the polypeptide having protease activity comprises an amino acid sequence of SEQ ID NO: 907. In some embodiments, the polypeptide having protease activity comprises an amino acid sequence of SEQ ID NO: 908. In some embodiments, the polypeptide having protease activity comprises an amino acid sequence of SEQ ID NO: 909. In some embodiments, the polypeptide having protease activity comprises an amino acid sequence of SEQ ID NO: 910. In some embodiments, the polypeptide having protease activity comprises an amino acid sequence of SEQ ID NO: 911. In some embodiments, the polypeptide having protease activity comprises an amino acid sequence of SEQ ID NO: 912. In some embodiments, the polypeptide having protease activity comprises an amino acid sequence of SEQ ID NO: 913. In some embodiments, the polypeptide having protease activity comprises an amino acid sequence of SEQ ID NO: 914. In some embodiments, the polypeptide having protease activity comprises an amino acid sequence of SEQ ID NO: 915. In some embodiments, the polypeptide having protease activity comprises an amino acid sequence of SEQ ID NO: 916.

### Dimer Molecules

In some embodiments, two (or more) polypeptides associate, either covalently or non-covalently, e.g., to form a hetero or homo-dimeric therapeutic compound. In some embodiments, the polypeptide comprises a linker. In some embodiments, the polypeptide comprises an Fc domain. In some embodiments, the polypeptide comprises a linker and an Fc domain. In some embodiments, the linker may comprise an Fc domain. In some embodiments, two Fc domains may associate with one another. In some embodiments of a therapeutic compound comprising two linker regions, the linker regions can self-associate, e.g., as two identical Fc domains. In some embodiments of a therapeutic compound comprising two linker regions, the linker regions are not capable of, or not capable of substantial, self-association, e.g., the two Fc domains can be members of a knob and hole pair.

In some embodiments, an Fc domain comprises an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 721, 724, or 725. In some embodiments, an Fc domain comprises an amino acid sequence of SEQ ID NO: 721, 724, or 725.

In some embodiments, a polypeptide can associate with another polypeptide. In some embodiments, the polypeptide associated with another polypeptide forms a dimer molecule. In some embodiments, the polypeptide comprises a first polypeptide and a second polypeptide. In some embodiments, the first polypeptide comprises a knob mutation, and the second polypeptide comprises a hole mutation. In some embodiments, the first polypeptide comprises a hole mutation, and the second polypeptide comprises a knob mutation. In some embodiments, the knob mutation is such as those provided herein. In some embodiments, the hole mutation is such as those provided herein. In some embodiments, the knob mutation is any knob mutation known in the art. In some embodiments, the hole mutation is any hole mutation known in the art.

In some embodiments, non-limiting examples of knob mutations include T366W, T366Y, Y407R, or S354C mutations as compared to:

(SEQ ID NO: 252)
ASTKGPSVFPLAPSSKSTSGGTAALGCLVKDYFPEPVTVSWNSGAL
TSGVHTFPAVLQSSGLYSLSSVVTVPSSSLGTQTYICNVNHKPSNT
KVDKKVEPKSCDKTHTCPPCPAPELLGGPSVFLFPPKPKDTLMISR
TPEVTCVVVDVSHEDPEVKFNWYVDGVEVHNAKTKPREEQYNSTYR
VVSVLTVLHQDWLNGKEYKCKVSNKALPAPIEKTISKAKGQPREPQ
VYTLPPSRDELTKNQVSLTCLVKGFYPSDIAVEWESNGQPENNYKT
TPPVLDSDGSFFLYSKLTVDKSRWQQGNVFSCSVMHEALHNHYTQK
SLSLSPGK,

according to EU numbering.

In some embodiments, non-limiting examples of hole mutations include Y349C, E357T, Y407A, Y407D, or Y407S mutations as compared to SEQ ID NO: 252, according to EU numbering.

In some embodiments, the Fc domain comprises a set of at least one knob mutation selected from T366W, T366Y, Y407R, S354C mutations, or any know mutation known in the art, as compared to SEQ ID NO: 252, according to EU numbering, and at least one hole mutation selected from Y349C, E357T, Y407A, Y407D, Y407S mutations, or any hole mutation known in the art, as compared to SEQ ID NO: 252, according to EU numbering.

In some embodiments, the knob mutation is such as those provided in:

(SEQ ID NO: 253)
DKTHTCPPCPAPEAAGAPSVFLFPPKPKDTLMISRTPEVTCVVVDV
SHEDPEVKFNWYVDGVEVHNAKTKPREEQQNSTYRVVSVLTVLHQD
WLNGKEYKCKVSNKALKAPIEKTISKAKGQPREPQVYTLPPSRDEL
TKNQVSLWCLVKGFYPSDIAVEWESNGQPENNYKTTPPVLDSDGSF
FLYSKLTVDKSRWQQGNVFSCSVMHEALHNHYTQKSLSLSPG,
(SEQ ID NO: 254)
DKTHTCPPCPAPEAAGAPSVFLFPPKPKDTLMISRTPEVTCVVVDV
SHEDPEVKFNWYVDGVEVHNAKTKPREEQQNSTYRVVSVLTVLHQD
WLNGKEYKCKVSNKALKAPIEKTISKAKGQPREPQVYTLPPCRDEL
TKNQVSLWCLVKGFYPSDIAVEWESNGQPENNYKTTPPVLDSDGSF
FLYSKLTVDKSRWQQGNVFSCSVMHEALHNHYTQKSLSLSPG,
or
(SEQ ID NO: 722)
DKTHTCPPCPAPEAAGAPSVFLFPPKPKDTLMISRTPEVTCVVVDV
SHEDPEVKFNWYVDGVEVHNAKTKPREEQQNSTYRVVSVLIVLHQD
WLNGKEYKCKVSNKALKAPIEKTISKAKGQPREPQVYTLPPSRDEL
TKNQVSLWCLVKGFYPSDIAVEWESNGQPENNYKTTPPVLDSDGSF
FLYSKLTVDKSRWQQGNVFSCSVLHEALHSHYTQKSLSLSPG.
In some embodiments, the hole mutation is such
as those provided in:
(SEQ ID NO: 255)
DKTHTCPPCPAPEAAGAPSVFLFPPKPKDTLMISRTPEVTCVVVDV
SHEDPEVKFNWYVDGVEVHNAKTKPREEQQNSTYRVVSVLTVLHQD
WLNGKEYKCKVSNKALKAPIEKTISKAKGQPREPQVYTLPPSRDEL
TKNQVSLSCAVKGFYPSDIAVEWESNGQPENNYKTTPPVLDSDGSF
FLVSKLTVDKSRWQQGNVFSCSVMHEALHNHYTQKSLSLSPG,
(SEQ ID NO: 256)
DKTHTCPPCPAPEAAGAPSVFLFPPKPKDTLMISRTPEVTCVVVDV
SHEDPEVKFNWYVDGVEVHNAKTKPREEQQNSTYRVVSVLTVLHQD
WLNGKEYKCKVSNKALKAPIEKTISKAKGQPREPQVCTLPPSRDEL
TKNQVSLSCAVKGFYPSDIAVEWESNGQPENNYKTTPPVLDSDGSF
FLVSKLTVDKSRWQQGNVFSCSVMHEALHNHYTQKSLSLSPG,
or
(SEQ ID NO: 723)
DKTHTCPPCPAPEAAGAPSVFLFPPKPKDTLMISRTPEVTCVVVDV
SHEDPEVKFNWYVDGVEVHNAKTKPREEQQNSTYRVVSVLTVLHQD
WLNGKEYKCKVSNKALKAPIEKTISKAKGQPREPQVYTLPPSRDEL
TKNQVSLSCAVKGFYPSDIAVEWESNGQPENNYKTTPPVLDSDGSF
FLVSKLTVDKSRWQQGNVFSCSVLHEALHSHYTQKSLSLSPG.

In some embodiments, an Fc domain comprises an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 253, 254, 255, 256, 722, or 723.

In some embodiments, a composition comprises an Fc dimer comprising a first Fc domain comprising an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 253, 254, or 722; and a second Fc domain comprising an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 255, 256, or 723.

In some embodiments, a composition comprises an Fc dimer comprising a first Fc domain comprising an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 253; and a second Fc domain comprising an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 255. In some embodiments, a composition comprises an Fc dimer comprising a first Fc domain comprising an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 254; and a second Fc domain comprising an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 256. In some embodiments, a composition comprises an Fc dimer comprising a first Fc domain comprising an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 722; and a second Fc domain comprising an amino acid sequence having at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 723.

In some embodiments, an Fc domain comprises an amino acid sequence of SEQ ID NO: 253, 254, 255, 256, 722, or 723.

In some embodiments, a composition comprises an Fc dimer comprising a first Fc domain comprising an amino acid sequence of SEQ ID NO: 253, 254, or 722; and a second Fc domain comprising an amino acid sequence of SEQ ID NO: 255, 256, or 723.

In some embodiments, a composition comprises an Fc dimer comprising a first Fc domain comprising an amino acid sequence of SEQ ID NO: 253; and a second Fc domain comprising an amino acid sequence of SEQ ID NO: 255. In some embodiments, a composition comprises an Fc dimer comprising a first Fc domain comprising an amino acid sequence of SEQ ID NO: 254; and a second Fc domain comprising an amino acid sequence of SEQ ID NO: 256. In some embodiments, a composition comprises an Fc dimer comprising a first Fc domain comprising an amino acid sequence of SEQ ID NO: 722; and a second Fc domain comprising an amino acid sequence of SEQ ID NO: 723.

In some embodiments, a first Fc polypeptide comprises a T366W substitution and the second Fc polypeptide comprises T366S, L368A, and Y407V substitutions. The numbering referenced here in Fc domains is according to EU numbering, which is a standard used for numbering/identifying residues in Fc domains. In some embodiments, the first and second polypeptides that form a heterodimer also comprise cysteine substitutions (a residue on each is changed to a cysteine). In some embodiments, the substitution is at position 354 (EU numbering) and/or position 349 (EU numbering). In some embodiments, the substitution is a S354C substitution. In some embodiments, the substitution is Y349C substitution. Without being bound to any theory, the presence of cysteines on the first and second polypeptides can facilitate the formation of the dimer (homodimer or heterodimer by allowing the formation of disulfide bond. Thus, in some embodiments, the first polypeptide comprises S354C and T366W substitutions and the second polypeptide comprises Y349C, T366S, L368A, and Y407V substitutions. Other knob-in-hole Fc heterodimer mutations can be used and the cysteine substitutions can also be made in combination with those known knob-in-hole formats.

Thus, in some embodiments, a dimer molecule comprises a first polypeptide and a second polypeptide, which can be the same or different. If different, the dimer can be referred to as a heterodimer. If the Fc domains are the same, the dimer can be referred to as a heterodimer. In some embodiments, the first molecule comprises a protease conjugated to an Fc domain, or an Fc domain. In some embodiments, the second molecule comprises a protease conjugated to an Fc domain, or an Fc domain. In some embodiments, the first polypeptide comprises a protease conjugated to an Fc domain, and the second polypeptide comprises an Fc domain. Thus, in some embodiments, the second polypeptide comprising an Fc does not contain or promise a protease, such as those provided for herein. A molecule that has two Fc polypeptides and only one protease polypeptide linked to one of the Fc molecules is considered to be a monovalent protease molecule because it only has one protease domain or polypeptide. Thus, in some embodiments, a monovalent protease polypeptide is provided for herein, wherein the polypeptide comprises two Fc domains, which can be as provided for herein, and one protease polypeptide domain linked to one of the Fc domains. In some embodiments, the first polypeptide comprises an Fc domain, and the second polypeptide comprises a protease conjugated to an Fc domain. In some embodiments, the Fc domain comprises knob in hole mutations. In some embodiments, the Fc domain forms a dimer. In some embodiments, the Fc domain of the first polypeptide and the second polypeptide form a dimer.

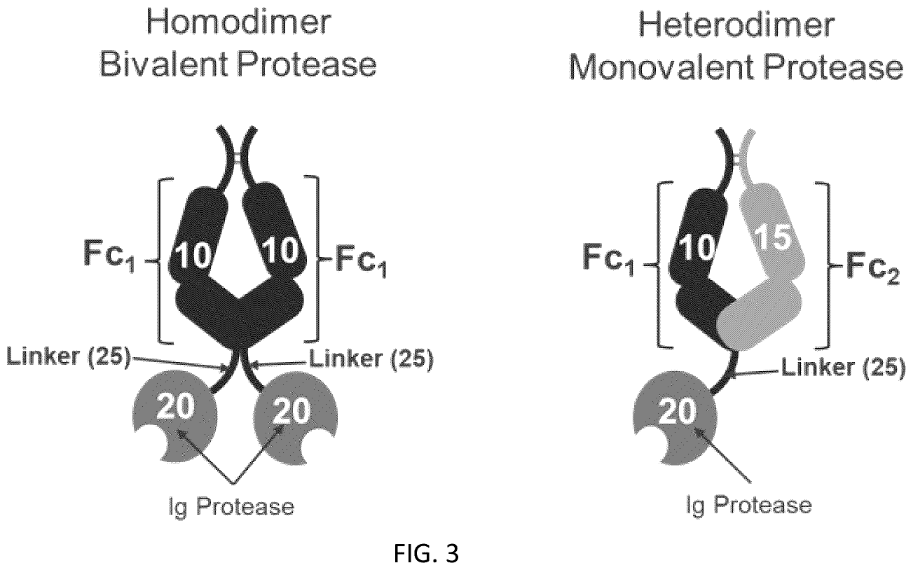

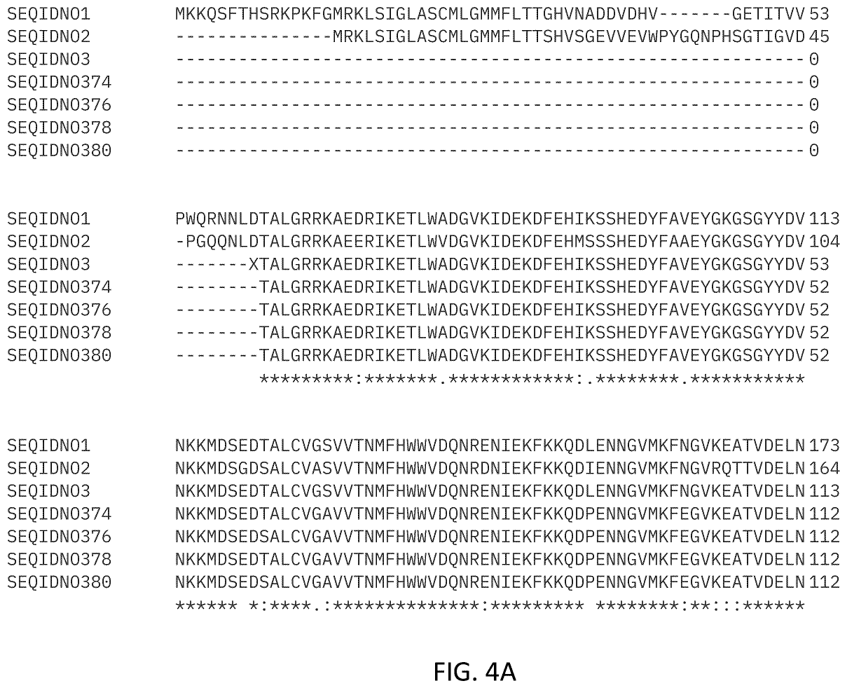

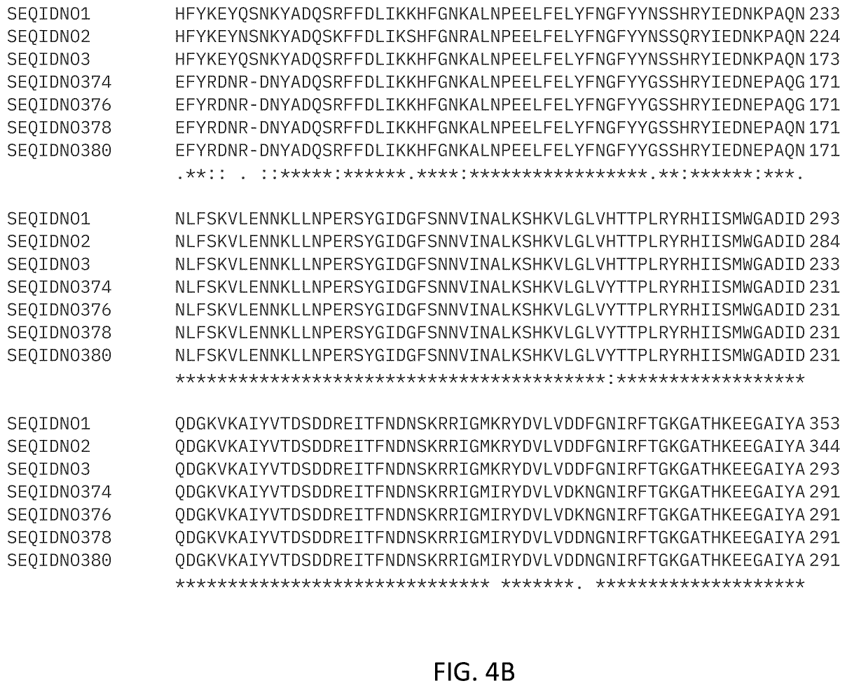

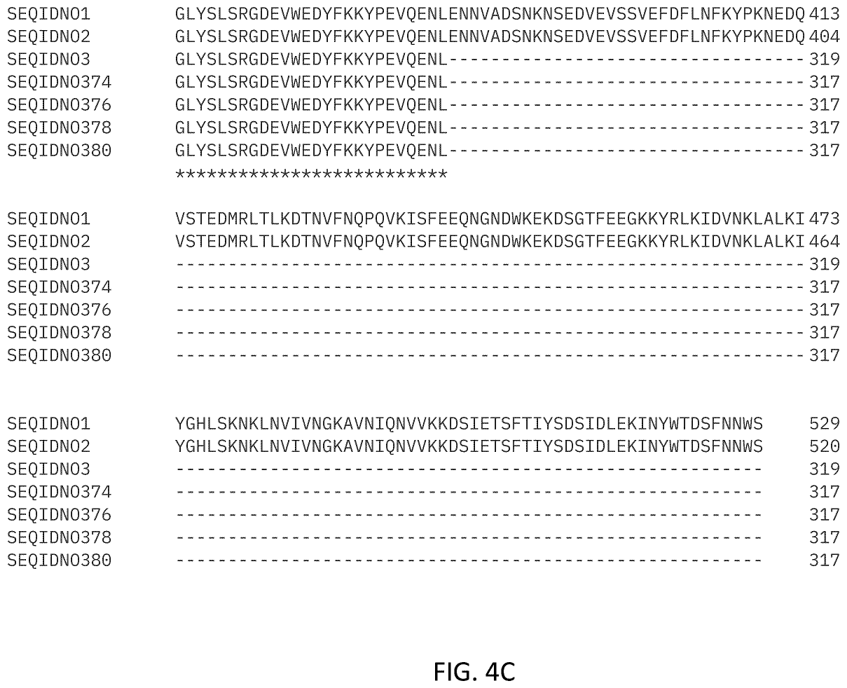

A protease molecule that is monovalent is illustrated in a non-limiting embodiment shown below, which can be referred to as FIG. 3. In reference to FIG. 3, the left panel illustrates a non-limiting example of a bivalent protease, which is formed by having a Fc homodimer, where each member of the homodimer is a Fc polypeptide (10). In the homodimer the Fc polypeptides are identical, which is illustrated by them both being labeled as Fc1 (the subscript for an illustrated Fc domain, whether it is Fc1 or Fc2, is for notation purposes only to show that the Fc domains form a homodimer or heterodimer and is not meant to make a reference to different immunoglobulin subclasses or allotypes). The Ig Protease (20) is linked to the Fc domain, which can be direct, or as illustrated above and provided for herein, via a peptide linker (25). The left panel illustrates the bivalent protease having two Ig Protease domains (20). As used herein, the term “bivalent protease” refers to a polypeptide molecule having two protease polypeptide domains. In the right panel, a non-limiting example of a monovalent protease polypeptide is illustrated. As illustrated in the right panel, the monovalent protease polypeptide comprises a heterodimer Fc polypeptide, where the heterodimer is made up of a first Fc domain, Fc1 (10), and a second, different, Fc domain Fc2 (15). The right panel illustrates one Ig protease domain (20) being linked to only one of the Fc polypeptides. As provided for herein, this linkage can be via a peptide linker (25). In some embodiments, the peptide linker (25) is any peptide linker, such as but not limited to those provided herein. Additionally, although as illustrated in FIG. 3, and in certain examples provided for herein, the protease is shown linked to one Fc polypeptide, such as the one containing what are referred to as “knob” substitutions, the protease could also be linked to the other Fc polypeptide, which can be referred to as having “hole” substitutions. Examples of such knob and hole substitutions are provided for herein and are known to one of skill in the art. Thus, the protease polypeptide domain in a monovalent protease can be linked to either of the Fc polypeptides that form the Fc heterodimer.

Although illustrated as being linked to the C-terminus of the Fc domain in FIG. 3 and in the table below, the Ig protease polypeptide could be linked to the N-terminus of the Fc domain directly or also through a peptide linker as provided for herein. Accordingly, in some embodiments, the polypeptide is conjugated to an Fc domain via a linker, such as a peptide linker provided for herein. In some embodiments, the polypeptide is conjugated to an Fc domain. In some embodiments, the N-terminal of the polypeptide is conjugated to the Fc domain. In some embodiments, the C-terminal of the polypeptide is conjugated to the Fc domain. In some embodiments, the N-terminal of the polypeptide is conjugated to an Fc domain via a linker, such as a peptide linker provided for herein. In some embodiments, the C-terminal of the polypeptide is conjugated to an Fc domain via a linker, such as a peptide linker provided for herein.

In some embodiments, the dimer is a homodimer molecule. In some embodiments, the dimer is a heterodimer molecule.

As used herein, the term “non-covalently conjugated” can mean that a polypeptide is tethered to another polypeptide through a linker. In some embodiments, the linker is a peptide linker. Non-limiting examples of peptide linkers that can be used are known in the art and are provide for herein.

As discussed herein the different domains, molecules, or polypeptide can be linked together with a linker domain or region. Any linker region described herein can be used as a linker. Linkers can be for example, glycine/serine linkers. In some embodiments, the linker can comprise one or more repeats of GGGGS (SEQ ID NO: 260). In some embodiments, the linker comprises 1, 2, 3, 4, or 5 repeats. In some embodiments, the linker comprises the sequence of GGGGSGGGGS (SEQ ID NO: 261). In some embodiments, the linker comprises the sequence of GGGGSGGGGSGGGGS (SEQ ID NO: 262). In some embodiments, the linker comprises the sequence of GGGGSGGGGSGGGGSG (SEQ ID NO: 263). In some embodiments, the linker comprises the amino acid sequence of GGGSK (SEQ ID NO: 264), which can be repeated 1-4 times, which can be represented as (GGGSK) n, where n is 1-4. Thus, in some embodiments, the linker can comprise the amino acid sequence of GGGSKGGGSK (SEQ ID NO: 265), GGGSKGGGSKGGGSK (SEQ ID NO: 266), or GGGSKGGGSKGGGSKGGGSK (SEQ ID NO: 267). In some embodiments, the linker comprises the sequence of GGGGSGGGGSGGGGSGGGGSG (SEQ ID NO: 268). In some embodiments, the linker comprises the sequence of GGGGSGGGGSG (SEQ ID NO: 269). In some embodiments, the linker comprises: GGGGS (SEQ ID NO: 260), (GGGGS) 3 (SEQ ID NO: 262), (GGGGS) n (n=1, 2, 3, 4) (SEQ ID NO: 260, SEQ ID NO: 261, SEQ ID NO: 262, SEQ ID NO: 270, respectively), (Gly): (SEQ ID NO: 271), (Gly) 6 (SEQ ID NO: 272), (EAAAK) 3 (SEQ ID NO: 273), (EAAK) n (n=1-3) (SEQ ID NO: 274, SEQ ID NO: 275, SEQ ID NO: 276, respectively), A (EAAAK) 4ALEA (EAAAK) 4A (SEQ ID NO: 277), AEAAAKEAAAKA (SEQ ID NO: 278), or AEEEKAEEEKAEEEK (SEQ ID NO: 279). These linkers can be used in any of the compounds or compositions provided herein.

In some embodiments, a molecule, or a composition comprises a first polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to:

(SEQ ID NO: 257)
DKTHTCPPCPAPEAAGAPSVFLFPPKPKDTLMISRTPEVTCVVVDV
SHEDPEVKFNWYVDGVEVHNAKTKPREEQQNSTYRVVSVLTVLHQD
WLNGKEYKCKVSNKALKAPIEKTISKAKGQPREPQVYTLPPSRDEL
TKNQVSLWCLVKGFYPSDIAVEWESNGQPENNYKTTPPVLDSDGSF
FLYSKLTVDKSRWQQGNVFSCSVMHEALHNHYTQKSLSLSPGAEEE
KAEEEKAEEEKTALGRRKAEDRIKETLWADGVKIDEKDFEHIKSSH
EDYFAVEYGKGSGYYDVNKKMDSEDSALCVGAVVTNMFHWWVDQNR
ENIEKFKKQDPENNGVMKFEGVKEATVDELNEFYRDNRDNYADQSR
FFDLIKKHFGNKALNPEELFELYFNGFYYGSSHRYIEDNEPAQNNL
FSKVLENNKLLNPERSYGIDGFSNNVINALKSHKVLGLVYTTPLRY
RHIISMWGADIDQDGKVKAIYVTDSDDREITFNDNSKRRIGMIRYD
VLVDDNGNIRFTGKGATHKEEGAIYAGLYSLSRGDEVWEDYFKKYP
EVQENL;

and
a second polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 255.

In some embodiments, a molecule, or a composition comprises a first polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to:

(SEQ ID NO: 258)
TALGRRKAEDRIKETLWADGVKIDEKDFEHIKSSHEDYFAVEYGKG
SGYYDVNKKMDSEDSALCVGAVVTNMFHWWVDQNRENIEKFKKQDP
ENNGVMKFEGVKEATVDELNEFYRDNRDNYADQSRFFDLIKKHFGN
KALNPEELFELYFNGFYYGSSHRYIEDNEPAQNNLFSKVLENNKLL
NPERSYGIDGFSNNVINALKSHKVLGLVYTTPLRYRHIISMWGADI
DQDGKVKAIYVTDSDDREITFNDNSKRRIGMIRYDVLVDDNGNIRF
TGKGATHKEEGAIYAGLYSLSRGDEVWEDYFKKYPEVQENLDKTHT
CPPCPAPEAAGAPSVFLFPPKPKDTLMISRTPEVTCVVVDVSHEDP
EVKFNWYVDGVEVHNAKTKPREEQQNSTYRVVSVLIVLHQDWLNGK
EYKCKVSNKALKAPIEKTISKAKGQPREPQVYTLPPSRDELTKNQV
SLWCLVKGFYPSDIAVEWESNGQPENNYKTTPPVLDSDGSFFLYSK
LTVDKSRWQQGNVFSCSVMHEALHNHYTQKSLSLSPG;

and
a second polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 255.

In some embodiments, a molecule, or a composition comprises a first polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to:

(SEQ ID NO: 259)
TALGRRKAEDRIKETLWADGVKIDEKDFEHIKSSHEDYFAVEYGKG
SGYYDVNKKMDSEDSALCVGAVVTNMFHWWVDQNRENIEKFKKQDP
ENNGVMKFEGVKEATVDELNEFYRDNRDNYADQSRFFDLIKKHFGN
KALNPEELFELYFNGFYYGSSHRYIEDNEPAQNNLFSKVLENNKLL
NPERSYGIDGFSNNVINALKSHKVLGLVYTTPLRYRHIISMWGADI
DQDGKVKAIYVTDSDDREITFNDNSKRRIGMIRYDVLVDDNGNIRF
TGKGATHKEEGAIYAGLYSLSRGDEVWEDYFKKYPEVQENLDKTHT
CPPCPAPEAAGAPSVFLFPPKPKDTLMISRTPEVTCVVVDVSHEDP
EVKFNWYVDGVEVHNAKTKPREEQQNSTYRVVSVLTVLHQDWLNGK
EYKCKVSNKALKAPIEKTISKAKGQPREPQVYTLPPSRDELTKNQV
SLTCLVKGFYPSDIAVEWESNGQPENNYKTTPPVLDSDGSFFLYSK
LTVDKSRWQQGNVFSCSVMHEALHNHYTQKSLSLSPG;

and
a second polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 259.

In some embodiments, a molecule, or a composition comprises a first polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 380, 251, 5-243, 247, 280-379, 381-528, 737, 745-823, 828-864, or 885-916; and a second polypeptide comprising an Fc domain, such as those provided for herein. In some embodiments, a molecule, or a composition comprises a first polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 380, 251, 5-243, 247, 280-379, 381-528, 737, 745-823, 828-864, or 885-916; and a second polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 253, 254, 255, or 256. In some embodiments, a molecule, or a composition comprises a first polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 380, 251, 5-243, 247, 280-379, 381-528, 737, 745-823, 828-864, or 885-916; and a second polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 380, 251, 5-243, 247, 280-379, 381-528, 737, 745-823, 828-864, or 885-916.

In some embodiments, a molecule, or a composition comprises a first polypeptide comprising an amino acid sequence of SEQ ID NO: 380, 251, 5-243, 247, 280-379, 381-528, 737, 745-823, 828-864, or 885-916; and a second polypeptide comprising an Fc domain, such as those provided for herein. In some embodiments, a molecule, or a composition comprises a first polypeptide comprising an amino acid sequence of SEQ ID NO: 380, 251, 5-243, 247, 280-379, 381-528, 737, 745-823, 828-864, or 885-916, or 737; and a second polypeptide comprising an amino acid sequence of SEQ ID NO: 253, 254, 255, or 256. In some embodiments, a molecule, or a composition comprises a first polypeptide comprising an amino acid sequence of SEQ ID NO: 380, 251, 5-243, 247, 280-379, 381-528, 737, 745-823, 828-864, or 885-916; and a second polypeptide comprising an amino acid sequence of SEQ ID NO: 380, 251, 5-243, 247, 280-379, 381-528, 737, 745-823, 828-864, or 885-916.

In some embodiments, a molecule, or a composition comprises a first polypeptide comprising an amino acid sequence of SEQ ID NO: 257, or 258; and a second polypeptide comprising an amino acid sequence of SEQ ID NO: 253, 254, 255, or 256.

In some embodiments, a molecule, or a composition comprises a first polypeptide comprising an amino acid sequence of SEQ ID NO: 257; and a second polypeptide comprising an Fc domain, such as those provided for herein. In some embodiments, a molecule, or a composition comprises a first polypeptide comprising an amino acid sequence of SEQ ID NO: 257; and a second polypeptide comprising an amino acid sequence of SEQ ID NO: 255.

In some embodiments, a molecule, or a composition comprises a first polypeptide comprising an amino acid sequence of SEQ ID NO: 258; and a second polypeptide comprising an Fc domain, such as those provided for herein. In some embodiments, a molecule, or a composition comprises a first polypeptide comprising an amino acid sequence of SEQ ID NO: 258; and a second polypeptide comprising an amino acid sequence of SEQ ID NO: 255.

In some embodiments, a molecule, or a composition comprises a first polypeptide comprising an amino acid sequence of SEQ ID NO: 259; and a second polypeptide comprising an Fc domain, such as those provided for herein. In some embodiments, a molecule, or a composition comprises a first polypeptide comprising an amino acid sequence of SEQ ID NO: 259; and a second polypeptide comprising an amino acid sequence of SEQ ID NO: 259.

In some embodiments, a molecule, or a composition comprises an amino acid sequence selected from any one in Table 6 below.

TABLE 6

| | Fc | Linker | Protease | Full Molecule |
|---|---|---|---|---|
| Name | Sequence | Sequence | Sequence | Sequence |
| FP1 | SEQ ID NO: 721 | SEQ ID NO: 726 | SEQ ID NO: 745 | SEQ ID NO: 529 |
| FP2 | SEQ ID NO: 721 | SEQ ID NO: 727 | SEQ ID NO: 746 | SEQ ID NO: 530 |
| FP3 | SEQ ID NO: 721 | SEQ ID NO: 727 | SEQ ID NO: 749 | SEQ ID NO: 531 |
| FP4 | SEQ ID NO: 721 | SEQ ID NO: 727 | SEQ ID NO: 748 | SEQ ID NO: 532 |
| FP5 | SEQ ID NO: 721 | SEQ ID NO: 727 | SEQ ID NO: 747 | SEQ ID NO: 533 |
| FP6 | SEQ ID NO: 721 | SEQ ID NO: 727 | SEQ ID NO: 916 | SEQ ID NO: 534 |
| FP7 | SEQ ID NO: 721 | SEQ ID NO: 727 | SEQ ID NO: 753 | SEQ ID NO: 535 |
| FP8 | SEQ ID NO: 721 | SEQ ID NO: 727 | SEQ ID NO: 751 | SEQ ID NO: 536 |
| FP9 | SEQ ID NO: 721 | SEQ ID NO: 727 | SEQ ID NO: 823 | SEQ ID NO: 537 |
| FP10 | SEQ ID NO: 721 | SEQ ID NO: 727 | SEQ ID NO: 752 | SEQ ID NO: 538 |
| FP11 | SEQ ID NO: 721 | SEQ ID NO: 727 | SEQ ID NO: 754 | SEQ ID NO: 539 |
| FP12 | SEQ ID NO: 721 | SEQ ID NO: 727 | SEQ ID NO: 755 | SEQ ID NO: 540 |
| FP13 | SEQ ID NO: 721 | SEQ ID NO: 727 | SEQ ID NO: 756 | SEQ ID NO: 541 |
| FP14 | SEQ ID NO: 721 | SEQ ID NO: 727 | SEQ ID NO: 757 | SEQ ID NO: 542 |
| FP15 | SEQ ID NO: 721 | SEQ ID NO: 727 | SEQ ID NO: 758 | SEQ ID NO: 543 |
| FP16 | SEQ ID NO: 721 | SEQ ID NO: 727 | SEQ ID NO: 750 | SEQ ID NO: 544 |
| FP17 | SEQ ID NO: 721 | SEQ ID NO: 728 | SEQ ID NO: 750 | SEQ ID NO: 545 |
| FP18 | SEQ ID NO: 721 | SEQ ID NO: 727 | SEQ ID NO: 759 | SEQ ID NO: 546 |
| FP19 | SEQ ID NO: 721 | SEQ ID NO: 727 | SEQ ID NO: 760 | SEQ ID NO: 547 |
| FP20 | SEQ ID NO: 721 | SEQ ID NO: 727 | SEQ ID NO: 761 | SEQ ID NO: 548 |
| FP21 | SEQ ID NO: 721 | SEQ ID NO: 727 | SEQ ID NO: 762 | SEQ ID NO: 549 |
| FP22 | SEQ ID NO: 721 | SEQ ID NO: 727 | SEQ ID NO: 763 | SEQ ID NO: 550 |
| FP23 | SEQ ID NO: 721 | SEQ ID NO: 727 | SEQ ID NO: 764 | SEQ ID NO: 551 |
| FP24 | SEQ ID NO: 721 | SEQ ID NO: 727 | SEQ ID NO: 765 | SEQ ID NO: 552 |
| FP25 | SEQ ID NO: 721 | SEQ ID NO: 727 | SEQ ID NO: 766 | SEQ ID NO: 553 |
| FP26 | SEQ ID NO: 721 | SEQ ID NO: 727 | SEQ ID NO: 767 | SEQ ID NO: 554 |
| FP27 | SEQ ID NO: 721 | SEQ ID NO: 727 | SEQ ID NO: 768 | SEQ ID NO: 555 |
| FP28 | SEQ ID NO: 721 | SEQ ID NO: 727 | SEQ ID NO: 769 | SEQ ID NO: 556 |
| FP29 | SEQ ID NO: 721 | SEQ ID NO: 727 | SEQ ID NO: 770 | SEQ ID NO: 557 |
| FP30 | SEQ ID NO: 721 | SEQ ID NO: 727 | SEQ ID NO: 771 | SEQ ID NO: 558 |
| FP31 | SEQ ID NO: 721 | SEQ ID NO: 727 | SEQ ID NO: 772 | SEQ ID NO: 559 |
| FP32 | SEQ ID NO: 721 | SEQ ID NO: 727 | SEQ ID NO: 773 | SEQ ID NO: 560 |
| FP33 | SEQ ID NO: 721 | SEQ ID NO: 727 | SEQ ID NO: 774 | SEQ ID NO: 561 |
| FP34 | SEQ ID NO: 721 | SEQ ID NO: 727 | SEQ ID NO: 775 | SEQ ID NO: 562 |
| FP35 | SEQ ID NO: 721 | SEQ ID NO: 727 | SEQ ID NO: 776 | SEQ ID NO: 563 |
| FP36 | SEQ ID NO: 721 | SEQ ID NO: 727 | SEQ ID NO: 777 | SEQ ID NO: 564 |
| FP37 | SEQ ID NO: 721 | SEQ ID NO: 727 | SEQ ID NO: 778 | SEQ ID NO: 565 |
| FP38 | SEQ ID NO: 721 | SEQ ID NO: 727 | SEQ ID NO: 779 | SEQ ID NO: 566 |
| FP39 | SEQ ID NO: 721 | SEQ ID NO: 727 | SEQ ID NO: 780 | SEQ ID NO: 567 |
| FP40 | SEQ ID NO: 721 | SEQ ID NO: 727 | SEQ ID NO: 781 | SEQ ID NO: 568 |
| FP41 | SEQ ID NO: 721 | SEQ ID NO: 727 | SEQ ID NO: 782 | SEQ ID NO: 569 |
| FP42 | SEQ ID NO: 721 | SEQ ID NO: 727 | SEQ ID NO: 783 | SEQ ID NO: 570 |
| FP43 | SEQ ID NO: 721 | SEQ ID NO: 727 | SEQ ID NO: 784 | SEQ ID NO: 571 |
| FP44 | SEQ ID NO: 721 | SEQ ID NO: 727 | SEQ ID NO: 785 | SEQ ID NO: 572 |
| FP45 | SEQ ID NO: 721 | SEQ ID NO: 727 | SEQ ID NO: 786 | SEQ ID NO: 573 |
| FP46 | SEQ ID NO: 721 | SEQ ID NO: 727 | SEQ ID NO: 787 | SEQ ID NO: 574 |
| FP47 | SEQ ID NO: 721 | SEQ ID NO: 727 | SEQ ID NO: 788 | SEQ ID NO: 575 |
| FP48 | SEQ ID NO: 721 | SEQ ID NO: 727 | SEQ ID NO: 789 | SEQ ID NO: 576 |
| FP49 | SEQ ID NO: 721 | SEQ ID NO: 727 | SEQ ID NO: 790 | SEQ ID NO: 577 |
| FP50 | SEQ ID NO: 721 | SEQ ID NO: 727 | SEQ ID NO: 791 | SEQ ID NO: 578 |
| FP51 | SEQ ID NO: 721 | SEQ ID NO: 727 | SEQ ID NO: 792 | SEQ ID NO: 579 |
| FP52 | SEQ ID NO: 721 | SEQ ID NO: 727 | SEQ ID NO: 793 | SEQ ID NO: 580 |
| FP53 | SEQ ID NO: 721 | SEQ ID NO: 727 | SEQ ID NO: 794 | SEQ ID NO: 581 |
| FP54 | SEQ ID NO: 721 | SEQ ID NO: 727 | SEQ ID NO: 795 | SEQ ID NO: 582 |
| FP55 | SEQ ID NO: 721 | SEQ ID NO: 727 | SEQ ID NO: 796 | SEQ ID NO: 583 |
| FP56 | SEQ ID NO: 721 | SEQ ID NO: 727 | SEQ ID NO: 797 | SEQ ID NO: 584 |
| FP57 | SEQ ID NO: 721 | SEQ ID NO: 727 | SEQ ID NO: 798 | SEQ ID NO: 585 |
| FP58 | SEQ ID NO: 721 | SEQ ID NO: 727 | SEQ ID NO: 799 | SEQ ID NO: 586 |
| FP59 | SEQ ID NO: 721 | SEQ ID NO: 727 | SEQ ID NO: 800 | SEQ ID NO: 587 |
| FP60 | SEQ ID NO: 721 | SEQ ID NO: 727 | SEQ ID NO: 801 | SEQ ID NO: 588 |
| FP61 | SEQ ID NO: 721 | SEQ ID NO: 727 | SEQ ID NO: 802 | SEQ ID NO: 589 |
| FP62 | SEQ ID NO: 721 | SEQ ID NO: 727 | SEQ ID NO: 803 | SEQ ID NO: 590 |
| FP63 | SEQ ID NO: 721 | SEQ ID NO: 727 | SEQ ID NO: 804 | SEQ ID NO: 591 |
| FP64 | SEQ ID NO: 721 | SEQ ID NO: 727 | SEQ ID NO: 805 | SEQ ID NO: 592 |
| FP65 | SEQ ID NO: 721 | SEQ ID NO: 727 | SEQ ID NO: 806 | SEQ ID NO: 593 |
| FP66 | SEQ ID NO: 721 | SEQ ID NO: 727 | SEQ ID NO: 807 | SEQ ID NO: 594 |
| FP67 | SEQ ID NO: 721 | SEQ ID NO: 727 | SEQ ID NO: 808 | SEQ ID NO: 595 |
| FP68 | SEQ ID NO: 721 | SEQ ID NO: 727 | SEQ ID NO: 809 | SEQ ID NO: 596 |
| FP69 | SEQ ID NO: 721 | SEQ ID NO: 727 | SEQ ID NO: 810 | SEQ ID NO: 597 |
| FP70 | SEQ ID NO: 721 | SEQ ID NO: 727 | SEQ ID NO: 811 | SEQ ID NO: 598 |
| FP71 | SEQ ID NO: 721 | SEQ ID NO: 727 | SEQ ID NO: 812 | SEQ ID NO: 599 |
| FP72 | SEQ ID NO: 721 | SEQ ID NO: 727 | SEQ ID NO: 813 | SEQ ID NO: 600 |
| FP73 | SEQ ID NO: 721 | SEQ ID NO: 727 | SEQ ID NO: 814 | SEQ ID NO: 601 |
| FP74 | SEQ ID NO: 721 | SEQ ID NO: 727 | SEQ ID NO: 815 | SEQ ID NO: 602 |
| FP75 | SEQ ID NO: 721 | SEQ ID NO: 727 | SEQ ID NO: 816 | SEQ ID NO: 603 |
| FP76 | SEQ ID NO: 721 | SEQ ID NO: 727 | SEQ ID NO: 817 | SEQ ID NO: 604 |
| FP77 | SEQ ID NO: 721 | SEQ ID NO: 727 | SEQ ID NO: 818 | SEQ ID NO: 605 |
| FP78 | SEQ ID NO: 721 | SEQ ID NO: 727 | SEQ ID NO: 819 | SEQ ID NO: 606 |
| FP79 | SEQ ID NO: 721 | SEQ ID NO: 727 | SEQ ID NO: 820 | SEQ ID NO: 607 |
| FP80 | SEQ ID NO: 721 | SEQ ID NO: 727 | SEQ ID NO: 821 | SEQ ID NO: 608 |
| FP81 | SEQ ID NO: 721 | SEQ ID NO: 727 | SEQ ID NO: 822 | SEQ ID NO: 609 |
| FP82 | SEQ ID NO: 721 | SEQ ID NO: 727 | SEQ ID NO: 828 | SEQ ID NO: 610 |
| FP83 | SEQ ID NO: 721 | SEQ ID NO: 727 | SEQ ID NO: 829 | SEQ ID NO: 611 |
| FP84 | SEQ ID NO: 721 | SEQ ID NO: 727 | SEQ ID NO: 830 | SEQ ID NO: 612 |
| FP85 | SEQ ID NO: 721 | SEQ ID NO: 727 | SEQ ID NO: 831 | SEQ ID NO: 613 |
| FP86 | SEQ ID NO: 721 | SEQ ID NO: 727 | SEQ ID NO: 832 | SEQ ID NO: 614 |
| FP87 | SEQ ID NO: 721 | SEQ ID NO: 727 | SEQ ID NO: 833 | SEQ ID NO: 615 |
| FP88 | SEQ ID NO: 721 | SEQ ID NO: 727 | SEQ ID NO: 834 | SEQ ID NO: 616 |
| FP89 | SEQ ID NO: 721 | SEQ ID NO: 727 | SEQ ID NO: 835 | SEQ ID NO: 617 |
| FP90 | SEQ ID NO: 721 | SEQ ID NO: 727 | SEQ ID NO: 836 | SEQ ID NO: 618 |
| FP91 | SEQ ID NO: 721 | SEQ ID NO: 727 | SEQ ID NO: 837 | SEQ ID NO: 619 |
| FP92 | SEQ ID NO: 721 | SEQ ID NO: 727 | SEQ ID NO: 838 | SEQ ID NO: 620 |
| FP93 | SEQ ID NO: 721 | SEQ ID NO: 727 | SEQ ID NO: 839 | SEQ ID NO: 621 |
| FP94 | SEQ ID NO: 721 | SEQ ID NO: 727 | SEQ ID NO: 840 | SEQ ID NO: 622 |
| FP95 | SEQ ID NO: 721 | SEQ ID NO: 727 | SEQ ID NO: 841 | SEQ ID NO: 623 |
| FP96 | SEQ ID NO: 721 | SEQ ID NO: 727 | SEQ ID NO: 842 | SEQ ID NO: 624 |
| FP97 | SEQ ID NO: 721 | SEQ ID NO: 727 | SEQ ID NO: 843 | SEQ ID NO: 625 |
| FP98 | SEQ ID NO: 721 | SEQ ID NO: 727 | SEQ ID NO: 844 | SEQ ID NO: 626 |
| FP99 | SEQ ID NO: 721 | SEQ ID NO: 727 | SEQ ID NO: 845 | SEQ ID NO: 627 |
| FP100 | SEQ ID NO: 721 | SEQ ID NO: 727 | SEQ ID NO: 846 | SEQ ID NO: 628 |
| FP101 | SEQ ID NO: 721 | SEQ ID NO: 727 | SEQ ID NO: 847 | SEQ ID NO: 629 |
| FP102 | SEQ ID NO: 721 | SEQ ID NO: 727 | SEQ ID NO: 848 | SEQ ID NO: 630 |
| FP103 | SEQ ID NO: 721 | SEQ ID NO: 727 | SEQ ID NO: 849 | SEQ ID NO: 631 |
| FP104 | SEQ ID NO: 721 | SEQ ID NO: 727 | SEQ ID NO: 850 | SEQ ID NO: 632 |
| FP105 | SEQ ID NO: 721 | SEQ ID NO: 727 | SEQ ID NO: 851 | SEQ ID NO: 633 |
| FP106 | SEQ ID NO: 721 | SEQ ID NO: 727 | SEQ ID NO: 852 | SEQ ID NO: 634 |
| FP107 | SEQ ID NO: 721 | SEQ ID NO: 727 | SEQ ID NO: 853 | SEQ ID NO: 635 |
| FP108 | SEQ ID NO: 721 | SEQ ID NO: 727 | SEQ ID NO: 854 | SEQ ID NO: 636 |
| FP109 | SEQ ID NO: 721 | SEQ ID NO: 727 | SEQ ID NO: 855 | SEQ ID NO: 637 |
| FP110 | SEQ ID NO: 721 | SEQ ID NO: 727 | SEQ ID NO: 856 | SEQ ID NO: 638 |
| FP111 | SEQ ID NO: 721 | SEQ ID NO: 727 | SEQ ID NO: 857 | SEQ ID NO: 639 |
| FP112 | SEQ ID NO: 721 | SEQ ID NO: 727 | SEQ ID NO: 858 | SEQ ID NO: 640 |
| FP113 | SEQ ID NO: 721 | SEQ ID NO: 727 | SEQ ID NO: 859 | SEQ ID NO: 641 |
| FP114 | SEQ ID NO: 721 | SEQ ID NO: 727 | SEQ ID NO: 860 | SEQ ID NO: 642 |
| FP115 | SEQ ID NO: 721 | SEQ ID NO: 727 | SEQ ID NO: 861 | SEQ ID NO: 643 |
| FP116 | SEQ ID NO: 721 | SEQ ID NO: 727 | SEQ ID NO: 862 | SEQ ID NO: 644 |
| FP117 | SEQ ID NO: 721 | SEQ ID NO: 727 | SEQ ID NO: 863 | SEQ ID NO: 645 |
| FP118 | SEQ ID NO: 721 | SEQ ID NO: 727 | SEQ ID NO: 864 | SEQ ID NO: 646 |
| FP119 | SEQ ID NO: 721 | SEQ ID NO: 727 | SEQ ID NO: 885 | SEQ ID NO: 647 |
| FP120 | SEQ ID NO: 721 | SEQ ID NO: 727 | SEQ ID NO: 886 | SEQ ID NO: 648 |
| FP121 | SEQ ID NO: 721 | SEQ ID NO: 727 | SEQ ID NO: 378 | SEQ ID NO: 649 |
| FP122 | SEQ ID NO: 721 | SEQ ID NO: 727 | SEQ ID NO: 887 | SEQ ID NO: 650 |
| FP123 | SEQ ID NO: 721 | SEQ ID NO: 727 | SEQ ID NO: 888 | SEQ ID NO: 651 |
| FP124 | SEQ ID NO: 721 | SEQ ID NO: 727 | SEQ ID NO: 889 | SEQ ID NO: 652 |
| FP125 | SEQ ID NO: 721 | SEQ ID NO: 727 | SEQ ID NO: 890 | SEQ ID NO: 653 |
| FP126 | SEQ ID NO: 721 | SEQ ID NO: 727 | SEQ ID NO: 891 | SEQ ID NO: 654 |
| FP127 | SEQ ID NO: 721 | SEQ ID NO: 727 | SEQ ID NO: 892 | SEQ ID NO: 656 |
| FP128 | SEQ ID NO: 721 | SEQ ID NO: 727 | SEQ ID NO: 893 | SEQ ID NO: 657 |
| FP129 | SEQ ID NO: 721 | SEQ ID NO: 727 | SEQ ID NO: 894 | SEQ ID NO: 658 |
| FP130 | SEQ ID NO: 721 | SEQ ID NO: 727 | SEQ ID NO: 895 | SEQ ID NO: 659 |
| FP131 | SEQ ID NO: 721 | SEQ ID NO: 727 | SEQ ID NO: 896 | SEQ ID NO: 660 |
| FP132 | SEQ ID NO: 721 | SEQ ID NO: 261 | SEQ ID NO: 378 | SEQ ID NO: 661 |
| FP133 | SEQ ID NO: 721 | SEQ ID NO: 265 | SEQ ID NO: 378 | SEQ ID NO: 662 |
| FP134 | SEQ ID NO: 721 | SEQ ID NO: 266 | SEQ ID NO: 378 | SEQ ID NO: 663 |
| FP135 | SEQ ID NO: 721 | SEQ ID NO: 267 | SEQ ID NO: 378 | SEQ ID NO: 664 |
| FP136 | SEQ ID NO: 721 | SEQ ID NO: 729 | SEQ ID NO: 378 | SEQ ID NO: 665 |
| FP137 | SEQ ID NO: 721 | SEQ ID NO: 730 | SEQ ID NO: 378 | SEQ ID NO: 666 |
| FP138 | SEQ ID NO: 721 | SEQ ID NO: 731 | SEQ ID NO: 378 | SEQ ID NO: 667 |
| FP139 | SEQ ID NO: 721 | SEQ ID NO: 732 | SEQ ID NO: 378 | SEQ ID NO: 668 |
| FP140 | SEQ ID NO: 721 | SEQ ID NO: 733 | SEQ ID NO: 378 | SEQ ID NO: 669 |
| FP141 | SEQ ID NO: 253; | SEQ ID NO: 727 | SEQ ID NO: 378 | SEQ ID NO: 670 |
| | and | | | |
| | SEQ ID NO: 255 | | | |
| FP142 | SEQ ID NO: 254; | SEQ ID NO: 727 | SEQ ID NO: 378 | SEQ ID NO: 671 |
| | and | | | |
| | SEQ ID NO: 256 | | | |
| FP143 | SEQ ID NO: 724 | SEQ ID NO: 727 | SEQ ID NO: 378 | SEQ ID NO: 672 |
| FP144 | SEQ ID NO: 725 | SEQ ID NO: 727 | SEQ ID NO: 378 | SEQ ID NO: 673 |
| FP145 | SEQ ID NO: 721 | SEQ ID NO: 727 | SEQ ID NO: 380 | SEQ ID NO: 674 |
| FP146 | SEQ ID NO: 721 | SEQ ID NO: 727 | SEQ ID NO: 897 | SEQ ID NO: 675 |
| FP147 | SEQ ID NO: 721 | SEQ ID NO: 727 | SEQ ID NO: 898 | SEQ ID NO: 676 |
| FP148 | SEQ ID NO: 721 | SEQ ID NO: 727 | SEQ ID NO: 899 | SEQ ID NO: 677 |
| FP149 | SEQ ID NO: 721 | SEQ ID NO: 727 | SEQ ID NO: 900 | SEQ ID NO: 678 |
| FP150 | SEQ ID NO: 721 | SEQ ID NO: 727 | SEQ ID NO: 901 | SEQ ID NO: 679 |
| FP151 | SEQ ID NO: 721 | SEQ ID NO: 727 | SEQ ID NO: 902 | SEQ ID NO: 680 |
| FP152 | SEQ ID NO: 721 | SEQ ID NO: 727 | SEQ ID NO: 903 | SEQ ID NO: 681 |
| FP153 | SEQ ID NO: 721 | SEQ ID NO: 727 | SEQ ID NO: 904 | SEQ ID NO: 682 |
| FP154 | SEQ ID NO: 721 | SEQ ID NO: 727 | SEQ ID NO: 905 | SEQ ID NO: 683 |
| FP155 | SEQ ID NO: 721 | SEQ ID NO: 727 | SEQ ID NO: 906 | SEQ ID NO: 684 |
| FP156 | SEQ ID NO: 721 | SEQ ID NO: 727 | SEQ ID NO: 907 | SEQ ID NO: 685 |
| FP157 | SEQ ID NO: 721 | SEQ ID NO: 727 | SEQ ID NO: 908 | SEQ ID NO: 686 |
| FP158 | SEQ ID NO: 721 | SEQ ID NO: 727 | SEQ ID NO: 909 | SEQ ID NO: 687 |
| FP159 | SEQ ID NO: 721 | SEQ ID NO: 727 | SEQ ID NO: 910 | SEQ ID NO: 688 |
| FP160 | SEQ ID NO: 721 | SEQ ID NO: 727 | SEQ ID NO: 911 | SEQ ID NO: 689 |
| FP161 | SEQ ID NO: 721 | SEQ ID NO: 727 | SEQ ID NO: 912 | SEQ ID NO: 690 |
| FP162 | SEQ ID NO: 721 | SEQ ID NO: 727 | SEQ ID NO: 913 | SEQ ID NO: 691 |
| FP163 | SEQ ID NO: 721 | SEQ ID NO: 727 | SEQ ID NO: 914 | SEQ ID NO: 692 |
| FP164 | SEQ ID NO: 721 | SEQ ID NO: 727 | SEQ ID NO: 915 | SEQ ID NO: 693 |
| FP165 | SEQ ID NO: 722; | SEQ ID NO: 727 | SEQ ID NO: 378 | SEQ ID NO: 694 |
| | and | | | |
| | SEQ ID NO: 723 | | | |
| FP166 | SEQ ID NO: 724 | SEQ ID NO: 733 | SEQ ID NO: 378 | SEQ ID NO: 695 |
| FP167 | SEQ ID NO: 253; | SEQ ID NO: 733 | SEQ ID NO: 378 | SEQ ID NO: 696 |
| | and | | | |
| | SEQ ID NO: 255 | | | |
| FP168 | SEQ ID NO: 722; | SEQ ID NO: 733 | SEQ ID NO: 378 | SEQ ID NO: 697 |
| | and | | | |
| | SEQ ID NO: 723 | | | |
| FP169 | SEQ ID NO: 724 | SEQ ID NO: 727 | SEQ ID NO: 380 | SEQ ID NO: 698 |
| FP170 | SEQ ID NO: 253; | SEQ ID NO: 727 | SEQ ID NO: 380 | SEQ ID NO: 699 |
| | and | | | |
| | SEQ ID NO: 255 | | | |
| FP171 | SEQ ID NO: 722; | SEQ ID NO: 727 | SEQ ID NO: 380 | SEQ ID NO: 700 |
| | and | | | |
| | SEQ ID NO: 723 | | | |
| FP172 | SEQ ID NO: 721 | SEQ ID NO: 733 | SEQ ID NO: 380 | SEQ ID NO: 701 |
| FP173 | SEQ ID NO: 724 | SEQ ID NO: 733 | SEQ ID NO: 380 | SEQ ID NO: 702 |
| FP174 | SEQ ID NO: 253; | SEQ ID NO: 733 | SEQ ID NO: 380 | SEQ ID NO: 703 |
| | and | | | |
| | SEQ ID NO: 255 | | | |
| FP175 | SEQ ID NO: 722; | SEQ ID NO: 733 | SEQ ID NO: 380 | SEQ ID NO: 704 |
| | and | | | |
| | SEQ ID NO: 723 | | | |
| FP176 | SEQ ID NO: 721 | N/A | SEQ ID NO: 378 | SEQ ID NO: 705 |
| FP177 | SEQ ID NO: 253; | N/A | SEQ ID NO: 378 | SEQ ID NO: 706 |
| | and | | | |
| | SEQ ID NO: 255 | | | |
| FP178 | SEQ ID NO: 721 | N/A | SEQ ID NO: 380 | SEQ ID NO: 707 |
| FP179 | SEQ ID NO: 253; | N/A | SEQ ID NO: 380 | SEQ ID NO: 708 |
| | and | | | |
| | SEQ ID NO: 255 | | | |
| FP180 | SEQ ID NO: 253; | SEQ ID NO: 279 | SEQ ID NO: 378 | SEQ ID NO: 709 |
| | and | | | |
| | SEQ ID NO: 255 | | | |
| FP181 | SEQ ID NO: 253; | SEQ ID NO: 734 | SEQ ID NO: 378 | SEQ ID NO: 710 |
| | and | | | |
| | SEQ ID NO: 255 | | | |
| FP182 | SEQ ID NO: 253; | SEQ ID NO: 735 | SEQ ID NO: 378 | SEQ ID NO: 711 |
| | and | | | |
| | SEQ ID NO: 255 | | | |
| FP183 | SEQ ID NO: 253; | SEQ ID NO: 279 | SEQ ID NO: 380 | SEQ ID NO: 712 |
| | and | | | |
| | SEQ ID NO: 255 | | | |
| FP184 | SEQ ID NO: 253; | SEQ ID NO: 734 | SEQ ID NO: 380 | SEQ ID NO: 713 |
| | and | | | |
| | SEQ ID NO: 255 | | | |
| FP185 | SEQ ID NO: 253; | SEQ ID NO: 735 | SEQ ID NO: 380 | SEQ ID NO: 714 |
| | and | | | |
| | SEQ ID NO: 255 | | | |
| FP186 | SEQ ID NO: 253; | SEQ ID NO: 727 | SEQ ID NO: 374 | SEQ ID NO: 715 |
| | and | | | |
| | SEQ ID NO: 255 | | | |
| FP187 | SEQ ID NO: 253; | SEQ ID NO: 733 | SEQ ID NO: 374 | SEQ ID NO: 716 |
| | and | | | |
| | SEQ ID NO: 255 | | | |
| FP188 | SEQ ID NO: 253; | SEQ ID NO: 727 | SEQ ID NO: 376 | SEQ ID NO: 717 |
| | and | | | |
| | SEQ ID NO: 255 | | | |
| FP189 | SEQ ID NO: 253; | SEQ ID NO: 733 | SEQ ID NO: 376 | SEQ ID NO: 718 |
| | and | | | |
| | SEQ ID NO: 255 | | | |
| FP190 | SEQ ID NO: 253; | SEQ ID NO: 736 | SEQ ID NO: 378 | SEQ ID NO: 719 |
| | and | | | |
| | SEQ ID NO: 255 | | | |
| FP191 | SEQ ID NO: 253; | SEQ ID NO: 736 | SEQ ID NO: 380 | SEQ ID NO: 720 |
| | and | | | |
| | SEQ ID NO: 255 | | | |

In some embodiments, the molecule self-associates to form a dimer molecule. In some embodiments, the dimer molecule is a homodimer or a heterodimer. In some embodiments, a molecule, or a composition comprises a first polypeptide and a second polypeptide.

In some embodiments, a first polypeptide comprises an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 380, 251, 5-243, 247, 280-379, 381-528, 737, 745-823, 828-864, or 885-916, wherein said amino acid sequence is conjugated to an Fc domain, such as those provided for herein, for example, in SEQ ID NO: 721, 724, or 725. In some embodiments, a first polypeptide comprises an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 380, 251, 5-243, 247, 280-379, 381-528, 737, 745-823, 828-864, or 885-916, wherein said amino acid sequence is conjugated to an Fc domain comprising knob in hole mutations, such as those provided herein, for example, and without limitation, SEQ ID NO: 253, 254, 255, 256, 722, and/or 723. In some embodiments, a first polypeptide comprises an amino acid sequence of SEQ ID NO: 380, 251, 5-243, 247, 280-379, 381-528, 737, 745-823, 828-864, or 885-916, wherein said amino acid sequence is conjugated to an Fc domain, such as those provided for herein, for example, in SEQ ID NO: 721, 724, or 725. In some embodiments, a first polypeptide comprises an amino acid sequence of SEQ ID NO: 380, 251, 5-243, 247, 280-379, 381-528, 737, 745-823, 828-864, or 885-916, wherein said amino acid sequence is conjugated to an Fc domain comprising knob in hole mutations, such as those provided herein, for example, and without limitation, SEQ ID NO: 253, 254, 255, 256, 722, and/or 723. In some embodiments, a first polypeptide comprises an Fc domain, such as those provided for herein, for example, in SEQ ID NO: 721, 724, or 725. In some embodiments, a first polypeptide comprises an Fc domain comprising knob in hole mutations, such as those provided herein, for example, and without limitation, SEQ ID NO: 253, 254, 255, 256, 722, and/or 723.

In some embodiments, a second polypeptide comprises an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 380, 251, 5-243, 247, 280-379, 381-528, 737, 745-823, 828-864, or 885-916, wherein said amino acid sequence is conjugated to an Fc domain, such as those provided for herein, for example, in SEQ ID NO: 721, 724, or 725. In some embodiments, a second polypeptide comprises an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 380, 251, 5-243, 247, 280-379, 381-528, 737, 745-823, 828-864, or 885-916, wherein said amino acid sequence is conjugated to an Fc domain comprising knob in hole mutations, such as those provided herein, for example, and without limitation, SEQ ID NO: 253, 254, 255, 256, 722, and/or 723. In some embodiments, a second polypeptide comprises an amino acid sequence of SEQ ID NO: 380, 251, 5-243, 247, 280-379, 381-528, 737, 745-823, 828-864, or 885-916, wherein said amino acid sequence is conjugated to an Fc domain, such as those provided for herein, for example, in SEQ ID NO: 721, 724, or 725. In some embodiments, a second polypeptide comprises an amino acid sequence of SEQ ID NO: 380, 251, 5-243, 247, 280-379, 381-528, 737, 745-823, 828-864, or 885-916, wherein said amino acid sequence is conjugated to an Fc domain comprising knob in hole mutations, such as those provided herein, for example, and without limitation, SEQ ID NO: 253, 254, 255, 256, 722, and/or 723. In some embodiments, a second polypeptide comprises an Fc domain, such as those provided for herein, for example, in SEQ ID NO: 721, 724, or 725. In some embodiments, a second polypeptide comprises an Fc domain comprising knob in hole mutations, such as those provided herein, for example, and without limitation, SEQ ID NO: 253, 254, 255, 256, 722, and/or 723.

In some embodiments, a first polypeptide comprises an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 257, 258, 259, 529-720.

In some embodiments, a second polypeptide comprises an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 257, 258, 259, 529-720.

In some embodiments, a molecule, or a composition comprises first polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 257, 258, 259, 529-720; and a second polypeptide comprising an Fc domain, such as those provided for herein.

In some embodiments, a molecule, or a composition comprises first polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 257, 258, 259, 529-720; and a second polypeptide comprising an amino acid sequence having at least 50%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100% sequence identity to any one sequence of SEQ ID NO: 257, 258, 259, 529-720.

In some embodiments, a molecule, or a composition comprises first polypeptide comprising an amino acid sequence of SEQ ID NO: 257, 258, 259, 529-720; and a second polypeptide comprising an Fc domain, such as those provided for herein

In some embodiments, a molecule, or a composition comprises first polypeptide comprising an Fc domain, such as those provided for herein; and a second polypeptide comprising an amino acid sequence of SEQ ID NO: 257, 258, 259, 529-720.

In some embodiments, a molecule, or a composition comprises first polypeptide comprising an amino acid sequence of SEQ ID NO: 257, 258, 259, 529-720; and a second polypeptide comprising an amino acid sequence of SEQ ID NO: 257, 258, 259, 529-720.

In some embodiments, the polypeptide having protease activity is a polypeptide having IgE protease activity which binds to IgE. In some embodiments, the polypeptide having IgE protease activity binds to amino acids of an epitope of IgE. The epitopes are described herein.

In some embodiments, polypeptides having IgE protease activity, such as those provided herein, bind to an epitope on IgE. IgE has the amino acid sequence as set forth in SEQ ID NO: 246:

(SEQ ID NO: 246)
ASTQSPSVFPLTRCCKNIPSNATSVTLGCLATGYFPEPVMVTWDTG
SLNGTTMTLPATTLTLSGHYATISLLTVSGAWAKQMFTCRVAHTPS
STDWVDNKTFSVCSRDFTPPTVKILQSSCDGGGHFPPTIQLLCLVS
GYTPGTINITWLEDGQVMDVDLSTASTTQEGELASTQSELTLSQKH
WLSDRTYTCQVTYQGHTFEDSTKKCADSNPRGVSAYLSRPSPFDLF
IRKSPTITCLVVDLAPSKGTVNLTWSRASGKPVNHSTRKEEKQRNG
TLTVTSTLPVGTRDWIEGETYQCRVTHPHLPRALMRSTTKTSGPRA
APEVYAFATPEWPGSRDKRTLACLIQNFMPEDISVQWLHNEVQLPD
ARHSTTQPRKTKGSGFFVFSRLEVTRAEWEQKDEFICRAVHEAASP
SQTVQRAVSVNPGLAGGSAQSQRAPDRVLCHSGQQQGLPRAAGGSV
PHPRCHCGAGRADWPGPPELDVCVEEAEGEAPWTWTGLCIFAALFL
LSVSYSAAITLLMVQRFLSATRQGRPQTSLDYTNVLQPHA.

In some embodiments, polypeptides having IgE protease activity, such as those provided herein, bind to an epitope on IgE. In some embodiments, polypeptides having IgE protease activity, such as those provided herein, bind to an epitope on IgE that include IgE (SEQ ID NO: 246) residues S332, N333, P334, R335, G336, V337, S338, A339, D363, L364, A365, P366, K368, A378, K392, R394, N395, G396, T397, R420, H423, P424, H425, L426, P427, R428, A429, M431, R432, S433, or any combination thereof. In some embodiments, the epitope includes residues D363, A365, P366, K368, K392, R394, N395, G396, T397, and H425 of IgE. In some embodiments, the epitope includes residues S332, N333, P334, R335, G336, V337, S338, A339, D363, L364, A365, K368, A378, R420, H423, P424, H425, L426, P427, R428, A429, M431, R432, and S433 of IgE.

In some embodiments, polypeptides having IgE protease activity, such as those provided herein, bind to an epitope on IgE, wherein the IgE is a non-cleaved Fc. In some embodiments, polypeptides having IgE protease activity, such as those provided herein, bind to an epitope on IgE, wherein the IgE is a cleaved Fc. In some embodiments, the non-cleaved Fc epitope includes residues D363, A365, P366, K368, K392, R394, N395, G396, T397, and H425 of IgE. In some embodiments, the cleaved Fc epitope includes residues S332, N333, P334, R335, G336, V337, S338, A339, D363, L364, A365, K368, A378, R420, H423, P424, H425, L426, P427, R428, A429, M431, R432, and S433 of IgE.

In some embodiments, polypeptides having protease activity described herein are as effective, or more effective at cleaving IgE than the parental protease. In some embodiments, polypeptides having protease activity described herein are as effective, or more effective at cleaving IgE than the parental protease of SEQ ID NO: 1, 2, 3, 4, or 91. In some embodiments, polypeptides having protease activity described herein are more selective for cleaving IgE than the parental protease of SEQ ID NO: 1, 2, 3, 4, or 91.

In some embodiments, PRT-1 through PRT-493 is as effective, or more effective at cleaving IgE than the parental protease. In some embodiments, PRT-1 through PRT-493 is as effective at cleaving IgE as the parental protease. In some embodiments, PRT-1 through PRT-493 is as effective, or more effective at cleaving IgE than the parental protease of SEQ ID NO: 1, 2, 3, 4, or 91. In some embodiments, PRT-1 through PRT-493 is as effective at cleaving IgE as the parental protease of SEQ ID NO: 1, 2, 3, 4, or 91.

In some embodiments, FP-1 through FP-191 is as effective, or more effective at cleaving IgE than the parental protease. In some embodiments, FP-1 through FP-191 is as effective at cleaving IgE as the parental protease. In some embodiments, FP-1 through FP-191 is as effective, or more effective at cleaving IgE than the parental protease of SEQ ID NO: 1, 2, 3, or 4. In some embodiments, FP-1 through FP-191 is as effective at cleaving IgE as the parental protease of SEQ ID NO: 1, 2, 3, or 4.

In some embodiments, the polypeptide, such as those provided for herein, is more effective or more selective at cleaving IgE than the polypeptide of SEQ ID NO: 1, 3, or 91 and is more selective for cleaving IgE than IgG.

In the context of nucleotide sequence, such as those encoding for the domains, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., an amino acid sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence. For example, a Fc variant can have the amino acid sequence of a Fc polypeptide but comprise a mutation that prevents or disrupts cleavage by a protease.

Calculations of homology or sequence identity between sequences (the terms are used interchangeably herein) can be performed as follows.

To determine the percent identity of two amino acid sequences, or of two nucleic acid sequences, the amino acid sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the amino acid sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any a nucleic acid sequence provided herein. BLAST protein searches can be performed with the XBLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The molecules and polypeptides provided for herein can be used to treat diseases and conditions mediated by IgE. Thus, embodiments are provided for methods of treating an IgE mediated disease or disorder in a subject. In some embodiments, the IgE mediated disease or disorder is an autoimmune disease or disorder. In some embodiments, the methods comprise administering to the subject a polypeptide, molecule, compound, or compositions as provided for herein. In some embodiments, the subject has or is at risk of having an IgE mediated disease or disorder. In some embodiments, the subject has or is at risk of having an autoimmune disease or disorder.

Antibody-mediated rejection (AMR) causes severe and rapid dysfunction and loss of allografts. Without wishing to be bound to a particular theory, the most common mechanism underlying AMR is an anamnestic response that originates from previous antigenic exposure. These donor specific antibody (DSA) responses are usually robust and result in the rapid production of high levels of DSA and acute allograft dysfunction. The mechanism of injury in AMR involves antigens that initiate the production of DSAs resulting in antigen-antibody interactions, complement activation and inflammation, and the resultant donor tissue damage. The impact of AMR on graft survival is dramatic and continues long after the initial inflammatory condition has resolved as was recently demonstrated in a study by LeFaucheur and Glotz. In this single center study of a large cohort of sensitized recipients, the investigators compared allograft survival for recipients successfully treated for AMR versus those that never experienced AMR. In some embodiments, the molecules and polypeptides provided for herein can be used to treat AMR.

In some embodiments, methods of treating a disease or disorder in a subject are provided. In some embodiments, the disease or disorder is an Ig mediated disease or disorder. In some embodiments, the disease or disorder is an autoimmune disease or disorder.

In some embodiments, methods of decreasing circulating immunoglobulins in a subject are provided. In some embodiments, the circulating immunoglobulins are selected from IgG (such as IgG1, IgG2, IgG3, IgG4), IgA, IgM, IgE, or IgD. In some embodiments, methods of decreasing circulating IgG immunoglobulins are provided. In some embodiments, methods of decreasing circulating IgG1 immunoglobulins are provided. In some embodiments, methods of decreasing circulating IgG2 immunoglobulins are provided. In some embodiments, methods of decreasing circulating IgG3 immunoglobulins are provided. In some embodiments, methods of decreasing circulating IgG4 immunoglobulins are provided. In some embodiments, methods of decreasing circulating IgA immunoglobulins are provided. In some embodiments, methods of decreasing circulating IgM immunoglobulins are provided. In some embodiments, methods of decreasing circulating IgE immunoglobulins are provided. In some embodiments, methods of decreasing circulating IgD immunoglobulins are provided. In some embodiments, methods of decreasing circulating immunoglobulins in a subject are provided, wherein the subject is a subject with an autoimmune disease or disorder.

In some embodiments, a method of reducing immunoglobulins is provided. In some embodiments, a method of reducing immunoglobulins, such as IgE, in a subject, is provided. In some embodiments, the method of reducing immunoglobulins, such as IgE, in a subject, comprises administering to the subject any one of the polypeptides or compositions provided for herein. In some embodiments, the method of reducing immunoglobulins, such as IgE, in a subject, comprises administering to the subject the polypeptide comprising the amino acid sequence of any one of SEQ ID NO: 380, 251, 5-243, 247, 280-379, 381-528, 737, 745-823, 828-864, or 885-916, or the composition thereof. In some embodiments, the method of reducing immunoglobulins, such as IgE, in a subject, comprises administering to the subject a molecule, or a composition comprising a first polypeptide and a second polypeptide, such as those provided herein, or the composition thereof. In some embodiments, the reduction of immunoglobulins may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% reduction. In some embodiments, the reduction of immunoglobulins may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction. In some embodiments, the reduction of immunoglobulins may be a 5%, 10%, 15%, 20%, 25%, 30%, 35%, 40%, 45%, 50%, 55%, 60%, 65%, 70%, 75%, 80%, 85%, 90%, 95%, or 100% reduction.

In some embodiments, a method of treating a disease or disorder in a subject is provided. In some embodiments, the method of treating a disease or disorder in a subject, comprises administering to the subject any one of the polypeptides or compositions provided for herein. In some embodiments, the method of treating a disease or disorder in a subject comprises administering to the subject the polypeptide comprising the amino acid sequence of any one of SEQ ID NO: 380, 251, 5-243, 247, 280-379, 381-528, 737, 745-823, 828-864, or 885-916, or the composition thereof. In some embodiments, the method of treating a disease or disorder in a subject comprises administering to the subject a molecule, or a composition comprising a first polypeptide and a second polypeptide, such as those provided herein, or the composition thereof.

In some embodiments, a method of improving a gene-therapy in subject is provided. In some embodiments, the method of improving a gene-therapy in subject comprises administering to the subject any one of the polypeptides or compositions provided for herein. In some embodiments, the method of improving a gene-therapy in subject comprises administering to the subject the polypeptide comprising the amino acid sequence of any one of SEQ ID NO: 380, 251, 5-243, 247, 280-379, 381-528, 737, 745-823, 828-864, or 885-916, or the composition thereof. In some embodiments, the method of improving a gene-therapy in subject comprises administering to the subject a molecule, or a composition comprising a first polypeptide and a second polypeptide, such as those provided herein, or the composition thereof.

In some embodiments, a method of treating a subject having, or at risk, or elevated risk, for having, an IgE mediated disease or disorder is provided. In some embodiments, the method of treating a subject having, or at risk, or elevated risk, for having, an IgE mediated disease or disorder comprises administering to the subject any one of the polypeptides or compositions provided for herein. In some embodiments, the method of treating a subject having, or at risk, or elevated risk, for having, an IgE mediated disease or disorder comprises administering to the subject the polypeptide comprising the amino acid sequence of any one of SEQ ID NO: 380, 251, 5-243, 247, 280-379, 381-528, 737, 745-823, 828-864, or 885-916, or the composition thereof. In some embodiments, the method of treating a subject having, or at risk, or elevated risk, for having, an IgE mediated disease or disorder comprises administering to the subject a molecule, or a composition comprising a first polypeptide and a second polypeptide, such as those provided herein, or the composition thereof.

Non-limiting examples of diseases and disorders, such as autoimmune diseases or disorders, that can be treated with the molecules and polypeptides described herein include, but are not limited to, chronic spontaneous urticaria (CSU), atopic dermatitis, eczema, angioedema, autoimmune bullous skin diseases, bullous pemphigoid (BP), graft vs. host disease (GVHD), sarcoidosis, hypersensitivity pneumonitis, chronic obstructive pulmonary disease (COPD), asthma, allergic asthma, allergic bronchopulmonary mycosis (ABPM), allergic bronchopulmonary aspergillosis (ABPA), chronic rhinosinusitis with nasal polyps (CRSwNP), chronic rhinosinusitis without nasal polyps (CRSsNP), allergic rhinitis, allergic conjunctivitis, vernal keratoconjunctivitis, food allergies, alpha galactosidase syndrome, pollen-food allergy syndrome/oral allergy syndrome (OAS), acute allergen exposure, human seminal fluid hypersensitivity, anaphylaxis, hypersensitivity, allergy, latex allergy, occupational exposure to skin mucosa and lung sensitizing agents and allergens, mastocytosis, mast cell activation disorders, prevention of drug-induced immediate hypersensitivity, drug desensitization (e.g., chemotherapy), allergen immunotherapy desensitization, eosinophilic granulomatosis with polyangiitis (EGPA)/Churg-Strauss, hyper-IgE syndrome (HIES), and the like.

In some embodiments, methods comprise administering a therapeutically effective amount of any one of the polypeptides provided herein, or the pharmaceutical composition comprising any one polypeptide provided herein, thereby treating the subject.

### Pharmaceutical Compositions and Kits

In some embodiments, the present embodiments provide compositions, e.g., pharmaceutically acceptable compositions, which include a therapeutic compound described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, local, ophthalmic, topical, spinal or epidermal administration (e.g. by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used. The carriers can be used in pharmaceutical compositions comprising the therapeutic compounds provided for herein.

The compositions and compounds of the embodiments provided for herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in transplant rejection or autoimmune disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

In some embodiments, the polypeptide having IgE protease activity, such as those provided herein, may be administered to a subject. In some embodiments, the polypeptide having IgE protease activity, such as those provided herein, may be administered to a subject in need thereof. In some embodiments, the polypeptide having IgE protease activity, such as those provided herein, may be administered more than once (e.g., two times, three times, four times, five times, or more). In some embodiments, the polypeptide having IgE protease activity, such as those provided herein, may be administered at least once, at least twice, at least three time, at least four times, or more. In some embodiments, the polypeptide having IgE protease activity, such as those provided herein, may be administered to a subject without inducing an immune response from the subject. In some embodiments, the polypeptide having IgE protease activity, such as those provided herein, does not induce an immune response from a subject after administration to the subject. In some embodiments, the polypeptide having IgE protease activity, such as those provided herein, may be administered to a subject more than once without inducing an immune response from the subject.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the a therapeutic molecule for administration; pharmaceutically acceptable carriers; and devices or other materials for administration to a subject.

In some embodiments, polypeptides having protease activity provided for herein may be conjugated to at least one, or more polypeptides. In some embodiments, the at least one, or more polypeptides, are the same as the polypeptides having protease activity provided for herein. In some embodiments, the at least one, or more polypeptides are an Fc domain. In some embodiments, the Fc domain comprises mutations that render the Fc domain effectorless. In some embodiments, the Fc domain comprises half-life extending mutations.

In some embodiments, the mutation in the Fc domain, which is according to the known numbering system, are selected from the group consisting of: L234A, L235A, L234F, L235E, P329G, P331S, N297A, N297G, N297Q, G236A, A330S, S239D, 1332E, S267E, H268F, S324T, Y296W, T299A, V308P, H310A, R409K, Y435H, T307A, T309A, T309K, K322A, K326W, K334W, K326A, K334A, G237A, P238S, H268A, M252Y, S254T, T256E, M428L, N434A, E380A, T250Q, or any combination thereof. In some embodiments, the Fc comprises a mutation at L234 and/or L235 and/or G237. In some embodiments, the Fc comprises L234A and/or L235A mutations, which can be referred to as “LALA” mutations. In some embodiments, the Fc comprises L234A, L235A, and G237A mutations, which can be referred to as “LALAGA” or “AAA”. In some embodiments, the Fc comprises L234F, and L235E mutations, which can be referred to as “LAFE” mutations. In some embodiments, the Fc comprises L234A, L235A, and P329G mutations, which can be referred to as “LALAPG” mutations. In some embodiments, the Fc comprises L234A, L235A, P329G, and P331S mutations, which can be referred to as “LALAPGS” mutations. In some embodiments, the Fc comprises L234A, L235A, and P329S mutations, which can be referred to as “LALAPS” mutations. In some embodiments, the Fc comprises a N297A mutation. In some embodiments, the Fc mutations comprises a N297G mutation. In some embodiments, the Fc comprises a N297Q mutation. In some embodiments, the Fc comprises a P329G mutation. In some embodiments, the Fc comprises G236A, A330S, and P331S mutations, which can be referred to as “GASDALIE” mutations. In some embodiments, the Fc comprises S239D and 1332E mutations, which can be referred to as “SIE” mutations. In some embodiments, the Fc comprises S267E, H268F, S324T, and I332E mutations, which can be referred to as “SEHE_STIE” mutations. In some embodiments, the Fc comprises Y296W, T299A, and V308P mutations, which can be referred to as “YTEV” mutations. In some embodiments, the Fc comprises H310A, R409K, and Y435H mutations, which can be referred to as “HRY” mutations. In some embodiments, the Fc comprises T307A and T309A mutations, which can be referred to as “TATA” mutations. In some embodiments, the Fc comprises T307A and T309K mutations, which can be referred to as “TAKA” mutations. In some embodiments, the Fc comprises a K322A mutation. In some embodiments, the Fc comprises K326W and K334W mutations, which can be referred to as “WKWK” mutations. In some embodiments, the Fc comprises K326A and K334A mutations, which can be referred to as “AA” mutations. In some embodiments, the Fc comprises L234A, L235A, G237A, P238S, H268A, A330S, and P331S mutations. In some embodiments, the Fc comprises T307A, E380A, and N434A mutations, which can be referred to as “AAA” mutations. In some embodiments, the Fc comprises T250Q, and M428L mutations, which can be referred to as “QL” mutations. In some embodiments, the Fc comprises M428L, and N434S mutations, which can be referred to as “LS” mutations. In some embodiments, the Fc comprises M252Y, S254T, and T256E mutations, which can be referred to as “YTE” mutations.

In some embodiments, the Fc comprises a H310A mutation. In some embodiments, the Fc comprises a H435A mutation. In some embodiments, the Fc comprises H310A and H435A mutations. In some embodiments, the Fc mutations comprise L234A, L235A, and H310A. In some embodiments, the Fc mutations comprise L234F, L235E, and H310A. In some embodiments, the Fc mutations comprise L234A, L235A, P329G, and H310A. In some embodiments, the Fc mutations comprise L234A, L235A, P329G, P331S, and H310A. In some embodiments, the Fc mutations comprise L234A, L235A, P329S, and H310A. In some embodiments, the Fc mutations comprise N297A, and H310A. In some embodiments, the Fc mutations comprise N297G, and H310A. In some embodiments, the Fc mutations comprise N297Q, and H310A. In some embodiments, the Fc mutations comprise P329G, and H310A. In some embodiments, the Fc mutations comprise G236A, A330S, P331S, and H310A. In some embodiments, the Fc mutations comprise S239D, 1332E, and H310A. In some embodiments, the Fc mutations comprise S267E, H268F, S324T, 1332E, and H310A. In some embodiments, the Fc mutations comprise Y296W, T299A, V308P, and H310A. In some embodiments, the Fc mutations comprise H310A, R409K, Y435H, and H310A. In some embodiments, the Fc mutations comprise T307A, T309A, and H310A. In some embodiments, the Fc mutations comprise T307A, T309K, and H310A. In some embodiments, the Fc mutations comprise K322A, and H310A. In some embodiments, the Fc mutations comprise K326W, K334W, and H310A. In some embodiments, the Fc mutations comprise K326A, K334A, and H310A. In some embodiments, the Fc mutations comprise L234A, L235A, and H435A. In some embodiments, the Fc mutations comprise L234F, L235E, and H435A. In some embodiments, the Fc mutations comprise L234A, L235A, P329G, and H435A. In some embodiments, the Fc mutations comprise L234A, L235A, P329G, P331S, and H435A. In some embodiments, the Fc mutations comprise L234A, L235A, P329S, and H435A. In some embodiments, the Fc mutations comprise N297A, and H435A. In some embodiments, the Fc mutations comprise N297G, and H435A. In some embodiments, the Fc mutations comprise N297Q, and H435A. In some embodiments, the Fc mutations comprise P329G, and H435A. In some embodiments, the Fc mutations comprise G236A, A330S, P331S, and H435A. In some embodiments, the Fc mutations comprise S239D, I332E, and H435A. In some embodiments, the Fc mutations comprise S267E, H268F, S324T, I332E, and H435A. In some embodiments, the Fc mutations comprise Y296W, T299A, V308P, and H435A. In some embodiments, the Fc mutations comprise H435A, R409K, Y435H, and H435A. In some embodiments, the Fc mutations comprise T307A, T309A, and H435A. In some embodiments, the Fc mutations comprise T307A, T309K, and H435A. In some embodiments, the Fc mutations comprise K322A, and H435A. In some embodiments, the Fc mutations comprise K326W, K334W, and H435A. In some embodiments, the Fc mutations comprise K326A, K334A, and H435A. In some embodiments, the Fc mutations comprise L234A, L235A, H310, and H435A. In some embodiments, the Fc mutations comprise L234F, L235E, H310, and H435A. In some embodiments, the Fc mutations comprise L234A, L235A, P329G, H310, and H435A. In some embodiments, the Fc mutations comprise L234A, L235A, P329G, P331S, H310, and H435A. In some embodiments, the Fc mutations comprise L234A, L235A, P329S, H310, and H435A. In some embodiments, the Fc mutations comprise N297A, H310, and H435A. In some embodiments, the Fc mutations comprise N297G, H310, and H435A. In some embodiments, the Fc mutations comprise N297Q, H310, and H435A. In some embodiments, the Fc mutations comprise P329G, H310, and H435A. In some embodiments, the Fc mutations comprise G236A, A330S, P331S, H310, and H435A. In some embodiments, the Fc mutations comprise S239D, 1332E, H310, and H435A. In some embodiments, the Fc mutations comprise S267E, H268F, S324T, I332E, H310, and H435A. In some embodiments, the Fc mutations comprise Y296W, T299A, V308P, H310, and H435A. In some embodiments, the Fc mutations comprise H435A, R409K, Y435H, H310, and H435A. In some embodiments, the Fc mutations comprise T307A, T309A, H310, and H435A. In some embodiments, the Fc mutations comprise T307A, T309K, H310, and H435A. In some embodiments, the Fc mutations comprise K322A, H310, and H435A. In some embodiments, the Fc mutations comprise K326W, K334W, H310, and H435A. In some embodiments, the Fc mutations comprise K326A, K334A, H310, and H435A.

The mutations and positions of the Fc domain, which can also be referred to as the Fc domain, are according to Kabat numbering.

As used herein, a Fc domain/domain comprising a mutation at a specific position is as compared to the wild-type Fc according to the numbering system (Kabat numbering) as referenced herein.

In some embodiments, a pharmaceutical composition comprising any one of the polypeptides provided herein is provided. In some embodiments, the composition comprises a dimer comprising a first polypeptide comprising any one of the polypeptides provided herein and a second polypeptide comprising any one of the polypeptides provided herein, wherein the first polypeptide and the second polypeptide are the same. In some embodiments, the composition comprises a dimer comprising a first polypeptide comprising any one of the polypeptides provided herein linked to a Fc domain, and a second polypeptide comprising any one of the polypeptides provided herein linked to a second Fc domain, wherein the first polypeptide and the second polypeptide are the same. In some embodiments, the first Fc domain and the second Fc domain comprise the same amino acid sequence. In some embodiments, the first Fc domain and the second Fc domain comprise different amino acid sequences, provided that the first and second Fc domains can form a dimer.

A protease or a polypeptide provided for herein may be produced by any suitable means. For example, the protease or the polypeptide may be synthesized directly using standard techniques known in the art.

As used herein, the terms “nucleic acid molecule” and “polynucleotide” may be used interchangeably and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the disclosure may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (N-) terminus and a translation stop codon at the 3′ (C-) terminus. For the purposes of the disclosure, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides encoding protease or polypeptides provided for herein may be synthesized according to methods well known in the art, as described by way of example in Sambrook et al (1989, Molecular Cloning—a laboratory manual; Cold Spring Harbor Press). The nucleic acid molecules of the present disclosure may be provided in the form of an expression cassette which includes control sequences operably linked to the inserted sequence, thus allowing for expression of the polypeptide of the disclosure in vivo. These expression cassettes, in turn, are typically provided within vectors (e.g., plasmids or recombinant viral vectors). Such an expression cassette may be administered directly to a host subject.

In some embodiments, the protease or the polypeptide provided for herein may be produced by transforming a cell, typically a bacterial cell, with a nucleic acid molecule or vector which encodes said protease or polypeptide. In some embodiments, a vector comprising a polynucleotide encoding a protease or a polypeptide provided for herein may be administered to a host subject. In some embodiments, the polynucleotide is prepared and/or administered using a vector. A suitable vector may be any vector which is capable of carrying a sufficient amount of genetic information, and allowing expression of a protease or a polypeptide of the disclosure. In some embodiments, nucleic acid molecules or a polynucleotide is provided that encodes a protease or a polypeptide as provided for herein. In some embodiments, the polynucleotide is DNA. In some embodiments, the polynucleotide is RNA, such as mRNA.

The present disclosure thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a protease or a polypeptide of the disclosure. Other suitable vectors would be apparent to persons skilled in the art.

In some embodiments, the host subject may be a cell that has been modified to express a protease or a polypeptide provided for herein. Such cells typically include prokaryotic cells such as bacterial cells, for example E. coli. In some embodiments, the host subject may be an eukaryotic cell. In some embodiments, the cell may be cultured using routine methods to produce a polypeptide of the disclosure.

In some embodiments, a vector is provided comprising the nucleic acid molecule or polynucleotide encoding a polypeptide as provided for herein. In some embodiments, the vector is a non-viral vector. In some embodiments, the vector is a viral vector, such as, but not limited to a lentivirus, an AAV vector, an AV vector, and the like. In some embodiments, the non-viral vector is a plasmid. In some embodiments, the non-viral vector is mRNA encoding the polypeptide. In some embodiments, the mRNA is encapsulated. In some embodiments, the mRNA is encapsulated in a liposome. The mRNA can contain modified nucleotides or a modified backbone to increase the mRNA half-life.

In some embodiments, a cell is provided comprising a nucleic acid molecule encoding a polypeptide as provided for herein. In some embodiments, a cell is provide comprising a vector as provided for herein. In some embodiments, the cell is a eukaryotic cell. In some embodiments, the cell is a human cell or a yeast cell. In some embodiments, the cell is not a bacterial cell. In some embodiments, the cell is a bacterial cell or a prokaryotic cell, such as, but not limited to E. Coli. In some embodiments, the cell is not E. Coli. In some embodiments, the cell is not a prokaryotic cell. In some embodiments, a cell is provide comprising a vector as provided for herein.

A polypeptide may be derivatized or modified to assist with their production, isolation or purification. For example, where a protease or a polypeptide of the disclosure is produced by recombinant expression in a host cell, the sequence of the polypeptide may include an additional methionine (M) residue at the N terminus to improve expression. As another example, the protease or the polypeptide of the disclosure may be derivatized or modified by addition of a leader sequence, as provided for herein.

The amino acid sequence of a polypeptide may be modified to include non-naturally occurring amino acids, for example to increase stability. When the polypeptides are produced by synthetic means, such amino acids may be introduced during production. The polypeptides may also be modified following either synthetic or recombinant production. Polypeptides may also be produced using D-amino acids. In such cases the amino acids will be linked in reverse sequence in the C- to N-orientation. This is conventional in the art for producing such polypeptides.

In some embodiments, methods of making a polypeptide as provided for herein are provided. In some embodiments, the methods comprise culturing a cell comprising a nucleic acid molecule or polynucleotide encoding the polypeptide. In some embodiments, the cell is as provided herein, such as the cells described above. In some embodiments, the cell is cultured under conditions suitable to facilitate the expression of the polypeptide.

In some embodiments, methods of making a nucleic acid sequence encoding the polypeptide are provided. In some embodiments, the methods comprise providing a vector comprising a polynucleotide sequence encoding a polypeptide having protease activity and inserting into the vector sequence encoding a variant Fc molecule to form an amino acid sequence encoding the polypeptide; or b) providing a vector comprising sequence encoding a variant Fc molecule and inserting into the vector sequence encoding a polypeptide having protease activity to form an amino acid sequence encoding the polypeptide, thereby making an nucleic acid sequence encoding the polypeptide.

The nucleic acid molecules can be transferred into a cell to produce the polypeptide by any means or methods, such as by transfection, electroporation, transduction, and the like.

The following examples are illustrative, but not limiting, of the compounds, compositions and methods described herein. Other suitable modifications and adaptations known to those skilled in the art are within the scope of the following embodiments.

### EXAMPLES

### Example 1: Polypeptides Having IgE Protease Activity Exhibit Stable Thermal Profiles and Improved Cleavage Activity Over Parental Protease

Melting temperature (Tm) was measured by nanoDSF. Tm of each polypeptide was found by observing the fluorescence of the polypeptide at 350 and 330 nm while ramping the temperature from 20-95° C. The approximate size and monodispersity was measured by size exclusion chromatography. The protease was purified with majority population as monomers on analytical SEC, showing favorable thermostability profile, as shown in Table 4.

Protease activity was measured by end-point assay analyzed by CE-SDS. In brief, purified polypeptides were added directly to a fixed amount of IgE (0.5 mg/mL of the polypepitide). Reactions were incubated at 37° C. overnight. The reactions were quenched with IAA, and products were measured using a PerkinElmer® LabChip® to perform CE-SDS. Both the appearance of new bands (i.e., single cut IgE (scIgE) and F(ab′) 2 IgE signals) and disappearance of intact Ig target bands were used to confirm activity/specificity of the proteases within this Ig target group Results were confirmed over several different reactions and CE-SDS measurements. PRT-4 was identified as a more stable and more active IgE cleaving protease than PRT-1.

TABLE 4

| Variant | Mutation(s) | Tₘ | Activity |
|---|---|---|---|
| PRT-1 | — | 46.4° C. | very weak |
| PRT-17 | T122S | 45.6° C. | single cleavage |
| PRT-4 | L153P, S128A, F333N | 53.2° C. | full cleavage |
| PRT-8 | T122S, L153P, S128A | 54.5° C. | single cleavage |
| PRT-10 | T122S, S128A, F333N | 51.7° C. | full cleavage |

In a separate experiment, cleavage activity against IgE and IgG1 was assessed using CE-SDS or MSD methods described herein. All polypeptides shown in Table 5 below displayed IgE cleavage activity as measured by SDS where the cleavage products of IgE were visualized. Table 5 also provides IgE Protease EC50 values for selected IgE proteases as indicated or if not calculated it is indicated by the term “n/a”. Tm was measured as described above, and polypeptides were also loaded to analytical SEC to check for monodispersity of Protein of Interest (POI). All test articles were purified with the majority population present as monomers as shown in Table 5 below.

TABLE 5

| | | | EC50 | EC50 | EC50 | IgE |
|---|---|---|---|---|---|---|
| | | | CE-SDS | CE-SDS | MSD | Cleavage |
| | Tm1 | % | IgE | IgG1 | IgE | in CE-SDS |
| Name | (° C.) | POI | (uM) | (uM) | (uM) | Assay |
| PRT-1 | 46.4 | n/a | n/a | n/a | n/a | Yes |
| PRT-2 | 45.9 | 68 | n/a | n/a | n/a | SC (single |
| | | | | | | cut) |
| PRT-3 | 52.3 | 65 | n/a | n/a | n/a | Yes |
| PRT-4 | 53.2 | 49 | 1.16 | 1.44 | 2.07 | Yes |
| PRT-5 | 46.9 | 62 | n/a | n/a | n/a | Yes |
| PRT-6 | 48.5 | 59 | n/a | n/a | n/a | Yes |
| PRT-7 | 48.0 | 62 | n/a | n/a | n/a | SC (single |
| | | | | | | cut) |
| PRT-8 | 54.5 | 74 | n/a | n/a | n/a | SC (single |
| | | | | | | cut) |
| PRT-9 | 47.2 | 63 | n/a | n/a | n/a | Yes |
| PRT-10 | 51.7 | 63 | n/a | n/a | n/a | Yes |
| PRT-11 | 45.4 | n/a | n/a | n/a | n/a | SC (single |
| | | | | | | cut) |
| PRT-12 | 45.3 | 50 | n/a | n/a | n/a | Yes |
| PRT-13 | 45.5 | n/a | n/a | n/a | n/a | SC (single |
| | | | | | | cut) |
| PRT-14 | 46.2 | 63 | n/a | n/a | n/a | Yes |
| PRT-15 | 45.3 | n/a | n/a | n/a | n/a | SC (single |
| | | | | | | cut) |
| PRT-16 | 44.6 | n/a | n/a | n/a | n/a | SC (single |
| | | | | | | cut) |
| PRT-17 | 46.5 | 81 | n/a | n/a | n/a | Yes |
| PRT-18 | 45.7 | 49 | n/a | n/a | n/a | Yes |
| PRT-19 | 46.5 | n/a | n/a | n/a | n/a | SC (single |
| | | | | | | cut) |
| PRT-20 | 46.5 | 62 | n/a | n/a | n/a | Yes |
| PRT-21 | 46.9 | n/a | n/a | n/a | n/a | SC (single |
| | | | | | | cut) |
| PRT-22 | 45.1 | 68 | n/a | n/a | n/a | Yes |
| PRT-240 | 44.6 | 51 | n/a | n/a | n/a | Yes |
| PRT-241 | 45.3 | n/a | n/a | n/a | n/a | SC (single |
| | | | | | | cut) |
| PRT-23 | 51.9407082 | n/a | n/a | n/a | n/a | Yes |
| PRT-24 | 53.1527863 | n/a | n/a | n/a | n/a | Yes |
| PRT-25 | 50.5786438 | n/a | n/a | n/a | n/a | Yes |
| PRT-26 | 51.730751 | n/a | n/a | n/a | n/a | Yes |
| PRT-27 | 51.9239807 | n/a | n/a | n/a | n/a | Yes |
| PRT-28 | 53.4722176 | n/a | n/a | n/a | n/a | Yes |
| PRT-29 | 51.1548538 | n/a | n/a | n/a | n/a | Yes |
| PRT-30 | 51.2497406 | n/a | n/a | n/a | n/a | Yes |
| PRT-31 | 50.7491608 | n/a | n/a | n/a | n/a | Yes |
| PRT-32 | 51.8809319 | n/a | n/a | n/a | n/a | Yes |
| PRT-33 | 49.4964218 | n/a | n/a | n/a | n/a | Yes |
| PRT-34 | 52.3817635 | n/a | n/a | n/a | n/a | Yes |
| PRT-35 | 53.3928337 | n/a | n/a | n/a | n/a | Yes |
| PRT-36 | 55.0777931 | n/a | n/a | n/a | n/a | Yes |
| PRT-37 | 53.485733 | n/a | n/a | n/a | n/a | Yes |
| PRT-38 | 53.5205574 | n/a | n/a | n/a | n/a | Yes |
| PRT-39 | 53.4476395 | n/a | n/a | n/a | n/a | Yes |
| PRT-40 | 53.2519264 | n/a | n/a | n/a | n/a | Yes |
| PRT-41 | 51.5434418 | n/a | 0.51 | 3.14 | n/a | Yes |
| PRT-42 | 52.2763786 | n/a | n/a | n/a | n/a | Yes |
| PRT-43 | 51.5486183 | n/a | 0.26 | 4.75 | 0.369 | Yes |
| PRT-44 | 55.2020988 | n/a | n/a | n/a | n/a | Yes |
| PRT-45 | 52.063118 | n/a | n/a | n/a | n/a | Yes |
| PRT-46 | 51.3904076 | n/a | 0.42 | 6.43 | n/a | Yes |
| PRT-47 | 52.5997353 | n/a | n/a | n/a | n/a | Yes |
| PRT-48 | 52.2065239 | n/a | n/a | n/a | n/a | Yes |
| PRT-49 | 52.317955 | n/a | n/a | n/a | n/a | Yes |
| PRT-50 | 53.4346657 | n/a | n/a | n/a | n/a | Yes |
| PRT-51 | 51.3877029 | n/a | n/a | n/a | n/a | Yes |
| PRT-52 | 50.4173393 | n/a | n/a | n/a | n/a | Yes |
| PRT-53 | 53.7835388 | n/a | n/a | n/a | n/a | Yes |
| PRT-54 | 55.3129883 | n/a | n/a | n/a | n/a | Yes |
| PRT-55 | 55.4130745 | n/a | n/a | n/a | n/a | Yes |
| PRT-56 | 55.0503502 | n/a | n/a | n/a | n/a | Yes |
| PRT-57 | 56.1044083 | n/a | n/a | n/a | n/a | SC (single |
| | | | | | | cut) |
| PRT-58 | 54.2344627 | n/a | n/a | n/a | n/a | Yes |
| PRT-59 | 52.7204781 | n/a | 0.69 | 18.67 | n/a | Yes |
| PRT-60 | 53.1412735 | n/a | n/a | n/a | n/a | Yes |
| PRT-61 | 54.0746651 | n/a | n/a | n/a | n/a | Yes |
| PRT-62 | 53.145607 | n/a | 0.42 | 5.1 | n/a | Yes |
| PRT-63 | 53.4345207 | n/a | n/a | n/a | n/a | Yes |
| PRT-64 | 54.0387344 | n/a | n/a | n/a | n/a | Yes |
| PRT-65 | 52.5188217 | n/a | 0.49 | 4 | n/a | Yes |
| PRT-66 | 53.605732 | n/a | n/a | n/a | n/a | Yes |
| PRT-67 | 52.8675232 | n/a | n/a | n/a | n/a | Yes |
| PRT-68 | 53.9576263 | n/a | n/a | n/a | n/a | SC (single |
| | | | | | | cut) |
| PRT-69 | 52.2696648 | n/a | n/a | n/a | n/a | Yes |
| PRT-70 | 51.6251831 | n/a | n/a | n/a | n/a | Yes |
| PRT-71 | 50.9279747 | n/a | n/a | n/a | n/a | Yes |
| PRT-72 | 51.3287468 | n/a | n/a | n/a | n/a | Yes |
| PRT-73 | 51.6389732 | n/a | n/a | n/a | n/a | Yes |
| PRT-74 | 51.5259933 | n/a | n/a | n/a | n/a | Yes |
| PRT-75 | 53.1374321 | n/a | n/a | n/a | n/a | Yes |
| PRT-76 | 53.4998703 | n/a | n/a | n/a | n/a | Yes |
| PRT-77 | 53.2173042 | n/a | n/a | n/a | n/a | Yes |
| PRT-78 | 52.9008942 | n/a | n/a | n/a | n/a | Yes |
| PRT-79 | 54.0034332 | n/a | n/a | n/a | n/a | Yes |
| PRT-80 | 55.2028923 | n/a | n/a | n/a | n/a | Yes |
| PRT-81 | 53.4392586 | n/a | 0.36 | 2.31 | 0.407 | Yes |
| PRT-82 | 53.8643684 | n/a | n/a | n/a | n/a | Yes |
| PRT-83 | 53.5775528 | n/a | n/a | n/a | n/a | Yes |
| PRT-84 | 52.6416435 | n/a | n/a | n/a | n/a | Yes |
| PRT-85 | 52.3788872 | n/a | 0.36 | 1.96 | n/a | Yes |
| PRT-86 | 51.1456528 | n/a | n/a | n/a | n/a | Yes |
| PRT-87 | 53.3139343 | n/a | n/a | n/a | n/a | Yes |
| PRT-88 | 51.6169701 | n/a | 0.17 | 0.99 | 0.366 | Yes |
| PRT-89 | 51.5808563 | n/a | 0.07 | 37.25 | 0.042 | Yes |
| PRT-90 | 54.8071022 | n/a | n/a | n/a | n/a | Yes |
| PRT-91 | 54.0097847 | n/a | n/a | n/a | n/a | SC (single |
| | | | | | | cut) |
| PRT-94 | 52.5397873 | n/a | n/a | n/a | n/a | Yes |
| PRT-95 | 54.6420822 | n/a | n/a | n/a | n/a | Yes |
| PRT-96 | 53.9702492 | n/a | n/a | n/a | n/a | Yes |
| PRT-97 | 54.8722763 | n/a | n/a | n/a | n/a | Yes |
| PRT-98 | 53.8322334 | n/a | n/a | n/a | n/a | Yes |
| PRT-99 | 52.5295067 | n/a | n/a | n/a | n/a | Yes |
| PRT-100 | 53.5640297 | n/a | n/a | n/a | n/a | Yes |
| PRT-101 | 51.5430946 | n/a | n/a | n/a | n/a | Yes |
| PRT-102 | 52.969532 | n/a | n/a | n/a | n/a | Yes |
| PRT-103 | 57.7853355 | n/a | n/a | n/a | n/a | Yes |
| PRT-104 | 51.8296776 | n/a | n/a | n/a | n/a | Yes |
| PRT-105 | 52.3402939 | n/a | n/a | n/a | n/a | Yes |
| PRT-106 | 53.492775 | n/a | n/a | n/a | n/a | Yes |
| PRT-107 | 52.9401703 | n/a | n/a | n/a | n/a | Yes |
| PRT-108 | 52.2916222 | n/a | n/a | n/a | n/a | Yes |
| PRT-109 | 52.995163 | n/a | n/a | n/a | n/a | Yes |
| PRT-110 | 51.8660774 | n/a | n/a | n/a | n/a | Yes |
| PRT-111 | 47.8136139 | n/a | n/a | n/a | n/a | Yes |
| PRT-112 | 48.7256622 | n/a | n/a | n/a | n/a | Yes |
| PRT-113 | 52.3498077 | n/a | n/a | n/a | n/a | Yes |
| PRT-114 | 53.7827377 | n/a | n/a | n/a | n/a | Yes |
| PRT-115 | 51.4999504 | n/a | n/a | n/a | n/a | Yes |
| PRT-116 | 50.4459763 | n/a | n/a | n/a | n/a | Yes |
| PRT-117 | 50.1363907 | n/a | n/a | n/a | n/a | Yes |
| PRT-118 | 52.6067047 | n/a | n/a | n/a | n/a | Yes |

### Example 2: Polypeptides Having IgE Protease Activity Show Specificity for IgE

Purified polypeptides were added directly to a fixed amount of IgE at different concentrations. Reactions were incubated at 37° C. overnight. The reactions were quenched with IAA, and products were measured using a PerkinElmer® LabChip® to perform CE-SDS. Both the appearance of new bands (i.e., single cut IgE (scIgE) and F(ab′) 2 IgE signals) and disappearance of intact Ig target bands were used to confirm activity/specificity of the polypeptides within this Ig target group. Results were confirmed over several different reactions and CE-SDS measurements.

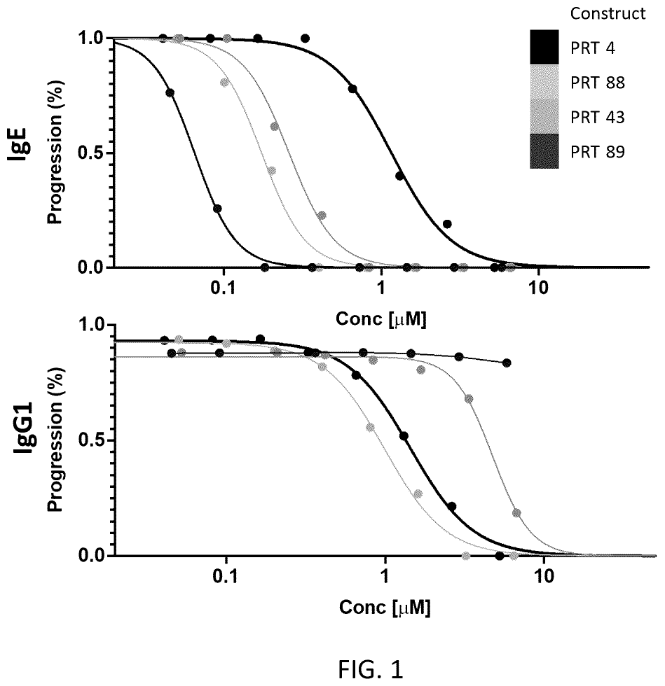

As shown in FIG. 1, the polypeptides cleave soluble intact IgE.

### Example 3: Polypeptides Having IgE Protease Activity Fully Cleave IgE and Show Improved Potency

Human IgE antibody was incubated with IgE cleaving protease PRT-4 for 24 hours and 48 hours at 37° C. Digestion samples were loaded to JESS to separate the bands of intact IgE (substrate), single cut IgE (intermediate product), and F(ab′) 2 (final product). Detection of bands was achieved using a mixture of anti-kappa light chain and anti-lambda light chain antibodies. Full cleavage of IgE was observed with PRT-4 at both timepoints.

In next experiment, recombinant human IgE antibody (concentration-0.1 mg/ml) was incubated with IgE cleaving polypeptides PRT-4, PRT-88, PRT-43, PRT-81 and PRT-89 in a dose-dependent manner for 24 hours at 37° C. Digested samples were added to MSD plate that was captured with anti-human IgE capture antibody and blocked appropriately. Signal was detected using anti-human light chain antibody and potency was depicted as EC50 values.

Full cleavage of IgE was observed with all the IgE cleaving polypeptides. PRT-89 showed improved potency compared to all other polypeptides.

TABLE 7

| | PRT-4 | PRT-88 | PRT-43 | PRT-81 | PRT-89 |
|---|---|---|---|---|---|
| EC50 (μM) | 2.070 | 0.366 | 0.369 | 0.407 | 0.042 |

### Example 4: Polypeptides Having IgE Protease Activity Cleave Soluble Human IgE

Human IgE antibody (concentration-0.1 mg/ml) was incubated with polypeptides PRT-4, PRT-88, PRT-43, PRT-81 and PRT-89 (each at 0.35 μM concentration) for 24 hours at 37° C. Digested samples were loaded to JESS to separate the bands of intact IgE (substrate), single cut IgE (intermediate product), and F(ab′) 2 (final product). Detection of bands was achieved using a mixture of anti-kappa light chain and anti-lambda light chain antibodies.

Full cleavage of IgE was observed with PRT-89 at the concentration tested compared to others which showed single cleavage.

### Example 5: Identification of IgE Residues for Polypeptide Binding

Structure of protease bound to IgE Fc was determined by single particle cryo-electron microscopy using a Cys to Ser inactive protease. Data was collected on a Titan Krios (ThermoFisher Scientific) electron microscope. The data was processed using CryoSPARC software (Structura Biotechnology) and the structural models were built in Coot (free software under GNU General Public License). The complex structures were analyzed using ChimeraX software (UCSF) to determine epitope contact residues, which are defined as amino acid residues from the protease that are within 4 Å of any atoms in IgE Fc. Thus, the epitope for the polypeptides described herein includes residues S332, N333, P334, R335, G336, V337, S338, A339, D363, L364, A365, P366, K368, A378, K392, R394, N395, G396, T397, R420, H423, P424, H425, L426, P427, R428, A429, M431, R432, S433 of IgE. The residue numbering is as set forth in SEQ ID NO: 246. The epitope on the non-cleaved Fc fragment of IgE includes D363, A365, P366, K368, K392, R394, N395, G396, T397, and H425 of IgE. The residue numbering is as set forth in SEQ ID NO: 246. The epitope on the cleaved Fc fragment of IgE includes S332, N333, P334, R335, G336, V337, S338, A339, D363, L364, A365, K368, A378, R420, H423, P424, H425, L426, P427, R428, A429, M431, R432, and S433 of IgE. The residue numbering is as set forth in SEQ ID NO: 246.

### Example 6: N-Terminal Sequencing Confirms Cleavage Site on IgE

IgE (7 μg) was digested with PRT-17 (1:1) for 24 hours at 37° C. The reaction mixture was loaded and separated on a 10% mini-protean TGX gel. The resulting gel was blotted onto a PVDF membrane (Trans-Blot Turbo Midi 0.2 μM PVDF). Membrane was stained using InstantBlue coomassie stain and destained with 40% methanol and 5% acetic acid. The bands corresponding to the product were cut from the membrane and analyzed for N-terminal sequencing. N-terminal sequencing confirmed the cleavage site of GVSAYLS (SEQ ID NO: 250) on IgE (SEQ ID NO: 246).

### Example 7: Molecules Comprising Polypeptides Having IgE Protease Activity Cleave Soluble IgE but not IgG

Human IgE or IgG antibody was incubated with a control or molecules comprising polypeptides having IgE protease activity, such as the dimer molecules provided herein. Digested samples were loaded to JESS automated Western Blott system. Cleavage of human IgE was observed with the molecules comprising polypeptides having IgE protease activity, such as the dimer molecules provided herein. No cleavage of human IgG was observed.

Human IgE antibody was incubated with IgE cleaving protease PRT-4 for 24 hours and 48 hours at 37° C. Digestion samples were loaded to JESS to separate the bands of intact IgE (substrate), single cut IgE (intermediate product), and F(ab′) 2 (final product). Detection of bands was achieved using a mixture of anti-kappa light chain and anti-lambda light chain antibodies. Full cleavage of IgE was observed with PRT-4 at both timepoints.

In next experiment, human IgE antibody was incubated with a control or molecules comprising polypeptides having IgE protease activity, such as the dimer molecules provided herein in a dose-dependent manner. Digested samples were added to MSD plate that was captured with anti-human IgE capture antibody and blocked appropriately. Signal was detected using anti-human light chain antibody and potency was depicted as EC50 values.

Cleavage of IgE was observed with all molecules comprising polypeptides having IgE protease activity, such as the dimer molecules provided herein.

### Example 8: a Molecule Comprising Polypeptides Having IgE Protease Activity Demonstrates Ability to Cleave B-Cell Receptors (BCR)

IgE+B-cell myeloma cells were incubated with a molecule comprising polypeptides having IgE protease activity, such as the dimer molecules provided herein in a dose-dependent manner. Digested samples were added to MSD plate, captured, and blocked appropriately. Signal was detected using anti-human light chain antibody and potency was depicted as EC50 values.

BCR cleavage was observed in a dose dependent manner. Accordingly, the molecule comprising a polypeptide having IgE protease activity demonstrates ability to cleave BCR.

### Example 9: a Molecule Comprising Polypeptides Having IgE Protease Activity Reduces FcεRI Expression in IgE Spiked Bone Marrow-Derived Mast Cells from Humanized Mice

FcεRI is a receptor on mast cells and basophils that binds to the Fc domain of IgE and plays a critical role in allergic responses. Treatment of mast cell with a molecule comprising polypeptides having IgE protease activity, such as the dimer molecules provided herein in a dose-dependent manner showed a rapid reduction in expression of FcεRI.

### Example 10: a Molecule Comprising Polypeptides Having IgE Protease Activity Prevented and Treated Systemic Anaphylactic Reactions In Vivo, with Similar Efficacy to Omalizumab

Humanized IgE/FcεRI mice were treated on day 1 with a vehicle, omalizumab, or a molecule comprising polypeptides having IgE protease activity, such as the dimer molecules provided herein, administered intravenously at a dose of 0.3 mpk. On day 2 the mice were sensitized using anti-NIP IgE administered intraperitoneally. Baseline core temperature was measured. On day 3 the mice were challenged with NIP BSA administered intraperitoneally. Core body temperature was measured every 2 hours following challenge. The molecule comprising polypeptides having IgE protease activity, such as the dimer molecules provided herein prevented the systematic anaphylactic reaction.

In another experiment, humanized IgE/FcεRI mice were sensitized using anti-NIP IgE administered intraperitoneally. Baseline core temperature was measured. On day 2 the mice were treated on day 1 with a vehicle, omalizumab, or a molecule comprising polypeptides having IgE protease activity, such as the dimer molecules provided herein, administered intravenously at a dose of 0.3 mpk. On day 3 the mice were challenged with NIP BSA administered intraperitoneally. Core body temperature was measured every 2 hours following challenge. The molecule comprising polypeptides having IgE protease activity, such as the dimer molecules provided herein rapidly treated systematic anaphylactic reaction in vivo in a therapeutic mouse model.

### Example 11: Molecules Comprising Polypeptides Having IgE Protease Activity Show Comparable Efficacy to Omalizumab in Acute Passive Cutaneous Anaphylaxis Model

Humanized FcεRI mice were treated on day 1 with S+C, PBS+C, omalizumab, or molecules comprising polypeptides having IgE protease activity, such as the dimer molecules provided herein administered intravenously at a dose of 1 mpk. On day 2 the mice were sensitized with anti-NIP IgE administered intradermally. On day 3 the mice were challenged with NIP BSA with Evan's blue dye administered intravenously. 2 hours following challenge Evan's blue dye leakage was measured. Efficacy of molecules comprising polypeptides having IgE protease activity, such as the dimer molecules provided herein to prevent dye leakage was comparable to clinically relevant doses of omalizumab in this model.

### Example 12: Molecules Comprising Polypeptides Having IgE Protease Activity Exhibit Favorable PK in Mice

Wild-type Balb/c mice were treated with a vehicle, or molecules comprising polypeptides having IgE protease activity, such as the dimer molecules provided herein administered intravenously at a dose of 1 mpk. 15 minutes following treatment the mice were administered intravenously recombinant human IgE at a dose of 50 μg, and IVIG at a dose of 7.5 mg. Blood was collected at 0.5 hours, 2 hours, 4 hours, 24 hours, 48 hours, 72 hours, 5 days, 7 days, and 10 days following initial treatment. Plasma levels of the vehicle or the molecules, and IgE cleavage were assessed. The molecules comprising polypeptides having IgE protease activity, such as the dimer molecules provided herein showed favorable PK and half-life of 212, 149, and 207 hours.

### Example 13: Molecules Comprising Polypeptides Having IgE Protease Activity Demonstrate No Binding to Pre-Existing Anti-Drug Antibodies (ADA) in IVIg and Plasma

Molecules comprising polypeptides having IgE protease activity, such as the dimer molecules provided herein were evaluated for binding to pre-existing antibodies in IVIG and plasma relative to positive control, through an electrochemiluminescence (ECL) immunoassay. The molecules comprising polypeptides having IgE protease activity, such as the dimer molecules provided herein exhibited no binding to pre-existing ADA, measured by ECL signal.

### Example 14: Molecules Comprising Polypeptides Having IgE Protease Activity Exhibit Attributes Favorable of Long-Term Storage, Subcutaneous Administration, and Low Off-Target Activity

Molecules comprising polypeptides having IgE protease activity, such as the dimer molecules provided herein showed high stability with no formation of aggregates over 1 month at 4° C., supportive of favorable long-term storage. Molecules comprising polypeptides having IgE protease activity, such as the dimer molecules provided herein also showed viscosity levels that were suitable for subcutaneous injection, as shown in the table below.

TABLE 8

| Protease | Average Viscosity (mPa*s) | n | % CV |
|---|---|---|---|
| Molecule 1 | 1.84 | 10 | 7.02 |
| Molecule 2 | 1.76 | 10 | 0.60 |
| Molecule 3 | 1.58 | 10 | 2.53 |

Molecules comprising polypeptides having IgE protease activity, such as the dimer molecules provided herein also showed low polyreactivity to baculovirus particle, cardiolipin, keyhole limpet hemocyanin, and double stranded DNA, predictive of favorable PK.

### Example 15: Polypeptides Having IgE Protease Activity Exhibit Stable Thermal Profiles and Improved Cleavage Activity Over Parental Protease

Cleavage activity against IgE and IgG1 was assessed using CE-SDS or MSD methods described herein. All polypeptides shown in Table 9 and Table 10 below displayed IgE cleavage activity as measured by SDS where the cleavage products of IgE were visualized. Table 9 and Table 10 also provides IgE Protease EC50 values for selected IgE proteases as indicated or if not calculated it is indicated by the term “n/a”. Tm was measured as described above, and polypeptides were also loaded to analytical SEC to check for monodispersity of Protein of Interest (POI). All test articles were purified with the majority population present as monomers as shown in Table 9 and Table 10 below.

TABLE 9

| | | | EC50 | EC50 | EC50 | IgE |
|---|---|---|---|---|---|---|
| | | | CE-SDS | CE-SDS | MSD | Cleavage |
| | Tm1 | % | IgE | IgG1 | IgE | in CE-SDS |
| Name | (° C.) | POI | (uM) | (uM) | (uM) | Assay |
| PRT-492 | n/a | n/a | n/a | n/a | n/a | n/a |
| PRT-493 | n/a | n/a | n/a | n/a | n/a | n/a |
| PRT-244 | 50.98 | n/a | n/a | n/a | n/a | sc (single cut) |
| PRT-245 | 50.68 | n/a | n/a | n/a | n/a | sc (single cut) |
| PRT-246 | 51.5 | n/a | n/a | n/a | n/a | sc (single cut) |
| PRT-247 | 49.67 | n/a | n/a | n/a | n/a | yes |
| PRT-248 | 49.3 | n/a | n/a | n/a | n/a | yes |
| PRT-249 | 47.27 | n/a | n/a | n/a | n/a | yes |
| PRT-250 | 49.76 | n/a | n/a | n/a | n/a | yes |
| PRT-251 | 49.96 | n/a | n/a | n/a | n/a | sc (single cut) |
| PRT-252 | 47.93 | n/a | n/a | n/a | n/a | sc (single cut) |
| PRT-253 | 50.03 | n/a | n/a | n/a | n/a | sc (single cut) |
| PRT-254 | n/a | n/a | n/a | n/a | n/a | n/a |
| PRT-255 | n/a | n/a | n/a | n/a | n/a | n/a |
| PRT-256 | n/a | n/a | n/a | n/a | n/a | n/a |
| PRT-257 | n/a | n/a | n/a | n/a | n/a | n/a |
| PRT-258 | n/a | n/a | n/a | n/a | n/a | n/a |
| PRT-259 | 43.76198196 | n/a | n/a | n/a | n/a | yes |
| PRT-260 | 40.83691025 | n/a | n/a | n/a | n/a | yes |
| PRT-261 | 48.13053513 | n/a | n/a | n/a | n/a | yes |
| PRT-262 | 38.44819641 | n/a | n/a | n/a | n/a | yes |
| PRT-263 | 43.80036926 | n/a | n/a | n/a | n/a | yes |
| PRT-264 | 51.95593262 | n/a | 1.008 | n/a | n/a | yes |
| PRT-265 | 49.4007988 | n/a | n/a | n/a | n/a | yes |
| PRT-266 | 49.84073639 | n/a | n/a | n/a | n/a | yes |
| PRT-267 | 46.30564499 | n/a | n/a | n/a | n/a | yes |
| PRT-268 | 46.28196335 | n/a | n/a | n/a | n/a | yes |
| PRT-269 | 44.04050827 | n/a | 4.093 | n/a | n/a | yes |
| PRT-270 | 41.60387421 | n/a | n/a | n/a | n/a | yes |
| PRT-271 | 49.88454056 | n/a | 0.1055 | n/a | n/a | yes |
| PRT-272 | 50.59305954 | n/a | 0.6144 | n/a | n/a | yes |
| PRT-273 | 48.86257172 | n/a | 0.9515 | n/a | n/a | yes |
| PRT-274 | 38.25500488 | n/a | n/a | n/a | n/a | yes |
| PRT-275 | 48.31901932 | n/a | n/a | n/a | n/a | yes |
| PRT-276 | 49.16300583 | n/a | 1.43 | n/a | n/a | yes |
| PRT-277 | 53.43740845 | n/a | 0.07473 | n/a | n/a | yes |
| PRT-278 | 47.74253082 | n/a | 1.613 | n/a | n/a | yes |
| PRT-279 | 41.28607559 | n/a | n/a | n/a | n/a | yes |
| PRT-280 | 33.78106308 | n/a | n/a | n/a | n/a | yes |
| PRT-281 | 39.57804871 | n/a | n/a | n/a | n/a | yes |
| PRT-282 | 42.87959671 | n/a | 0.8608 | n/a | n/a | yes |
| PRT-283 | 40.88740921 | n/a | 0.8135 | n/a | n/a | yes |
| PRT-284 | 44.02651596 | n/a | n/a | n/a | n/a | yes |
| PRT-285 | 43.68782425 | n/a | n/a | n/a | n/a | yes |
| PRT-286 | 39.54377365 | n/a | n/a | n/a | n/a | yes |
| PRT-287 | 39.71357727 | n/a | n/a | n/a | n/a | yes |
| PRT-288 | 42.24381638 | n/a | n/a | n/a | n/a | yes |
| PRT-289 | 39.13557053 | n/a | n/a | n/a | n/a | yes |
| PRT-290 | 41.00407791 | n/a | n/a | n/a | n/a | yes |
| PRT-291 | 43.2956543 | n/a | n/a | n/a | n/a | yes |
| PRT-292 | 40.5482254 | n/a | n/a | n/a | n/a | yes |
| PRT-293 | 43.41492462 | n/a | n/a | n/a | n/a | yes |
| PRT-294 | 50.12894821 | n/a | 0.7602 | n/a | n/a | yes |
| PRT-295 | 48.63828659 | n/a | n/a | n/a | n/a | yes |
| PRT-296 | 47.57891846 | n/a | n/a | n/a | n/a | yes |
| PRT-297 | 49.10969162 | n/a | n/a | n/a | n/a | yes |
| PRT-298 | 48.3764267 | n/a | 0.1069 | n/a | n/a | yes |
| PRT-299 | 43.78216553 | n/a | n/a | n/a | n/a | yes |
| PRT-300 | 48.02518082 | n/a | n/a | n/a | n/a | yes |
| PRT-301 | 48.58825684 | n/a | n/a | n/a | n/a | yes |
| PRT-302 | 42.35677338 | n/a | n/a | n/a | n/a | yes |
| PRT-303 | 49.93935013 | n/a | 0.104 | n/a | n/a | yes |
| PRT-304 | 42.65013885 | n/a | n/a | n/a | n/a | yes |
| PRT-305 | 49.57157898 | n/a | n/a | n/a | n/a | yes |
| PRT-306 | 38.70973587 | n/a | n/a | n/a | n/a | yes |
| PRT-307 | 40.05797195 | n/a | n/a | n/a | n/a | yes |
| PRT-308 | 42.0752182 | n/a | n/a | n/a | n/a | yes |
| PRT-309 | 54.21295166 | n/a | 0.3312 | n/a | n/a | yes |
| PRT-310 | 45.64842224 | n/a | n/a | n/a | n/a | yes |
| PRT-311 | 44.28898621 | n/a | n/a | n/a | n/a | yes |
| PRT-312 | 43.80886459 | n/a | n/a | n/a | n/a | yes |
| PRT-313 | 51.13744354 | n/a | 0.4075 | n/a | n/a | yes |
| PRT-314 | 49.00503159 | n/a | 0.2328 | n/a | n/a | yes |
| PRT-315 | 44.56128311 | n/a | n/a | n/a | n/a | yes |
| PRT-316 | 44.14808655 | n/a | n/a | n/a | n/a | yes |
| PRT-317 | 51.2220993 | n/a | 1.794 | n/a | n/a | yes |
| PRT-318 | 43.91475677 | n/a | n/a | n/a | n/a | yes |
| PRT-319 | 49.3368721 | n/a | 0.143 | n/a | n/a | yes |
| PRT-320 | 42.33901215 | n/a | n/a | n/a | n/a | yes |
| PRT-321 | 46.95602417 | n/a | n/a | n/a | n/a | yes |
| PRT-322 | 48.54349136 | n/a | n/a | n/a | n/a | yes |
| PRT-323 | 39.25563812 | n/a | n/a | n/a | n/a | yes |
| PRT-324 | 50.44552612 | n/a | n/a | n/a | n/a | yes |
| PRT-325 | 53.13976288 | n/a | n/a | n/a | n/a | yes |
| PRT-326 | 49.12893295 | n/a | n/a | n/a | n/a | yes |
| PRT-327 | 51.58704758 | n/a | n/a | n/a | n/a | yes |
| PRT-328 | 53.40829468 | n/a | n/a | n/a | n/a | yes |
| PRT-329 | 51.02653885 | n/a | n/a | n/a | n/a | yes |
| PRT-330 | 50.95039749 | n/a | n/a | n/a | n/a | yes |
| PRT-331 | 47.05178452 | n/a | n/a | n/a | n/a | yes |
| PRT-332 | 55.97952271 | n/a | 0.2172 | n/a | n/a | yes |
| PRT-333 | 53.2 | 86 | 0.7 | n/a | n/a | yes |
| PRT-334 | 56.9 | 86 | 0.7 | n/a | n/a | yes |
| PRT-335 | 57.6 | 100 | 2 | n/a | n/a | yes |
| PRT-336 | 50.9 | 95 | 2 | n/a | n/a | yes |
| PRT-337 | 54.7 | 88 | 0.6 | n/a | n/a | yes |

TABLE 10

| | | | | EC50 | | IgE |
|---|---|---|---|---|---|---|
| | | | EC50 | CE- | EC50 | Cleavage |
| | | | CE-SDS | SDS | MSD | in |
| | Tm1 | % | IgE | IgG1 | IgE | CE-SDS |
| Name | (° C.) | POI | (uM) | (uM) | (uM) | Assay |
| FP1 | 47.09824753 | 55 | n/a | n/a | n/a | yes |
| FP2 | 52.58 | n/a | 0.2119 | n/a | 0.1401 | yes |
| FP3 | 49.69 | n/a | 0.07149 | n/a | 0.1137 | yes |
| FP4 | 49.07 | n/a | n/a | n/a | n/a | yes |
| FP5 | 50.73 | n/a | n/a | n/a | n/a | yes |
| FP6 | 47.31 | n/a | n/a | n/a | 0.09943 | yes |
| FP7 | 52.66 | n/a | n/a | n/a | n/a | yes |
| FP8 | 52.32 | n/a | n/a | n/a | n/a | yes |
| FP9 | 54.72 | n/a | n/a | n/a | n/a | yes |
| FP10 | n/a | n/a | n/a | n/a | n/a | n/a |
| FP11 | 52.31 | 75.44 | 0.06852 | n/a | | yes |
| FP12 | 54.92 | 69.6 | 0.04333 | n/a | 0.000753 | yes |
| FP13 | 52.4 | 75.77 | 0.03185 | 21.33 | n/a | yes |
| FP14 | 53.45 | 71.07 | 0.03185 | n/a | n/a | yes |
| FP15 | 51.99 | 74.5 | 0.05468 | n/a | n/a | yes |
| FP16 | 50.55 | 79.45 | 0.04703 | 8.716 | n/a | yes |
| FP17 | 51.45 | 72.99 | 0.05631 | n/a | n/a | yes |
| FP18 | 54.52 | 79 | n/a | n/a | n/a | yes |
| FP19 | 56.7 | 92 | 0.02176 | 7.236 | 0.001204 | yes |
| FP20 | 54.83 | 90 | n/a | n/a | n/a | yes |
| FP21 | 53.33 | 67 | n/a | n/a | n/a | yes |
| FP22 | 56.45 | 94 | n/a | n/a | n/a | yes |
| FP23 | 55.85 | 89 | n/a | n/a | n/a | yes |
| FP24 | 56.03 | 84 | n/a | n/a | n/a | yes |
| FP25 | 51.89 | 72 | n/a | n/a | n/a | yes |
| FP26 | 54.48 | 75 | n/a | n/a | n/a | yes |
| FP27 | 54.63 | 88 | n/a | n/a | n/a | yes |
| FP28 | 53.34 | 76 | n/a | n/a | n/a | yes |
| FP29 | 53.18 | 88 | n/a | n/a | n/a | yes |
| FP30 | 51.68 | 78 | n/a | n/a | n/a | yes |
| FP31 | 55.91 | 93 | n/a | n/a | n/a | yes |
| FP32 | 53.89 | 75 | n/a | n/a | n/a | yes |
| FP33 | 50.94 | 64 | n/a | n/a | n/a | yes |
| FP34 | 55.77 | 92 | n/a | n/a | n/a | yes |
| FP35 | 54.98 | 90 | n/a | n/a | n/a | yes |
| FP36 | 54.61 | 78 | n/a | n/a | n/a | yes |
| FP37 | 53.96 | 90 | n/a | n/a | n/a | yes |
| FP38 | 56.55 | 95 | n/a | n/a | n/a | yes |
| FP39 | 46.11 | 86.77 | n/a | n/a | n/a | sc (single |
| | | | | | | cut) |
| FP40 | n/a | n/a | n/a | n/a | n/a | yes |
| FP41 | 52.31 | 75.25 | n/a | n/a | n/a | yes |
| FP42 | 47.76 | 66.25 | n/a | n/a | n/a | yes |
| FP43 | 51.13 | 64.85 | n/a | n/a | n/a | yes |
| FP44 | 47.52 | 53.3 | n/a | n/a | n/a | yes |
| FP45 | 49.07 | 48.07 | n/a | n/a | n/a | yes |
| FP46 | n/a | n/a | n/a | n/a | n/a | yes |
| FP47 | 43.62 | 50.14 | n/a | n/a | n/a | sc (single |
| | | | | | | cut) |
| FP48 | 51.66 | 62.17 | n/a | n/a | n/a | yes |
| FP49 | n/a | n/a | n/a | n/a | n/a | yes |
| FP50 | n/a | n/a | n/a | n/a | n/a | yes |
| FP51 | 45.76 | 51.32 | n/a | n/a | n/a | yes |
| FP52 | n/a | n/a | n/a | n/a | n/a | yes |
| FP53 | 42.8 | 38.83 | n/a | n/a | n/a | yes |
| FP54 | 39.66 | 79.01 | n/a | n/a | n/a | sc (single |
| | | | | | | cut) |
| FP55 | 45.64 | 47.12 | n/a | n/a | n/a | sc (single |
| | | | | | | cut) |
| FP56 | n/a | n/a | n/a | n/a | n/a | yes |
| FP57 | 47.83 | 72.47 | n/a | n/a | n/a | yes |
| FP58 | 45.93 | 61.23 | n/a | n/a | n/a | yes |
| FP59 | 40.92 | 19.87 | n/a | n/a | n/a | sc (single |
| | | | | | | cut) |
| FP60 | 51.11 | 58.4 | n/a | n/a | n/a | yes |
| FP61 | 45.47 | 65.08 | n/a | n/a | n/a | sc (single |
| | | | | | | cut) |
| FP62 | n/a | n/a | n/a | n/a | n/a | yes |
| FP63 | 55.42 | 81.61 | n/a | n/a | n/a | sc (single |
| | | | | | | cut) |
| FP64 | 49.84 | 79.53 | n/a | n/a | n/a | yes |
| FP65 | 53.97 | 70.99 | n/a | n/a | n/a | sc (single |
| | | | | | | cut) |
| FP66 | 51.77 | 59.81 | n/a | n/a | n/a | yes |
| FP67 | 52.52 | 58.54 | n/a | n/a | n/a | yes |
| FP68 | n/a | n/a | n/a | n/a | n/a | yes |
| FP69 | 48.28 | 72.31 | n/a | n/a | n/a | sc (single |
| | | | | | | cut) |
| FP70 | n/a | n/a | n/a | n/a | n/a | yes |
| FP71 | n/a | n/a | n/a | n/a | n/a | yes |
| FP72 | n/a | n/a | n/a | n/a | n/a | yes |
| FP73 | 48.36 | 83.79 | n/a | n/a | n/a | yes |
| FP74 | 50.41 | 64.65 | n/a | n/a | n/a | sc (single |
| | | | | | | cut) |
| FP75 | n/a | n/a | n/a | n/a | n/a | yes |
| FP76 | 46.44 | 77.77 | n/a | n/a | n/a | sc (single |
| | | | | | | cut) |
| FP77 | n/a | n/a | n/a | n/a | n/a | yes |
| FP78 | 50.7 | 75.93 | n/a | n/a | n/a | yes |
| FP79 | 48.78 | 81.47 | n/a | n/a | n/a | sc (single |
| | | | | | | cut) |
| FP80 | 46.41 | 57.11 | n/a | n/a | n/a | yes |
| FP81 | 47.55 | 59 | n/a | n/a | n/a | yes |
| FP82 | n/a | n/a | n/a | n/a | n/a | yes |
| FP83 | 48.79 | 80.48 | n/a | n/a | n/a | yes |
| FP84 | 50.4 | 80.83 | n/a | n/a | n/a | yes |
| FP85 | 50.05 | 70.71 | n/a | n/a | n/a | yes |
| FP86 | 49.19 | 74.51 | n/a | n/a | n/a | yes |
| FP87 | n/a | n/a | n/a | n/a | n/a | yes |
| FP88 | 44.36 | 78.75 | n/a | n/a | n/a | yes |
| FP89 | 46.74 | 91.07 | n/a | n/a | n/a | sc (single |
| | | | | | | cut) |
| FP90 | 54.77 | 84.4 | n/a | n/a | n/a | yes |
| FP91 | 43.65 | 68.57 | n/a | n/a | n/a | sc (single |
| | | | | | | cut) |
| FP92 | 49.13 | 80.57 | n/a | n/a | n/a | yes |
| FP93 | 46.22 | 81.14 | n/a | n/a | n/a | yes |
| FP94 | 44.59 | 63.85 | n/a | n/a | n/a | yes |
| FP95 | 45.01 | 85.93 | n/a | n/a | n/a | sc (single |
| | | | | | | cut) |
| FP96 | 47.27 | 84.16 | n/a | n/a | n/a | yes |
| FP97 | n/a | n/a | n/a | n/a | n/a | yes |
| FP98 | 53.69 | 87.87 | n/a | n/a | n/a | yes |
| FP99 | 51.85 | 87.13 | n/a | n/a | n/a | yes |
| FP100 | 48.76 | 82.09 | n/a | n/a | n/a | yes |
| FP101 | 47.86 | 75.61 | n/a | n/a | n/a | yes |
| FP102 | n/a | n/a | n/a | n/a | n/a | yes |
| FP103 | 44.96 | 82.2 | n/a | n/a | n/a | yes |
| FP104 | not detected | 93.27 | n/a | n/a | n/a | yes |
| FP105 | 47.19 | 88.1 | n/a | n/a | n/a | yes |
| FP106 | n/a | n/a | n/a | n/a | n/a | yes |
| FP107 | 48.9 | 84.2 | n/a | n/a | n/a | yes |
| FP108 | 47.33 | 86.41 | n/a | n/a | n/a | yes |
| FP109 | n/a | n/a | n/a | n/a | n/a | yes |
| FP110 | 49.59 | 91.71 | n/a | n/a | n/a | yes |
| FP111 | 47.48 | 94.06 | n/a | n/a | n/a | yes |
| FP112 | n/a | n/a | n/a | n/a | n/a | yes |
| FP113 | 50 | 80.89 | n/a | n/a | n/a | yes |
| FP114 | 54.04 | 79.22 | n/a | n/a | n/a | yes |
| FP115 | 48.59 | 69.64 | n/a | n/a | n/a | sc (single |
| | | | | | | cut) |
| FP116 | 51.19 | 63.46 | n/a | n/a | n/a | yes |
| FP117 | n/a | n/a | n/a | n/a | n/a | yes |
| FP118 | 48.14 | 80.51 | n/a | n/a | n/a | yes |
| FP119 | 53.38 | 85.61 | n/a | n/a | n/a | yes |
| FP120 | 54.65 | 87.12 | n/a | n/a | n/a | yes |
| FP121 | 55.43 | 86.16 | 0.00122 | n/a | 0.001245 | yes |
| FP122 | 55.9 | 87 | n/a | n/a | n/a | yes |
| FP123 | 52.1 | 80.77 | n/a | n/a | n/a | yes |
| FP124 | 53.61 | 84.82 | n/a | n/a | n/a | yes |
| FP125 | 51.18 | 71.23 | n/a | n/a | n/a | yes |
| FP126 | 53.03 | 89.59 | n/a | n/a | n/a | yes |
| FP127 | 53.75 | 81.5 | n/a | n/a | n/a | yes |
| FP128 | 55.81 | 83.45 | n/a | n/a | n/a | yes |
| FP129 | 51.75 | 87.66 | n/a | n/a | n/a | yes |
| FP130 | 58.32 | 93.42 | n/a | n/a | n/a | yes |
| FP131 | 53.63 | 84.33 | n/a | n/a | n/a | yes |
| FP132 | 55.14 | 91 | 0.00127 | n/a | n/a | yes |
| FP133 | 52.37 | 99 | 0.00115 | n/a | n/a | yes |
| FP134 | 52 | 80 | 0.00236 | n/a | n/a | yes |
| FP135 | n/a | 78.58 | n/a | n/a | n/a | n/a |
| FP136 | 52.36 | 98 | 0.00061 | n/a | n/a | yes |
| FP137 | 52.54 | 94 | 0.00128 | n/a | n/a | yes |
| FP138 | n/a | 76.76 | n/a | n/a | n/a | n/a |
| FP139 | 53.16 | 95 | 0.00011 | n/a | n/a | yes |
| FP140 | 53.74 | 98 | 0.00043 | n/a | 0.001353 | yes |
| FP141 | 55.3 | 90 | 0.00075 | n/a | 0.0008745 | yes |
| FP142 | 55.37 | 95 | 0.0038 | n/a | | yes |
| FP143 | 53.17 | 96 | 0.000035 | n/a | 0.001629 | yes |
| FP144 | 53.64 | 98 | 0.00066 | n/a | n/a | yes |
| FP145 | 55.08 | 98 | 0.00145 | n/a | 0.0006542 | yes |
| FP146 | 51.84 | 97 | 0.00158 | n/a | n/a | yes |
| FP147 | 56.61 | 98 | 0.0031 | n/a | n/a | sc (single |
| | | | | | | cut) |
| FP148 | n/a | n/a | n/a | n/a | n/a | n/a |
| FP149 | 52.05 | 97 | 0.00019 | n/a | 0.0007076 | yes |
| FP150 | 53.66 | 93 | 0.00115 | n/a | 0.002203 | yes |
| FP151 | 51.94 | 81.85 | n/a | n/a | n/a | n/a |
| FP152 | n/a | 97.69 | n/a | n/a | n/a | n/a |
| FP153 | n/a | 83.75 | n/a | n/a | n/a | n/a |
| FP154 | n/a | 83.16 | n/a | n/a | n/a | n/a |
| FP155 | n/a | 75.01 | n/a | n/a | n/a | n/a |
| FP156 | n/a | 84.01 | n/a | n/a | n/a | n/a |
| FP157 | n/a | 71.45 | n/a | n/a | n/a | n/a |
| FP158 | 51.74 | 97 | 0.0002 | n/a | n/a | yes |
| FP159 | 51.69 | 95 | 0.00245 | n/a | n/a | yes |
| FP160 | 46.09 | 91 | 0.00404 | n/a | n/a | yes |
| FP161 | n/a | 93.62 | n/a | n/a | n/a | n/a |
| FP162 | 53.81 | 69.64 | n/a | n/a | n/a | n/a |
| FP163 | 56.21 | 72.11 | 0.0305 | n/a | n/a | n/a |
| FP164 | 54.75 | 74.27 | 0.0174 | n/a | n/a | n/a |
| FP165 | 55.3 | 95 | 0.0293 | n/a | 0.0008219 | yes |
| FP166 | n/a | n/a | n/a | n/a | n/a | n/a |
| FP167 | 55.3 | 96 | n/a | n/a | 0.000721 | yes |
| FP168 | 55.3 | 95 | 0.0176 | n/a | 0.0006368 | yes |
| FP169 | n/a | n/a | n/a | n/a | n/a | n/a |
| FP170 | 56.31 | 98 | 0.0146 | n/a | 0.001018 | yes |
| FP171 | 56.39 | 95 | 0.0178 | n/a | 0.00135 | yes |
| FP172 | n/a | n/a | n/a | n/a | n/a | n/a |
| FP173 | n/a | n/a | n/a | n/a | n/a | n/a |
| FP174 | 56.37 | 97 | 0.0122 | n/a | 0.000511 | yes |
| FP175 | 56.62 | 98 | 0.179 | n/a | 0.001875 | yes |
| FP176 | n/a | n/a | n/a | n/a | n/a | n/a |
| FP177 | n/a | n/a | n/a | n/a | n/a | n/a |
| FP178 | 53.6 | 99 | 0.00261 | n/a | 0.0003171 | yes |
| FP179 | 56 | 97 | 0.00664 | n/a | 0.0004236 | yes |
| FP180 | n/a | n/a | n/a | n/a | n/a | n/a |
| FP181 | n/a | n/a | n/a | n/a | n/a | n/a |
| FP182 | n/a | n/a | n/a | n/a | n/a | n/a |
| FP183 | 56.6 | 98 | 0.00763 | n/a | 0.0005482 | yes |
| FP184 | 56.7 | 99 | n/a | n/a | 0.0004748 | yes |
| FP185 | n/a | n/a | n/a | n/a | n/a | n/a |
| FP186 | n/a | n/a | n/a | n/a | n/a | n/a |
| FP187 | n/a | n/a | n/a | n/a | n/a | n/a |
| FP188 | n/a | n/a | n/a | n/a | n/a | n/a |
| FP189 | n/a | n/a | n/a | n/a | n/a | n/a |
| FP190 | n/a | n/a | n/a | n/a | n/a | n/a |
| FP191 | 56.7 | 97 | 0.001506 | n/a | 0.0005555 | yes |

### Example 16: Pharmacokinetics (PK) and Pharmacodynamics (PD) of IgE Cleaving Protease by Intravenous and Subcutaneous Injection to Cynomolgus Monkeys

Cynomolgus monkeys were injected with single intravenous injections of human IgE cleaving protease (FP183) at doses 0.1 mg/kg and 1 mg/kg. Blood samples were collected at defined timepoints and serum was separated. PK (Table 11) and PD (Table 12) was determined using MSD-based assays on serum samples.

TABLE 11
Pharmacokinetics.

| Time after | | | | | | |
|---|---|---|---|---|---|---|
| dosing (days) | 0.1 mpk | | | 1 mpk | | |
| 1.02 | 3241.8 | 3534.8 | 3828 | 45891.8 | 41185.1 | 50995.5 |
| 1.25 | 1767.5 | 1896.6 | 2351.2 | 29060 | 22798 | 30686.3 |
| 2 | 889.2 | 1439 | 1393.3 | 11952.6 | 11007.5 | 13622.4 |
| 3 | 739.3 | 528.7 | 832.8 | 10844.6 | 9440.5 | 11834.5 |
| 4 | 497.3 | 560.6 | 753 | 7739.9 | 7083.3 | 8892.8 |
| 5 | 483.6 | 617.1 | 461.9 | 7034.1 | 6056.3 | 7909.2 |
| 8 | 304.6 | 236.2 | 303.8 | 5425.1 | 4382.2 | 4960.1 |
| 10 | 400.4 | 323.8 | 480.8 | 4558.7 | 3550.2 | 3253.6 |

TABLE 12
Pharmacodynamics.

| | | | |
|---|---|---|---|
| Days | Vehicle | 0.1 mpk | 1 mpk |

| | | | | | | | | | |
|---|---|---|---|---|---|---|---|---|---|
| −3.00 | 100.00 | 100.00 | 100.00 | 100.00 | 100.00 | 100.00 | 100.00 | 100.00 | 100.00 |
| 1.02 | 81.72 | 128.93 | 117.63 | 3.19 | 4.02 | 4.95 | 5.05 | 0.49 | 0.51 |
| 1.25 | 98.53 | 149.53 | 146.06 | 1.36 | 1.44 | 2.39 | 1.29 | 0.41 | 0.05 |
| 2.00 | 83.60 | 132.63 | 132.11 | 0.00 | 0.00 | 0.00 | 0.00 | 0.00 | 0.00 |
| 3.00 | 74.02 | 101.61 | 119.66 | 1.24 | 0.22 | 0.00 | 0.00 | 0.00 | 0.00 |
| 4.00 | 88.44 | 109.49 | 160.57 | 4.47 | 5.55 | 1.36 | 0.00 | 0.00 | 0.00 |
| 5.00 | 124.07 | 112.20 | 138.41 | 20.31 | 9.32 | 0.00 | 0.00 | 0.00 | 0.00 |
| 8.00 | 154.25 | 53.78 | 107.79 | 46.70 | 28.16 | 28.94 | 2.53 | 0.00 | 0.00 |
| 10.00 | 160.02 | 46.59 | 113.42 | 74.07 | 56.11 | 50.40 | 9.19 | 0.00 | 0.00 |

FP183 showed dose linear exposure and rapid reduction of cyno soluble IgE at both doses.

### Example 17

Binding ability of FP183, FP178, and FP179 to Clq was evaluated via enzyme-linked immunoassay. As shown in FIG. 29, test articles displayed no binding to Clq.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1-78. (canceled)

79. A polypeptide having IgE protease activity, wherein the polypeptide:
a) has at least 85% identity to SEQ ID NO: 1;
b) comprises:
i) a cysteine (C) at a position in the polypeptide that corresponds to position 125 of SEQ ID NO: 1;
ii) a histidine (H) at a position in the polypeptide that corresponds to position 283 of SEQ ID NO: 1;
iii) an aspartic acid (D) at a position in the polypeptide that corresponds to position 305 of SEQ ID NO: 1; and
iv) at least one mutation in the polypeptide as compared to SEQ ID NO: 1 that corresponds to position 68, 71, 75, 79, 84, 86, 88, 91, 93, 94, 102, 112, 115, 116, 117, 120, 122, 125, 126, 128, 139, 142, 143, 148, 149, 150, 152, 153, 160, 162, 164, 165, 166, 167, 174, 175, 177_183, 180, 182, 183, 185, 189, 196, 197, 199, 200_202, 201, 205, 206, 212, 219, 220, 221, 222, 224, 228, 229, 232, 233, 241, 242, 243, 244, 250, 251, 252, 254, 260, 262, 263, 268, 270, 271_287, 274, 275, 276, 279, 280, 281, 282, 293, 294, 295, 297, 301, 303, 303_326, 306, 307_322, 308_324, 309, 310, 310_320, 311_319, 311_321, 313, 314, 315_317, 316, 318, 319, 320, 324, 327, 329, 332, 333, 336, 343, 346, 347, 348, 351, 360, 363, 364, 366, 374, and 380 of SEQ ID NO: 1.

80. The polypeptide of claim 79, wherein the polypeptide comprises:
an aspartic acid (D) at a position in the polypeptide that corresponds to position 307 of SEQ ID NO: 1; and/or
a lysine (K) at a position in the polypeptide that corresponds to position 115 of SEQ ID NO: 1.

81. The polypeptide of claim 79, wherein the polypeptide comprises the amino acid sequence having the formula of:
(SEQ ID NO: 917)
X₁TALGRRKAEDRIKETLWADGVKIDEKDFEHIX₉₃SSHEDYFAVEY
GKGSGYYDVNKKMDSEDX₁₂₂ALCVGX₁₂₈VVTNMFHWWVX₁₃₉QNREN
IEKFKX₁₅₀QDX₁₅₃ENNGVMKFX₁₆₂GVKEATVDELNX₁₇₄FYX₁₇₇YAD
QSRFFDLIKX₁₉₆HFX₁₉₉NKALNPEELFELYFNGFYYX₂₁₉SSX₂₂₂RY
IEDNX₂₂₉PAQNNLFSKVLX₂₄₁NNKLLNPERSYGIDGFSNNVX₂₆₂NA
LKSHKVLGLVX₂₇₅TTPLX₂₈₀YRHIISMWGADIDQDGKVKAIYVTDS
DDREITFNDNSKRRIGMX₃₂₄RYDVLVDDX₃₃₃GNIRFTGKGX₃₄₃THK
EEGAIYAGLYSLSX₃₆₀GDEVWEDYFKKYPEVQENLX₃₈₀,
wherein:
at least one X₉₃, X₁₂₂, X₁₂₈, X₁₃₉, X₁₅₀, X₁₅₃, X₁₆₂, X₁₇₄, X₁₇₇, X₁₉₆, X₁₉₉, X₂₁₉, X₂₂₂, X₂₂₉, X₂₄₁, X₂₆₂, X₂₇₅, X₂₈₀, X₃₂₄, X₃₃₃, X₃₄₃, and X₃₆₀ is mutated as compared to SEQ ID NO: 1,
X₃₈₀ comprises the amino acid sequence having the formula as set forth in SEQ ID NO: 744 or is absent, and
X₁ comprises the amino acid sequence of SEQ ID NO: 248, or is absent.

82. The polypeptide of claim 81, wherein X₁₂₈ is alanine (A), X₁₅₃ is proline (P), X₁₆₂ is glutamic acid (E), X₂₂₉ is glutamic acid (E), X₃₂₄ is isoleucine (I), and X₃₃₃ is asparagine (N).

83. The polypeptide of claim 81, wherein X₁₂₈ is alanine (A), X₁₅₃ is proline (P), X₁₆₂ is glutamic acid (E), X₁₇₄ is glutamic acid (E), X₁₇₇ is an amino acid sequence RDNRDN (SEQ ID NO: 743), X₂₁₉ is glycine (G), X₂₂₉ is glutamic acid (E), X₂₇₅ is tyrosine (Y), X₃₂₄ is isoleucine (I), and X₃₃₃ is asparagine (N).

84. The polypeptide of claim 81, wherein X₉₃ is aspartic acid (D), X₁₂₂ is serine(S), X₁₂₈ is alanine (A), X₁₅₀ is glutamic acid (E), X₁₅₃ is proline (P), X₁₆₂ is glutamic acid (E), X₁₇₄ is glutamic acid (E), X₁₇₇ is an amino acid sequence RDNRDN (SEQ ID NO: 743), X₁₉₉ is proline (P), X₂₁₉ is glycine (G), X₂₂₉ is glutamic acid (E), X₂₄₁ is aspartic acid (D), X₂₇₅ is tyrosine (Y), X₃₂₄ is isoleucine (I), and X₃₃₃ is asparagine (N).

85. The polypeptide of claim 81, wherein X₉₃ is aspartic acid (D), X₁₂₂ is serine(S), X₁₂₈ is alanine (A), X₁₃₉ is glutamic acid (E), X₁₅₀ is glutamic acid (E), X₁₅₃ is proline (P), X₁₆₂ is glutamic acid (E), X₁₇₄ is glutamic acid (E), X₁₇₇ is an amino acid sequence RDNRDN (SEQ ID NO: 743), X₁₉₆ is serine(S), X₁₉₉ is proline (P), X₂₁₉ is glycine (G), X₂₂₂ is glutamine (Q), X₂₂₉ is glutamic acid (E), X₂₄₁ is aspartic acid (D), X₂₆₂ is lysine (K), X₂₇₅ is tyrosine (Y), X₂₈₀ is threonine (T), X₃₂₄ is isoleucine (I), X₃₃₃ is asparagine (N), X₃₄₃ is lysine (K), and X₃₆₀ is leucine (L).

86. The polypeptide of claim 79, wherein the polypeptide comprises an amino acid sequence having at least 95%, identity to an amino acid sequence of SEQ ID NOs: 380, 251, 5-243, 247, 280-379, 381-528, 737, 745-823, 828-864, or 885-916.

87. The polypeptide of claim 79, wherein the polypeptide comprises the amino acid sequence of any one of SEQ ID NO: 380, 251, 5-243, 247, 280-379, 381-528, 737, 745-823, 828-864, or 885-916.

88. The polypeptide of claim 79, wherein the polypeptide does not comprise the contiguous sequence of amino acid residues 1-52 (1_52del) or 1-61 (1_61del) of SEQ ID NO: 1.

89. The polypeptide of claim 79, wherein the polypeptide does not comprise contiguous sequence of amino acid residues 380-529 (380_529del) of SEQ ID NO: 1.

90. The polypeptide of claim 79, wherein the polypeptide does not comprise the contiguous sequence of amino acid residues 1-52 and 380-529 of SEQ ID NO: 1.

91. The polypeptide of claim 79, wherein the polypeptide does not comprise the contiguous sequence of amino acid residues 1-61 and 380-529 of SEQ ID NO: 1.

92. The polypeptide of claim 79, wherein the polypeptide is more selective at cleaving IgE as compared to IgG.

93. The polypeptide of claim 79, wherein the polypeptide comprises any one of mutation sets as set forth in Table 3A or Table 3B as compared to SEQ ID NO: 1.

94. The polypeptide of claim 79, wherein the polypeptide comprises a Fc domain linked to the N-terminus or the C-terminus of the polypeptide.

95. The polypeptide of claim 94, wherein the Fc domain comprises an amino acid sequence that is least 95% identical to the amino acid sequence of SEQ ID NO: 255, SEQ ID NO: 721, SEQ ID NO: 724, SEQ ID NO: 725, SEQ ID NO: 256, or SEQ ID NO: 273.

96. The polypeptide of claim 94, wherein the Fc domain comprises the amino acid sequence of SEQ ID NO: 255, SEQ ID NO: 721, SEQ ID NO: 724, SEQ ID NO: 725, SEQ ID NO: 256, or SEQ ID NO: 273.

97. The polypeptide of claim 94, wherein the polypeptide comprises an amino acid sequence that is least 95% identical to the amino acid sequence of any one of SEQ ID NOs: 257, 258, 259, or 529-720.

98. The polypeptide of claim 94, wherein the polypeptide comprises the amino acid sequence of any one of SEQ ID NOs: 257, 258, 259, or 529-720.

99. A polypeptide comprising:
a first polypeptide comprising the polypeptide of claim 79 linked to an Fc domain;
and a second polypeptide comprising a second Fc domain.

100. The polypeptide of claim 99, wherein:
the first Fc domain comprises a Fc domain comprising an amino acid sequence that is at least 95% identical to SEQ ID NO: 255, SEQ ID NO: 721, SEQ ID NO: 724, SEQ ID NO: 725, SEQ ID NO: 256, or SEQ ID NO: 273; and
the second Fc domain comprises a Fc domain comprising an amino acid sequence that is at least 95% identical to SEQ ID NO: 253, 254, or 722.

101. The polypeptide of claim 100, wherein:
the first Fc domain comprises a Fc domain comprising the amino acid sequence of SEQ ID NO: 255, SEQ ID NO: 721, SEQ ID NO: 724, SEQ ID NO: 725, SEQ ID NO: 256, or SEQ ID NO: 273; and
the second Fc domain comprises a Fc domain comprising the amino acid sequence of SEQ ID NO: 253, 254, or 722.

102. The polypeptide of claim 99, wherein:
the first polypeptide comprises an amino acid sequence that is least 95% identical to the amino acid sequence of any one of SEQ ID NOs: 257, 258, 259, or 529-720; and
the second polypeptide comprises an amino acid sequence that is at least 95% identical to SEQ ID NO: 253, SEQ ID NO: 254, or SEQ ID NO: 722.

103. The polypeptide of claim 102, wherein the first polypeptide comprises the amino acid sequence of any one of SEQ ID NOs: 257, 258, 259, or 529-720.

104. The polypeptide of claim 103, wherein the second polypeptide comprises the amino acid sequence of SEQ ID NO: 253, SEQ ID NO: 254, or SEQ ID NO: 722.

105. A pharmaceutical composition comprising the polypeptide of claim 79 and a pharmaceutically acceptable excipient.

106. A pharmaceutical composition comprising the polypeptide of claim 95 and a pharmaceutically acceptable excipient.

107. A method of reducing IgE immunoglobulins in a subject, the method comprising administering a pharmaceutical composition comprising the polypeptide of claim 79 to the subject to reduce IgE immunoglobulins in the subject.

108. A method of reducing IgE immunoglobulins in a subject, the method comprising administering a pharmaceutical composition comprising the polypeptide of claim 95 to the subject to reduce IgE immunoglobulins in the subject.
